(19) United States
(12) Patent Application Publication
(10) Pub. No.: US 2009/0192154 A1
(43) Pub. Date: Jul. 30, 2009

(54) SULTAM DERIVATIVES

(75) Inventors: Naomi MAEKAWARA, Tokyo (JP); Masakazu Atobe, Tokyo (JP); Masashi Kawanishi, Tokyo (JP); Eiichi Tanaka, Tokyo (JP)

Correspondence Address: BIRCH STEWART KOLASCH & BIRCH, PO BOX 747, FALLS CHURCH, VA, 22040-0747, US

(73) Assignee: Asahi Kasei Pharma Corporation, Tokyo (JP)

(21) Appl. No.: 12/342,274

(22) Filed: Dec. 23, 2008

Related U.S. Application Data
(60) Provisional application No. 61/016,990, filed on Dec. 27, 2007.

(51) Int. Cl. A61K 31/429; C07D 513/00; A61K 31/5377; A61K 31/4439; A61K 31/496; A61P 19/00

(52) U.S. Cl. 514/234.2; 548/207; 544/333; 544/133; 546/271.1; 514/338; 514/373; 514/254.04

(57) ABSTRACT

A sultam derivative of Formula (1) described below having inhibitory action for aggrecanase activity is provided. The sultam derivative of Formula (1) and a salt thereof exhibit strong inhibitory action for aggrecanase activity in a living body of a mammalian animal including human, and is useful, for example, as an active ingredient of a pharmaceutical agent for preventing and/or treating various diseases caused by degradation of aggrecan.

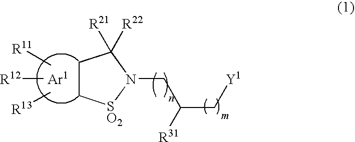

### TECHNICAL FIELD

The present invention is related to novel sultam derivatives that have an inhibitory activity on degradation of an extracellular matrix and are useful as an active ingredient for a pharmaceutical agent.

### BACKGROUND ART

Osteoarthritis is characterized by loss of a joint cartilage and thickening of a bone, and may form bony outgrowths. Joint cartilage functions as a material which absorbs an impact when pressure is applied on a joint or a joint is put into motion. As such, it plays an important role for a function of a joint. Two most important components of cartilage extracellular matrix are Type II collagen and aggrecan, and they provide a joint cartilage with a characteristic of said impact absorption. Enzymatic degradation of aggrecan and Type II collagen is involved with loss of cartilage found in osteoarthritis.

Aggrecan is a major glycoprotein which is expressed in cartilage tissues. Many glycosaminoglycan molecules bonded to its core protein have a negative charge and water attached thereto. Therefore, aggrecan forms a highly hydrated gel, which can fill empty spaces, resulting in compressibility and elasticity of a cartilage.

Aggrecanase is a protease which digests aggrecan molecule. Specifically, it digests a peptide bond between Glu373 and Ala374 that are present in an aggrecan interglobular domain. Two types of aggrecanase, aggrecanase-1 and aggrecanase-2, are found in a joint tissue. These enzymes belong to a family of A Disintegrin and Metalloproteinase with ThromboSpondin motifs (ADAMTS) and also referred to as ADAMTS-4 and ADAMTS-5, respectively.

Loss of aggrecan from a matrix of a joint cartilage contributes to progressive degradation of a joint. With respect to arthritis, aggrecan is one of cartilage matrix components that are decreased as a first measurable loss (Non-patent Document 1).

In addition, aggrecan has a protective effect against degradation of collagen. As for its mechanism, the followings have been suggested; Type II collagen is exposed due to degradation of aggrecan, the exposed collagen becomes an easy target for enzymatic degradation by collagenase, etc. which is a proteolytic enzyme specific to a collagen chain, and absorptivity of a cartilage matrix is reduced by loss of aggrecan, resulting in reduced elasticity of the matrix and an increased mechanical stress on a collagen chain, thus causing break down of the collagen chain. (Non-patent Document 2).

In addition, for a mouse genetically modified to have resistance to cleavage in interglobular domain of aggrecan, a model having osteoarthritis or inflammatory arthritis that is surgically induced showed reduced loss of aggrecan, thus not only destruction of cartilage is inhibited but also regeneration of cartilage is promoted (Non-patent Document 3).

At present, for a therapeutic method for treating osteoarthritis, only a limited symptomatic treatment exists and there is no pharmaceutical agent available for inhibiting the destruction of cartilage in bone joints. Non-steroidal anti-inflammatory drugs (NSAIDs) such as acetaminophen, etc. function as an inhibitor of synthesis of prostaglandin which induces pain or swelling. As such, NSAIDs do not directly prevent the destruction of a cartilage. Also, since the administration of a corticoteroid has a negative impact on metabolism of a cartilage, a risk of having acute joint degradation was reported (Non-patent Document 4). Further, regarding the inhibitory effect by intra-articular administration of hyaluronic acid on destruction of a joint, there are both agreement and opposition (Non-patent Document 5). Meanwhile, an inhibitory agent for aggrecanase has a cartilage preserving effect by preventing destruction of cartilage and/or promoting regeneration of cartilage, and thus is more useful than conventional drugs.

Although there are several compounds reported to have an inhibitory action on aggrecanase (Patent Document 1 and Patent Document 2), they are not satisfactory in terms of efficacy and side effect. In addition, although some compounds having a similar structure have been reported (Patent Document 3), no explanation regarding the inhibitory activity on an aggrecanase is described.

### [Patent Document 1] WO 2005/060456

### [Patent Document 2] WO 03/080042

### [Patent Document 3] WO 91/15486

[Non-patent Document 1] Mankin et al., J. Bone Joint surg., 52A, 424-434 (1970).
[Non-patent Document 2] Michael et al., J. Biol. Chem., 278(46), 45539-45545 (2003).
[Non-patent Document 3] Christopher et al., J. Clin. Invest., 1-10 (2007).

### [Non-patent Document 4] Torisu et al., Joint Surgery, 21, 180-184 (2002).

### [Non-patent Document 5] Lo et al., JAMA., 290(23), 3115-21 (2003).

### DISCLOSURE OF THE INVENTION

### Problems to be Solved by the Invention

The purpose of the present invention is to provide a novel compound which has an inhibitory activity on aggrecanase. Specifically, the present invention provides a novel compound which has an excellent inhibitory activity on aggrecanase and has little side effects.

### Means for Solving Problems

Inventors of the present invention extensively studied to solve the problems described above, and as a result found that the novel compounds that are represented by the following formula (1) have an excellent inhibitory effect on aggrecanase activity. The present invention was completed based on said findings.

Specifically, the present invention includes those described below.

[A1] A compound of formula (1), or a stereoisomer, racemate, pharmaceutically acceptable salt, hydrate, or a prodrug thereof;

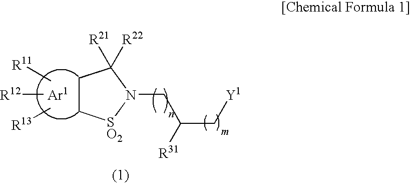

[wherein,

Ar¹ represents a heteroaryl ring,

R¹¹ represents —R¹¹¹—R¹¹²—R¹¹³—R¹¹⁴

(R¹¹¹ represents a single bond, —O—, —NR¹¹¹¹—, —NR¹¹¹¹CO—, —CONR¹¹¹¹—, —NR¹¹¹¹SO₂—, —SO₂NR¹¹¹¹— (R¹¹¹¹ represents a hydrogen atom or an optionally substituted alkyl group), —(C═O)—, —C≡C— or —CH═CH—,

R¹¹² represents an optionally substituted alkylene group or an optionally substituted arylene group, or may be bonded to R¹¹¹¹ to form a ring structure,

R¹¹³ represents a single bond, —O—, —NR¹¹³¹—, —NR¹¹³¹CO—, —CONR¹³¹¹—, —NR¹¹³¹SO₂— or —SO₂NR¹¹³¹— (R¹¹³¹ represents a hydrogen atom or an optionally substituted alkyl group), —(C═O)—, —C≡C— or —CH═CH—, and

R¹¹⁴ represents an optionally substituted aryl group, an optionally substituted alkyl group or a hydrogen atom, or may be bonded to R¹¹³¹ to form a ring structure),

provided that in the case that R¹¹² is a nitrogen atom-containing group, and the bonding position of R¹¹¹ is on the nitrogen atom of R¹¹², R¹¹¹ represents a single bond, —NR¹¹¹¹CO—, —NR¹¹¹¹SO₂—, —(C═O)—, —C≡C— or —CH═CH—,

in the case that R¹¹² is a nitrogen atom-containing group, and the bonding position of R¹¹³ is on the nitrogen atom of R¹¹², R¹¹³ represents a single bond, —CONR¹¹³¹—, —SO₂NR¹¹³¹—, —(C═O)—, —C≡C— or —CH═CH—, and

in the case that R¹¹⁴ is a nitrogen atom-containing group, and the bonding position of R¹¹³ is on the nitrogen atom of R¹¹⁴, R¹¹³ represents a single bond, —NR¹¹³¹CO—, —NR¹¹³¹SO—, —(C═O)—, —C≡C— or —CH═CH—;

provided that in the case that Ar¹ is a nitrogen atom-containing group, and the substituted position of R¹¹ is the nitrogen atom on Ar¹, R¹¹¹ represents a single bond, —CONR¹¹¹¹—, —SO₂NR¹¹¹¹—, —(C═O)—, —C≡C— or —CH═CH—;

R¹² and R¹³ may be the same or different and independently represent,

a hydrogen atom, a halogen atom, a hydroxyl group, an optionally substituted alkyl group, an optionally substituted alkenyl group, an optionally substituted alkynyl group, an optionally substituted alkoxy group or an optionally substituted amino group;

or may not be present;

R²¹ and R²² may be the same or different and independently represent,

a hydrogen atom, an optionally substituted alkyl group, an optionally substituted aryl group, an optionally substituted alkoxy group, an optionally substituted aryloxy group or an optionally substituted amino group;

R³¹ represents

a hydrogen atom, an optionally substituted alkyl group, an optionally substituted aryl group, an optionally substituted alkoxy group or an optionally substituted aryloxy group;

R²¹ and R³¹, or R²² and R³¹ each may form together a ring;

n and m may be the same or different and independently represent 0 or 1;

Y¹ represents —COY¹¹ (Y¹¹ represents —NHOH, —OH, —NH₂, an amino group substituted with one or two optionally substituted alkyl groups, an amino group substituted with one or two optionally substituted aryl groups or an amino group substituted with an optionally substituted aryl group and an optionally substituted alkyl group)].

[A2] The compound, or a stereoisomer, racemate, pharmaceutically acceptable salt, hydrate, or a prodrug thereof according to [A1], wherein Ar¹ represents a thiophene ring.
[A3] The compound, or a stereoisomer, racemate, pharmaceutically acceptable salt, hydrate, or a prodrug thereof according to [A1] or [A2] wherein R¹¹¹ represents a single bond.
[A4] The compound, or a stereoisomer, racemate, pharmaceutically acceptable salt, hydrate, or a prodrug thereof according to [A1] or [A2] wherein R¹¹¹ represents —CH═CH—.
[A5] The compound, or a stereoisomer, racemate, pharmaceutically acceptable salt, hydrate, or a prodrug thereof according to any one of [A1] to [A4] wherein R¹¹² represents an optionally substituted arylene group.
[A6] The compound, or a stereoisomer, racemate, pharmaceutically acceptable salt, hydrate, or a prodrug thereof according to any one of [A1] to [A5] wherein R¹¹² represents an optionally substituted phenylene group.
[A6-2] The compound, or a stereoisomer, racemate, pharmaceutically acceptable salt, hydrate, or a prodrug thereof according to any one of [A1] to [A5] wherein R¹¹² represents a phenylene group optionally substituted with a halogen atom.
[A6-3] The compound, or a stereoisomer, racemate, pharmaceutically acceptable salt, hydrate, or a prodrug thereof according to any one of [A1] to [A5] wherein R¹¹² represents an unsubstituted phenylene group.
[A7] The compound, or a stereoisomer, racemate, pharmaceutically acceptable salt, hydrate, or a prodrug thereof according to any one of [A1] to [A6] wherein R¹¹³ represents any one of a single bond, —CH═CH— or —C≡C—.
[A7-2] The compound, or a stereoisomer, racemate, pharmaceutically acceptable salt, hydrate, or a prodrug thereof according to any one of [A1] to [A6-3] wherein R¹¹³ represents a single bond.

Incidentally, when the item number that is referred to is described as a range such as [A1] to [A6-3] and a sub-numbered item such as [A6-2] is included in the range, the sub-numbered item such as [A6-2] is also referred to. The same is true herein below.

[A7-3] The compound, or a stereoisomer, racemate, pharmaceutically acceptable salt, hydrate, or a prodrug thereof according to any one of [A1] to [A6-3] wherein R¹¹³ represents —CH═CH—.
[A7-4] The compound, or a stereoisomer, racemate, pharmaceutically acceptable salt, hydrate, or a prodrug thereof according to any one of [A1] to [A6-3] wherein R¹¹³ represents —C≡C—.
[A8] The compound, or a stereoisomer, racemate, pharmaceutically acceptable salt, hydrate, or a prodrug thereof according to any one of [A1] to [A7-4] wherein R¹¹⁴ represents an optionally substituted aryl group.
[A8-2] The compound, or a stereoisomer, racemate, pharmaceutically acceptable salt, hydrate, or a prodrug thereof according to any one of [A1] to [A7-4] wherein R¹¹⁴ represents an optionally substituted phenyl group.
[A8-3] The compound, or a stereoisomer, racemate, pharmaceutically acceptable salt, hydrate, or a prodrug thereof according to any one of [A1] to [A7-4] wherein R¹¹⁴ represents a phenyl group optionally substituted with a halogen atom or a lower alkoxy group.
[A8-4] The compound, or a stereoisomer, racemate, pharmaceutically acceptable salt, hydrate, or a prodrug thereof according to any one of [A1] to [A7-4] wherein R¹⁴ represents an unsubstituted phenyl group.
[A8-5] The compound, or a stereoisomer, racemate, pharmaceutically acceptable salt, hydrate, or a prodrug thereof according to any one of [A1] to [A7-4] wherein R¹⁴ represents an optionally substituted alkyl group.
[A9] The compound, or a stereoisomer, racemate, pharmaceutically acceptable salt, hydrate, or a prodrug thereof according to any one of [A1] to [A8-5] wherein R¹², if present, is a hydrogen atom and R¹³, if present, is a hydrogen atom.
[A10] The compound, or a stereoisomer, racemate, pharmaceutically acceptable salt, hydrate, or a prodrug thereof according to any one of [A1] to [A9] wherein both R²¹ and R²² represent a hydrogen atom.
[A11] The compound, or a stereoisomer, racemate, pharmaceutically acceptable salt, hydrate, or a prodrug thereof according to any one of [A1] to [A10] wherein R³¹ represents an optionally substituted alkyl group.
[A12] The compound, or a stereoisomer, racemate, pharmaceutically acceptable salt, hydrate, or a prodrug thereof according to any one of [A1] to [A11] wherein R³¹ represents an isopropyl group.
[A13] The compound, or a stereoisomer, racemate, pharmaceutically acceptable salt, hydrate, or a prodrug thereof according to any one of [A1] to [A12] wherein the asymmetric carbon to which R³¹ is bonded has steric configuration of formula (5) below:

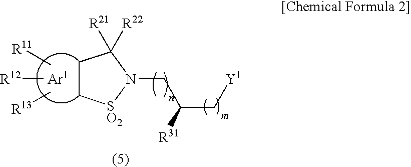

[A14] The compound, or a stereoisomer, racemate, pharmaceutically acceptable salt, hydrate, or a prodrug thereof described in any one of [A1] to [A13], wherein R²¹ and R³¹, and R²² and R³¹ do not form a ring.
[A15] The compound, or a stereoisomer, racemate, pharmaceutically acceptable salt, hydrate, or a prodrug thereof described in any one of [A1] to [A14], wherein both n and m represent 0.
[A16] The compound, or a stereoisomer, racemate, pharmaceutically acceptable salt, hydrate, or a prodrug thereof described in any one of [A1] to [A15], wherein Y¹¹ represents —NHOH.
[A17] The compound, or a stereoisomer, racemate, pharmaceutically acceptable salt, hydrate, or a prodrug thereof described in any one of [A1] to [A15], wherein Y¹¹ represents —OH.
[A18] A pharmaceutical agent which comprises the compound, or a stereoisomer, racemate, pharmaceutically acceptable salt, hydrate, or a prodrug thereof described in any one of [A1] to [A17] as an active ingredient.
[A19] The pharmaceutical agent described in [A18], which is used for prevention and/or treatment of a disease characterized by destruction of a joint cartilage.
[A20] The pharmaceutical agent described in [A18], which is used for prevention and/or treatment of osteoarthritis.
[A21] An inhibitor of aggrecanase activity which comprises the compound, or a stereoisomer, racemate, pharmaceutically acceptable salt, hydrate, or a prodrug thereof described in any one of [A1] to [A17] as an active ingredient.
[A22] A method for prevention and/or treatment of a disease characterized by destruction of a joint cartilage of a mammal, comprising administering an effective amount of the compound, or a stereoisomer, racemate, pharmaceutically acceptable salt, hydrate, or a prodrug thereof described in any one of [A1] to [A17] to the mammal.
[A23] Use of the compound, or a stereoisomer, racemate, pharmaceutically acceptable salt, hydrate, or a prodrug thereof described in any one of [A1] to [A17] for preparation of a pharmaceutical agent which is used for prevention and/or treatment of a disease characterized by destruction of a joint cartilage.
[B1] A compound of formula (1), or a stereoisomer, racemate, pharmaceutically acceptable salt, hydrate, or a prodrug thereof represented by the following formula (B);

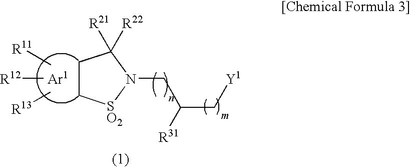

[wherein,

Ar¹ represents a heteroaryl ring,

R¹¹ represents —R¹¹¹—R¹¹²—R¹¹³—R¹¹⁴

(R¹¹¹ represents a single bond, —O—, —NR¹¹¹¹—, —NR¹¹¹¹CO—, —CONR¹¹¹¹— (R¹¹¹¹ represents a hydrogen atom or an optionally substituted alkyl group), —(C═O)—, —C≡C— or —C═C—,

R¹¹² represents an optionally substituted alkylene group or an optionally substituted arylene group,

R¹¹³ represents a single bond, —NR¹¹³¹—, —NR¹¹³¹CO—, —CONR¹¹³¹— (R¹¹³¹ represents a hydrogen atom or an optionally substituted alkyl group), —(C═O)—, —C≡C— or —C═C—,

R¹¹⁴ represents an optionally substituted aryl group, an optionally substituted alkyl group, or a hydrogen atom);

provided that, if the substituted position of R¹¹ corresponds to the nitrogen atom of Ar¹, R¹¹¹ represents a single bond, —CONR¹¹¹¹—, —(C═O)—, —C≡C— or —C═C—;

R¹² and R¹³ may be the same or different and independently represent,

a hydrogen atom, a halogen atom, a hydroxyl group, an optionally substituted alkyl group, an optionally substituted alkenyl group, an optionally substituted alkynyl group, an optionally substituted alkoxy group, or an optionally substituted amino group;

or may be absent;

R²¹ and R²² may be the same or different and independently represent,

a hydrogen atom, an optionally substituted alkyl group, an optionally substituted aryl group, an optionally substituted alkoxy group, an optionally substituted aryloxy group or an optionally substituted amino group;

R³¹ represents

a hydrogen atom, an optionally substituted alkyl group, an optionally substituted aryl group, an optionally substituted alkoxy group, or an optionally substituted aryloxy group;

R²¹ and R³¹, or R²² and R³¹ each may form a ring together;

n and m may be the same or different and independently represent 0 or 1;

Y¹ represents —COY¹¹ (Y¹¹ represents —NHOH, —OH, —NH₂, an amino group substituted with one or two optionally substituted alkyl groups, an amino group substituted with one or two optionally substituted aryl groups or an amino group substituted with an optionally substituted aryl group and an optionally substituted alkyl group)].

[B2] The compound, or a stereoisomer, racemate, pharmaceutically acceptable salt, hydrate, or a prodrug thereof described in [B1], wherein Ar¹ represents a thiophene ring.
[B3] The compound, or a stereoisomer, racemate, pharmaceutically acceptable salt, hydrate, or a prodrug thereof described in [B1] or [B2], wherein R¹¹¹ represents a single bond.
[B4] The compound, or a stereoisomer, racemate, pharmaceutically acceptable salt, hydrate, or a prodrug thereof described in any one of [B1] to [B3], wherein R¹¹² represents an optionally substituted phenylene group.
[B5] The compound, or a stereoisomer, racemate, pharmaceutically acceptable salt, hydrate, or a prodrug thereof described in any one of [B1] to [B4], wherein R¹², if present, is a hydrogen atom and R¹³, if present, is a hydrogen atom.
[B6] The compound, or a stereoisomer, racemate, pharmaceutically acceptable salt, hydrate, or a prodrug thereof described in any one of [B1] to [B5], wherein both R²¹ and R²² represent a hydrogen atom.
[B7] The compound, or a stereoisomer, racemate, pharmaceutically acceptable salt, hydrate, or a prodrug thereof described in any one of [B1] to [B6], wherein R³¹ represents an optionally substituted alkyl group.
[B8] The compound, or a stereoisomer, racemate, pharmaceutically acceptable salt, hydrate, or a prodrug thereof described in any one of [B1] to [B6], wherein R³¹ is an isopropyl group.
[B9] The compound, or a stereoisomer, racemate, pharmaceutically acceptable salt, hydrate, or a prodrug thereof described in any one of [B1] to [B8], wherein the asymmetric carbon to which R³¹ is bonded has steric configuration of formula (5) below:

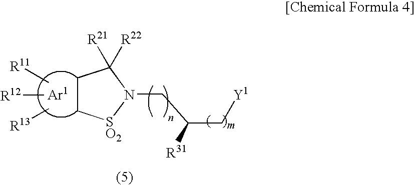

[B10] The compound, or a stereoisomer, racemate, pharmaceutically acceptable salt, hydrate, or a prodrug thereof described in any one of [B1] to [B9], wherein R²¹ and R³¹, and R²² and R³¹ do not form a ring.
[B11] The compound, or a stereoisomer, racemate, pharmaceutically acceptable salt, hydrate, or a prodrug thereof described in any one of [B1] to [B10], wherein both n and m represent 0.
[B12] The compound, or a stereoisomer, racemate, pharmaceutically acceptable salt, hydrate, or a prodrug thereof described in any one of [B1] to [B11], wherein Y¹¹ represents —NHOH.
[B13] The compound, or a stereoisomer, racemate, pharmaceutically acceptable salt, hydrate, or a prodrug thereof described in any one of [B1] to [B11], wherein Y¹¹ represents —OH.
[B13-2] The compound, or a stereoisomer, racemate, pharmaceutically acceptable salt, hydrate, or a prodrug thereof described in [B1]; wherein

Ar¹ represents a thiophene ring;

R¹¹ represents any one of the substituents ER11-1 to ER11-5 described below;

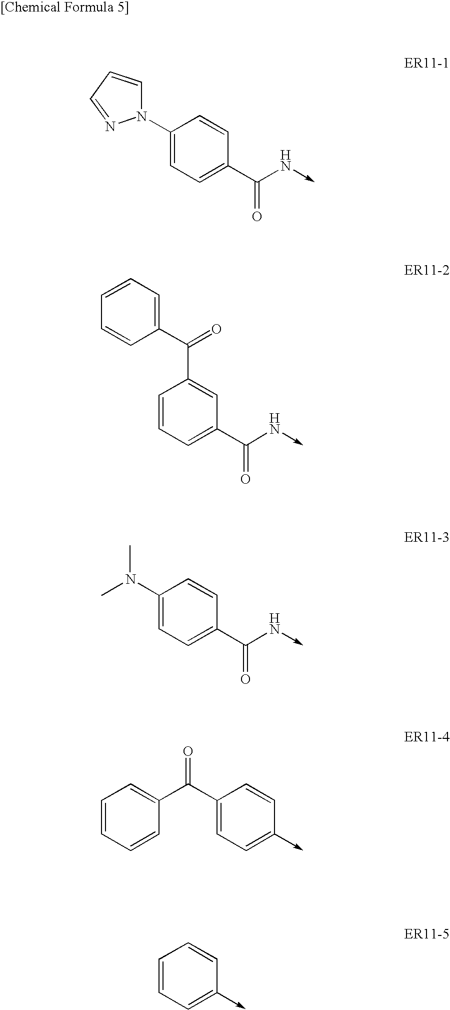

R¹² represents a hydrogen atom;

R²¹ and R²² both represent a hydrogen atom;

R³¹ represents an optionally substituted alkyl group;

the asymmetric carbon to which R³¹ is bonded has steric configuration of formula (5) below;

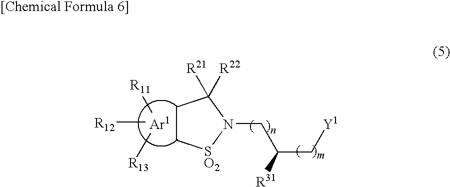

R²¹ and R³¹, and R²² and R³¹ do not form a ring;

n and m both represent 0; and,

Y¹¹ represents —NHOH.

[B13-3] The compound, or a stereoisomer, racemate, pharmaceutically acceptable salt, hydrate, or a prodrug thereof described in [B1]; wherein

Ar¹ represents a thiophene ring;

R¹¹ represents anyone of the substituents ER11-1 to ER11-5 described below;

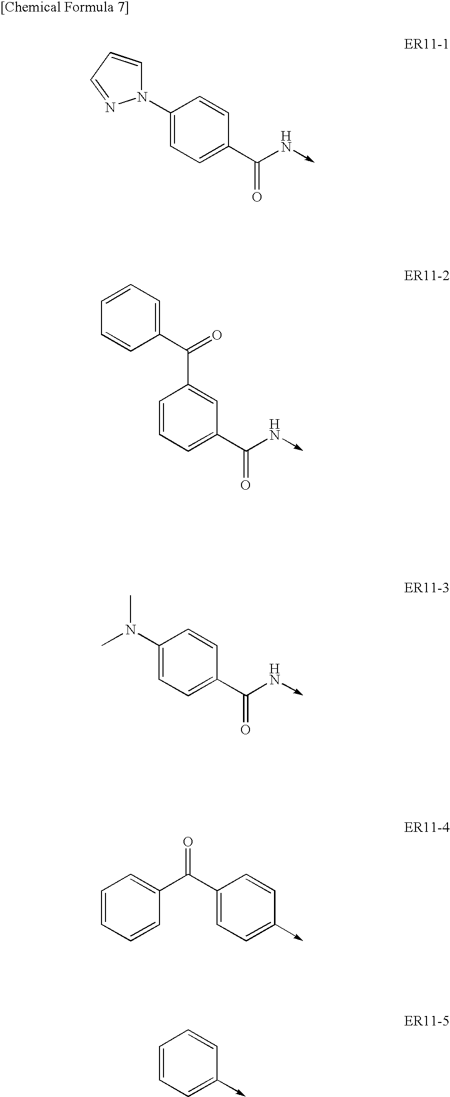

R¹² represents a hydrogen atom;

R²¹ and R²² both represent a hydrogen atom;

R³¹ represents an optionally substituted alkyl group;

the asymmetric carbon to which R³¹ is bonded has steric configuration of formula (5) below;

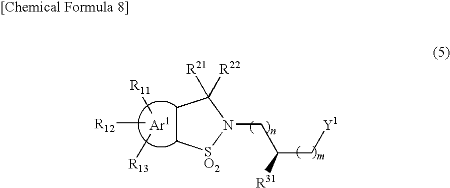

R²¹ and R³¹, and R²² and R³¹ do not form a ring;

n and m both represent 0; and,

Y¹¹ represents —OH.

[B14] A pharmaceutical agent which comprises the compound, or a stereoisomer, racemate, pharmaceutically acceptable salt, hydrate, or a prodrug thereof described in any one of [B1] to [B13-3] as an active ingredient.
[B15] The pharmaceutical agent described in [B14], which is used for prevention and/or treatment of a disease characterized by destruction of a joint cartilage.
[B16] The pharmaceutical agent described in [B14], which is used for prevention and/or treatment of osteoarthritis.
[B17] An inhibitor of aggrecanase activity which comprises the compound, or a stereoisomer, racemate, pharmaceutically acceptable salt, hydrate, or a prodrug thereof described in any one of [B1] to [B13-3] as an active ingredient.
[B18] A method for prevention and/or treatment of a disease characterized by destruction of a joint cartilage of a mammal, comprising administering an effective amount of the compound, or a stereoisomer, racemate, pharmaceutically acceptable salt, hydrate, or a prodrug thereof described in any one of [B1] to [B13-3] to the mammal.
[B18-2] A method for prevention and/or treatment of osteoarthritis of a mammal, comprising administering an effective amount of the compound, or a stereoisomer, racemate, pharmaceutically acceptable salt, hydrate, or a prodrug thereof described in any one of [B1] to [B13-3] to the mammal.
[B19] Use of the compound, or a stereoisomer, racemate, pharmaceutically acceptable salt, hydrate, or a prodrug thereof described in any one of [B1] to [B13-3] for preparation of a pharmaceutical agent which is used for prevention and/or treatment of a disease characterized by destruction of a joint cartilage.
[B19-2] Use of the compound, or a stereoisomer, racemate, pharmaceutically acceptable salt, hydrate, or a prodrug thereof described in any one of [B1] to [B13-3] for preparation of a pharmaceutical agent which is used for prevention and/or treatment of osteoarthritis.

### EFFECT OF THE INVENTION

The compounds of the present invention or salts thereof exhibit a potent inhibitory effect on aggrecanase activity in a body of a mammal including human. Thus, they are useful as, for example, an active ingredient of a pharmaceutical agent for prevention and/or treatment of various diseases that are caused by breakdown of aggrecan. Further, having very low toxicity, the compounds of the present invention or salts thereof can be safely used as an active ingredient of a pharmaceutical agent.

### BEST MODE FOR CARRYING OUT THE INVENTION

Hereinbelow, the present invention is explained in greater detail.

Unless specifically described otherwise in the specification, examples of a halogen atom include a fluorine atom, a chlorine atom, a bromine atom, or an iodine atom.

In the present specification, the term “lower” substituent indicates a substituent, the number of the carbon atoms of which is at most ten. Examples thereof include a substituent having 1 to 6 carbon atoms, and preferred examples thereof include a substituent having 1 to 3 carbon atoms.

An alkyl group indicates a linear, branched, or cyclic saturated hydrocarbon group, or a saturated hydrocarbon group having a combination thereof. A lower alkyl group is preferred. The lower alkyl group indicates an alkyl group having 1 to 10 carbon atoms. Preferably, it is an alkyl group having 1 to 6 carbon atoms. More preferably, it is an alkyl group having 1 to 3 carbon atoms. The same is true for an alkyl group which is comprised in other substituents as a substituent (e.g., an alkoxy group, etc.).

Preferred examples of the alkyl group having 1 to 3 carbon atoms include a methyl group, an ethyl group, a n-propyl group, an isopropyl group and a cyclopropyl group. In addition, preferred examples of an alkyl group having 4 to 6 carbon atoms include a n-butyl group, an isobutyl group, a s-butyl group, a t-butyl group, a cyclobutyl group, a cyclopropylmethyl group, a n-pentyl group, a cyclopentyl group, a cyclopropylethyl group, a cyclobutylmethyl group, a n-hexyl group, a cyclohexyl group, a cyclopropylpropyl group, a cyclobutylethyl group and a cyclopentylmethyl group. As the alkyl group, a methyl group, an ethyl group, a n-propyl group, or an isopropyl group is more preferred. A still more preferred alkyl group is a methyl group. In addition, there is other embodiment in which an ethyl group is exemplified as a still more preferred alkyl group.

Examples of an alkenyl group include a lower alkenyl group which has one or at least two double bonds. A lower alkenyl group comprising one or two double bonds is preferred. The term lower alkenyl group indicates an alkenyl group comprising 2 to 10 carbon atoms. An alkenyl group having 2 to 5 carbon atoms is preferred. An alkenyl group having 2 to 4 carbon atoms is more preferred. Preferred examples of the alkenyl group having 2 to 4 carbon atoms include a vinyl group, an allyl group, a propenyl group, a butylidene group, a but-1-enyl group, a but-2-enyl group and a but-3-enyl group. In addition, preferred examples of an alkenyl group having 5 carbon atoms include a pentylidene group, a pent-1-enyl group, a pent-2-enyl group, a pent-3-enyl group, and a pent-4-enyl. More preferred examples of the alkenyl group include a vinyl group, an allyl group and a propenyl group, and even more preferred examples of the alkenyl group include a vinyl group and an allyl group. An allyl group is still even more preferred.

Examples of an alkynyl group include a lower alkynyl group which has one or at least two triple bonds. A lower alkynyl group comprising one triple bond is preferred. The alkynyl group comprising 2 to 5 carbon atoms is preferred as the lower alkynyl group. Specifically, preferred examples thereof include an ethynyl group, a prop-1-ynyl group, a prop-2-ynyl group, a but-1-ynyl group, a but-2-ynyl group, a but-3-ynyl group, a pent-1-ynyl group, a pent-2-ynyl group, a pent-3-ynyl group and a pent-4-ynyl group. An ethynyl group, a prop-2-ynyl group, or a but-3-ynyl group is more preferred. An ethynyl group, or a prop-2-ynyl group is still more preferred. A prop-2-ynyl group is even still more preferred.

As an alkylene group, a divalent residue that is produced by removing any single hydrogen atom from the above-described alkyl group can be exemplified. A linear, branched, or cyclic saturated divalent hydrocarbon group, or a saturated divalent hydrocarbon group having a combination thereof can be mentioned. A lower alkylene group is preferred. The term lower alkylene group indicates an alkylene group comprising 1 to 10 carbon atoms. An alkylene group having 1 to 6 carbon atoms is preferred. An alkylene group having 1 to 3 carbon atoms is more preferred. An alkylene group having one carbon atom is even more preferred. Preferred examples of the alkylene group having 1 carbon atom include a methylene group, examples of an alkylene group having 2 to 3 carbon atoms include an ethylene group, a n-propylene group, an isopropylene group and a cyclopropylene group. Further, with respect to preferred examples of an alkylene group having 4 to 6 carbon atoms, a divalent residue that is produced by removing any single hydrogen atom from the groups that are described in the above as the preferred examples of the alkyl group having 4 to 6 carbon atoms can be exemplified. More preferred examples of the alkylene group include a methylene group, an ethylene group, a n-propylene group and an isopropylene group. Still more preferred examples of the alkylene group include a methylene group. In addition, there is other embodiment in which an ethylene group is exemplified as a still more preferred alkylene group.

As an alkenylene group, a divalent residue that is produced by removing any single hydrogen atom from the above-described alkenyl group can be exemplified. A lower alkenylene group and the like which comprises one or at least two double bonds can be mentioned. A lower alkenylene group comprising one double bond is preferred. The term lower alkenylene group indicates an alkenylene group comprising 2 to 10 carbon atoms. An alkenylene group having 2 to 5 carbon atoms is preferred. An alkenylene group having 2 to 4 carbon atoms is more preferred. Preferred examples of the alkenylene group having 2 to 4 carbon atoms include a vinylene group, a propenylene group, a but-1-enylene group, a but-2-enylene group and a but-3-enylene group, and examples of an alkenylene group having 5 carbon atoms include a divalent residue that is produced by removing any single hydrogen atom from the group that is described in the above as the preferred examples of the alkenyl group having 5 carbon atoms can be exemplified. More preferred examples of the alkenylene group include a vinylene group and a propenylene group. Still more preferred examples of the alkenylene group include a vinylene group.

As an alkoxy group, a linear, branched, or cyclic saturated alkyl ether group, or a saturated alkyl ether group having a combination thereof can be mentioned. A lower alkoxy group is preferred. As the lower alkoxy group, an alkoxy group comprising 1 to 6 carbon atoms can be mentioned. An alkoxy group comprising 1 to 4 carbon atoms is preferred. Preferred examples of the alkoxy group comprising 1 to 4 carbon atoms include a methoxy group, an ethoxy group, a n-propoxy group, an isopropoxy group, a cyclopropoxy group, a n-butoxy group, an isobutoxy group, a s-butoxy group, a t-butoxy group, a cyclobutoxy group and a cyclopropylmethoxy group. In addition, preferred examples of an alkoxy group comprising 5 or 6 carbon atoms include a n-pentyloxy group, a cyclopentyloxy group, a cyclopropylethyloxy group, a cyclobutylmethyloxy group, a n-hexyloxy group, a cyclohexyloxy group, a cyclopropylpropyloxy group, a cyclobutylethyloxy group and a cyclopentylmethyloxy group.

As an alkylthio group, a linear, branched, or cyclic saturated alkylthio ether group, or a saturated alkylthio ether group having a combination thereof can be mentioned. A lower alkylthio group is preferred. As the lower alkylthio group, an alkylthio group comprising 1 to 4 carbon atoms is preferred. Specifically, preferred examples thereof include a methylthio group, an ethylthio group, a n-propylthio group, an isopropylthio group, a cyclopropylthio group, a n-butylthio group, an isobutylthio group, a s-butylthio group, a t-butylthio group, a cyclobutylthio group and a cyclopropylmethylthio group.

As an alkoxycarbonyl group, a group in which a carbonyl group is added to the terminal of the alkoxy group can be mentioned. A lower alkoxycarbonyl group is preferred. As the lower alkoxycarbonyl group, a group in which a carbonyl group is added to the terminal of an alkoxy group comprising 1 to 6 carbon atoms can be mentioned. A group in which a carbonyl group is added to the terminal of an alkoxy group comprising 1 to 4 carbon atoms is preferred. Specifically, preferred examples thereof include a methoxycarbonyl group, an ethoxycarbonyl group, a n-propoxycarbonyl group, an isopropoxycarbonyl group, a cyclopropoxycarbonyl group, a n-butoxycarbonyl group, an isobutoxycarbonyl group, a s-butoxycarbonyl group, a t-butoxycarbonyl group, a cyclobutoxycarbonyl group and a cyclopropylmethoxycarbonyl group.

As an alkenyloxy group, an alkenyl ether group in which an oxygen atom is added to the terminal of the alkenyl group described in the above can be mentioned. A lower alkenyloxy group comprising one or at least two double bonds is preferred. A lower alkenyloxy group comprising one double bond is more preferred. As the lower alkenyloxy group, an alkenyloxy group comprising 2 to 4 carbon atoms is preferred. Specifically, a vinyloxy group, an allyloxy group, or a propenyloxy group is preferred. An allyloxy group is more preferred.

As an alkenylthio group, an alkenylthio ether group in which a sulfur atom is added to the terminal of the alkenyl group described in the above can be mentioned. A lower alkenylthio group comprising one or at least two double bonds is preferred. A lower alkenylthio group comprising one double bond is more preferred. As the lower alkenylthio group, an alkenylthio group comprising 2 to 4 carbon atoms is preferred. Specifically, a vinylthio group, an allylthio group, or a propenylthio group is preferred. An allylthio group is more preferred.

As an acyl group, an alkanoyl group or an arylcarbonyl group can be mentioned as a preferred example. An alkanoyl group is preferred. A lower alkanoyl group is more preferred. As the alkanoyl group, a linear, branched, or cyclic saturated alkylcarbonyl group, or a saturated alkylcarbonyl group having a combination thereof can be mentioned. In this case, the alkyl moiety thereof may comprise one or at least two unsaturated bonds. As the lower alkanoyl group, an acyl group comprising 2 to 5 carbon atoms is preferred. Specifically, preferred examples thereof include an acetyl group, a propanoyl group, a butanoyl group, a 2-methylpropanoyl group, a cyclopropylcarbonyl group, a pentanoyl group, a 3-methylbutanoyl group, a 2,2-dimethylpropanoyl group and a cyclobutylcarbonyl group.

As an acyloxy group, an alkanoyloxy group (alkylcarbonyloxy group) or an arylcarbonyloxy group can be mentioned as a preferred example. An alkanoyloxy group is preferred and a lower alkanoyloxy group is more preferred. The alkyl moiety of the alkanoyloxy group may comprise one or at least two unsaturated bonds. As the lower alkanoyloxy group, an acyloxy group comprising 2 to 5 carbon atoms is preferred. Specifically, preferred examples thereof include an acetoxy group, a propanoyloxy group, a butanoyloxy group, a 2-methylpropanoyloxy group, a cyclopropylcarbonyloxy group, a pentanoyloxy group, a 3-methylbutanoyloxy group, a 2,2-dimethylpropanoyloxy group and a cyclobutylcarbonyloxy group.

As an alkylsulfinyl group, a lower alkylsulfinyl group can be mentioned as a preferred example. As the lower alkylsulfinyl group, an alkylsulfinyl group comprising 1 to 4 carbon atoms is preferred. Specifically, preferred examples thereof include a methylsulfinyl group, an ethylsulfinyl group, a n-propylsulfinyl group, an isopropylsulfinyl group, a cyclopropylsulfinyl group, a n-butylsulfinyl group, an isobutylsulfinyl group, a s-butylsulfinyl group, a t-butylsulfinyl group, a cyclobutylsulfinyl group and a cyclopropylmethylsulfinyl group.

As an alkylsulfonyl group, a lower alkylsulfonyl group can be mentioned as a preferred example. As the lower alkylsulfonyl group, an alkylsulfonyl group comprising 1 to 4 carbon atoms is preferred. Specifically, preferred examples thereof include a methylsulfonyl group, an ethylsulfonyl group, a n-propylsulfonyl group, an isopropylsulfonyl group, a cyclopropylsulfonyl group, a n-butylsulfonyl group, an isobutylsulfonyl group, a s-butylsulfonyl group, a t-butylsulfonyl group, a cyclobutylsulfonyl group and a cyclopropylmethylsulfonyl group.

As an alkylcarbamoyl group, a lower alkylcarbamoyl group can be mentioned as a preferred example. As the lower alkylcarbamoyl group, an alkylcarbamoyl group comprising 1 to 4 carbon atoms is preferred. Specifically, preferred examples thereof include a methylcarbamoyl group, an ethylcarbamoyl group, a n-propylcarbamoyl group, an isopropylcarbamoyl group, a cyclopropylcarbamoyl group, a n-butylcarbamoyl group, an isobutylcarbamoyl group, a s-butylcarbamoyl group, a t-butylcarbamoyl group, a cyclobutylcarbamoyl group and a cyclopropylmethylcarbamoyl group.

As an amino group, an —NH₂ group can be mentioned.

As an aryl ring, a monocyclic aromatic ring or a fused polycyclic aromatic ring and the like can be mentioned. The aryl ring can be a hydrocarbon ring or it may comprise at least one, for example 1 to 3, of one or two kinds of heteroatoms that are selected from the group consisting of a nitrogen atom, a sulfur atom and an oxygen atom as a ring-forming atom other than a carbon atom.

Examples of the monocyclic aromatic ring include a monocyclic aromatic hydrocarbon or a monocyclic aromatic heterocycle which comprises one or at least two heteroatoms. As the monocyclic aromatic hydrocarbon, a benzene ring can be mentioned. As the monocyclic aromatic heterocycle, a 5- or 6-membered aromatic heterocycle comprising one or at least two heteroatoms can be mentioned. Specifically, preferred examples of the 5- or 6-membered aromatic heterocycle include thiophene, pyridine, furane, thiazole, oxazole, pyrazole, pyrazine, pyrimidine, pyrrole, imidazole, pyridazine, isothiazole, isoxazole, 1,2,4-oxadiazole, 1,3,4-oxadiazole, 1,2,4-thiadiazole, 1,3,4-thiadiazole and furazane.

Examples of the fused polycyclic aromatic ring include a fused polycyclic aromatic hydrocarbon or a fused polycyclic aromatic heterocycle which comprises one or at least two heteroatoms. As the fused polycyclic aromatic hydrocarbon, a fused polycyclic aromatic hydrocarbon comprising 9 to 14 carbon atoms, i.e., two- or three-ring aromatic hydrocarbon can be mentioned. Specifically, preferred examples thereof include naphthalene, indene, fluorene and anthracene. As the fused polycyclic aromatic heterocycle, a 9 to 14 membered, preferably 9- or 10-membered, fused polycyclic aromatic heterocycle comprising at least one hetero atom, for example one to four hetero atoms, can be mentioned. Specifically, preferred examples thereof include benzofuran, benzothiophene, benzimidazole, benzoxazole, benzothiazole, benzisothiazole, naphtho[2,3-b]thiophene, quinoline, isoquinoline, indole, indazole, quinoxaline, phenanthridine, phenothiazine, phenoxazine, phthalazine, naphthyridine, quinazoline, cinnoline, carbazole, β-carboline, acridine, phenazine, phthalimide and thioxanthen.

As an aryl group, a monocyclic aromatic group or a fused polycyclic aromatic group and the like can be mentioned, for example. In addition, a monovalent residue that is produced by removing any single hydrogen atom from the above-described aryl ring can be exemplified.

As the monocyclic aromatic group, a monovalent residue that is produced by removing any single hydrogen atom from a monocyclic aromatic group can be exemplified. More preferred examples of the monocyclic aromatic group include a phenyl group, a thienyl group (a 2- or 3-thienyl group), a pyridyl group (a 2-, 3- or 4-pyridyl group), a furyl group (a 2- or 3-furyl group), a thiazolyl group (a 2-, 4- or 5-thiazolyl group), an oxazolyl group (a 2-, 4- or 5-oxazolyl group), a pyrazolyl group (a 1-, 3- or 4-pyrazolyl group), a 2-pyrazinyl group, a pyrimidinyl group (a 2-, 4- or 5-pyrimidinyl group), a pyrrolyl group (a 1-, 2- or 3-pyrrolyl group), an imidazolyl group (a 1-, 2- or 4-imidazolyl group), a pyridazinyl group (a 3- or 4-pyridazinyl group), a 3-isothiazolyl group, a 3-isoxazolyl group, a 1,2,4-oxadiazol-5-yl group and a 1,2,4-oxadiazol-3-yl group.

As the fused polycyclic aromatic group, a monovalent residue that is produced by removing any single hydrogen atom from a fused polycyclic aromatic ring comprising 2 to 4, preferably 2 or 3, rings can be exemplified.

Specifically, preferred examples of the fused polycyclic aromatic group include a 1-naphthyl group, a 2-naphthyl group, a 2-indenyl group, a 2-anthryl group, a quinolyl group (a 2-, 3-, 4-, 5-, 6-, 7- or 8-quinolyl group), an isoquinolyl group (a 1-, 3-, 4-, 5-, 6-, 7- or 8-isoquinolyl group), an indolyl group (a 1-, 2-, 3-, 4-, 5-, 6- or 7-indolyl group), an isoindolyl group (a 1-, 2-, 4- or 5-isoindolyl group), an indazolyl group (a 1-, 2-, 3-, 4-, 5-, 6-, or 7-indazolyl group), a phthalazinyl group (a 1-, 5- or 6-phthalazinyl group), a quinoxalinyl group (a 2-, 3- or 5-quinoxalinyl group), a benzofuranyl group (a 2-, 3-, 4-, 5- or 6-benzofuranyl group), a benzothiazolyl group (a 2-, 4-, 5- or 6-benzothiazolyl group), a benzimidazolyl group (a 1-, 2-, 4-, 5- or 6-benzimidazolyl group), a fluorenyl group (a 1-, 2-, 3- or 4-fluorenyl group) and a thioxanthenyl group.

As an aryloxy group, an aryl ether group can be mentioned. As the aryl ring of the aryloxy group, the above-described aryl ring can be exemplified. Preferred examples of the aryl ring of the aryloxy group include the aforementioned preferred examples of the aryl ring.

As an arylene group, a divalent residue that is produced by removing any two hydrogen atoms from the aforementioned aryl ring can be exemplified. Specific examples thereof include a divalent group that is represented by the following formulae. In addition, in the following formulae, V¹ and V² represent a bonding arm that is bonded to the arylene group.

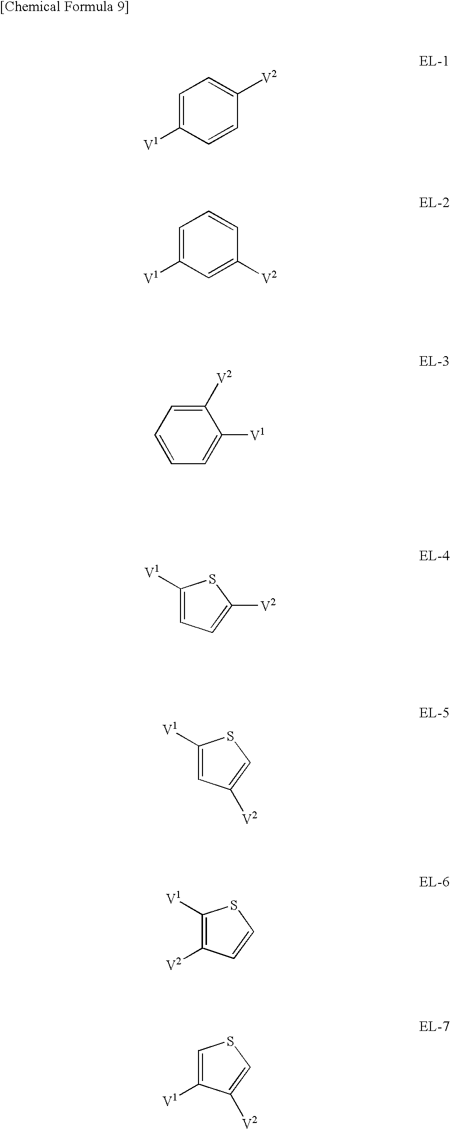

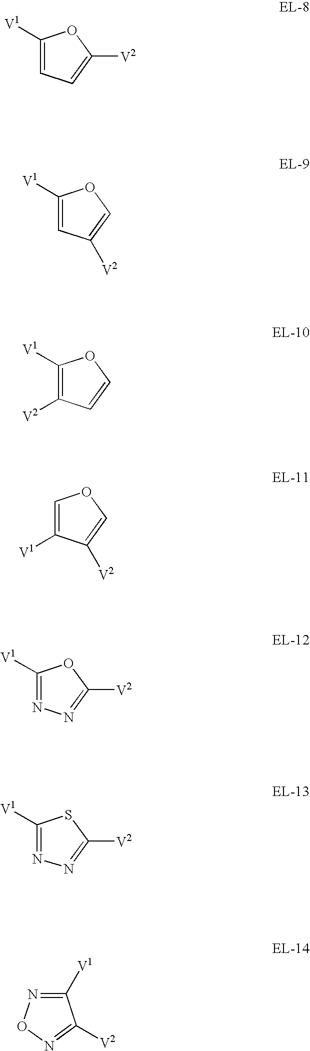

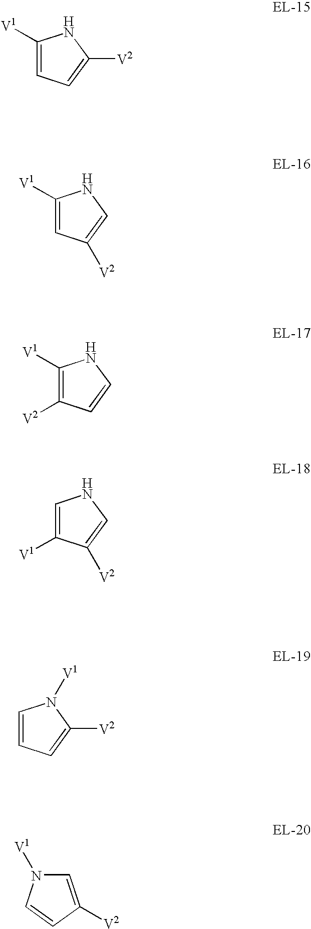

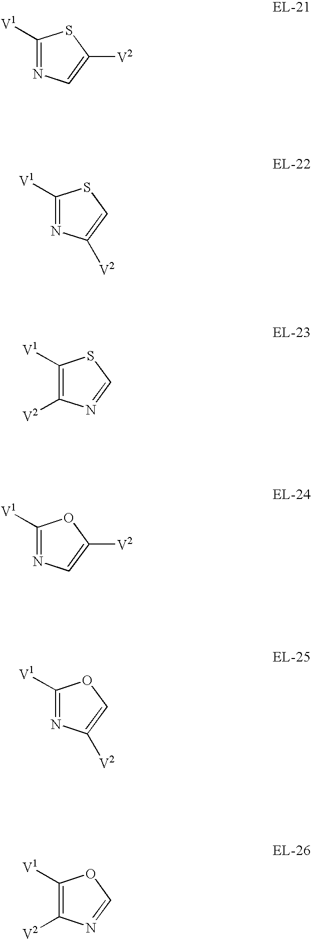

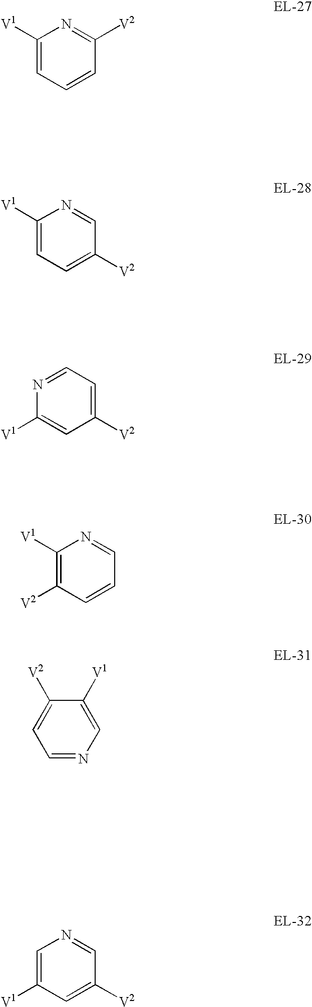

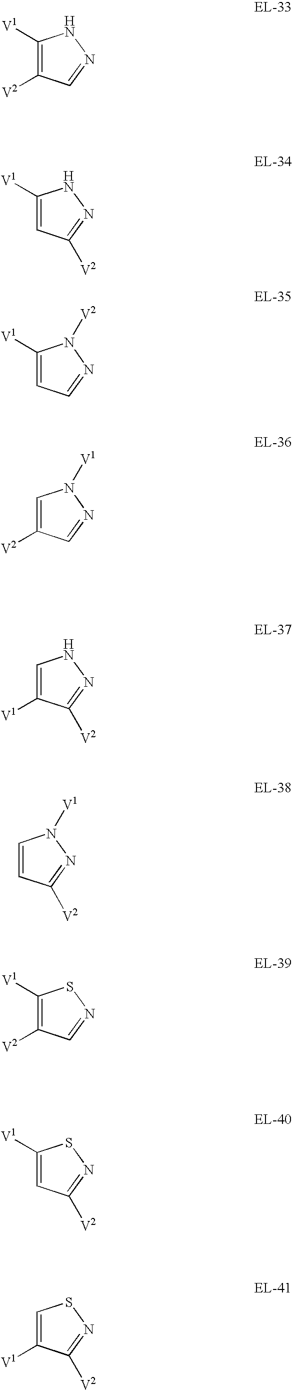

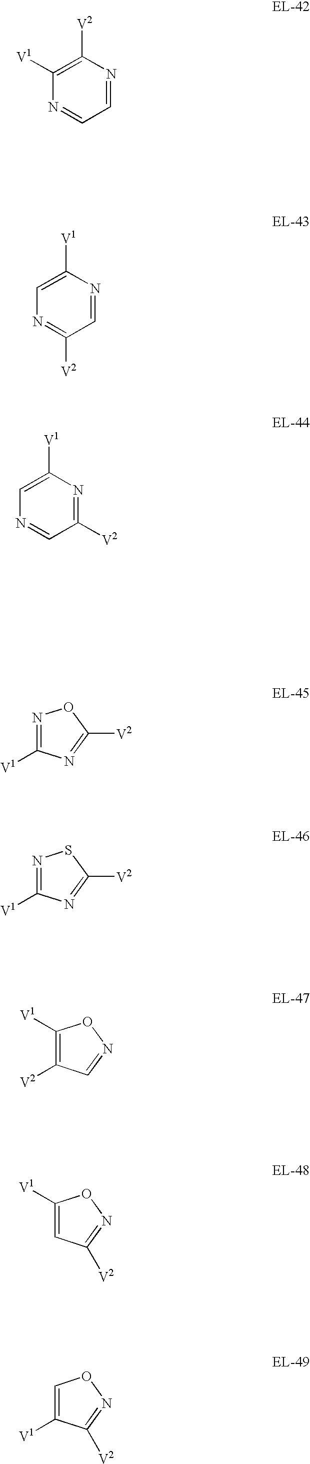

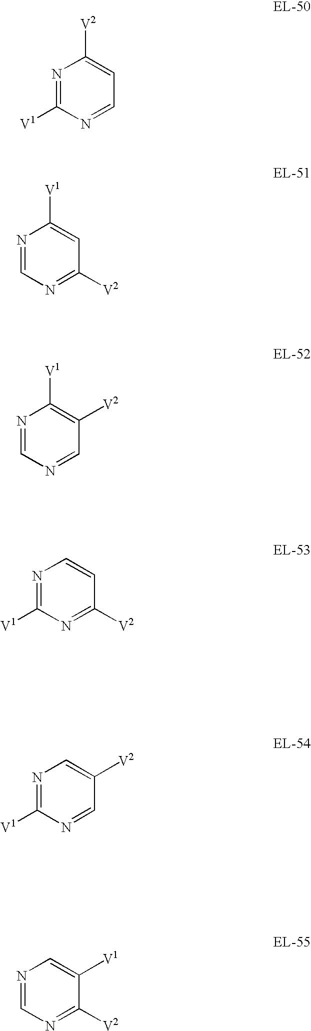

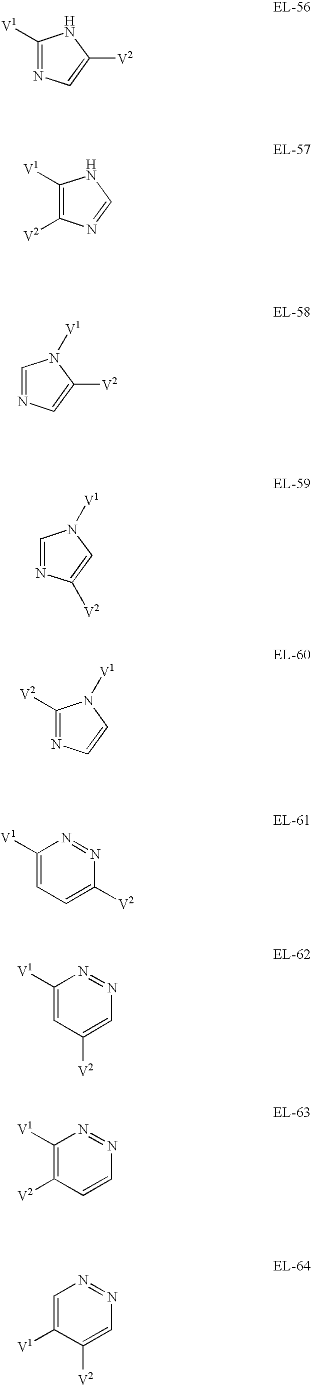

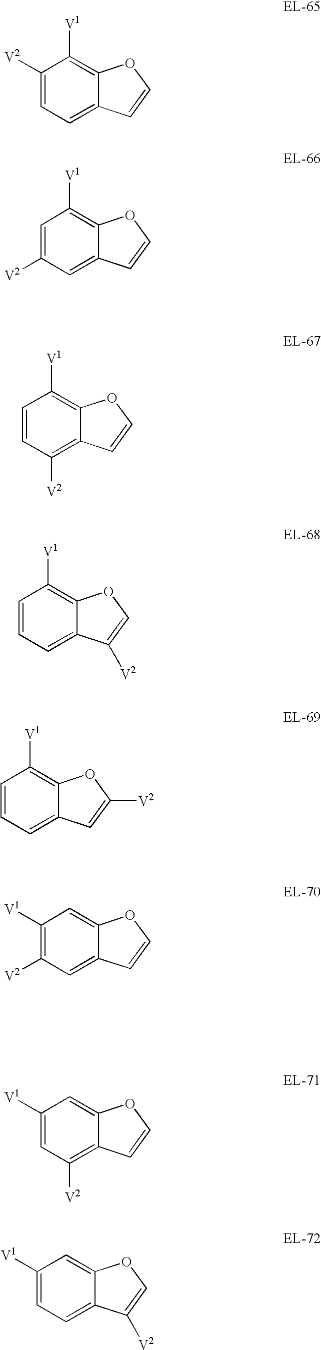

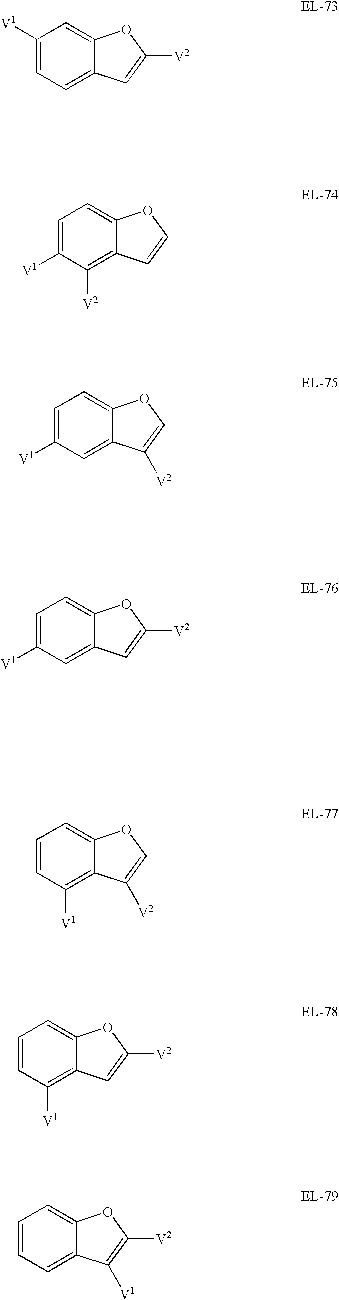

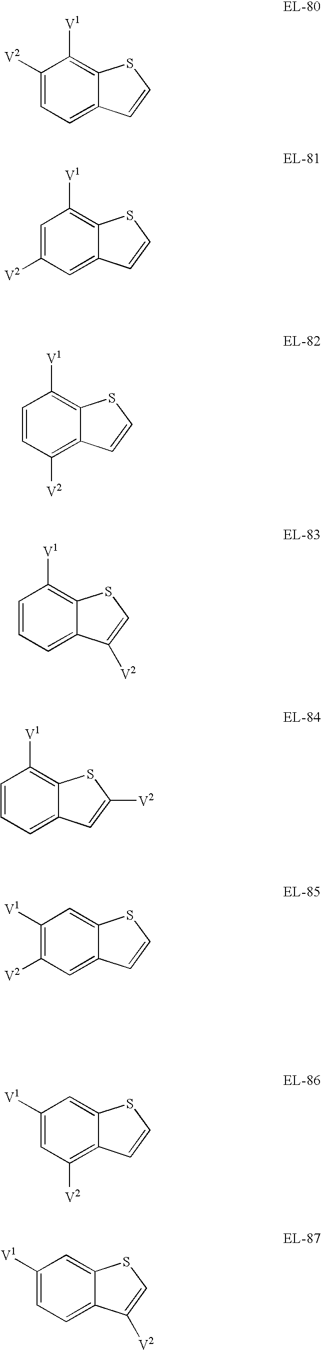

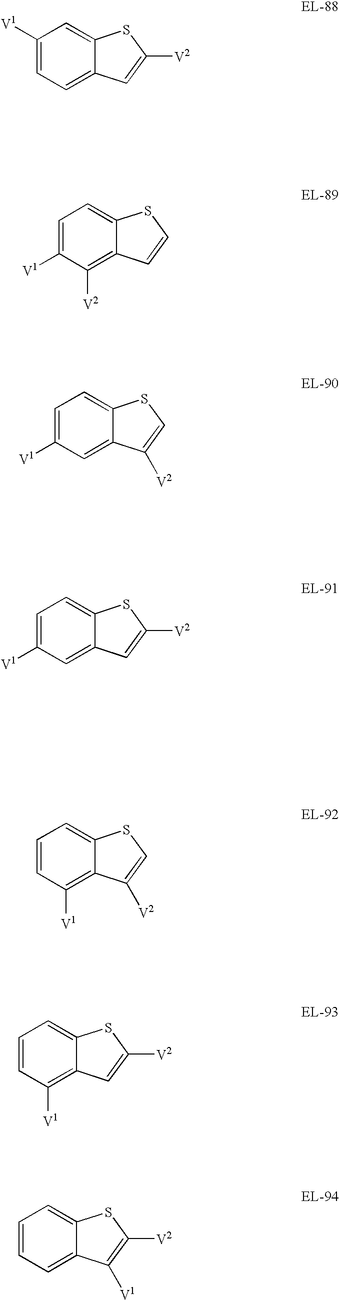

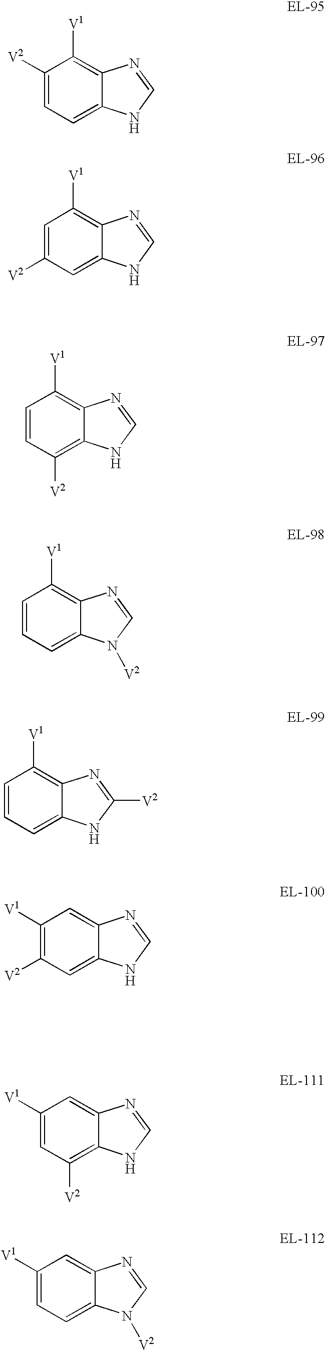

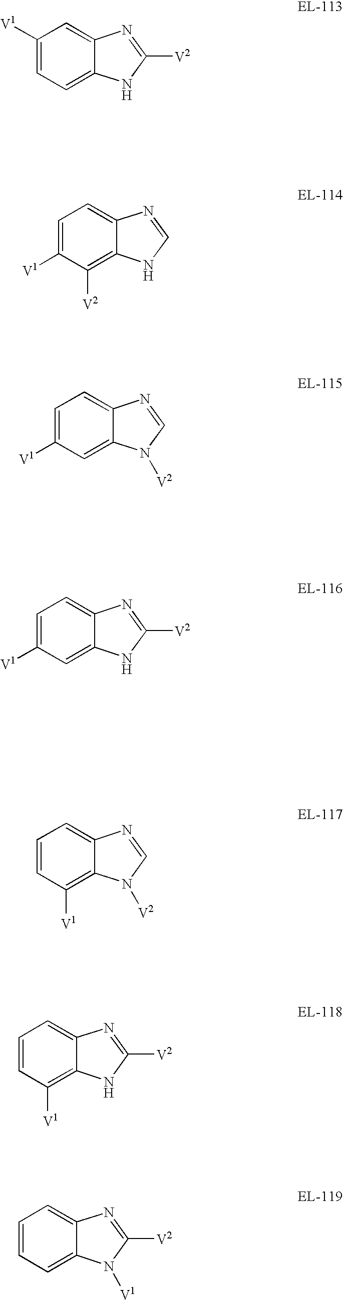

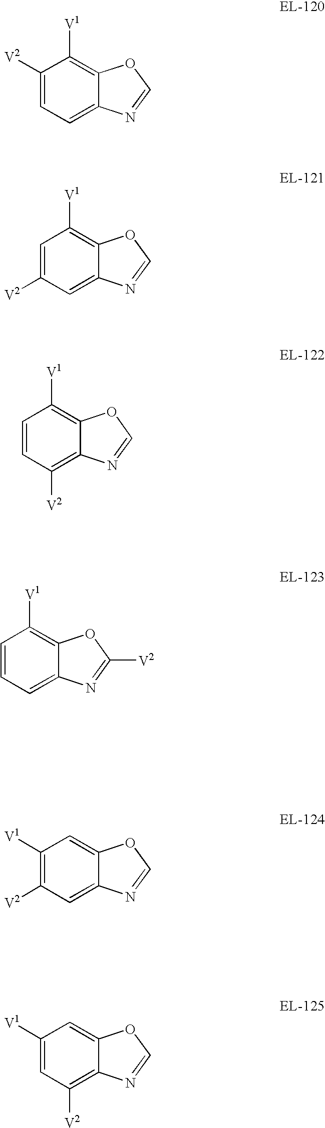

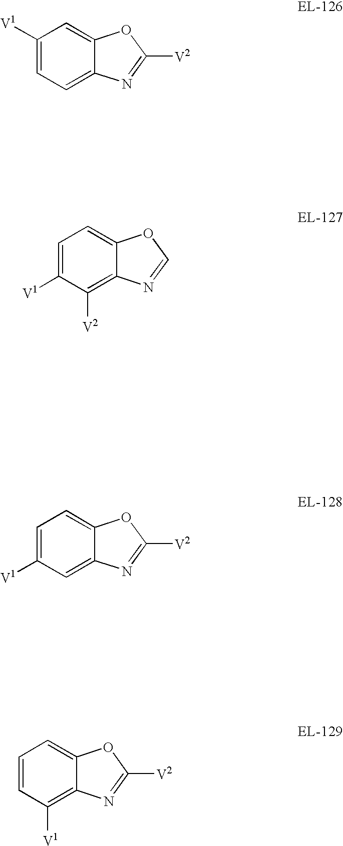

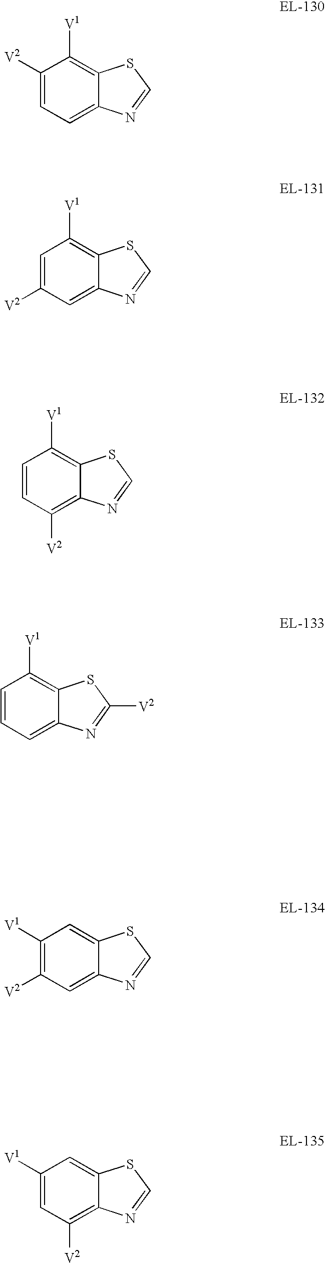

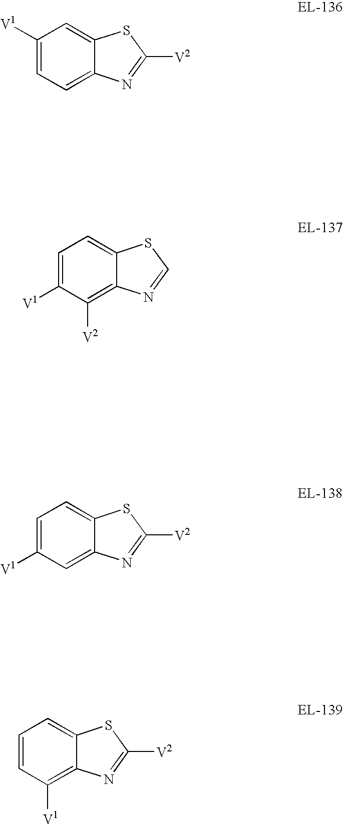

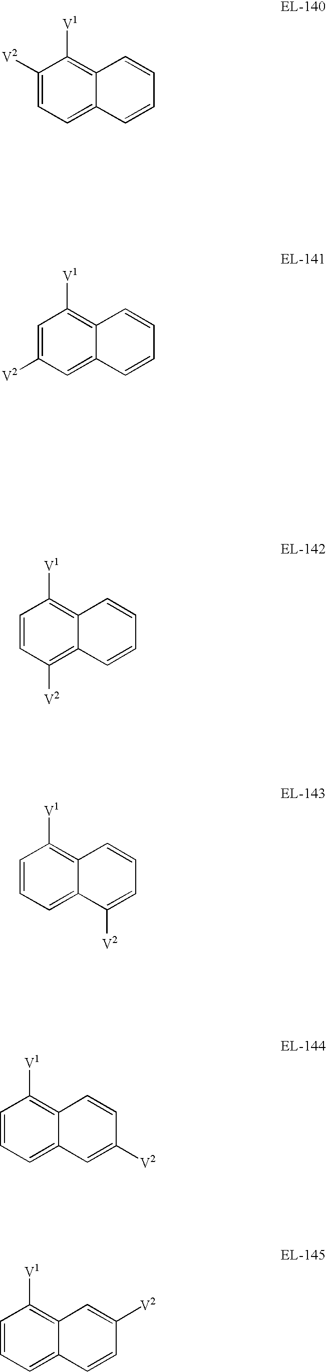

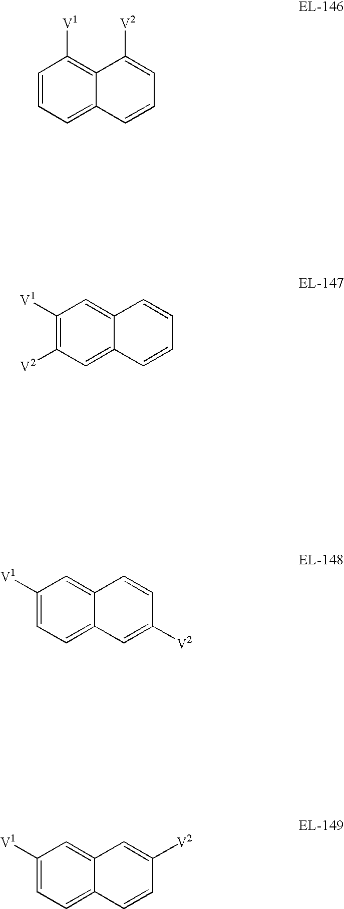

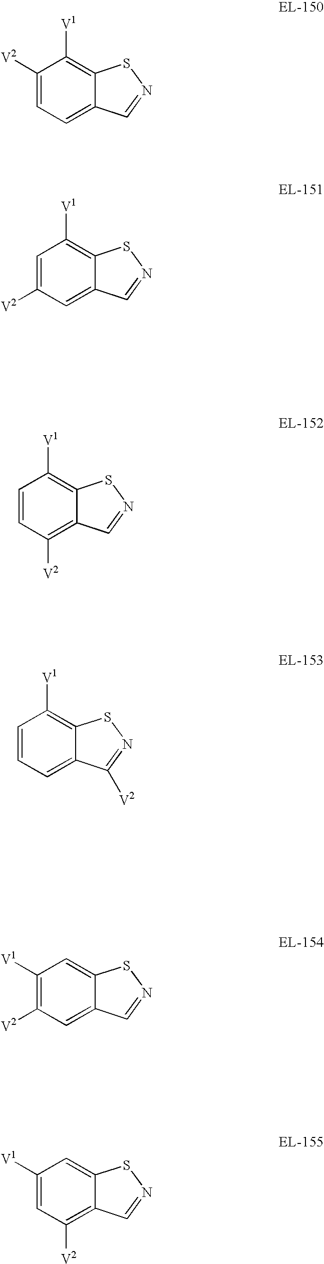

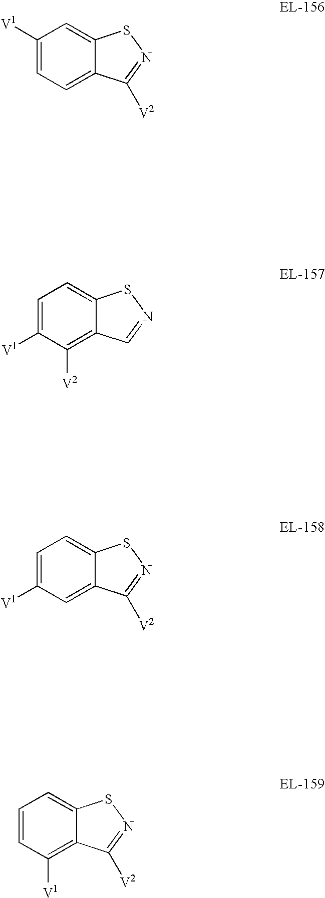

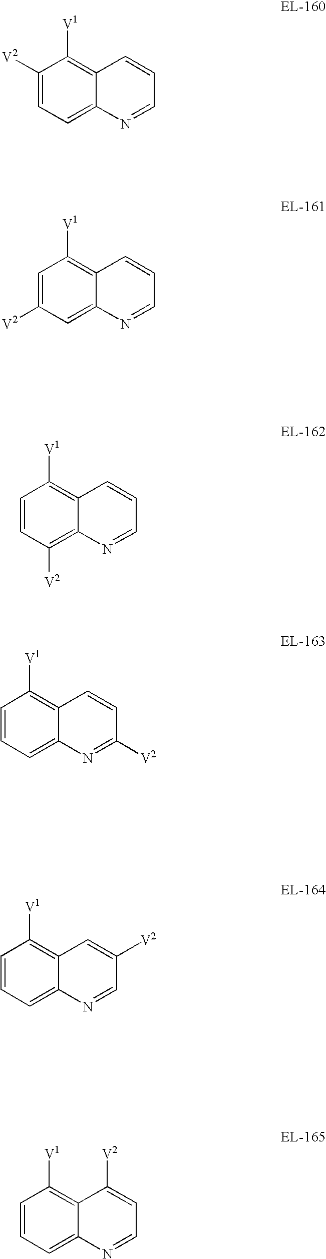

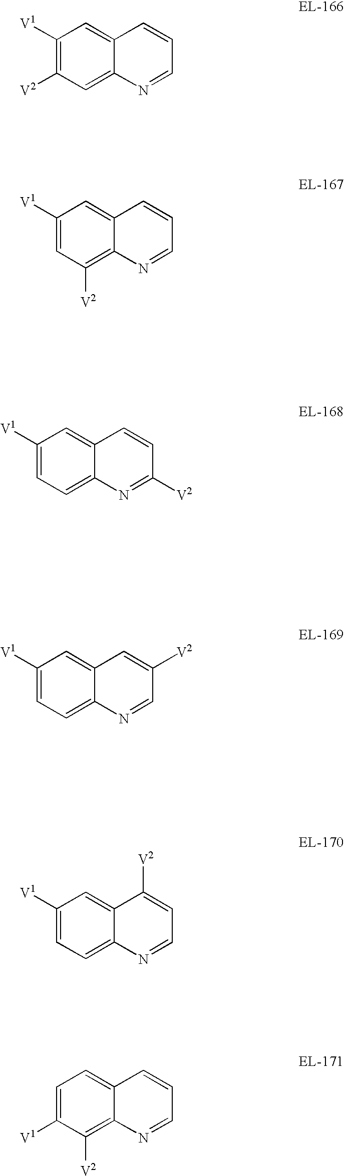

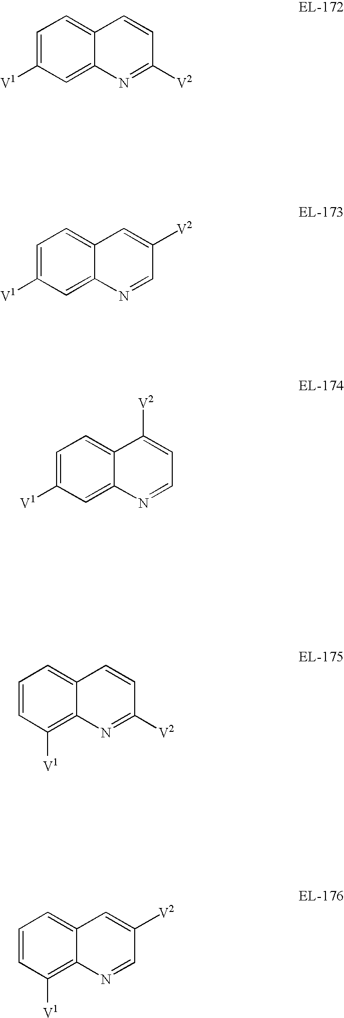

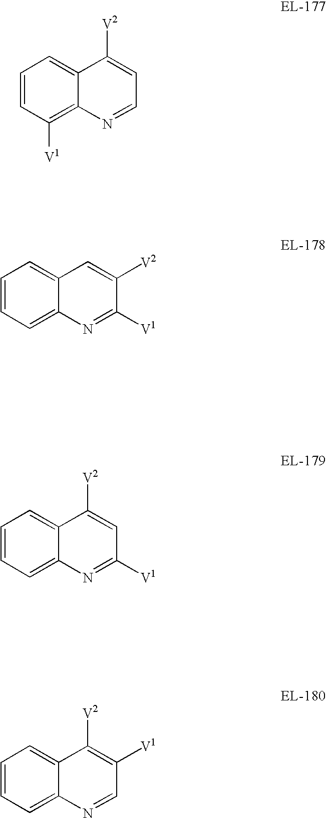

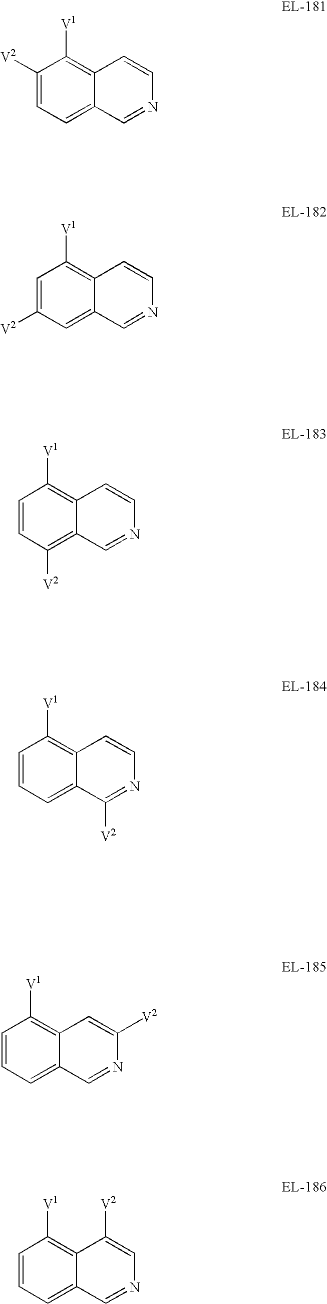

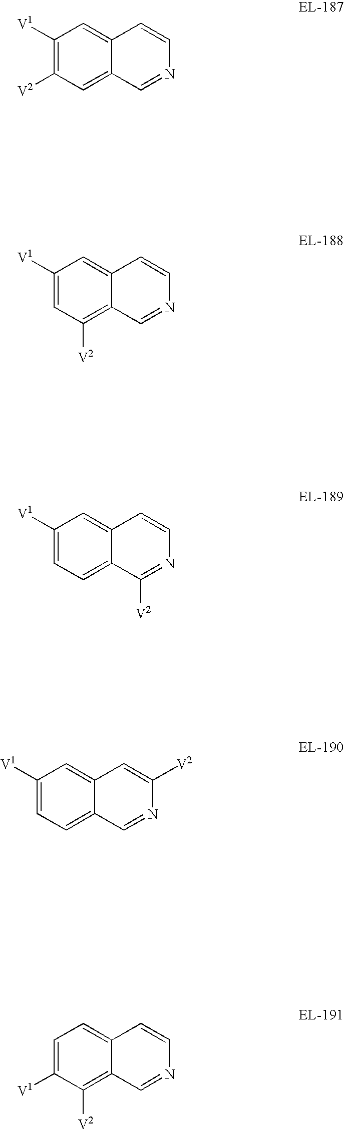

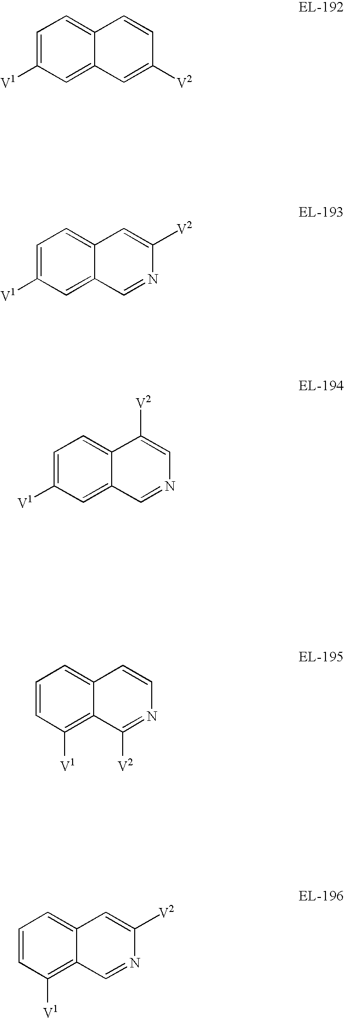

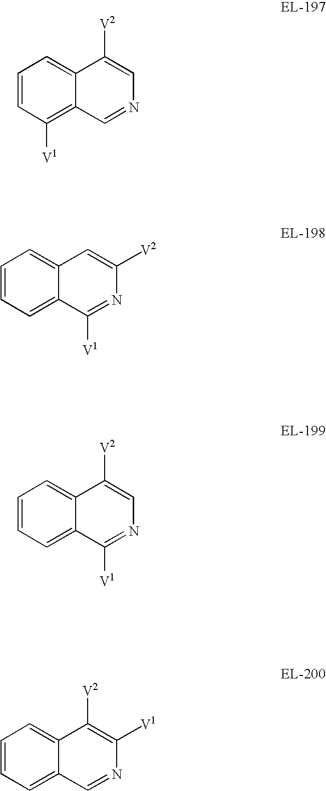

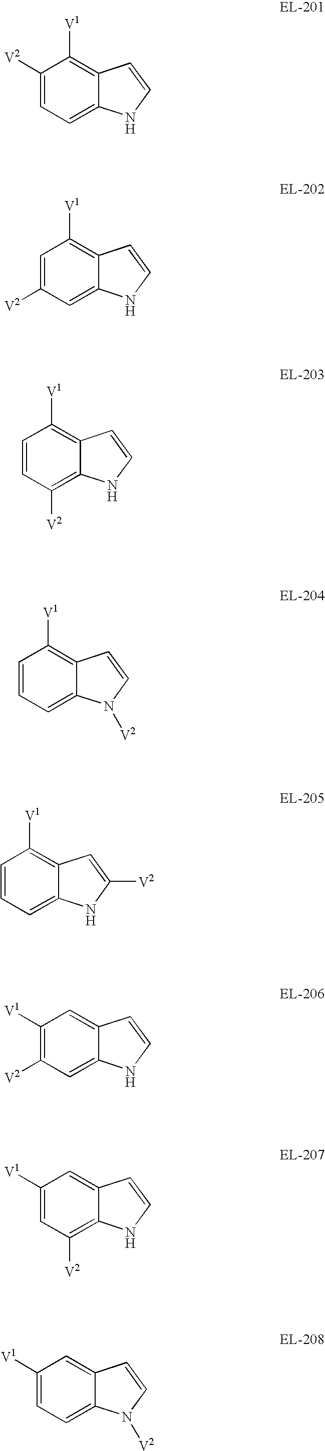

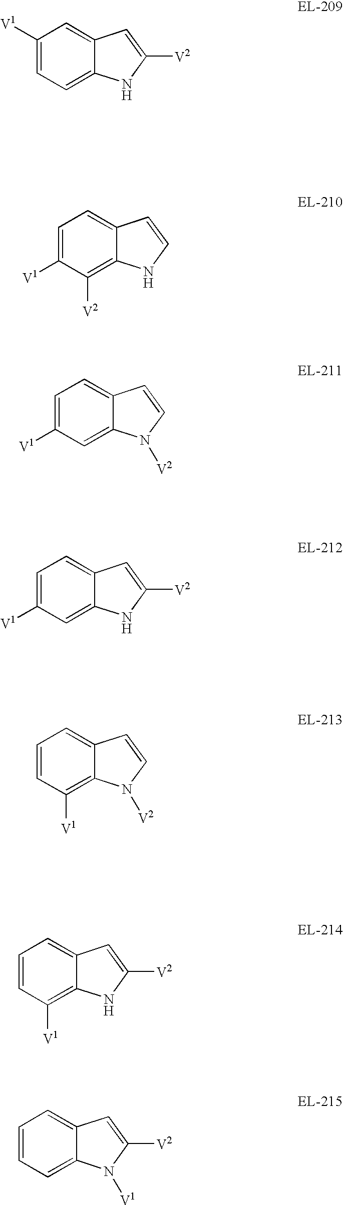

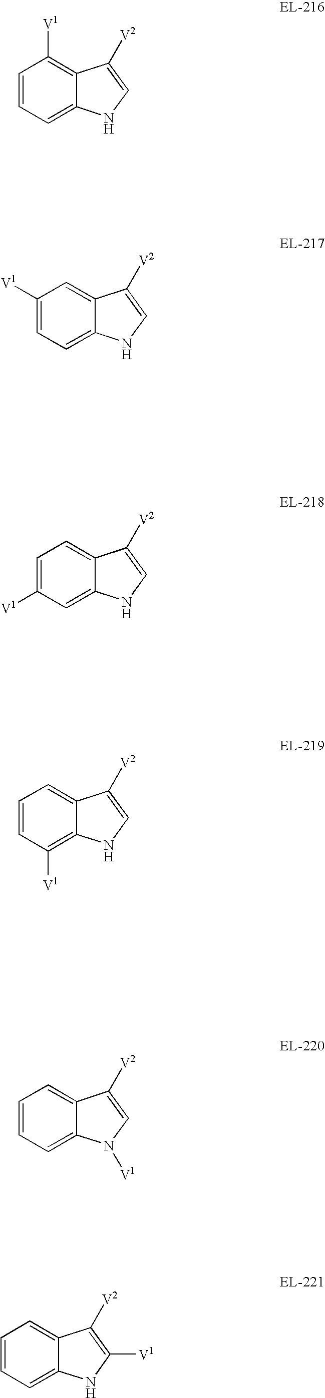

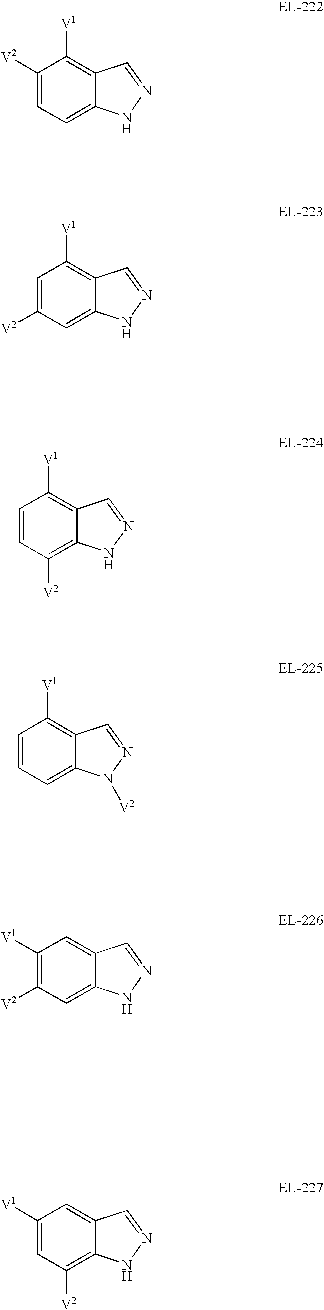

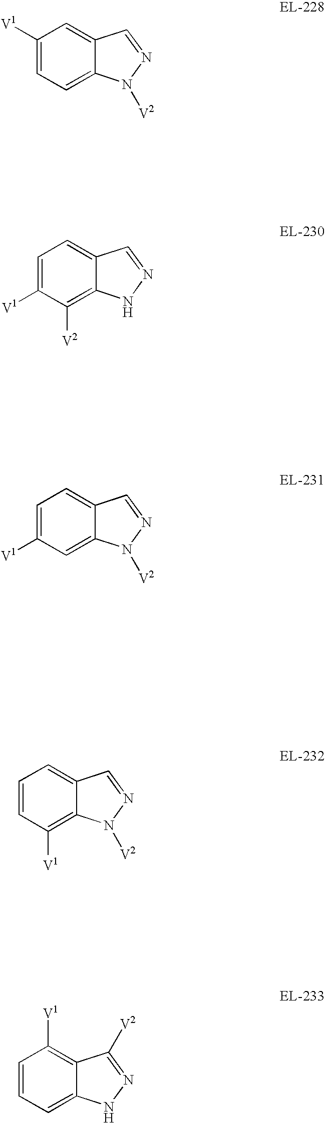

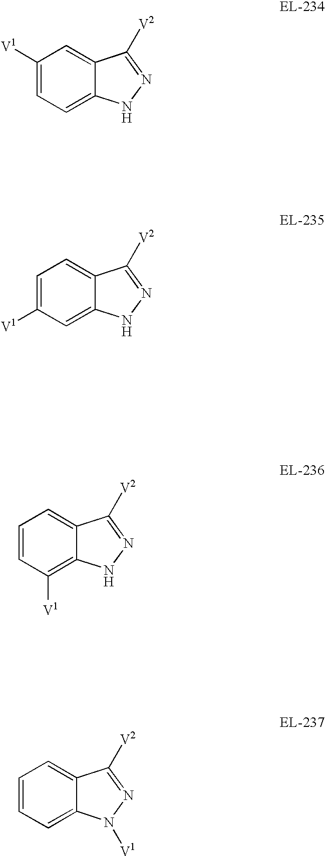

The above-described examples of the arylene group are a part of examples of the arylene group, and a divalent group obtained by removing two hydrogen atoms from quioxaline, phenanthridine, phenothiazine, phenoxazine, phthalazine, naphthyridine, quinazoline, cinnoline, carbazole, β-carboline, acridine, phenazine, phthalimide, or thioxanthene can be used as an arylene group.

As the substituent of an optionally substituted alkyl group, a hydroxyl group, a cyano group, a halogen atom, an alkoxy group, an alkylthio group, an alkoxy group which is optionally substituted with at least one halogen atom, an alkenyloxy group, an alkenylthio group, an acyl group, an acyloxy group, an alkylsulfinyl group, an alkylsulfonyl group, an alkylcarbamoyl group, an alkylamino group, a dialkylamino group, an alkylamino group which is optionally substituted with at least one halogen atom, an acylamino group, an acyl(alkyl)amino group, an alkylsulfonylamino group, an alkylsulfonyl(alkyl)amino group, an alkylsulfonylamino group which is optionally substituted with at least one halogen atom, an alkylsulfonyl(alkyl)amino group which is optionally substituted with at least one halogen atom, an amino group which is substituted with one or two optionally substituted alkyl groups, an amino group which is substituted with one or two optionally substituted aryl groups, an amino group which is substituted with an optionally substituted aryl group and an optionally substituted alkyl group, an optionally substituted alkoxy group, an optionally substituted aryloxy group, or an optionally substituted aryl group can be exemplified as an preferred example. More preferred examples thereof include a hydroxyl group, a halogen atom and an alkoxy group. Still more preferred examples thereof include an alkoxy group and a fluorine atom. Still more preferred examples include an alkoxy group. In addition, there is other embodiment in which a fluorine atom is still more preferred.

In the present specification, the number of the substituents of an optionally substituted group (i.e., an alkyl group, an alkenyl group, an alkynyl group, an alkoxy group and the like) is not specifically limited, but is generally 1 to several, and 1 is preferred. In addition, in another embodiment wherein the substituent of an optionally substituted group corresponds to a halogen atom, 1 to 3 substituents are preferred.

As the optionally substituted alkyl group, those described as the preferred examples of the alkyl group are also preferred. In another embodiment, as the optionally substituted alkyl group, an alkyl group which is substituted by the preferred substituent(s) of the optionally substituted alkyl group is preferred. Preferred examples of the optionally substituted alkyl group include a methyl group, an ethyl group, a n-propyl group, an isopropyl group, a cyclopropyl group, a n-butyl group, an isobutyl group, a s-butyl group, a t-butyl group, a cyclobutyl group, a cyclopropylmethyl group, a n-pentyl group, a cyclopentyl group, a cyclopropylethyl group, a cyclobutylmethyl group, a n-hexyl group, a cyclohexyl group, a cyclopropylpropyl group, a cyclobutylethyl group, a cyclopentylmethyl group, a trifluoromethyl group, a hydroxymethyl group, a 2-hydroxyethyl group, a methoxymethyl group and a 2-methoxyethyl group. More preferred examples thereof include a methyl group, an ethyl group, a n-propyl group, an isopropyl group, a trifluoromethyl group, a hydroxymethyl group, a 2-hydroxyethyl group, a methoxymethyl group and a 2-methoxyethyl group. Still more preferred examples thereof include a methyl group, an ethyl group, a hydroxymethyl group, a 2-hydroxyethyl group and a methoxymethyl group.

As the substituent of the optionally substituted alkenyl group, and the substituent of the optionally substituted alkynyl group, the groups the same as the aforementioned examples of the substituent of the optionally substituted alkyl group can be mentioned. As the optionally substituted alkenyl group, the aforementioned preferred examples of the alkenyl group are preferred. In addition, as the optionally substituted alkynyl group, the aforementioned preferred examples of the alkynyl group are preferred.

As the substituent of the optionally substituted alkylene group, the groups the same as the aforementioned examples of the substituent of the optionally substituted alkyl group can be mentioned. As the optionally substituted alkylene group, the aforementioned preferred examples of an alkylene group are preferred.

As the substituent of the optionally substituted alkoxy group, the groups the same as the aforementioned examples of the substituent of the optionally substituted alkyl group can be mentioned. As the optionally substituted alkoxy group, the aforementioned preferred examples of the alkoxy group are preferred. In another embodiment, as the optionally substituted alkoxy group, the alkoxy group which is substituted by the group the same as the aforementioned preferred substituent of the optionally substituted alkyl group is preferred. Specifically, preferred examples thereof include a methoxy group, an ethoxy group, a n-propoxy group, an isopropoxy group, a n-butoxy group, an isobutoxy group, a s-butoxy group, a t-butoxy group, a trifluoromethoxy group and a 2-methoxyethoxy group. More preferred examples thereof include a methoxy group, an ethoxy group, a trifluoromethoxy group and a 2-methoxyethoxy group. Still more preferred examples thereof include a methoxy group and an ethoxy group. A methoxy group is the most preferred example.

As the substituent of the optionally substituted alkylthio group, the groups the same as the aforementioned examples of the substituent of the optionally substituted alkyl group can be mentioned. As the optionally substituted alkylthio group, the aforementioned preferred examples of the alkylthio group are preferred. Specifically, preferred examples thereof include a methylthio group, an ethylthio group, a n-propylthio group, an isopropylthio group, a cyclopropylthio group, a n-butylthio group, an isobutylthio group, a s-butylthio group, a t-butylthio group, a cyclobutylthio group, a cyclopropylmethylolthio group and a-methoxyethylthio group. More preferred examples thereof include a methylthio group, an ethylthio group and a 2-methoxyethylthio group. Still more preferred examples thereof include a methylthio group and an ethylthio group.

The preferred examples of the optionally substituted alkoxycarbonyl group include the group in which a carbonyl group is added to the terminal of the aforementioned optionally substituted alkoxy group. Specifically, preferred examples thereof include a methoxycarbonyl group, an ethoxycarbonyl group, a n-propoxycarbonyl group, an isopropoxycarbonyl group, a cyclopropoxycarbonyl group, a n-butoxycarbonyl group, an isobutoxycarbonyl group, a s-butoxycarbonyl group, a t-butoxycarbonyl group, a cyclobutoxycarbonyl group and a cyclopropylmethoxycarbonyl group. Still more preferred examples thereof include a methoxycarbonyl group, an ethoxycarbonyl group and a t-butoxycarbonyl group. Even still more preferred examples thereof include a methoxycarbonyl group and an ethoxycarbonyl group.

As the optionally substituted amino group, an —NH₂ group, an alkylamino group, a dialkylamino group, an acylamino group, an acyl(alkyl)amino group, an alkylsulfonylamino group, an alkylsulfonyl(alkyl)amino group, an alkylamino group which is arbitrarily substituted with at least one halogen atom, an alkylsulfonylamino group which is arbitrarily substituted with at least one halogen atom, or an alkylsulfonyl(alkyl)amino group which is arbitrarily substituted with at least one halogen atom and the like can be mentioned. In addition, as the optionally substituted amino group, an amino group which is substituted with one or two alkyl groups that may be substituted, an amino group which is substituted with one or two aryl groups that may be substituted, an amino group which is substituted with an aryl group that may be substituted and an alkyl group that may be substituted, an amino group which is substituted with an acyl group that may be substituted, an alkylamino group which is substituted with an acyl group that may be substituted, an amino group which is substituted with an alkylsulfonyl group that may be substituted, an alkylamino group which is substituted with an alkylsulfonyl group that may be substituted, an alkylcarbamoylamino group that may be substituted, an alkylthiocarbamoylamino group that may be substituted, an arylcarbamoylamino group that may be substituted, an arylthiocarbamoylamino group that may be substituted, an alkyloxycarbonylamino group that may be substituted, or an aryloxycarbonylamino group that may be substituted and the like can be mentioned.

As the alkylamino group, a lower alkylamino group can be exemplified as a preferred example. As the lower alkylamino group, an alkylamino group comprising 1 to 4 carbon atoms is preferred. Specifically, preferred examples thereof include a methylamino group, an ethylamino group, a n-propylamino group, an isopropylamino group, a cyclopropylamino group, a n-butylamino group, an isobutylamino group, a s-butylamino group, a t-butylamino group, a cyclobutylamino group and a cyclopropylmethylamino group. More preferred examples thereof include a methylamino group, an ethylamino group and an isopropylamino group. Even more preferred examples thereof include a methylamino group and an ethylamino group.

As the dialkylamino group, an amino group which is substituted with alkyl groups that are the same or different can be mentioned. In generally, a lower dialkylamino group is preferred. As the lower dialkylamino group, an amino group which is substituted with alkyl groups each having 1 to 4 carbon atoms is preferred. Specifically, preferred examples thereof include a dimethylamino group, an ethyl(methyl)amino group, a diethylamino group, a methyl(n-propyl)amino group, an isopropyl(methyl)amino group, a cyclopropyl(methyl)amino group, a n-butyl(methyl)amino group, an isobutyl(methyl)amino group, a s-butyl(methyl)amino group, a t-butyl(methyl)amino group, a cyclobutyl(methyl)amino group and a cyclopropylmethyl(methyl)amino group. More preferred examples thereof include a dimethylamino group, a diethylamino group and an ethyl(methyl)amino group. Even more preferred examples thereof include a dimethylamino group and a diethylamino group.

As the acylamino group, an amino group which is substituted with the aforementioned acyl group can be mentioned. In addition, preferred examples of the acyl group are the same as the aforementioned examples. Specifically, preferred examples thereof include an acetylamino group, a propanoylamino group, a butanoylamino group, a 2-methylpropanoylamino group, a cyclopropylcarbonylamino group, a pentanoylamino group, a 3-methylbutanoylamino group, a 2,2-dimethylpropanoylamino group and a cyclobutylcarbonylamino group. More preferred examples thereof include an acetylamino group, a propanoylamino group and a 2,2-dimethylpropanoylamino group, and even more preferred examples thereof include an acetylamino group and a propanoylamino group.

As the acyl(alkyl)amino group, an amino group which is simultaneously substituted with one acyl group and one alkyl group can be mentioned. Preferred examples of the acyl group and the alkyl group are the same as the aforementioned examples. Specifically, preferred examples thereof include an acetyl(methyl)amino group, a methyl(propanoyl)amino group, a butanoyl(methyl)amino group, a methyl (2-methylpropanoyl)amino group, a cyclopropylcarbonyl(methyl)amino group, a methyl(pentanoyl)amino group, a methyl (3-methylbutanoyl)amino group, a 2,2-dimethylpropanoyl(methyl)amino group and a cyclobutylcarbonyl(methyl)amino group. More preferred examples thereof include an acetyl(methyl)amino group and a methyl(propanoyl)amino group, and even more preferred examples thereof include an acetyl(methyl)amino group.

As the alkylsulfonylamino group, an amino group which is substituted with the aforementioned alkylsulfonyl group can be mentioned. Preferred examples of the alkylsulfonyl group are the same as the aforementioned examples. Specifically, preferred examples thereof include a methylsulfonylamino group, an ethylsulfonylamino group, a n-propylsulfonylamino group, an isopropylsulfonylamino group, a cyclopropylsulfonylamino group, a n-butylsulfonylamino group, an isobutylsulfonylamino group, a s-butylsulfonylamino group, a t-butylsulfonylamino group, a cyclobutylsulfonylamino group and a cyclopropylmethylsulfonylamino group. More preferred examples thereof include a methylsulfonylamino group, an ethylsulfonylamino group and a n-propylsulfonylamino group, and even more preferred examples thereof include a methylsulfonyl group and an ethylsulfonyl group.

As the alkylsulfonyl(alkyl)amino group, an amino group which is simultaneously substituted with one alkylsulfonyl group and one alkyl group can be mentioned. Preferred examples of the alkylsulfonyl group and the alkyl group are the same as the aforementioned examples. Specifically, preferred examples thereof include a methyl(methylsulfonyl)amino group, an ethylsulfonyl(methyl)amino group, a methyl(n-propylsulfonyl)amino group, an isopropylsulfonyl(methyl)amino group, a cyclopropylsulfonyl(methyl)amino group, a n-butylsulfonyl(methyl)amino group, an isobutylsulfonyl(methyl)amino group, a s-butylsulfonyl(methyl)amino group, a t-butylsulfonyl(methyl)amino group, a cyclobutylsulfonyl(methyl)amino group and a cyclopropylmethylsulfonyl(methyl)amino group. More preferred examples thereof include a methyl(methylsulfonyl)amino group and an ethylsulfonyl(methyl)amino group, and even more preferred examples thereof include a methyl(methylsulfonyl)amino group.

As the alkylamino group which is arbitrarily substituted with at least one halogen atom, the alkylamino group obtained by substituting one or more hydrogen atoms of the aforementioned alkylamino group with any type of a halogen atom can be mentioned. Preferably, it is an alkylamino group having 1 to 4 carbon atoms which is arbitrarily substituted with one or more halogen atoms. When the alkylamino group is substituted with two or more halogen atoms, the halogen atoms can be the same or different. Specifically, preferred examples thereof include a chloromethylamino group, a dichloromethylamino group, a trichloromethylamino group, a fluoromethylamino group, a difluoromethylamino group, a trifluoromethylamino group, a fluoroethylamino group and a 2,2,2-trifluoroethylamino group. More preferred examples thereof include a trifluoromethylamino group and a 2,2,2-trifluoroethylamino group, and even more preferred examples thereof include a trifluoromethylamino group.

As the alkylsulfonylamino group which is arbitrarily substituted with at least one halogen atom, the alkylsulfonylamino group obtained by substituting one or more hydrogen atoms of the aforementioned alkylsulfonylamino group with any type of a halogen atom can be mentioned. In general, an alkylsulfonylamino group having 1 to 4 carbon atoms which is arbitrarily substituted with one or more halogen atoms is preferred. When the alkylsulfonylamino is substituted with two or more halogen atoms, the halogen atoms can be the same or different. Specifically, preferred examples thereof include a trifluoromethylsulfonylamino group.

As the alkylsulfonyl(alkyl)amino group which is arbitrarily substituted with at least one halogen atom, the alkylsulfonyl(alkyl)amino group obtained by substituting one or more hydrogen atoms of the aforementioned alkylsulfonyl(alkyl)amino group with any type of a halogen atom can be mentioned. Preferably, it is an alkylsulfonyl(alkyl)amino group having 1 to 4 carbon atoms which is arbitrarily substituted with one or more halogen atoms. When the alkylsulfonyl(alkyl)amino group is substituted with two or more halogen atoms, the halogen atoms can be the same or different. Specifically, preferred examples thereof include a methyl(trifluoromethylsulfonyl)amino group.

With respect to the optionally substituted alkyl group of the amino group substituted with one or two optionally substituted alkyl groups, the groups the same as the preferred examples of the aforementioned optionally substituted alkyl group can be mentioned. In addition, with respect to the amino group substituted with one or two optionally substituted alkyl groups, the groups the same as the preferred examples of the aforementioned alkylamino group and dialkylamino group can be mentioned. As another embodiment, with respect to the amino group substituted with one or two optionally substituted alkyl groups, the amino group substituted with one or two alkyl groups that are substituted with the preferred substituent(s) of the aforementioned optionally substituted alkyl group is preferred. In addition, as another embodiment, with respect to the amino group substituted with one or two optionally substituted alkyl groups, the amino group substituted with one alkyl group that is substituted with the preferred substituent(s) of the aforementioned optionally substituted alkyl group is preferred. In addition, as another embodiment, with respect to the amino group substituted with one or two optionally substituted alkyl groups, the amino group substituted with one alkyl group that is substituted with the aforementioned preferred substituent (s) of the aforementioned optionally substituted alkyl group and with one lower alkyl group is preferred. Specifically, preferred examples thereof include a dimethylamino group, a diethylamino group, an ethylmethylamino group, a methoxyethylamino group, a methyl(methoxyethyl)amino group, a hydroxyethylamino group and a hydroxyethyl(methyl)amino group. More preferred examples thereof include a dimethylamino group, a methoxyethylamino group and a hydroxyethylamino group.

With respect to the optionally substituted aryl group of the amino group substituted with one or two optionally substituted aryl groups, the preferred examples of an optionally substituted aryl group described later can be mentioned. In addition, with respect to the amino group substituted with one or two optionally substituted aryl groups, the amino group substituted with one of the preferred examples of the aforementioned aryl group can be mentioned. More preferred examples thereof include a phenylamino group.

With respect to the amino group that is substituted with an optionally substituted aryl group and an optionally substituted alkyl group, preferred examples of the optionally substituted aryl group are the same as the aforementioned examples. In addition, with respect to the substituent of the optionally substituted alkyl group, the preferred examples of the aforementioned optionally substituted alkyl group are preferred. With respect to the amino group that is substituted with an optionally substituted aryl group and an optionally substituted alkyl group, an alkylamino group that is substituted with an optionally substituted aryl group is preferred. More preferred examples thereof include a methyl(phenyl)amino group.

With respect to the substituent of the acyl group of the amino group that is substituted with an optionally substituted acyl group and the alkylamino group that is substituted with an optionally substituted acyl group, later-described preferred examples of the substituent of an optionally substituted acyl group are preferred. More preferred examples of the amino group that is substituted with an optionally substituted acyl group include an acetylamino group, a propanoylamino group, a butanoylamino group and a phenylacetylamino group.

With respect to the substituent of the alkylsulfonyl group of the amino group that is substituted with an optionally substituted alkylsulfonyl group and the alkylamino group that is substituted with an optionally substituted alkylsulfonyl group, later-described preferred examples of the substituent of an optionally substituted alkylsulfonyl group are preferred. More preferred examples of the amino group that is substituted with an optionally substituted alkylsulfonyl group include a methanesulfonylamino group, a trifluoromethanesulfonylamino group, a methyl(methanesulfonyl)amino group and a methyl(trifluoromethanesulfonyl)amino group.

With respect to the substituent of the optionally substituted alkylcarbamoylamino group, when the substituent (s) is present on the alkyl group, the aforementioned preferred examples of the substituent of the optionally substituted alkyl group are preferred. In addition, when the substituent(s) is present on the nitrogen atom of the alkylcarbamoylamino group, the groups the same as the aforementioned preferred examples of the substituent of the optionally substituted alkyl group or a hydrogen atom is preferred. The number of the substituent(s) of the optionally substituted alkylcarbamoylamino group can be one or more and the substituents can be independently the same or different. As the optionally substituted alkylcarbamoylamino group, a group having one substitutent that is present on an alkyl group is preferred. More preferred examples thereof include an ethylcarbamoylamino group and a methylcarbamoylamino group.

With respect to the optionally substituted alkylthiocarbamoylamino group, preferred examples thereof are the same as those each corresponding to the examples of the aforementioned alkylcarbamoylamino group. More preferred examples thereof include an ethylthiocarbamoylamino group and a methylthiocarbamoylamino group.

With respect to the optionally substituted alkyloxycarbonylamino group, preferred examples thereof are the same as those each corresponding to the examples of the aforementioned alkylcarbamoylamino group. More preferred examples thereof include an ethoxycarbonylamino group and a methoxycarbonylamino group.

With respect to the substituent of the optionally substituted arylcarbamoylamino group, when the substituent(s) is present on the aryl group, later-described preferred examples of the substituent of an optionally substituted aryl group are preferred. In addition, when the substituent(s) is present on the nitrogen atom of the arylcarbamoylamino group, the groups the same as the aforementioned preferred examples of the substituent of the optionally substituted alkyl group or a hydrogen atom is preferred. The number of the substituent(s) of the optionally substituted arylcarbamoylamino group can be one or more and the substituents can be independently the same or different. As the optionally substituted arylcarbamoylamino group, an arylcarbamoylamino group having one substitutent that is present on an aryl group is preferred. More preferred examples thereof include a phenylcarbamoylamino group which may be substituted.

With respect to the optionally substituted arylthiocarbamoylamino group, preferred examples thereof are the same as those each corresponding to the examples of the arylcarbamoylamino group described above.

With respect to the optionally substituted aryloxycarbonylamino group, preferred examples thereof are the same as those each corresponding to the examples of the arylcarbamoylamino group described above. More preferred examples thereof include a phenylthiocarbamoylamino group which may be substituted.

With respect to the substituent of the optionally substituted aryloxy group, later-described preferred examples of the substituent of an optionally substituted aryl group are preferred. The number of the substituent(s) of the optionally substituted aryloxy group can be one or more and the substituents can be independently the same or different. As the optionally substituted aryloxy group, an aryloxy group having one substitutent that is present on an aryloxy group is preferred. More preferred examples thereof include a phenyloxy group, a 2-thienyloxy group, and a 3-thienyloxy group which may be substituted.

The substituent of the optionally substituted acyl group is the same as the aforementioned preferred examples of the substituent of the optionally substituted alkyl group. A preferred example thereof is at least one halogen atom.

With respect to the optionally substituted acyl group, an acyl group arbitrarily substituted with at least one halogen atom is preferred. More preferably, it is an acyl group having 2 to 5 carbon atoms and arbitrarily substituted with at least one halogen atom. When it is substituted with two or more halogen atoms, the halogen atoms can be either the same or different. Preferred examples thereof include a trifluoroacetyl group.

In addition, preferred examples of the optionally substituted acyl group include an acetyl group, a propanoyl group, a butanoyl group, a 2-methylpropanoyl group, a cyclopropylcarbonyl group, a pentanoyl group, a 3-methylbutanoyl group, a 2,2-dimethylpropanoyl group and a cyclobutylcarbonyl group. More preferred examples thereof include an acetyl group, a propanoyl group and a 2,2-dimethylpropanoyl group, and even more preferred examples thereof include an acetyl group and a propanoyl group.

With respect to the optionally substituted acyloxy group, the group in which an oxygen atom is added to the terminal of the optionally substituted acyl group is preferred. Preferred examples thereof include a trifluoroacetoxy group.

In addition, preferred examples of the optionally substituted acyloxy group include an acetoxy group, a propanoyloxy group, a butanoyloxy group, a 2-methylpropanoyloxy group, a cyclopropylcarbonyloxy group, a pentanoyloxy group, a 3-methylbutanoyloxy group, a 2,2-dimethylpropanoyloxy group and a cyclobutylcarbonyloxy group. More preferred examples thereof include an acetoxy group, a propanoyloxy group and a 2,2-dimethylpropanoyloxy group, and even more preferred examples thereof include an acetoxy group and a propanoyloxy group.

The substituent of the optionally substituted alkylsulfinyl group is the same as the substituent of the aforementioned optionally substituted alkyl group. However, at least one halogen atom is preferred.

With respect to the optionally substituted alkylsulfinyl group, an alkylsulfinyl group arbitrarily substituted with at least one halogen atom is preferred. More preferably, it is an alkylsulfinyl group having 1 to 4 carbon atoms and arbitrarily substituted with at least one halogen atom. When it is substituted with two or more halogen atoms, the halogen atoms can be either the same or different. Preferred examples thereof include a trifluoromethanesulfinyl group.

In addition, preferred examples of the optionally substituted alkylsulfinyl group include a methylsulfinyl group, an ethylsulfinyl group, a n-propylsulfinyl group, an isopropylsulfinyl group, a cyclopropylsulfinyl group, a n-butylsulfinyl group, an isobutylsulfinyl group, a s-butylsulfinyl group, a t-butylsulfinyl group, a cyclobutylsulfinyl group and a cyclopropylmethylsulfinyl group. More preferred examples thereof include a methylsulfinyl group and an ethylsulfinyl group, and even more preferred examples thereof include a methylsulfinyl group.

The substituent of the optionally substituted alkylsulfonyl group is the same as the substituent of the aforementioned optionally substituted alkyl group. However, at least one halogen atom is more preferred.

With respect to the substituted alkylsulfonyl group, an alkylsulfonyl group arbitrarily substituted with at least one halogen atom is preferred. More preferably, it is an alkylsulfonyl group having 1 to 4 carbon atoms and arbitrarily substituted with at least one halogen atom. When it is substituted with two or more halogen atoms, the halogen atoms can be either the same or different. Preferred examples thereof include a trifluoromethanesulfonyl group.

In addition, preferred examples of the optionally substituted alkylsulfonyl group include a methylsulfonyl group, an ethylsulfonyl group, a n-propylsulfonyl group, an isopropylsulfonyl group, a cyclopropylsulfonyl group, a n-butylsulfonyl group, an isobutylsulfonyl group, a s-butylsulfonyl group, a t-butylsulfonyl group, a cyclobutylsulfonyl group and a cyclopropylmethylsulfonyl group. More preferred examples thereof include a methylsulfonyl group and an ethylsulfonyl group, and even more preferred examples thereof include a methylsulfonyl group.

Preferred examples of the substituent of the optionally substituted aryl ring and the substituent of the optionally substituted aryl group include a hydroxyl group, an alkyl group arbitrarily substituted with at least one hydroxy group, a halogen atom, an alkyl group arbitrarily substituted with at least one halogen atom, an alkoxy group, an alkylthio group, an alkoxy group arbitrarily substituted with at least one halogen atom, an alkenyloxy group, an alkenylthio group, an acyl group, an acyloxy group, an alkylsulfinyl group, an alkylsulfonyl group, an alkylcarbamoyl group, an alkylamino group, a dialkylamino group, an alkylamino group arbitrarily substituted with at least one halogen atom, an acylamino group, an acyl(alkyl)amino group, an alkylsulfonylamino group, an alkylsulfonyl(alkyl)amino group, an alkylsulfonylamino group arbitrarily substituted with at least one halogen atom and an alkylsulfonyl(alkyl)amino group arbitrarily substituted with at least one halogen atom.

With respect to the optionally substituted aryl ring or the optionally substituted aryl group, the aforementioned preferred examples of the aryl ring or the aryl group can be mentioned. As another embodiment, an aryl ring which comprises the group the same as the substitutent of the aforementioned optionally substituted alkyl group or a lower alkyl group, or an aryl group which comprises the group the same as the substitutent of the aforementioned optionally substituted alkyl group or a lower alkyl group can be mentioned as a preferred example.

With respect to the substituent of the optionally substituted arylene group, the group the same as the substituent of the above-described optionally substituted aryl group can be mentioned. Preferred examples of the optionally substituted arylene group include those exemplified as the aforementioned arylene group.

In the present invention, unless specifically indicated otherwise, all of isomers are included. An isomer in a double bond, a ring or a fused ring (i.e., E, Z, cis, and trans), an isomer occurring due to the presence of an asymmetric carbon atom (R- and S-isomers, α and β configuration, enantiomer, and diastereomer), an optically active material having optical rotation (D-, L-, d- and l-isomers), a compound having polarity obtained by chromatographic separation (highly polar isomer and weakly polar isomer), an equilibrium compound, a rotationary isomer, a mixture comprising them in any ratio, and a racemic mixture are all included in the present invention.

In the present invention, unless specifically indicated otherwise, symbol

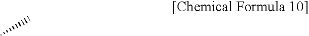

indicates a bonding below the plane of paper (i.e., α-configuration) and symbol

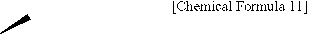

indicates a bonding in front of the plane of paper (i.e., β-configuration), and symbol

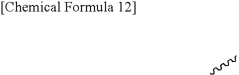

indicates α-configuration, β-configuration or a mixture thereof, or an E-isomer, a Z-isomer or a mixture thereof of the isomers in a double bond, and symbol

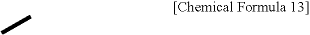

indicates a mixture of α-configuration and β-configuration.

In the above Formula (1), Ar¹ represents a heteroaryl ring. A monocyclic aromatic heterocycle can be mentioned as the heteroaryl ring. A 5- or 6-membered aromatic heterocycle comprising 1 or at least 2 heteroatoms can be mentioned as the monocyclic aromatic heterocycle. Specifically, preferred examples of the 5- or 6-membered aromatic heterocycle include thiophene, pyridine, furan, thiazole, oxazole, pyrazole, pyrazine, pyrimidine, pyrrole, imidazole, pyridazine, isothiazole, isoxazole, 1,2,3-triazole and 1,3,4-triazole.

More specifically, a compound having, as apart represented by formula (2), which is a partial structure of formula (1),

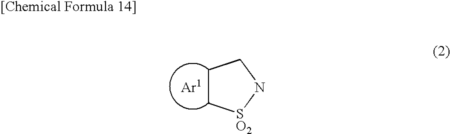

the following structure can be mentioned.

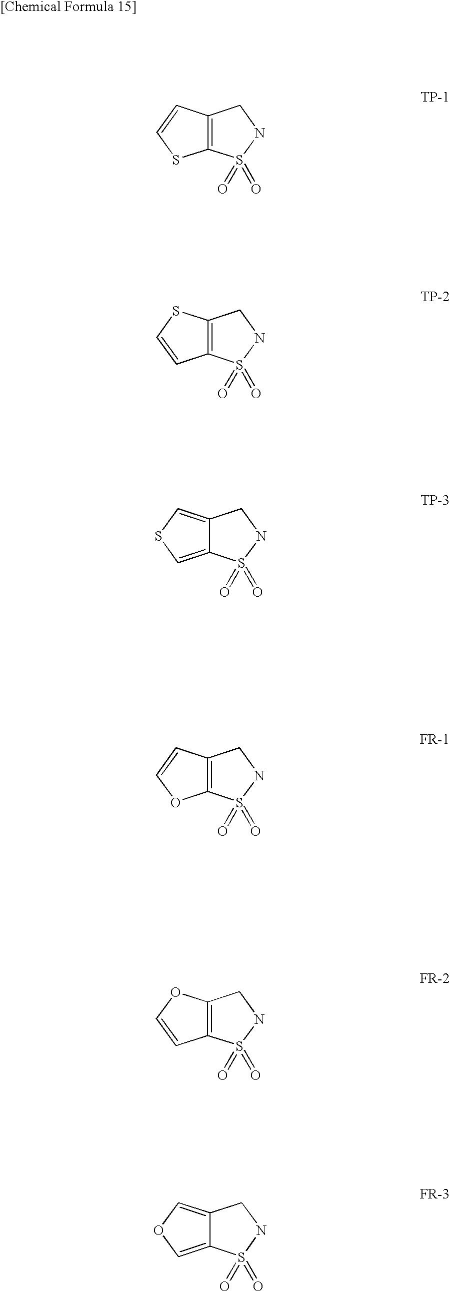

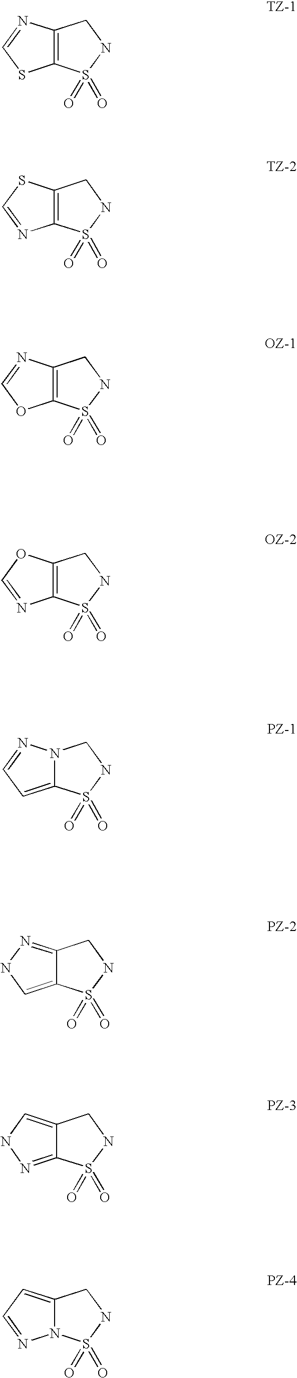

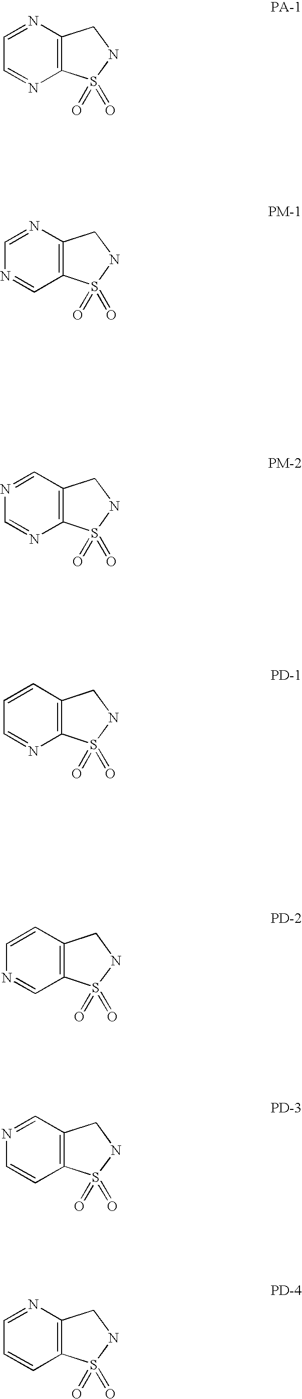

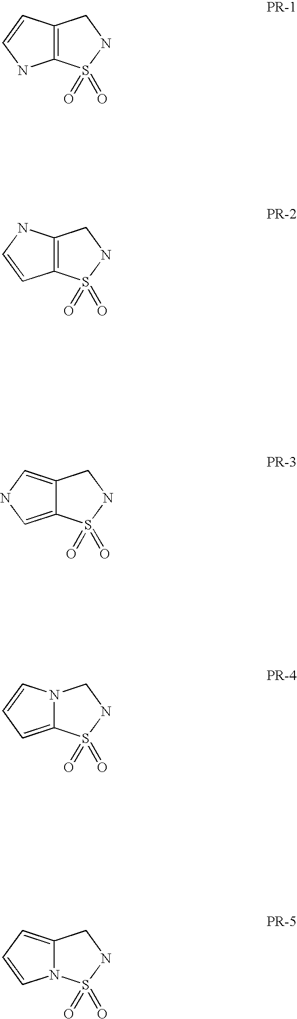

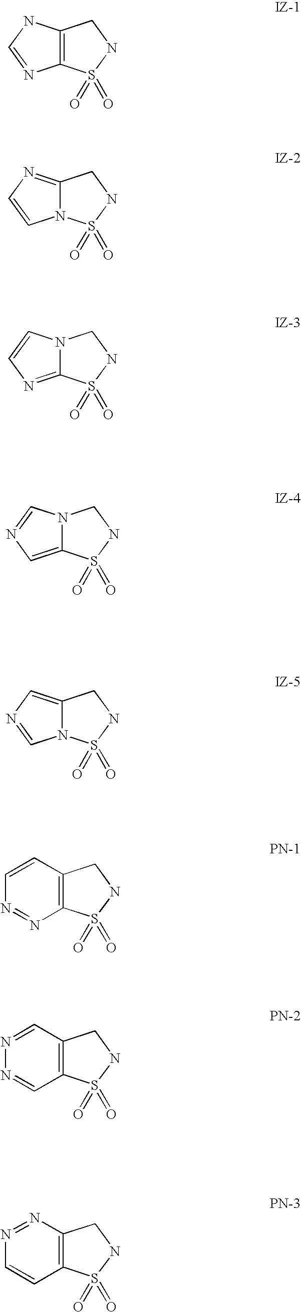

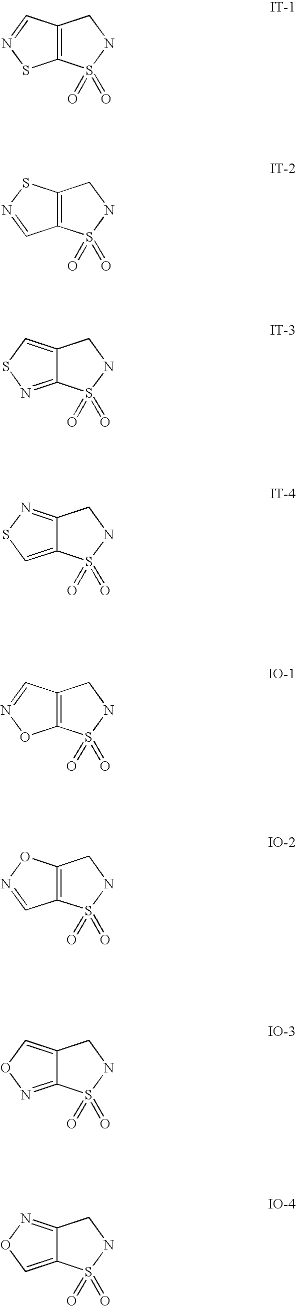

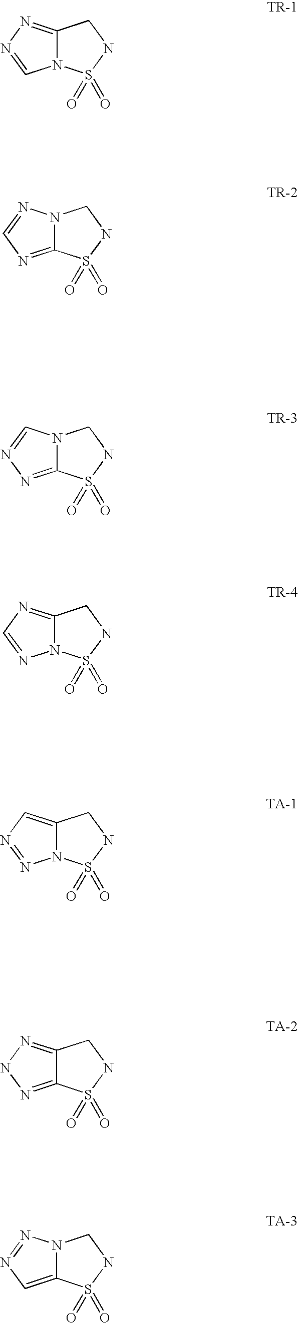

Ar¹ is preferably a thiophene ring. As a compound in which Ar¹ is a thiophene ring, a compound having the following formula (3) or formula (4) can be mentioned [in formula (3) and formula (4), R¹¹, R¹², R²¹, R²², R³¹, n, m, and Y¹ have the meanings the same as the aforementioned].

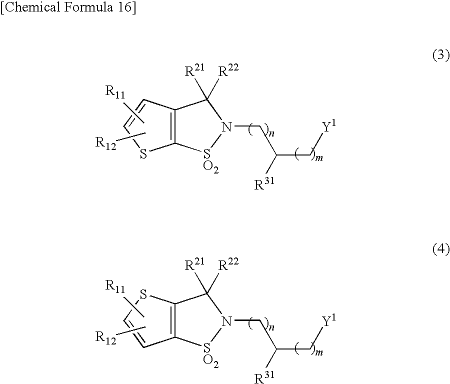

When Ar¹ is a thiophene ring, a compound having formula (3) is preferred. Alternatively, there is another embodiment in which a compound having formula (4) is preferred.

R¹¹ represents —R¹¹¹—R¹¹²—R¹¹³—R¹¹⁴ (R¹¹¹ represents a single bond, —O—, —NR¹¹¹¹—, —NR¹¹¹¹CO—, —CONR¹¹¹¹—, —NR¹¹¹¹SO₂—, —SO₂NR¹¹¹¹— (R¹¹¹¹ represents a hydrogen atom or an optionally substituted alkyl group), —(C═O)—, —C≡C— or —CH═CH—, R¹¹² represents an optionally substituted alkylene group or an optionally substituted arylene group, or R¹¹² may be bonded to R¹¹¹¹ to form a ring structure, R¹¹³ represents a single bond, —O—, —NR¹¹³¹—, —NR¹¹³¹CO—, —CONR¹¹³¹—, —NR¹¹³¹SO₂—, —SO₂NR¹¹³¹— (R¹¹³¹ represents a hydrogen atom or an optionally substituted alkyl group), —(C═O)—, —C≡C— or —CH═CH— and R¹¹⁴ represents an optionally substituted aryl group, an optionally substituted alkyl group or a hydrogen atom and R¹¹⁴ may be bonded to R¹¹¹³ to form a ring structure).

In the case where Ar¹ is a thiophene ring, examples of the substituted position of R¹¹ include those described below.

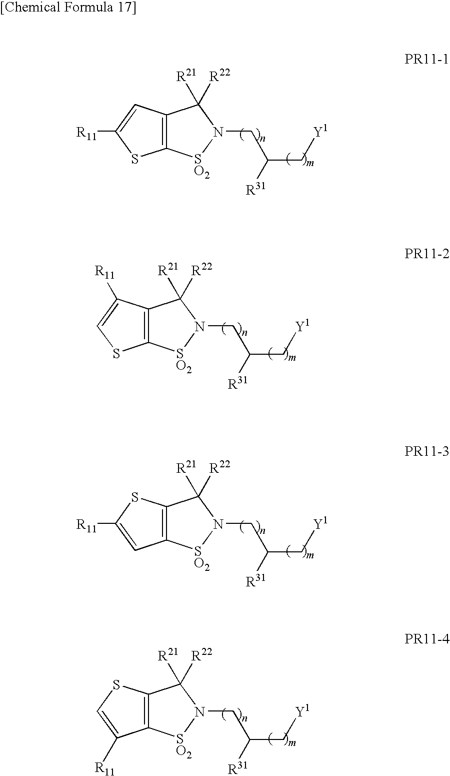

In the case where Ar¹ is a thiophene ring, the substituted position of R¹¹ is preferably

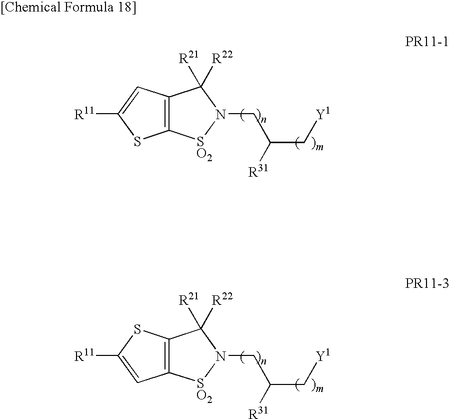

, and more preferably

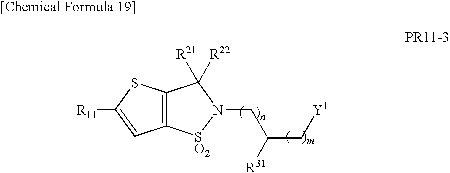

R¹¹¹ represents a single bond, —O—, —NR¹¹¹¹—, —NR¹¹¹¹CO—, —CONR¹¹¹¹—, —NR¹¹¹¹SO₂—, —SO₂NR¹¹¹¹— (R¹¹¹¹ represents a hydrogen atom or an optionally substituted alkyl group), —(C═O)—, —C≡C— or —CH═CH—. However, in the case where Ar¹ is a nitrogen atom-containing group, and the substituted position of R¹¹ is at the nitrogen atom on Ar¹, R¹¹ is a single bond, —CONR¹¹¹¹—, —SO₂NR¹¹¹¹—, —(C═O)—, —C≡C— or —CH═CH—. In addition, in the case where R¹¹² is a nitrogen atom-containing group, and the bonding position of R¹¹¹ is on the nitrogen atom of R¹¹², R¹¹¹ represents a single bond, —NR¹¹¹¹CO—, —NR¹¹¹¹SO₂—, —(C═O)—, —C≡C— or —CH═CH—. Examples of the compound wherein Ar¹ is a nitrogen atom-containing group, and the bonding position of R¹¹¹ is on the nitrogen atom of Ar¹ include compounds represented by Formulae described below.

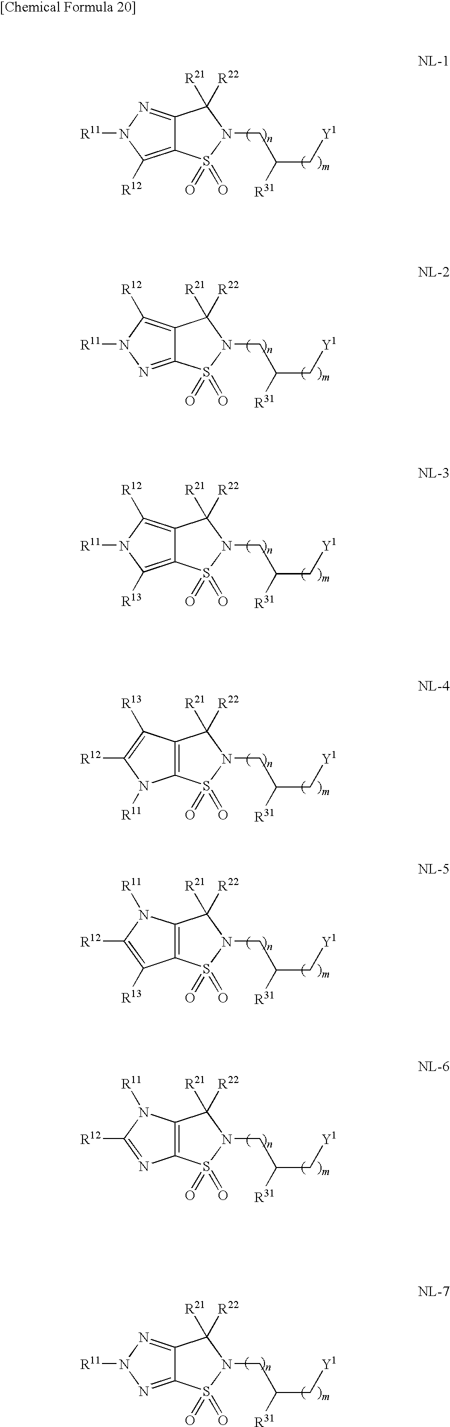

[In the Formulae NL-1 to NL-7, R¹¹, R¹², R¹³, R²¹, R²², R³¹, n, m, and Y¹ are the same as the aforementioned]

Examples of R¹¹ wherein R¹¹² is a nitrogen atom-containing group, and the bonding position of R¹¹¹ is on the nitrogen atom of R¹¹² include groups represented by Formulae described below.

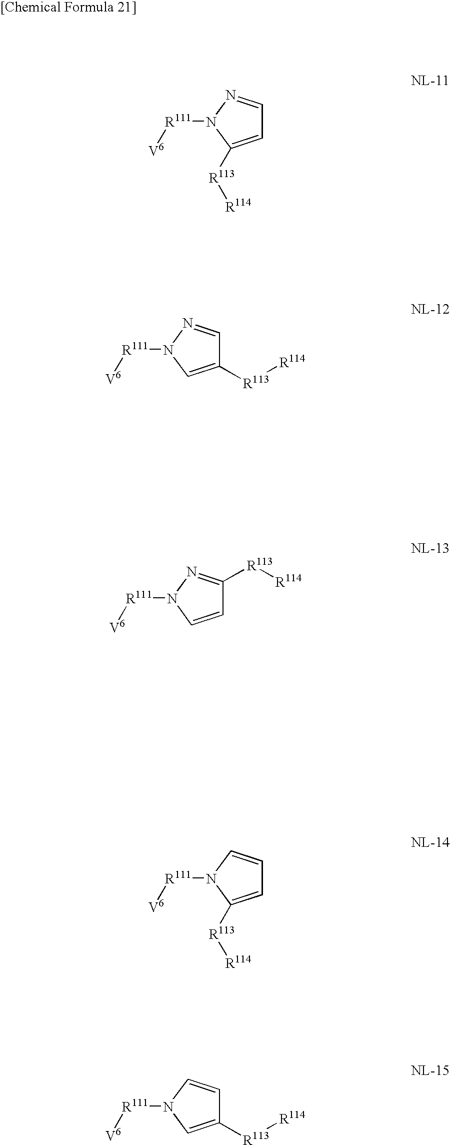

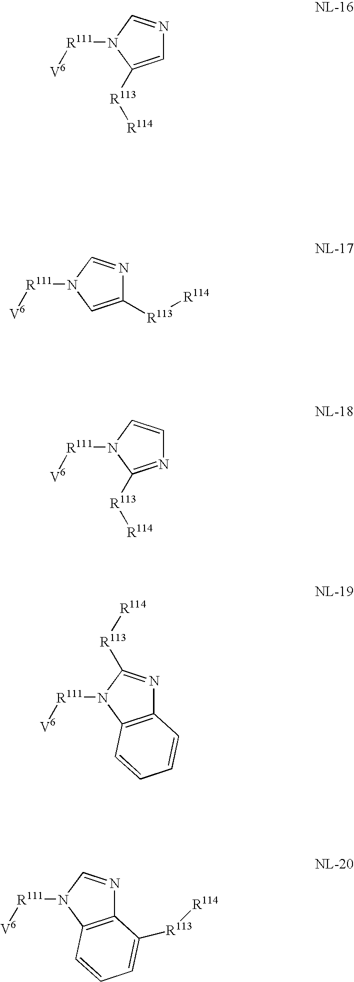

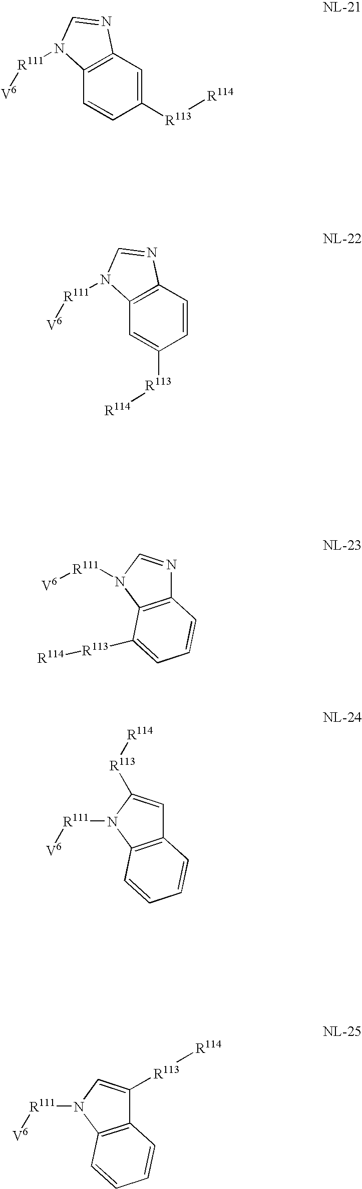

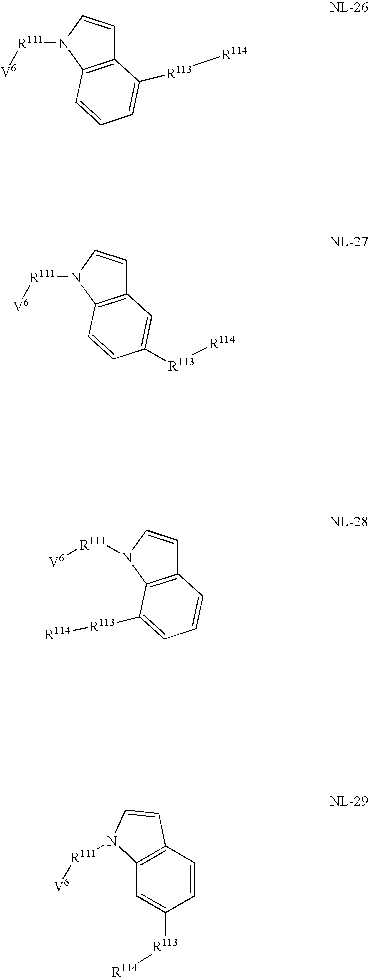

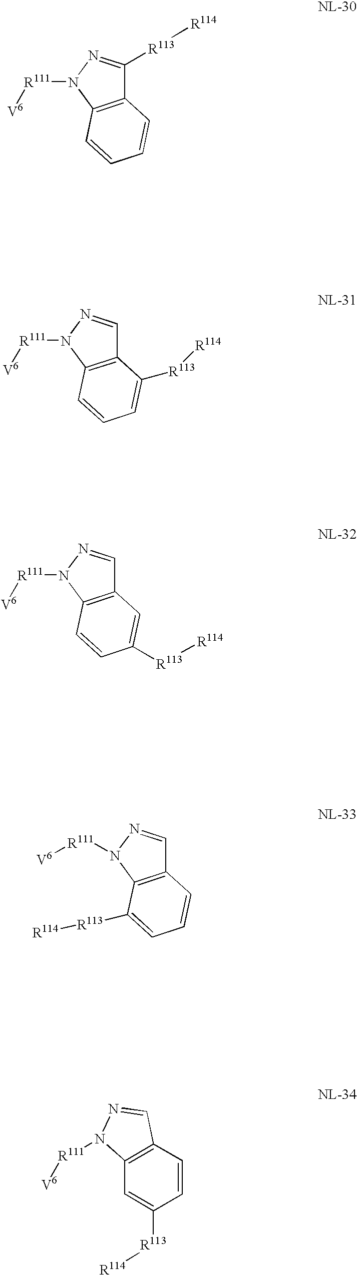

[In the Formulae NL-11 to NL-34, V⁶ represents a bonding arm with Ar¹ and R¹¹¹, R¹¹³ and R¹¹⁴ are the same as the aforementioned]

In the case where R¹¹¹ is —NR¹¹¹¹CO—, —NR¹¹¹¹ is bonded to Ar¹ and CO— is bonded to R¹¹². Similarly, in the case where R¹¹¹ is —CONR¹¹¹¹—, —CO is bonded to Ar¹ and NR¹¹¹¹— is bonded to R¹¹². In addition, in the case where R¹¹¹ is —NR¹¹¹¹SO₂—, —NR¹¹¹¹ is bonded to Ar¹ and SO₂— is bonded to R¹¹². Similarly, in the case where R¹¹¹ is —SO₂NR¹¹¹¹—, —SO₂ is bonded to Ar¹ and NR¹¹¹¹— is bonded to R¹¹². R¹¹¹ is preferably a single bond or —CH═CH—, and more preferably a single bond. In addition, in an alternative embodiment, R¹¹¹ is preferably —CH═CH—.

R¹¹² represents an optionally substituted arylene group or an optionally substituted alkylene group. R¹¹² is preferably an optionally substituted arylene group, more preferably the same as one of the preferable examples of the optionally substituted arylene group, and still more preferably an optionally substituted phenylene group.

In the case where R¹¹² is an optionally substituted arylene group, the arylene group in the optionally substituted arylene group is not particularly limited if it is one of the aforementioned arylene groups. However, the arylene group can be a bivalent residue formed by removing any two hydrogen atoms from benzene or thiophene.

In the examples of arylene, a bonding arm represented by V¹ is preferably bonded to R¹¹¹, and a bonding arm represented by V² is preferably bonded to R¹¹³. In addition, in an alternative preferable embodiment, a bonding arm represented by V¹ is bonded to R¹¹³, and a bonding arm represented by V² is bonded to R¹¹¹.

In addition, the substituent in the optionally substituted arylene group is not particularly limited if it is one of the favor able examples of the substituent in the aforementioned optionally substituted arylene group. However, it is preferably a halogen atom, an alkyl group, an alkoxy group or a cyano group, and more preferably a halogen atom or an alkyl group. The halogen atom is preferably a fluorine atom, a chlorine atom or a bromine atom, and more preferably a fluorine atom.

In the case where R¹¹¹¹ is present in R¹¹¹, R¹¹² may be bonded to R¹¹¹¹ to form a ring structure. Examples of a partial structure of —R¹¹¹—R¹¹²— in this case include the following.

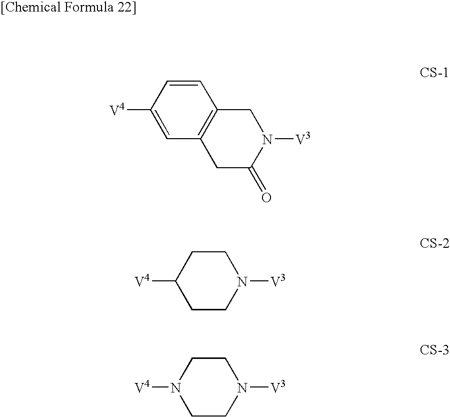

In the formulae, V³ represents a bonding arm to Ar¹, and V⁴ represents a bonding arm to R¹¹³

R¹¹³ represents a single bond, —O—, —NR¹¹³¹—, —NR¹¹³¹CO—, —CONR¹¹³¹—, —NR¹¹³¹SO₂—, —SO₂NR¹¹³¹— (R¹³¹ represents a hydrogen atom, or an optionally substituted alkyl group), —(C═O)—, —C≡C— or —CH═CH—. However, in the case where R¹¹² is a nitrogen atom-containing group, and the bonding position of R¹¹³ is on the nitrogen atom of R¹¹², R¹¹³ represents a single bond, —CONR¹¹³¹—, —SO₂NR¹¹³¹—, —(C═O)—, —C≡C— or —CH═CH—. Furthermore, in the case where R¹¹⁴ is a nitrogen atom-containing group, and the bonding position of R¹¹³ is on the nitrogen atom of R¹¹⁴, R¹¹³ represents a single bond, —NR¹¹³¹CO—, —NR¹¹³¹SO₂—, —(C═O)—, —C≡C— or —CH═CH—.

Examples of R¹¹ wherein R¹¹² is a nitrogen atom-containing group, and the bonding position of R¹¹³ is on the nitrogen atom of R¹¹² include groups represented by Formulae described below.

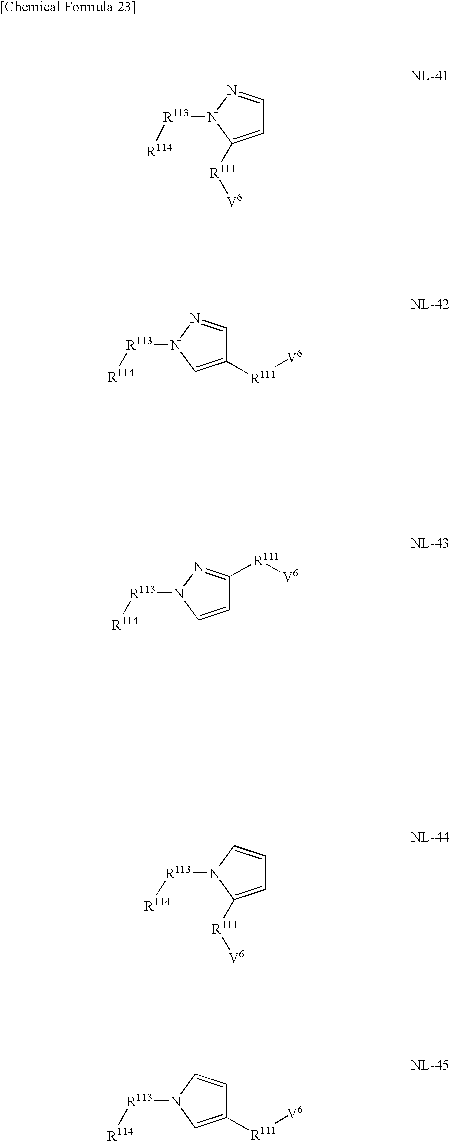

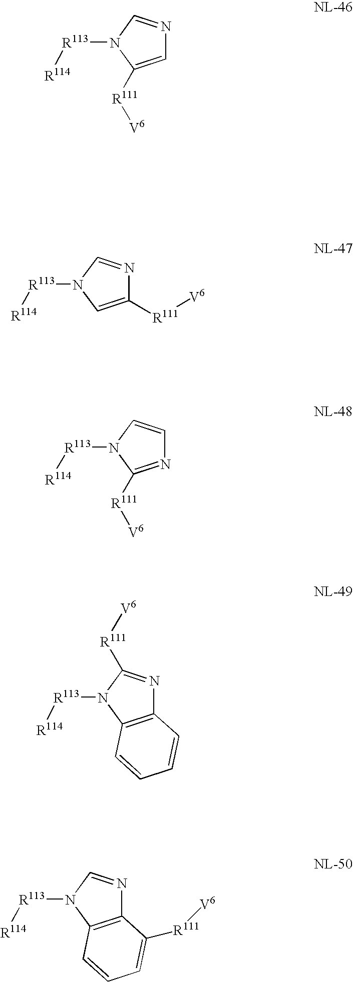

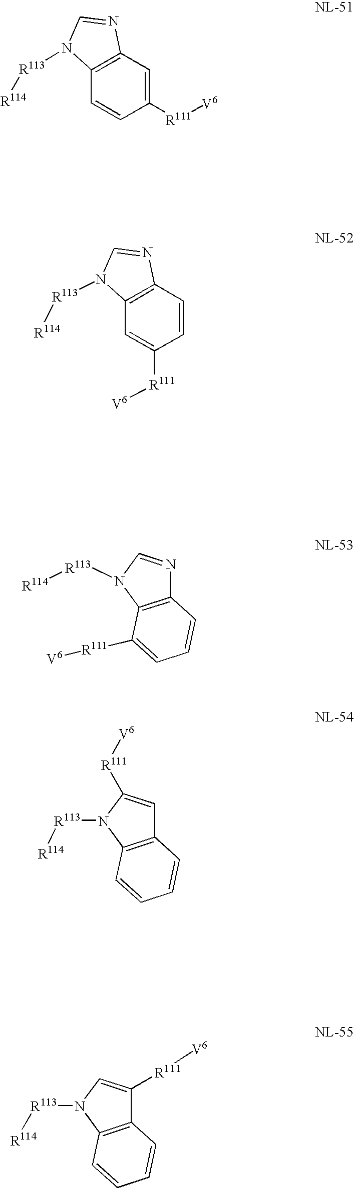

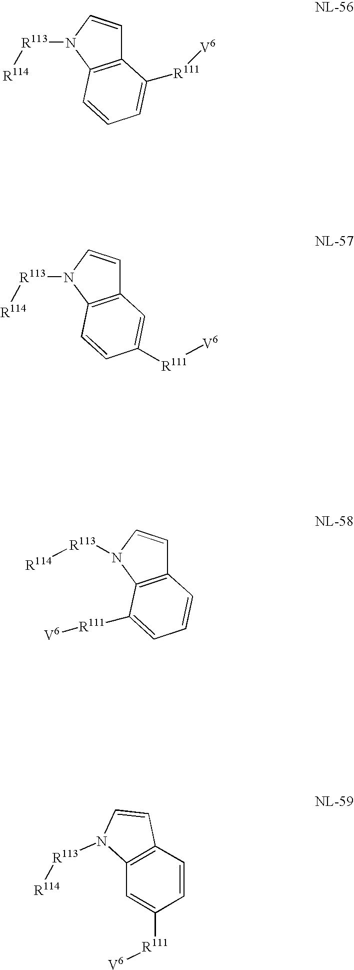

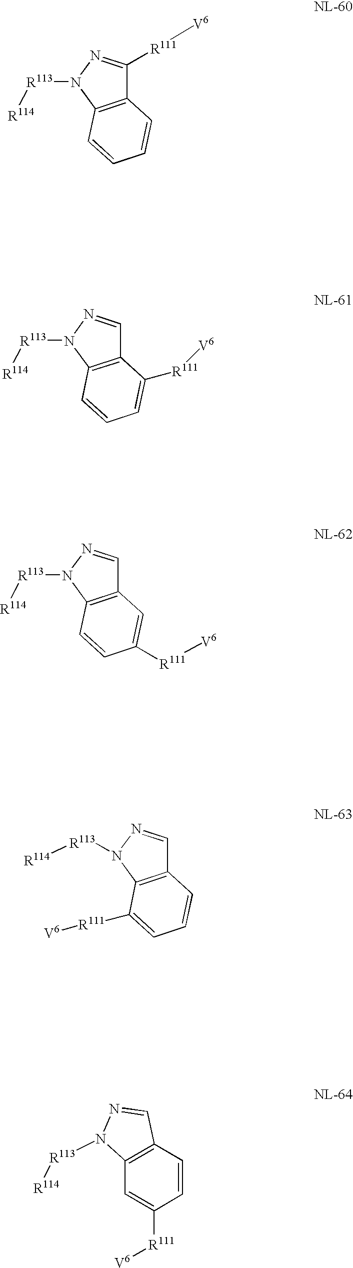

[In the Formulae NL-41 to NL-64, V⁶, R¹¹¹, R¹¹³ and R¹¹⁴ are the same as the aforementioned]

Examples of R¹¹⁴ wherein R¹¹⁴ is a nitrogen atom-containing group, and the bonding position of R¹¹³ is on the nitrogen atom of R¹¹⁴ include groups represented by Formulae described below.

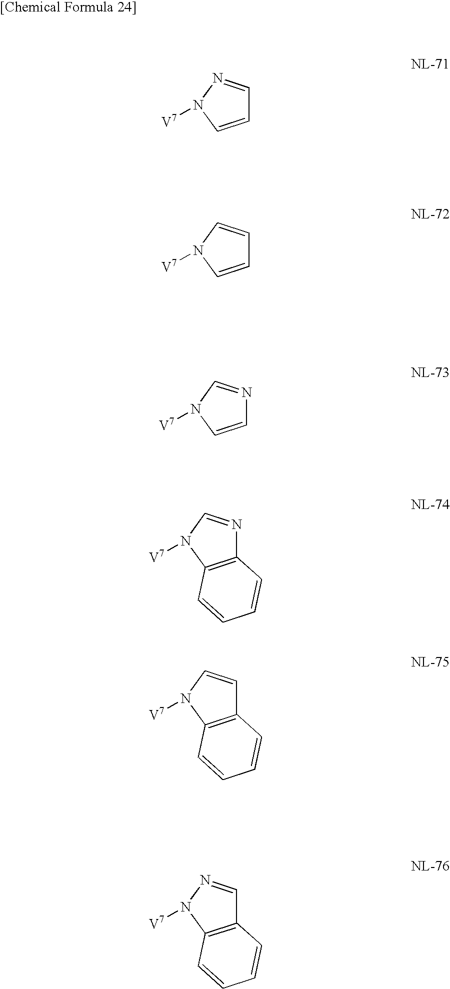

[In the Formulae NL-71 to NL-76, V⁷ represents a bonding arm with R¹¹³]

In the case that R¹¹³ is —NR¹¹³¹CO—, —NR¹¹³¹ is bonded to R¹¹² and CO— is bonded to R¹¹⁴. Similarly, in the case where R¹¹³ is —CONR¹¹³¹—, —CO is bonded to R¹¹² and NR¹¹³¹— is bonded to R¹¹⁴. In addition, in the case where R¹¹³ is —NR¹¹³¹SO₂—, —NR¹¹³¹ is bonded to R¹¹² and SO₂— is bonded to R¹¹⁴. Similarly, in the case where R¹¹³ is —SO₂NR¹¹³¹—, —SO₂ is bonded to R¹¹² and NR¹¹³¹— is bonded to R¹¹⁴. R¹¹³ is preferably a single bond or —(C═O)—, and more preferably a single bond. In addition, in an alternative embodiment, R¹¹³ is preferably —CH═CH— or —C≡C—, and more preferably —CH═CH—.

In the case where R¹¹³¹ is an optionally substituted alkyl group, the substituent in the optionally substituted alkyl group is not particularly limited if it is one of the favor able examples of the substituent in the aforementioned optionally substituted alkyl group. However, it is preferably a halogen atom or an alkoxy group, and more preferably a fluorine atom or an alkoxy group.

R¹¹⁴ represents an optionally substituted aryl group, an optionally substituted alkyl group or a hydrogen atom. R¹¹⁴ is preferably an optionally substituted aryl group or a hydrogen atom, more preferably the same as one of the preferable examples of the aforementioned aryl group, or a hydrogen atom, still more preferably a phenyl group, a 1-pyrazolyl group or a hydrogen atom, and still more preferably a phenyl group. In addition, in an alternative embodiment, R¹¹⁴ is preferably a 1-methyl-1H-indazol-5-yl group.

In the case where R¹¹⁴ is an optionally substituted aryl group, the aryl group in the optionally substituted aryl group is not particularly limited if it is one of the aforementioned aryl groups. R¹¹⁴ can be a monovalent residue formed by removing any one hydrogen atom from benzene or thiophene.

In addition, the substituent in the optionally substituted aryl group is not particularly limited if it is one of the favor able examples of the substituent in the aforementioned optionally substituted aryl group. However, it is preferably a halogen atom or an alkoxy group, and more preferably a fluorine atom or an alkoxy group. The substituent is still more preferably an alkoxy group. In addition, in an alternative embodiment, the substituent is preferably an optionally substituted amino group.

In the case where R¹¹⁴ is an optionally substituted alkyl group, the substituent in the optionally substituted alkyl group is not particularly limited if it is one of the favor able examples of the substituent in the aforementioned optionally substituted alkyl group. However, it is preferably a halogen atom or an alkoxy group, and more preferably a fluorine atom or an alkoxy group.

In the case where R¹¹³¹ is present in R¹¹³, R¹¹⁴ may be bonded to R¹¹¹³ to form a ring structure, and examples of the partial structure of —R¹¹³—R¹¹⁴ in this case include the following.

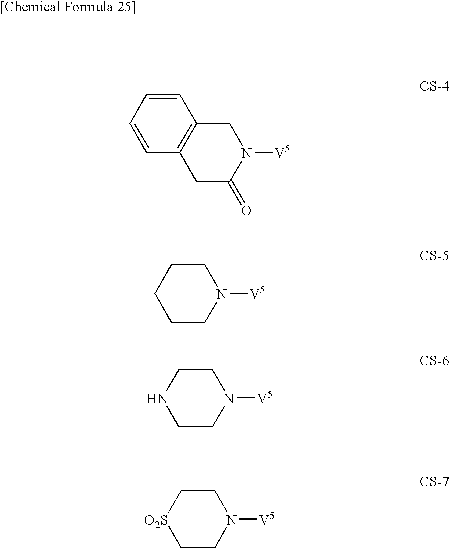

In the formula, V⁵ represents a bonding arm to R¹¹².

Favor able examples of R¹¹ as a combination of the R¹¹¹, R¹¹², R¹¹³ and R¹¹⁴ include substituents described below.

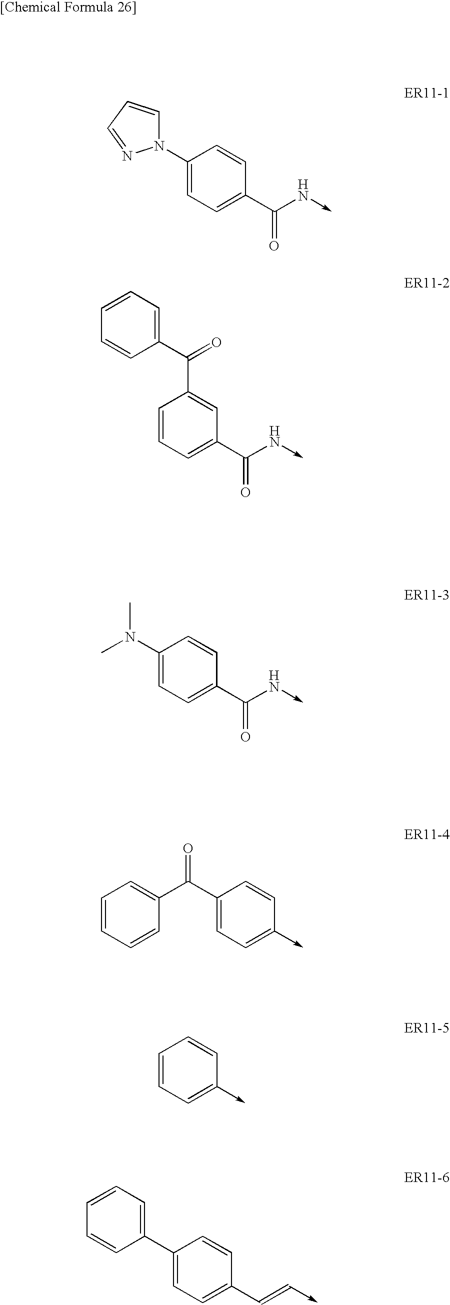

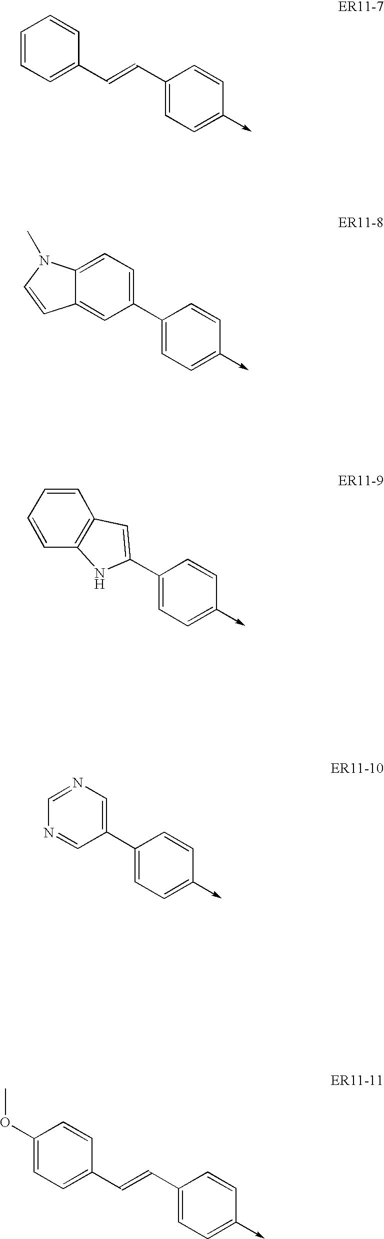

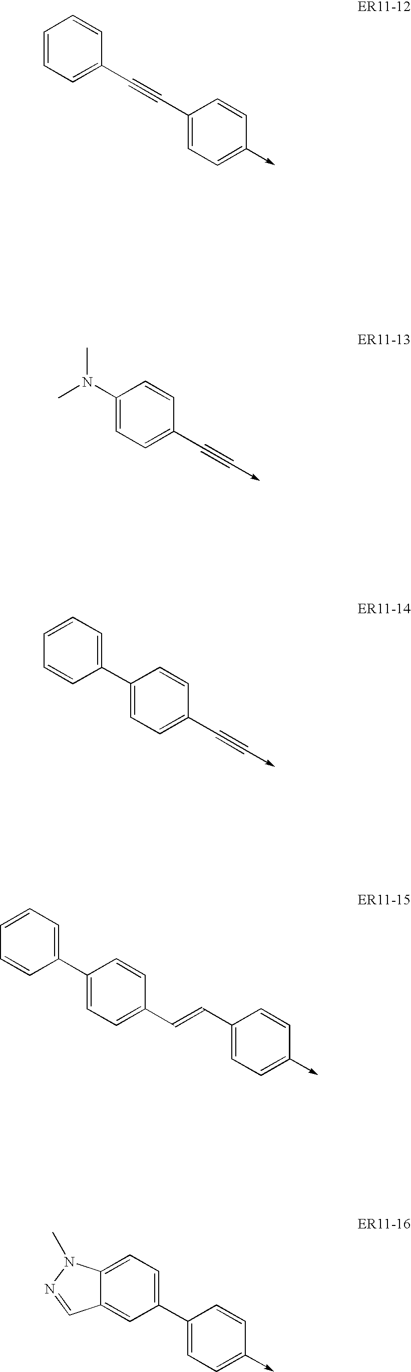

In the formulae, the arrow represents a bonding arm to Ar¹.

More favor able examples of R¹¹ include a substituent represented by the formula ER11-5. In addition, in an alternative embodiment, favor able examples thereof include a substituent represented by the formula ER11-11. In a further alternative embodiment, favor able examples thereof include a substituent represented by the formula ER11-15 or ER11-16.

R¹² and R¹³ can be the same or different, and independently represent a hydrogen atom, a halogen atom, a hydroxyl group, an optionally substituted alkyl group, an optionally substituted alkenyl group, an optionally substituted alkynyl group, an optionally substituted alkoxy group, or an optionally substituted amino group, or they can be absent. More specifically, the absence and the presence of R¹² and R¹³ are determined by formula (2), which is a partial structure of the formula (1).

1. When the structure of formula (2) corresponds to PD-1, PD-2, PD-3, PD-4, PR-1, PR-2, PR-3, PR-4, or PR-5 described in the above, both R¹² and R¹³ are present.
2. When the structure of formula (2) corresponds to TP-1, TP-2, TP-3, FR-1, FR-2, FR-3, PZ-1, PZ-2, PZ-3, PZ-4, PA-1, PM-1, PM-2, IZ-1, IZ-2, IZ-3, IZ-4, IZ-5, PN-1, PN-2, or PN-3 described in the above, R¹² is present and R¹³ is absent.
3. When the structure of formula (2) corresponds to TZ-1, TZ-2, OZ-1, OZ-2, IT-1, IT-2, IT-3, IT-4, IO-1, IO-2, IO-3, IO-4, TR-1, TR-2, TR-3, TR-4, TA-1, TA-2, or TA-3 described in the above, both R¹² and R¹³ are absent.

When both R¹² and R¹³ are present, they are preferably hydrogen atoms.

The optionally substituted alkyl group for R¹² and R¹³ is not specifically limited if each of them is one of the preferred examples of the aforementioned optionally substituted alkyl group. Preferably, it is a methyl group or an ethyl group.

The optionally substituted alkenyl group for R¹² and R¹³, is not specifically limited if each of them is one of the preferred examples of the aforementioned optionally substituted alkenyl group. Preferably, it is a vinyl group or an allyl group.

The optionally substituted alkynyl group for R¹² and R¹³ is not specifically limited if each of them is one of the preferred examples of the aforementioned optionally substituted alkynyl group. Preferably, it is an ethynyl group or a prop-2-yl group.

The optionally substituted alkoxy group for R¹² and R¹³ is not specifically limited if each of them is one of the preferred examples of the aforementioned optionally substituted alkoxy group. Preferably, it is a methoxy group or an ethoxy group.

The optionally substituted amino group for R¹² and R¹³ is not specifically limited if each of them is one of the preferred examples of the aforementioned optionally substituted amino group. Preferably, it is a methylamino group, or an acetylamino group.

R²¹ and R²² can be the same or different, and independently represent a hydrogen atom, an optionally substituted alkyl group, an optionally substituted aryl group, an optionally substituted alkoxy group, an optionally substituted aryloxy group, or an optionally substituted amino group. Both R²¹ and R²² are preferably hydrogen atoms. In another embodiment, both R²¹ and R²² are preferably methyl groups.

The optionally substituted alkyl group for R²¹ and R²² is not specifically limited if each of them is one of the preferred examples of the aforementioned optionally substituted alkyl group. Preferably, it is a methyl group, an ethyl group, or a benzyl group.

The optionally substituted aryl group for R²¹ and R²² is not specifically limited if each of them is one of the preferred examples of the aforementioned optionally substituted aryl group. Preferably, it is a phenyl group or a thienyl group.

The optionally substituted alkoxy group for R²¹ and R²² is not specifically limited if each of them is one of the preferred examples of the aforementioned optionally substituted alkoxy group. Preferably, it is a methoxy group or an ethoxy group.

The optionally substituted aryloxy group for R²¹ and R²² is not specifically limited if each of them is one of the preferred examples of the aforementioned optionally substituted aryloxy group. Preferably, it is a phenoxy group or a 2-thienyloxy group.

The optionally substituted amino group for R²¹ and R²² is not specifically limited if each of them is one of the preferred examples of the aforementioned optionally substituted amino group. Preferably, it is a methylamino group, or an acetylamino group.

R³¹ represents a hydrogen atom, an optionally substituted alkyl group, an optionally substituted aryl group, an optionally substituted alkoxy group or an optionally substituted aryloxy group. Preferable examples of R³¹ include an optionally substituted alkyl group and an optionally substituted aryl group. More preferable examples of R³¹ include the preferable examples of the optionally substituted alkyl group and optionally substituted aryl group. The most preferable examples of R³¹ include an isopropyl group, a cyclohexyl group and a phenyl group. In addition, in an alternative embodiment, preferable examples of R³¹ include an ethyl group.

The optionally substituted alkyl group for R³¹ is not particularly limited if it is one of the favor able examples of the aforementioned optionally substituted alkyl group. However, it is preferably an isopropyl group, a cyclohexyl group or an isobutyl group. In addition, in an alternative embodiment, preferable examples of the optionally substituted alkyl group include an ethyl group.

The optionally substituted aryl group for R³¹ is not particularly limited if it is one of the favor able examples of the aforementioned optionally substituted aryl group. However, it is preferably a phenyl group or a thienyl group.

The optionally substituted alkoxy group for R³¹ is not particularly limited if it is one of the favor able examples of the aforementioned optionally substituted alkoxy group. However, it is preferably a methoxy group or an ethoxy group.

The optionally substituted aryloxy group for R³¹ is not particularly limited if it is one of the favor able examples of the aforementioned optionally substituted aryloxy group. However, it is preferably a phenoxy group or a 2-thienyloxy group.

The carbon atom to which R³¹ is bonded may be an asymmetric center. The stereoisomers due to the asymmetric carbon are exemplified by Formula (5) and Formula (6) described below [In Formulae (5) and (6), Ar¹, R¹¹, R¹², R¹³, R²¹, R²², R³¹, n, m, and Y¹ are the same as the aforementioned].

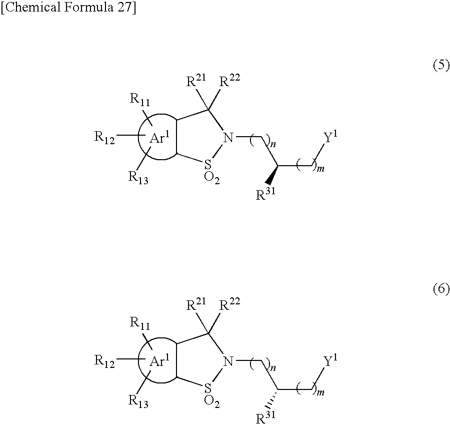

Among the isomers, a preferable example is the isomer represented by Formula (5).

R²¹, or R³¹ and R²² may form a ring. When a ring is formed, it is preferably a 5- to 7-membered saturated heterocycloalkane ring. A compound wherein R²¹ and R³¹, and R²² and R³¹ do not form a ring is preferable.

n and m may be the same or different, and independently represent 0 or 1. Preferably, both of n and m are 0.

Y¹ represents —COY¹¹ (Y¹¹ represents —NHOH, —OH, —NH₂, an amino group substituted with one or two optionally substituted alkyl groups, an amino group substituted with one or two optionally substituted aryl groups or an amino group substituted with an optionally substituted aryl group and an optionally substituted alkyl group). Preferable examples of Y¹¹ include —NHOH and —OH. More preferable examples of Y¹¹ include —NHOH. In addition, in an alternative embodiment, more preferable examples of Y¹¹ include —OH.

The combination of the substituents in the compound represented by Formula (1) is not particularly limited, but is, for example,

<1> a compound wherein Ar¹ is a thiophene ring;
<2> a compound wherein Ar¹ is a thiophene ring, and which is represented by formula (3);
<3> a compound wherein Ar¹ is a thiophene ring, and which is represented by formula (4);
<4> a compound wherein Ar¹ is a thiophene ring, and the substituted position of R¹¹ is represented by PR-11-1;
<5> a compound wherein Ar¹ is a thiophene ring, and the substituted position of R¹¹ is represented by PR-11-3;
<6> a compound wherein R¹¹¹ is a single bond;
<7> A compound wherein R¹¹¹ is —NHCO—;
<8> A compound wherein R¹¹¹ is —CH═CH—;
<9> A compound wherein R¹¹¹ is —C≡C—;
<10> A compound wherein R¹¹² is an optionally substituted aryl group;
<11> A compound wherein R¹¹² is an optionally substituted phenyl group;
<12> A compound wherein R¹¹³ is a single bond;
<13> A compound wherein R¹¹³ is —(C═O)—;
<14> A compound wherein R¹¹³ is —CH═CH—;
<15> A compound wherein R¹¹³ is —C≡C—;
<16> A compound wherein R¹¹⁴ is a hydrogen atom;
<17> A compound wherein R¹¹⁴ is an optionally substituted aryl group;
<18> A compound wherein R¹¹⁴ is an optionally substituted phenyl group;
<19> A compound wherein R¹¹⁴ is an optionally substituted indolyl group;
<20> A compound wherein R¹¹⁴ is an optionally substituted pyrimidinyl group;
<21> A compound wherein R¹¹⁴ is an optionally substituted indazolyl group;
<22> A compound wherein R¹² is a hydrogen atom;
<23> A compound wherein R¹³, if present, is a hydrogen atom;
<24> A compound wherein both of R²¹ and R²² are hydrogen atoms;
<25> A compound wherein both of R²¹ and R²² are methyl groups;
<26> A compound wherein R³¹ is an optionally substituted alkyl group;
<27> A compound wherein R³¹ is an optionally substituted aryl group;
<28> A compound wherein R³¹ is an isopropyl group;
<29> A compound wherein R³¹ is a phenyl group;
<30> A compound wherein R³¹ is an ethyl group;
<31> A compound wherein the asymmetric carbon to which R³¹ is bonded has the configuration represented by formula (5);
<32> A compound wherein R²¹ and R³¹, and R²² and R³¹ form no ring;
<33> A compound wherein both of n and m are 0;
<34> A compound wherein Y¹¹ is —NHOH;
<35> A compound wherein Y¹¹ is —OH;
<36> A compound according to any one of the <1> to <5> wherein the compound is further according to <6>;
<37> A compound according to any one of the <1> to <5> wherein the compound is further according to <7>;
<38> A compound according to any one of the <1> to <5> wherein the compound is further according to <8>;
<39> A compound according to any one of the <1> to <5> wherein the compound is further according to <9>;
<40> A compound according to any one of the <1> to <9> or <36> to <39> wherein the compound is further according to <10>;
<41> A compound according to any one of the <1> to <9> or <36> to <39> wherein the compound is further according to <11>;
<42> A compound according to any one of the <1> to <11> or <36> to <41> wherein the compound is further according to <12>;
<43> A compound according to any one of the <1> to <11> or <36> to <41> wherein the compound is further according to <13>;
<44> A compound according to any one of the <1> to <11> or <36> to <41> wherein the compound is further according to <14>;
<45> A compound according to any one of the <1> to <11> or <36> to <41> wherein the compound is further according to <15>;
<46> A compound according to any one of the <1> to <15> or <36> to <45> wherein the compound is further according to <16>;
<47> A compound according to any one of the <1> to <15> or <36> to <45> wherein the compound is further according to <17>;
<48> A compound according to any one of the <1> to <15> or <36> to <45> wherein the compound is further according to <18>;
<49> A compound according to any one of the <1> to <15> or <36> to <45> wherein the compound is further according to <19>;
<50> A compound according to any one of the <1> to <15> or <36> to <45> wherein the compound is further according to <20>;
<51> A compound according to any one of the <1> to <15> or <36> to <45> wherein the compound is further according to <21>;
<52> A compound according to any one of the <1> to <21> or <36> to <51> wherein the compound is further according to <22>;
<53> A compound according to any one of the <1> to <22> or <36> to <52> wherein the compound is further according to <23>;
<54> A compound according to any one of the <1> to <23> or <36> to <53> wherein the compound is further according to <24>;
<55> A compound according to any one of the <1> to <23> or <36> to <53> wherein the compound is further according to <25>;
<56> A compound according to any one of the <1> to <25> or <36> to <55> wherein the compound is further according to <26>;
<57> A compound according to any one of the <1> to <25> or <36> to <55> wherein the compound is further according to <27>;
<58> A compound according to any one of the <1> to <25> or <36> to <55> wherein the compound is further according to <28>;
<59> A compound according to any one of the <1> to <25> or <36> to <55> wherein the compound is further according to <29>;
<60> A compound according to any one of the <1> to <25> or <36> to <55> wherein the compound is further according to <30>;
<61> A compound according to any one of the <1> to <30> or <36> to <60> wherein the compound is further according to <31>;
<62> A compound according to any one of the <1> to <31> or <36> to <61> wherein the compound is further according to <32>;
<63> A compound according to any one of the <1> to <32> or <36> to <62> wherein the compound is further according to <33>;
<64> A compound according to any one of the <1> to <33> or <36> to <63> wherein the compound is further according to <34>;
<64> A compound according to any one of the <1> to <33> or <36> to <63> wherein the compound is further according to <35>;

a salt of the compound represented by Formula (1), or a salt of the compound according to of the <1> to <65> which has a restricted combination of the substituents in the compound represented by Formula (1);

a pharmacologically acceptable salt of the compound represented by Formula (1), or a pharmacologically acceptable salt of the compound according to the <1> to <65> which has a restricted combination of the substituents in the compound represented by Formula (1);

a prodrug of the compound represented by Formula (1), or

a prodrug of the compound according to the <1> to <65> which has a restricted combination of the substituents in the compound represented by Formula (1);

a prodrug of the salt of the compound represented by Formula (1), or a prodrug of the salt of the compound according to the <1> to <65> which has a restricted combination of the substituents in the compound represented by Formula (1); and

a prodrug of the pharmacologically acceptable salt of the compound represented by Formula (1), or a prodrug of the pharmacologically acceptable salt of the compound according to the <1> to <65> which has a restricted combination of the substituents in the compound represented by Formula (1).

Specifically, preferable examples of the compound of the present invention represented by Formula (1) include compounds described below:

TABLE 1

| | |
|---|---|
| AS1-001 | 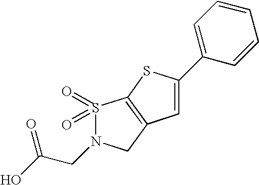 |
| AS1-002 | 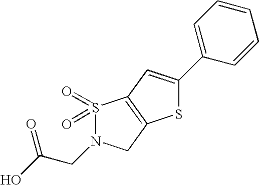 |
| AS1-003 | 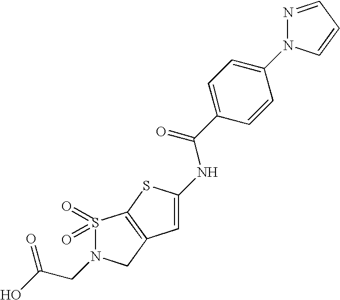 |
| AS1-004 | 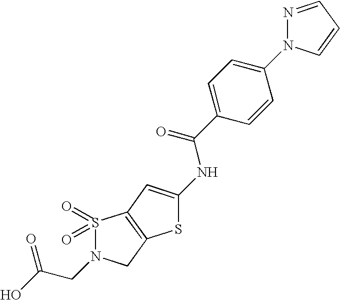 |
| AS1-005 | 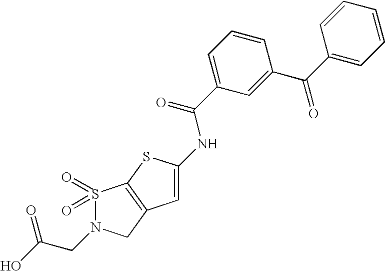 |
| AS1-006 | 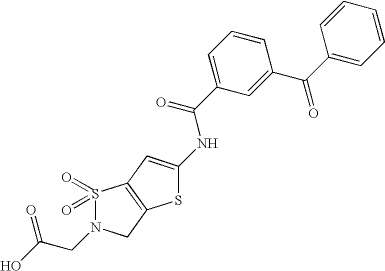 |
| AS1-007 | 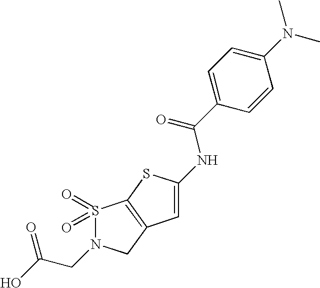 |
| AS1-008 | 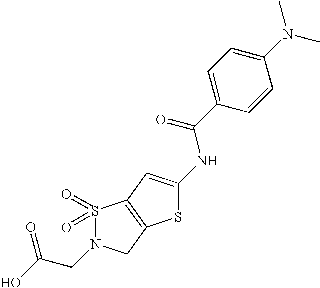 |
| AS1-009 | 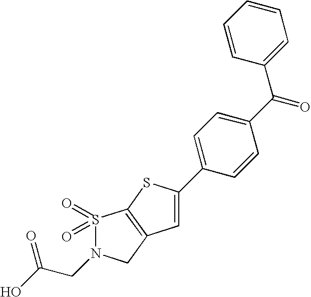 |
| AS1-010 | 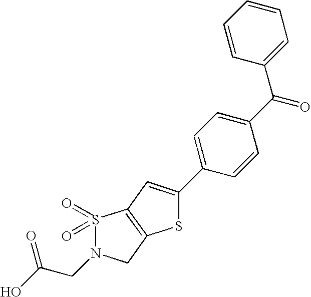 |
| AS1-011 | 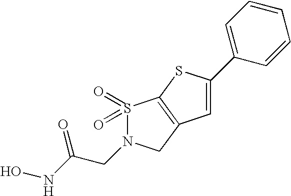 |
| AS1-012 | 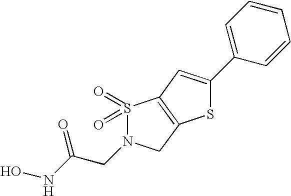 |
| AS1-013 | 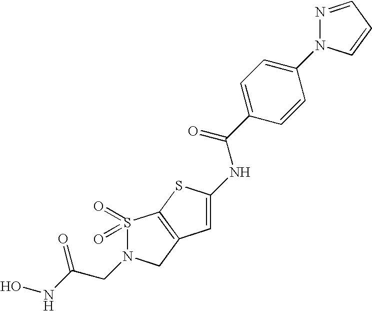 |
| AS1-014 | 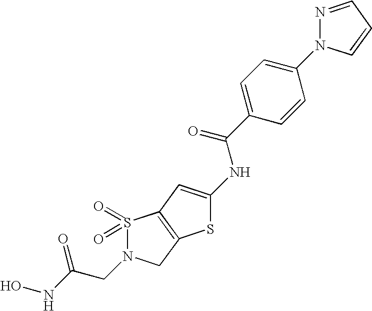 |
| AS1-015 | 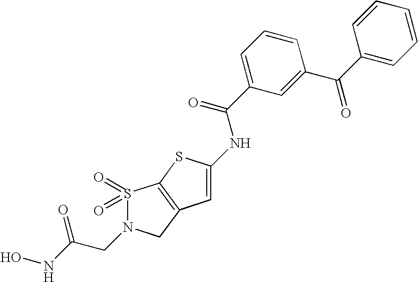 |
| AS1-016 | 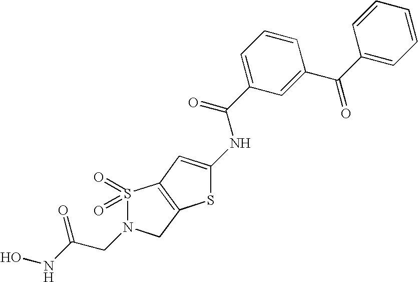 |
| AS1-017 | 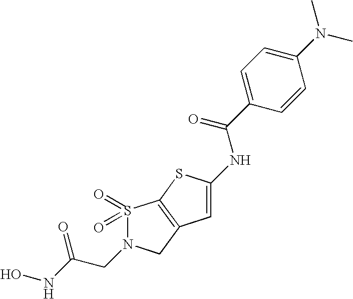 |
| AS1-018 | 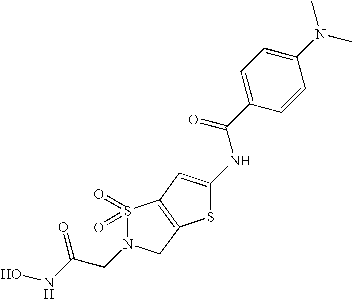 |
| AS1-019 | 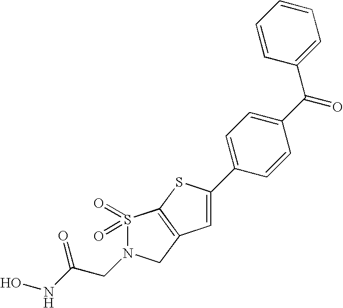 |
| AS1-020 | 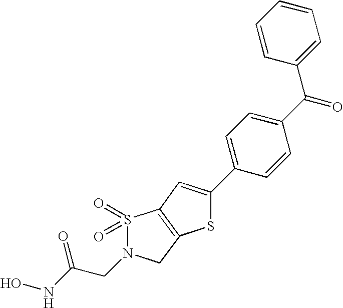 |
| AS1-021 | 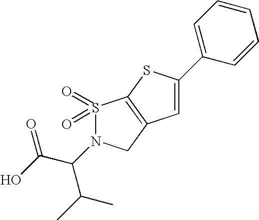 |
| AS1-022 | 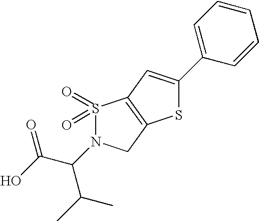 |
| AS1-023 | 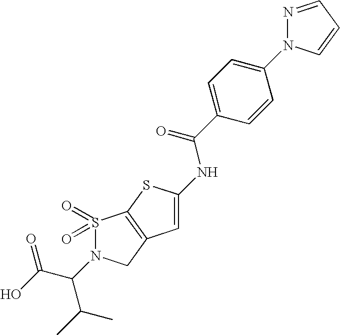 |
| AS1-024 | 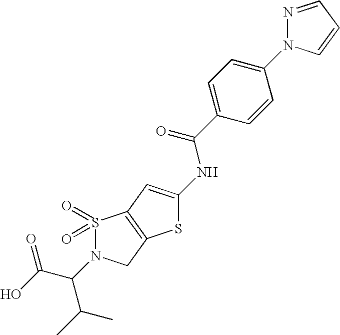 |
| AS1-025 | 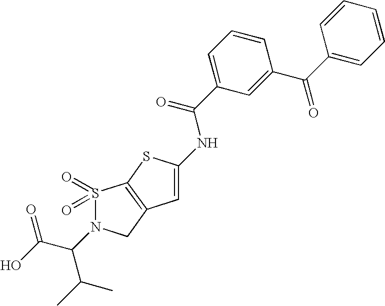 |
| AS1-026 | 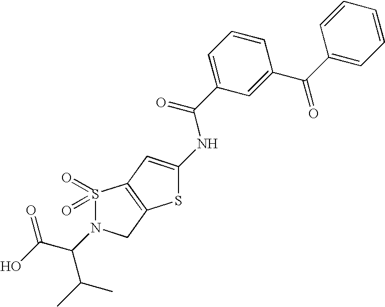 |
| AS1-027 | 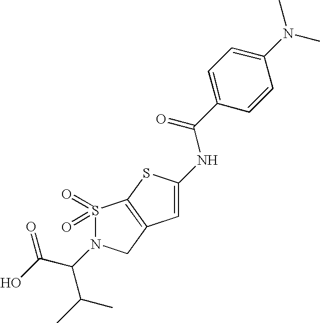 |
| AS1-028 | 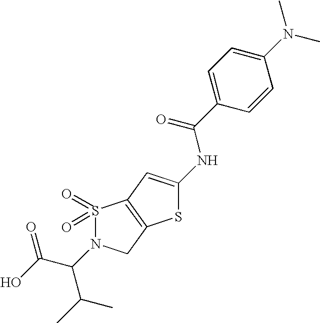 |
| AS1-029 | 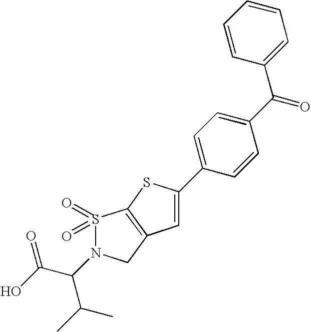 |
| AS1-030 | 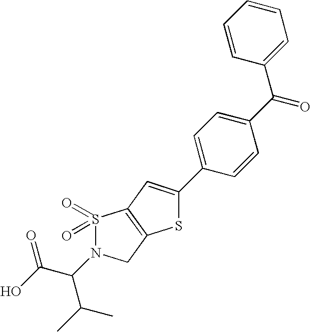 |
| AS1-031 | 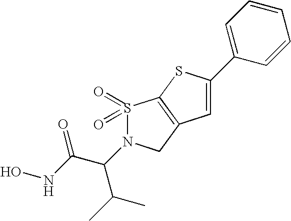 |
| AS1-032 | 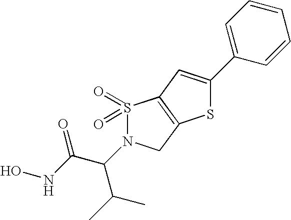 |
| AS1-033 | 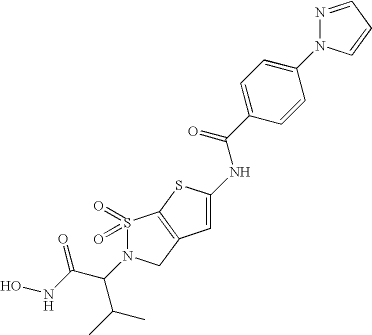 |
| AS1-034 | 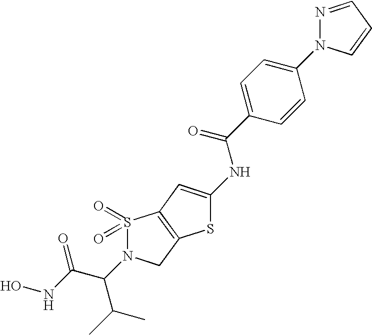 |
| AS1-035 | 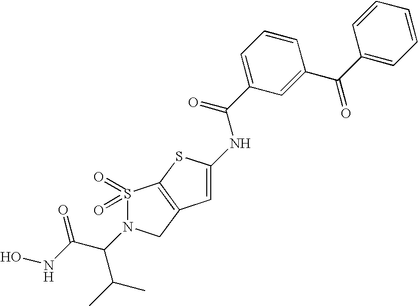 |
| AS1-036 | 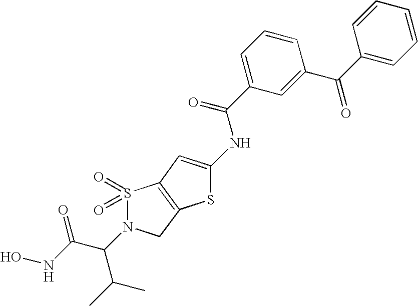 |
| AS1-037 | 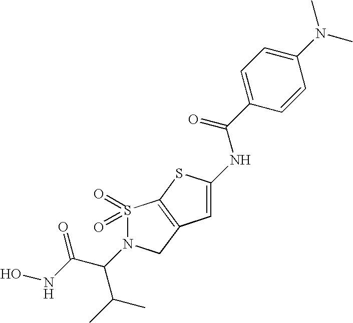 |
| AS1-038 | 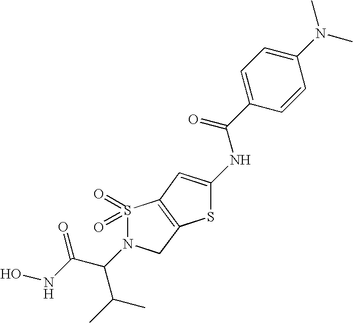 |
| AS1-039 | 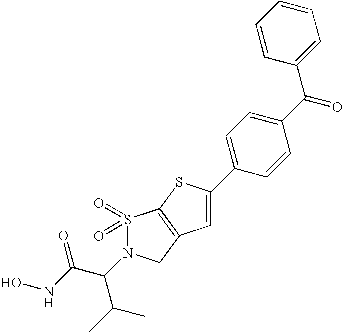 |
| AS1-040 | 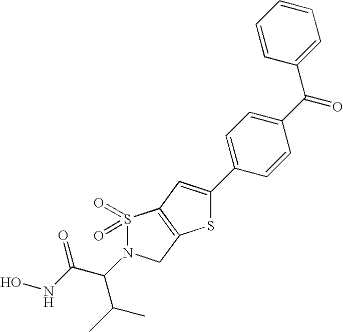 |
| AS1-041 | 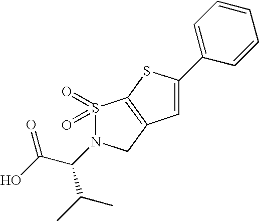 |
| AS1-042 | 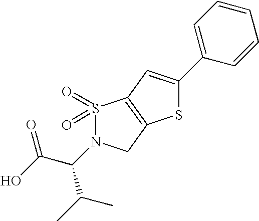 |
| AS1-043 | 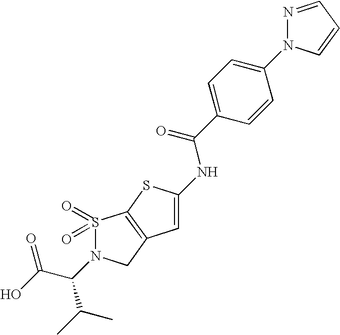 |
| AS1-044 | 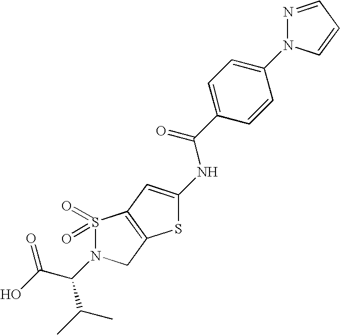 |
| AS1-045 | 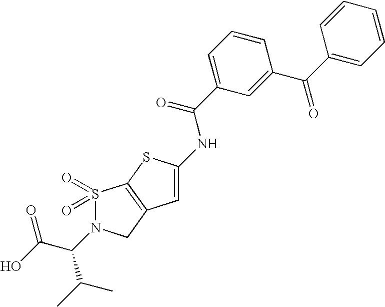 |
| AS1-046 | 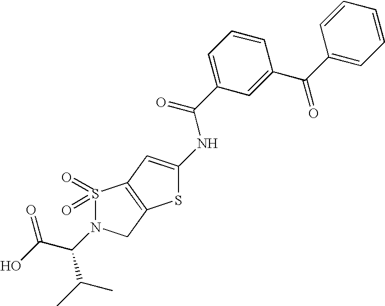 |
| AS1-047 | 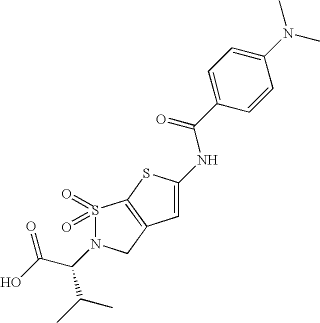 |
| AS1-048 | 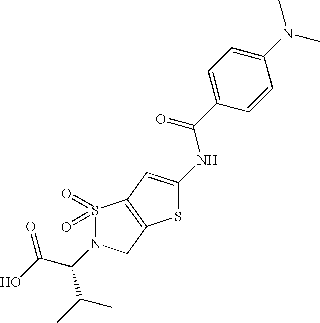 |
| AS1-049 | 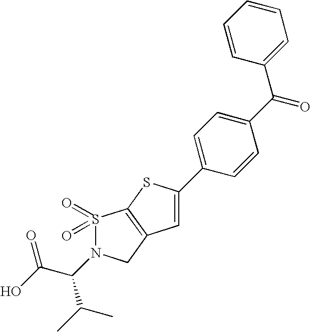 |
| AS1-050 | 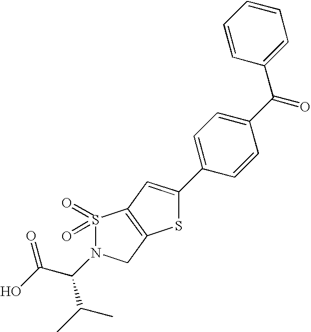 |
| AS1-051 | 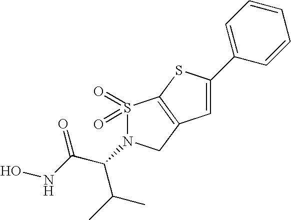 |
| AS1-052 | 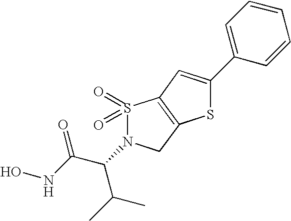 |
| AS1-053 | 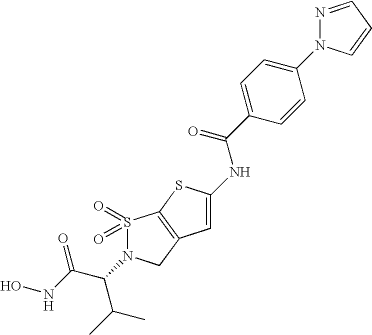 |
| AS1-054 | 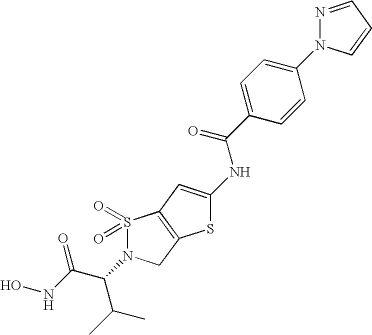 |
| AS1-055 | 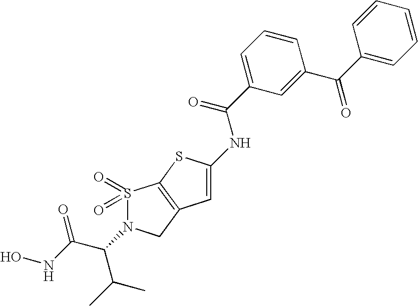 |
| AS1-056 | 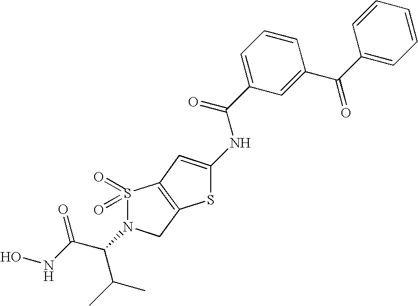 |
| AS1-057 | 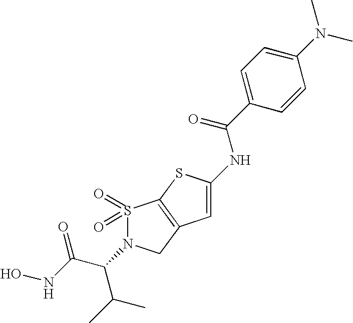 |
| AS1-058 | 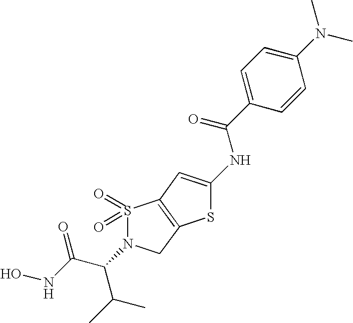 |
| AS1-059 | 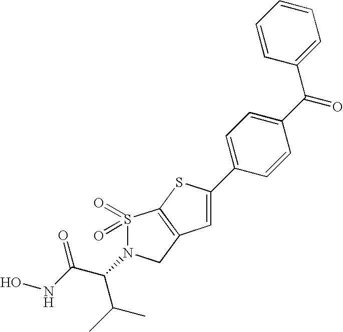 |
| AS1-060 | 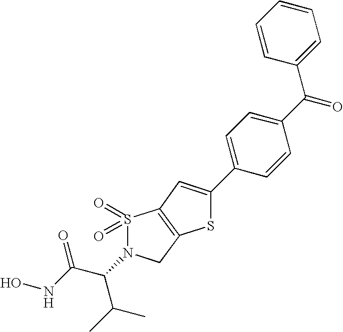 |
| AS1-061 | 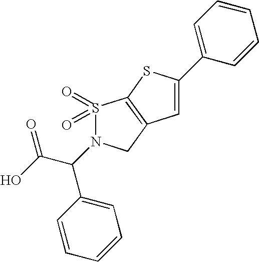 |
| AS1-062 | 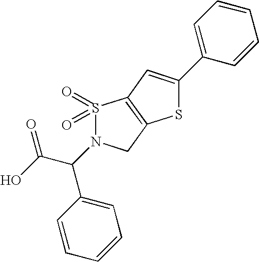 |
| AS1-063 | 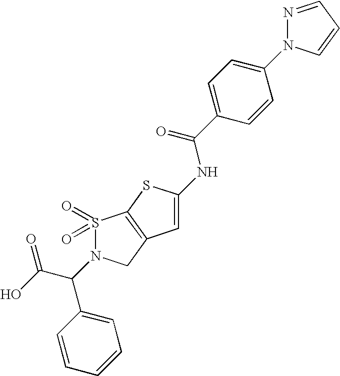 |
| AS1-064 | 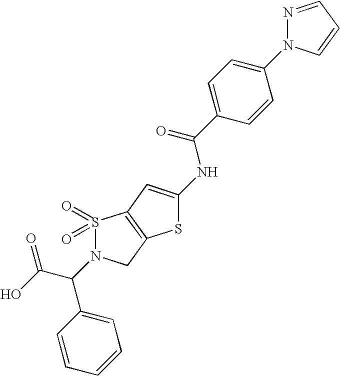 |
| AS1-065 | 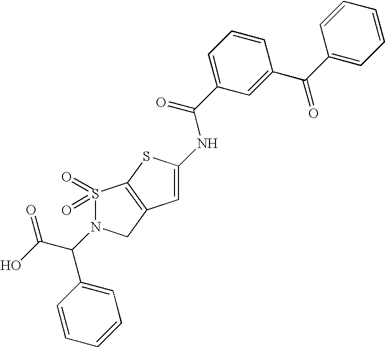 |
| AS1-066 | 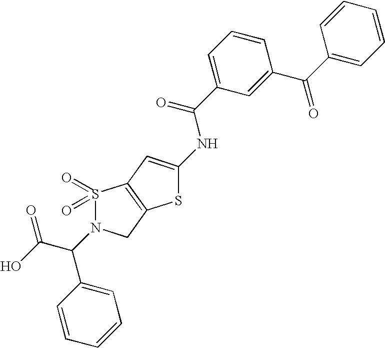 |
| AS1-067 | 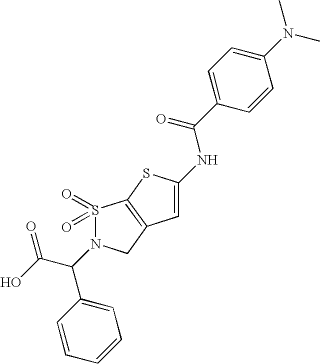 |
| AS1-068 | 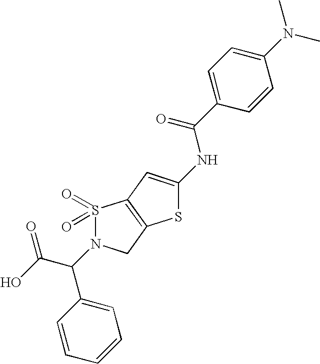 |
| AS1-069 | 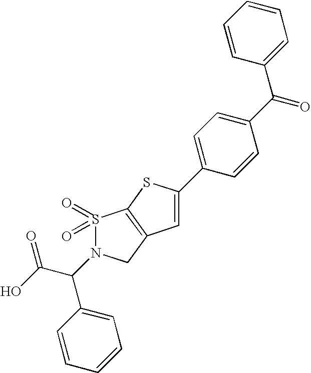 |
| AS1-070 | 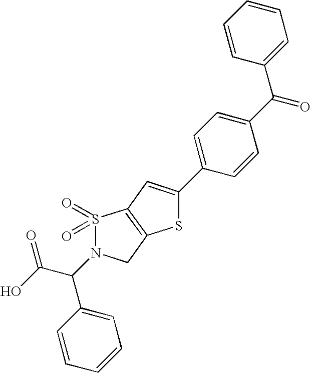 |
| AS1-071 | 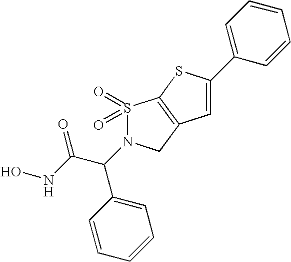 |
| AS1-072 | 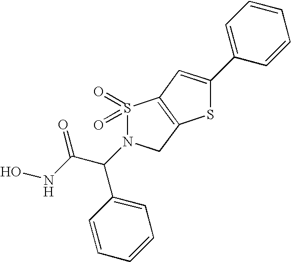 |
| AS1-073 | 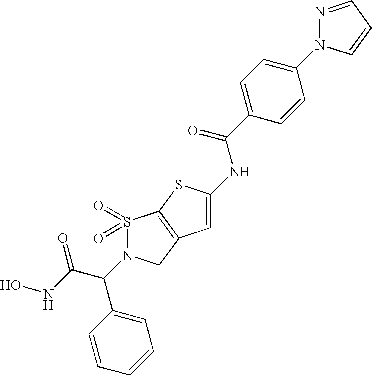 |
| AS1-074 | 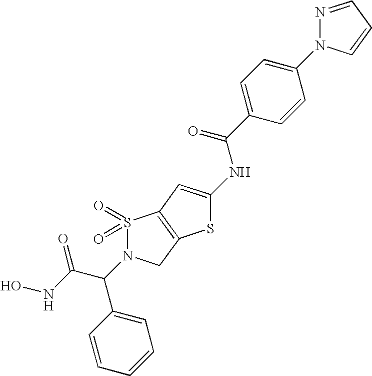 |
| AS1-075 | 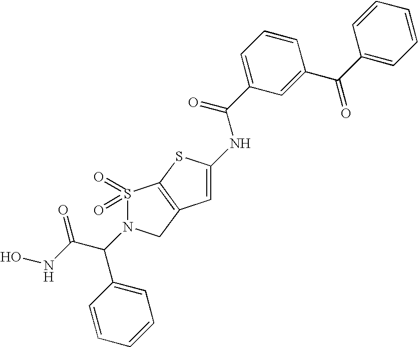 |
| AS1-076 | 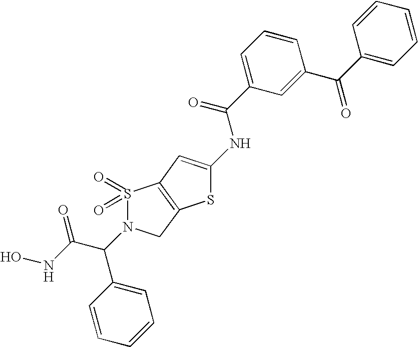 |
| AS1-077 | 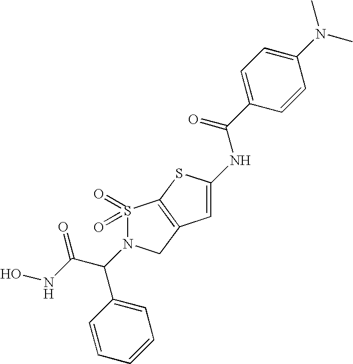 |
| AS1-078 | 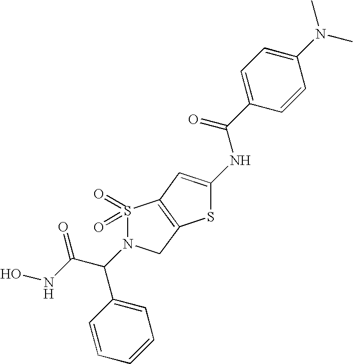 |
| AS1-079 | 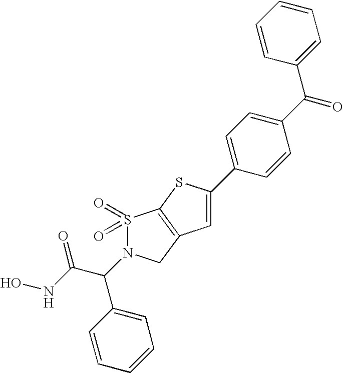 |
| AS1-080 | 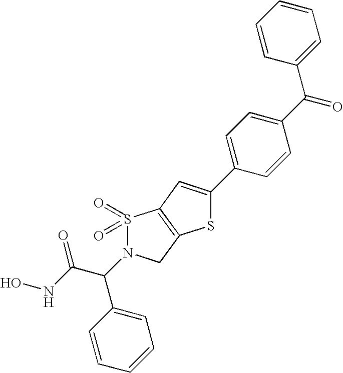 |
| AS1-081 | 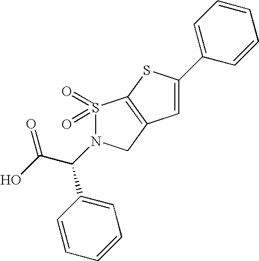 |
| AS1-082 | 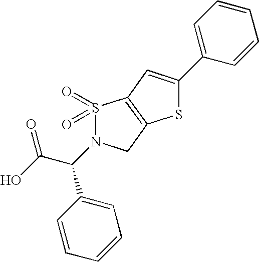 |
| AS1-083 | 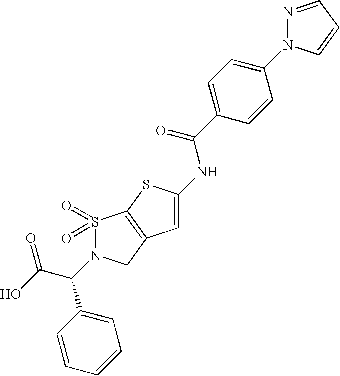 |
| AS1-084 | 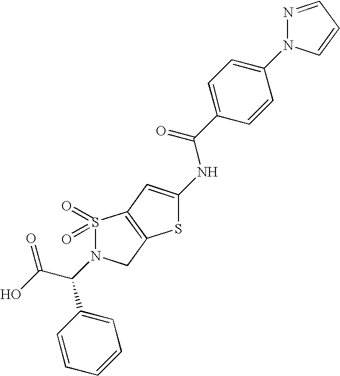 |
| AS1-085 | 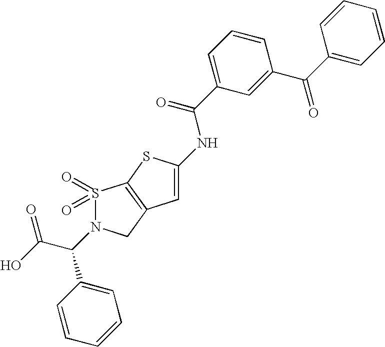 |
| AS1-086 | 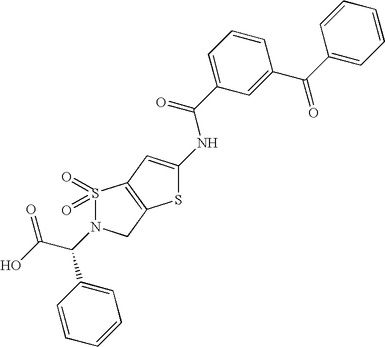 |
| AS1-087 | 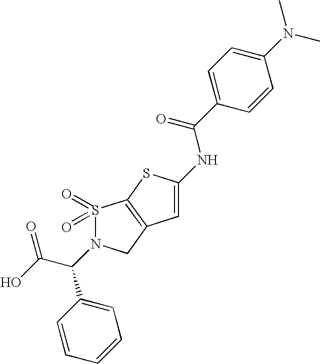 |
| AS1-088 | 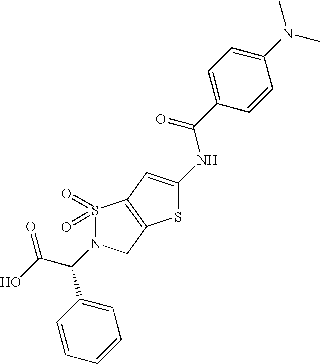 |
| AS1-089 | 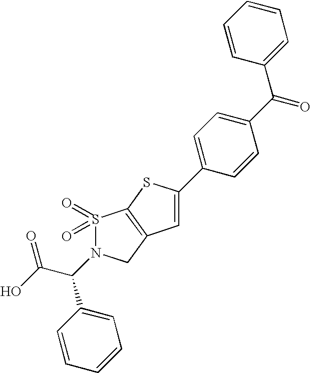 |
| AS1-090 | 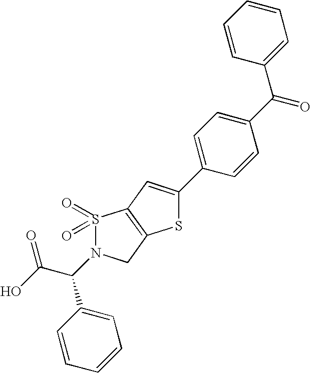 |
| AS1-091 | 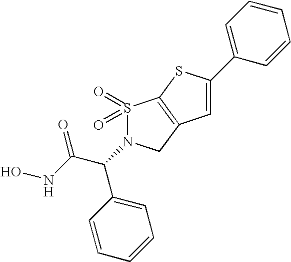 |
| AS1-092 | 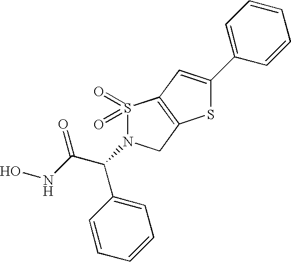 |
| AS1-093 | 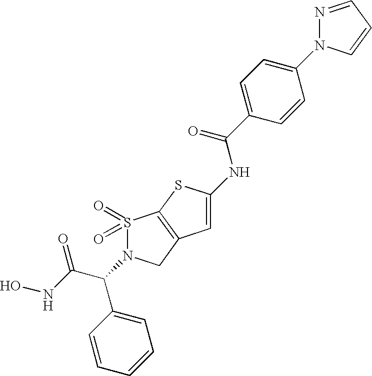 |
| AS1-094 | 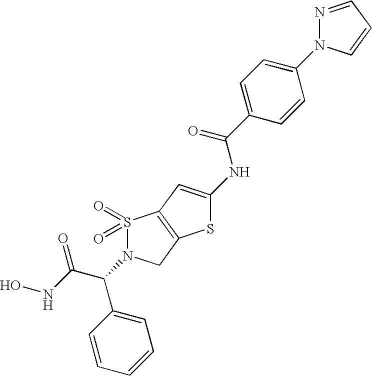 |
| AS1-095 | 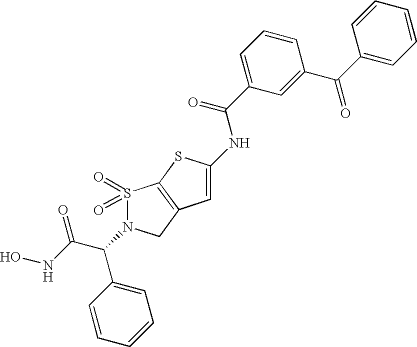 |
| AS1-096 | 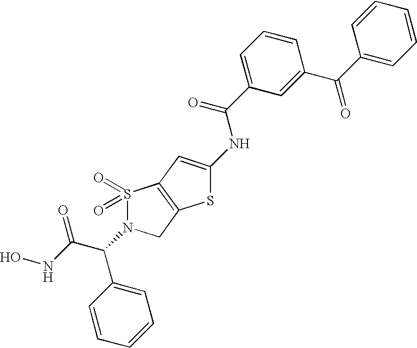 |
| AS1-097 | 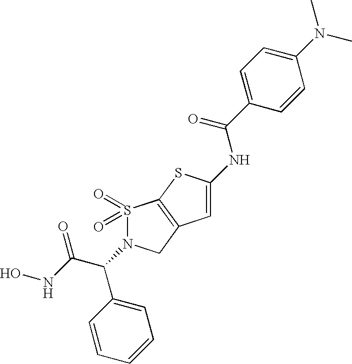 |
| AS1-098 | 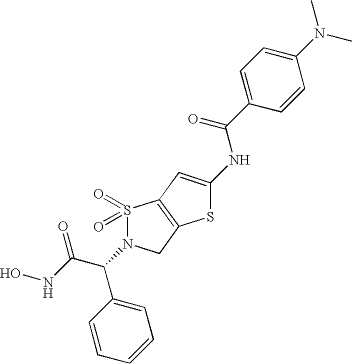 |
| AS1-099 | 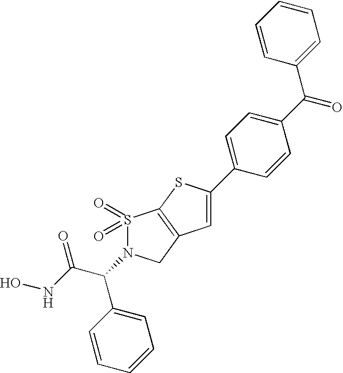 |
| AS1-100 | 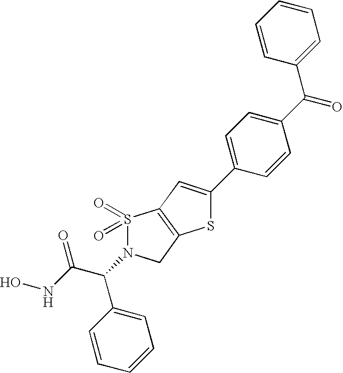 |
| AS2-001 | 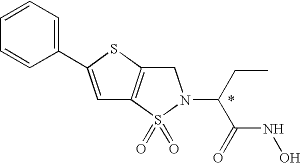 |
| AS2-002 | 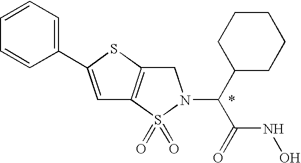 |
| AS2-003 | 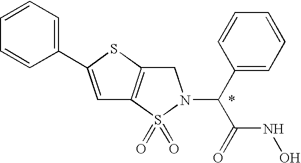 |
| AS2-004 | 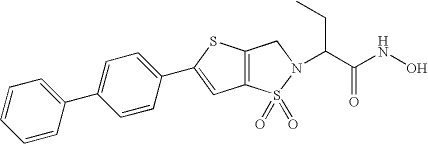 |
| AS2-005 | 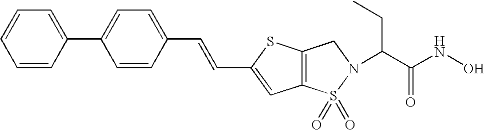 |
| AS2-006 | 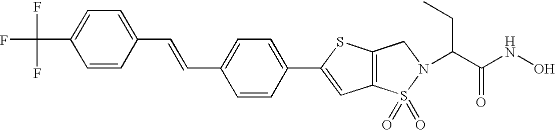 |
| AS2-007 | 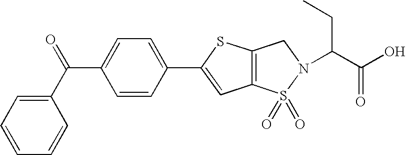 |
| AS2-008 | 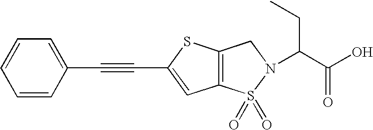 |
| AS2-009 | 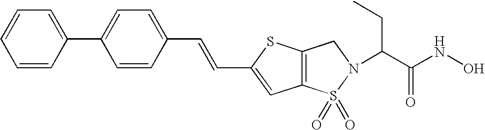 |
| AS2-010 | 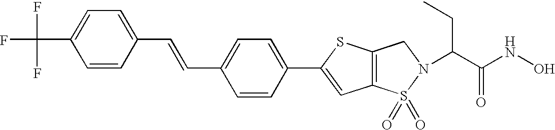 |
| AS2-011 | 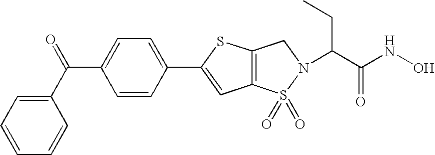 |
| AS2-012 | 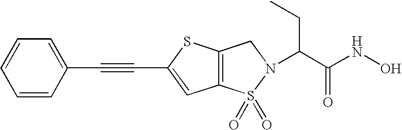 |
| AS2-013 | 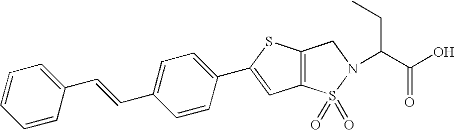 |
| AS2-014 | 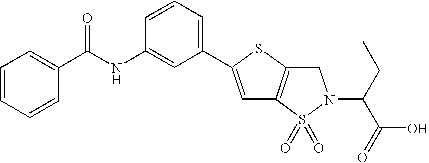 |
| AS2-015 | 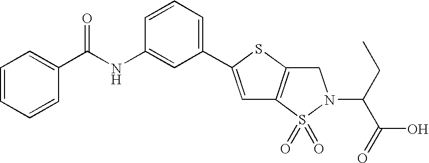 |
| AS2-016 | 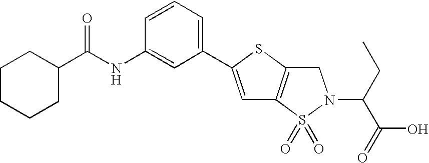 |
| AS2-017 | 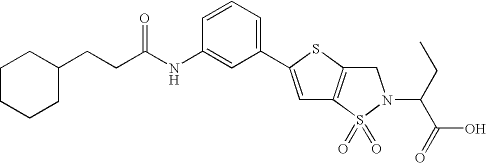 |
| AS2-018 | 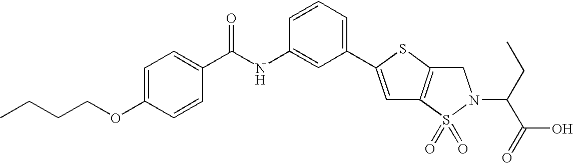 |
| AS2-019 | 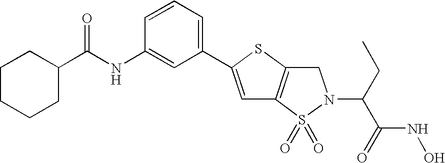 |
| AS2-020 | 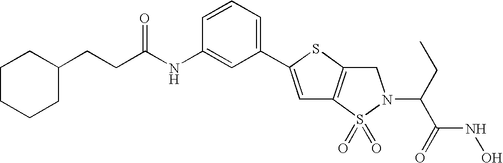 |
| AS2-021 | 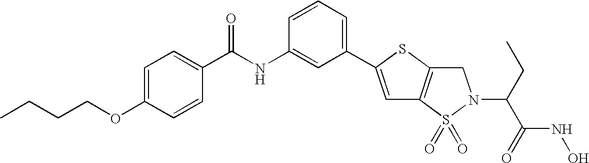 |
| AS2-022 | 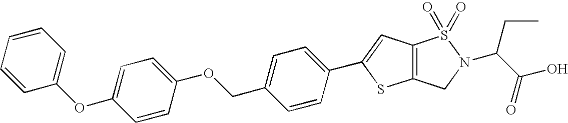 |
| AS2-023 | 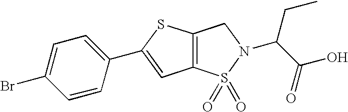 |
| AS2-024 | 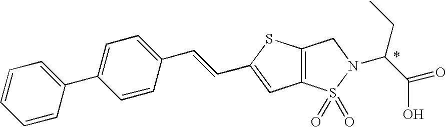 |
| AS2-025 | 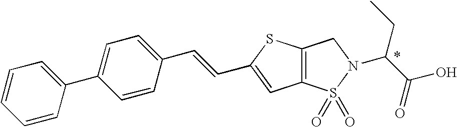 |
| AS2-026 | 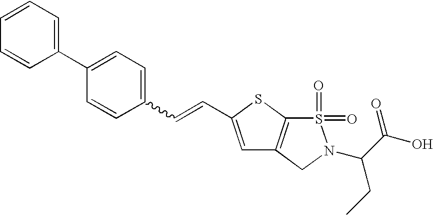 |
| AS2-027 | 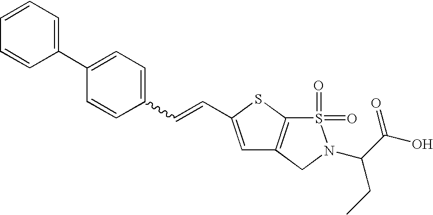 |
| AS2-028 | 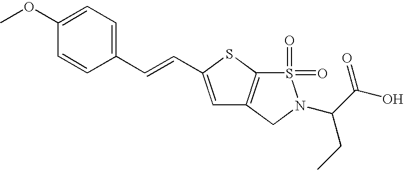 |
| AS2-029 | 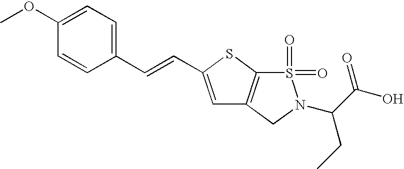 |
| AS2-030 | 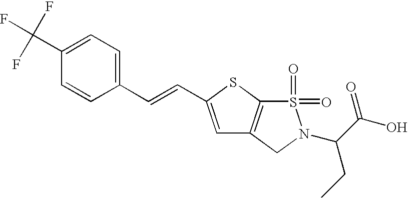 |
| AS2-031 | 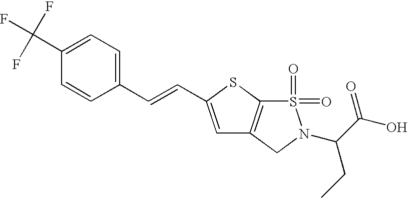 |
| AS2-032 | 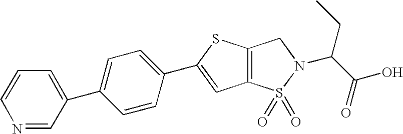 |
| AS2-033 | 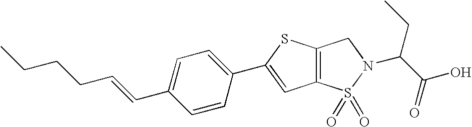 |
| AS2-034 | 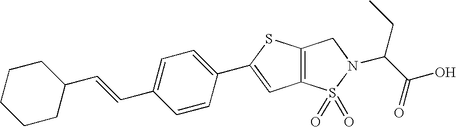 |
| AS2-035 | 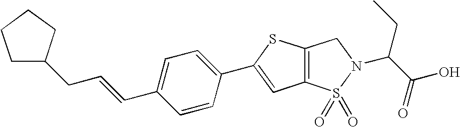 |
| AS2-036 | 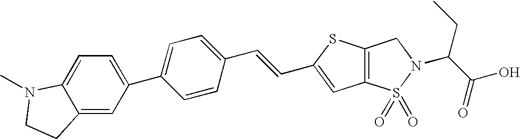 |
| AS2-037 | 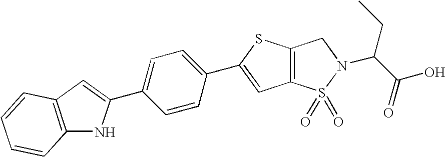 |
| AS2-038 | 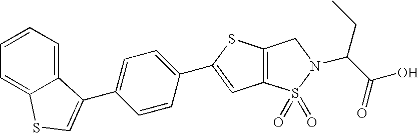 |
| AS2-039 | 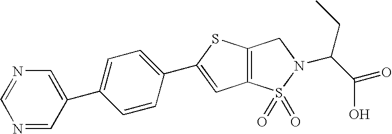 |
| AS2-040 | 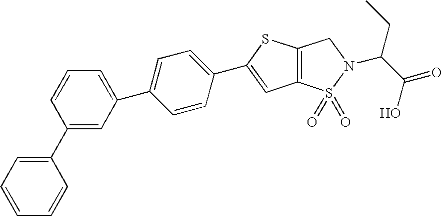 |
| AS2-041 | 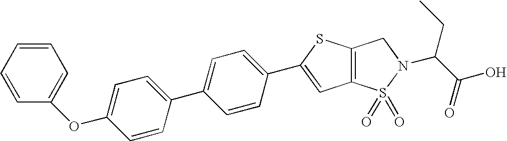 |
| AS2-042 | 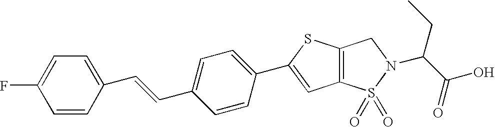 |
| AS2-043 | 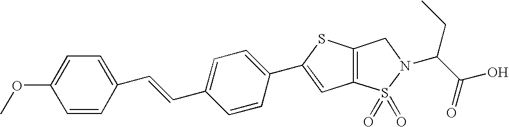 |
| AS2-044 | 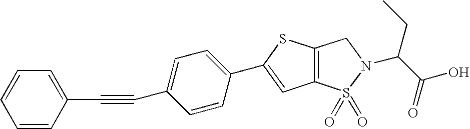 |
| AS2-045 | 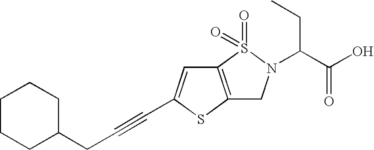 |
| AS2-046 | 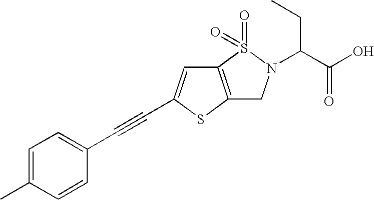 |
| AS2-047 | 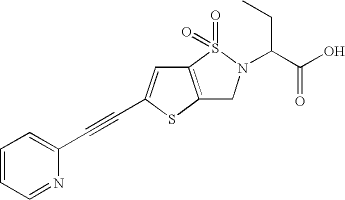 |
| AS2-048 | 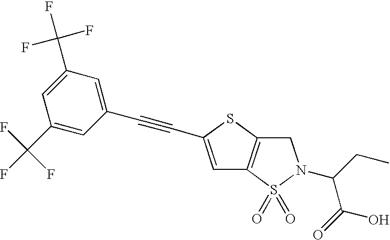 |
| AS2-049 | 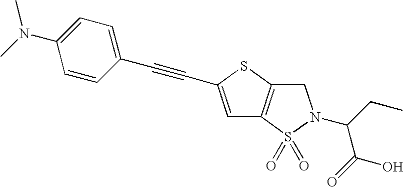 |
| AS2-050 | 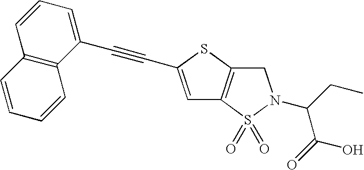 |
| AS2-051 | 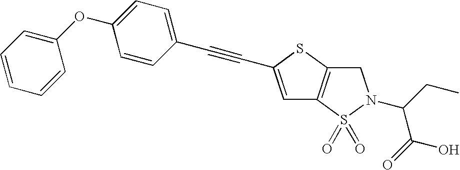 |
| AS2-052 | 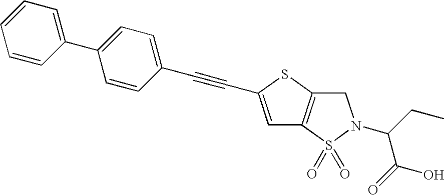 |
| AS2-053 | 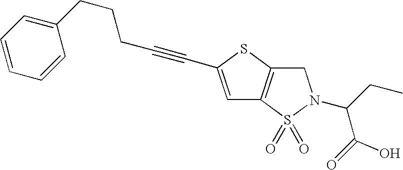 |
| AS2-054 | 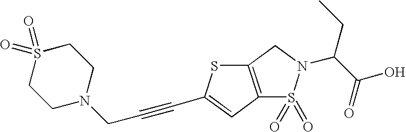 |
| AS2-055 | 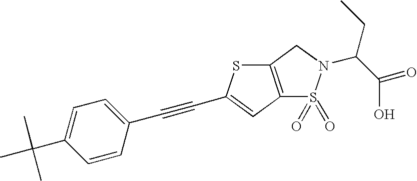 |
| AS2-056 | 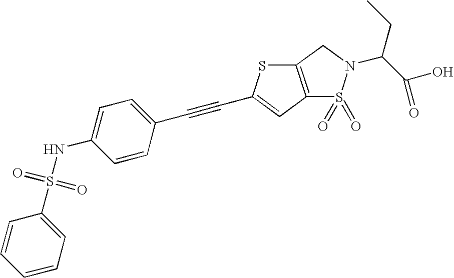 |
| AS2-057 | 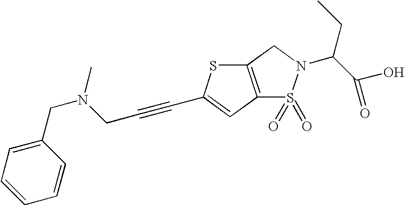 |
| AS2-058 | 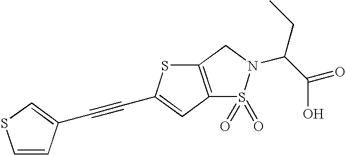 |
| AS2-059 | 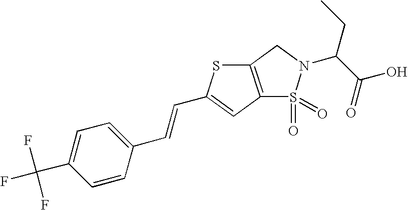 |
| AS2-060 | 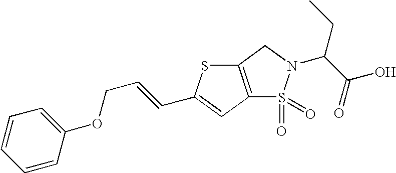 |
| AS2-061 | 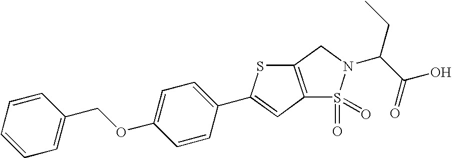 |
| AS2-062 | 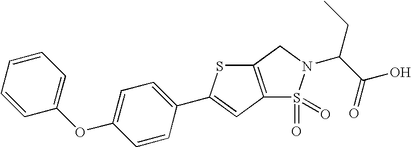 |
| AS2-063 | 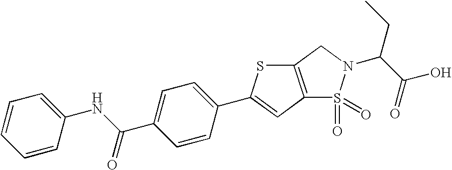 |
| AS2-064 | 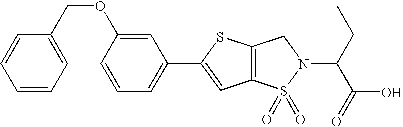 |
| AS2-065 | 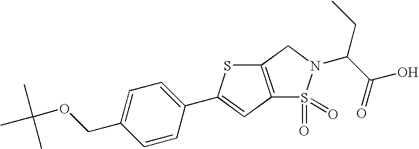 |
| AS2-066 | 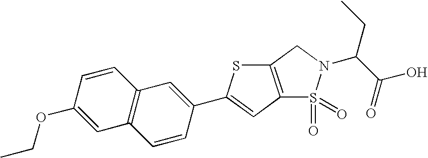 |
| AS2-067 | 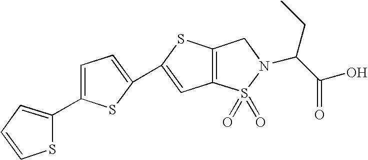 |
| AS2-068 | 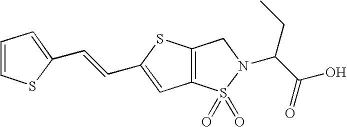 |
| AS2-069 | 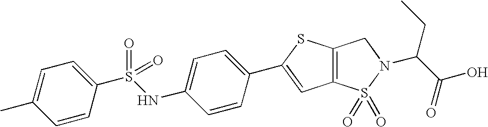 |
| AS2-070 | 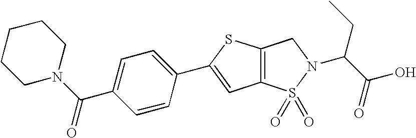 |
| AS2-071 | 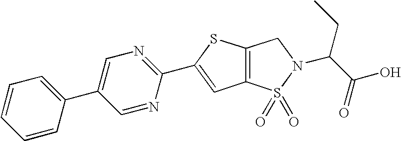 |
| AS2-072 | 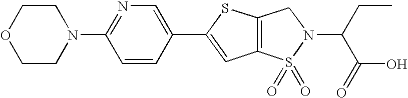 |
| AS2-073 | 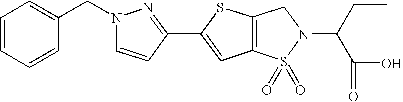 |
| AS2-074 | 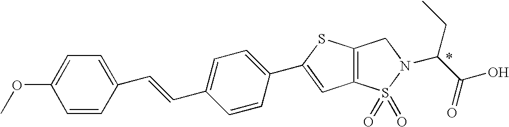 |
| AS2-075 | 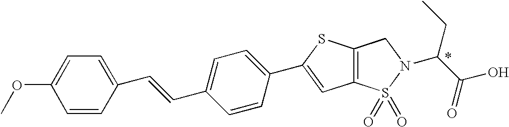 |
| AS2-076 | 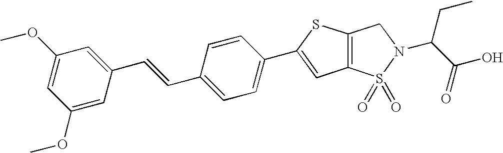 |
| AS2-077 | 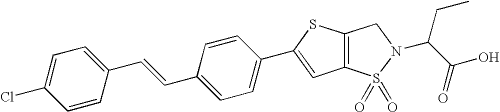 |
| AS2-078 | 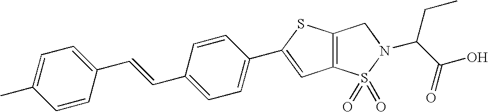 |
| AS2-079 | 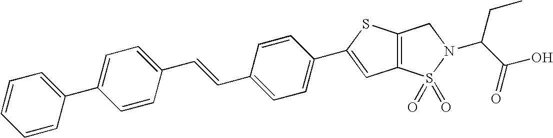 |
| AS2-080 | 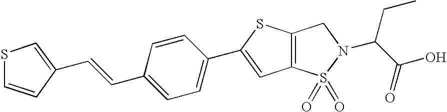 |
| AS2-081 | 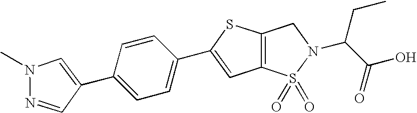 |
| AS2-082 | 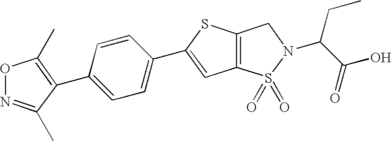 |
| AS2-083 | 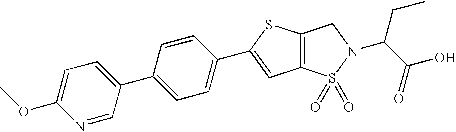 |
| AS2-084 | 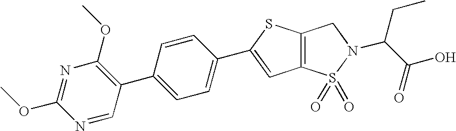 |
| AS2-085 | 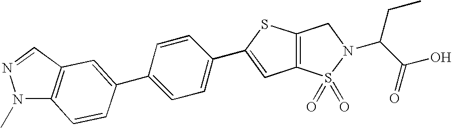 |
| AS2-086 | 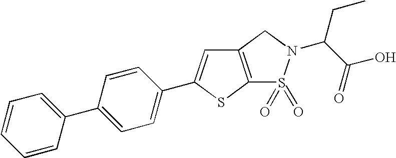 |
| AS2-087 | 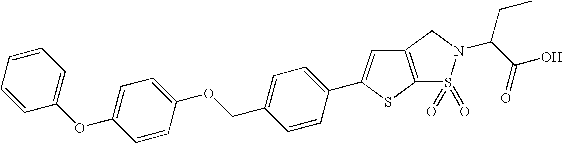 |
| AS2-088 | 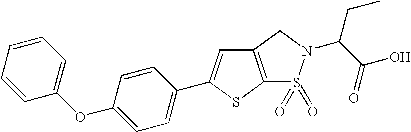 |
| AS2-089 | 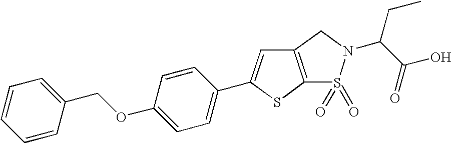 |
| AS2-090 | 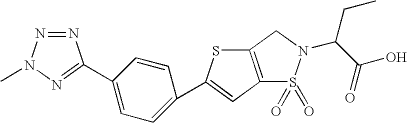 |
| AS2-091 | 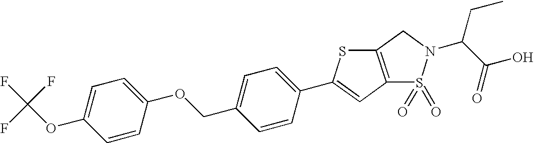 |
| AS2-092 | 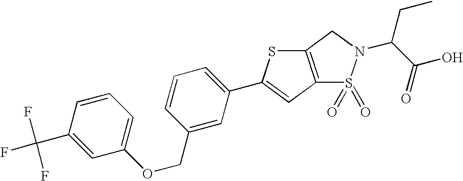 |
| AS2-093 | 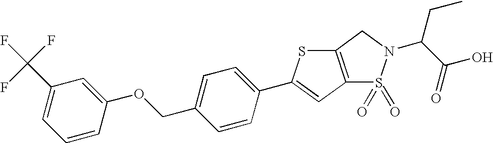 |
| AS2-094 | 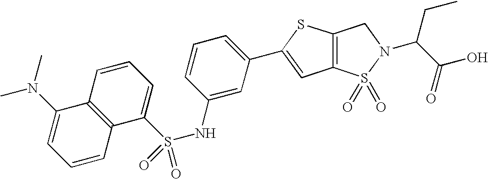 |
| AS2-095 | 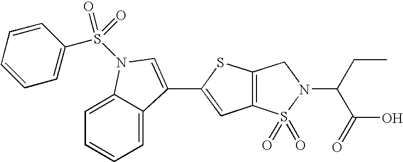 |
| AS2-096 | 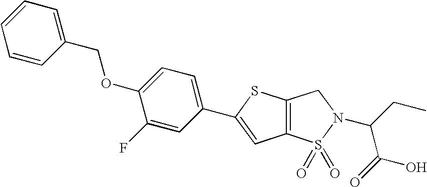 |
| AS2-097 | 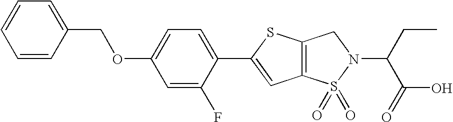 |
| AS2-098 | 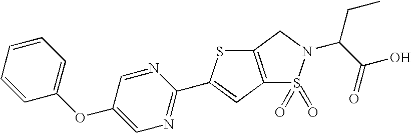 |
| AS2-099 | 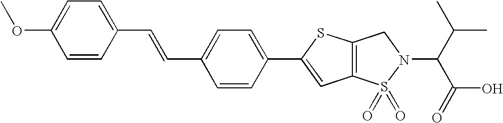 |
| AS2-100 | 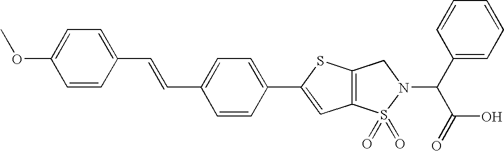 |
| AS2-101 | 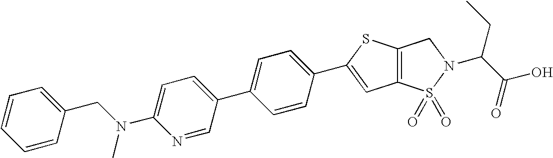 |
| AS2-102 | 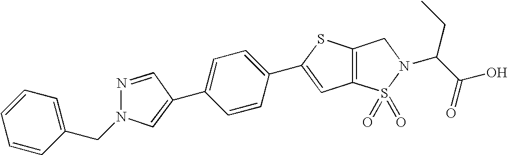 |
| AS2-103 | 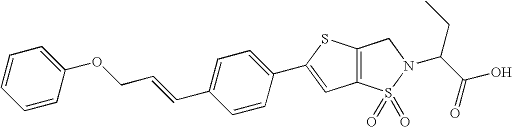 |
| AS2-104 | 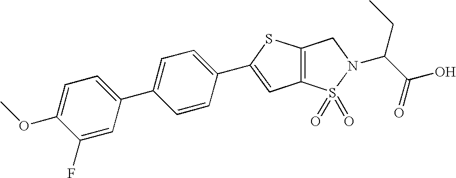 |
| AS2-105 | 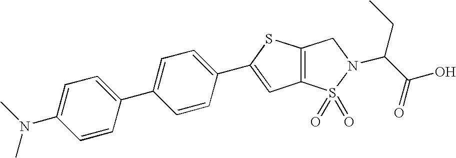 |
| AS2-106 | 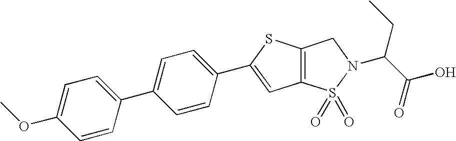 |
| AS2-107 | 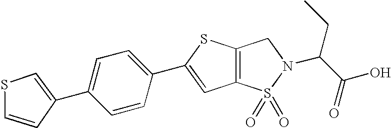 |
| AS2-108 | 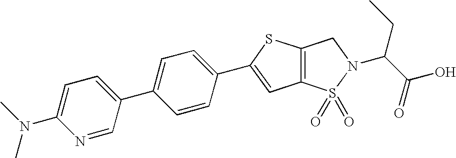 |
| AS2-109 | 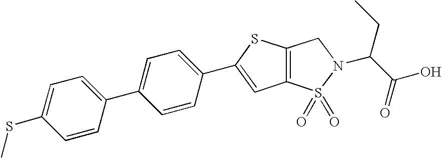 |
| AS2-110 | 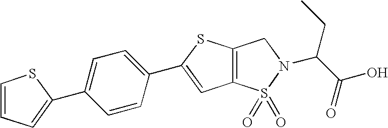 |
| AS2-111 | 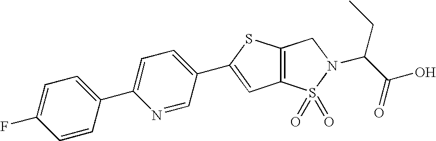 |
| AS2-112 | 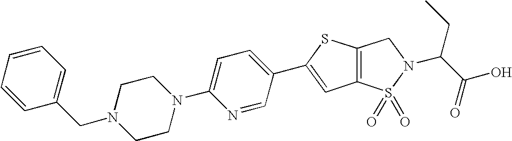 |
| AS2-113 | 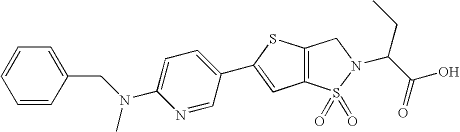 |
| AS2-114 | 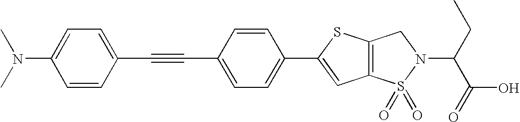 |
| AS2-115 | 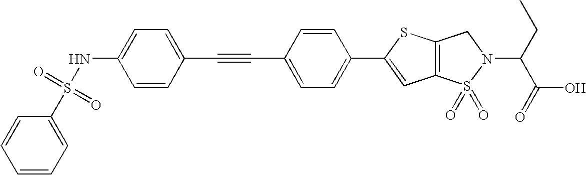 |
| AS2-116 | 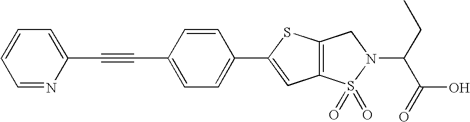 |
| AS2-117 | 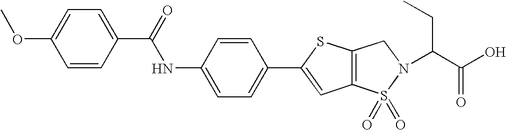 |
| AS2-118 | 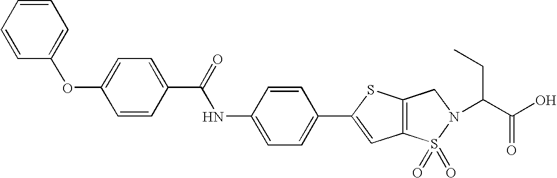 |
| AS2-119 | 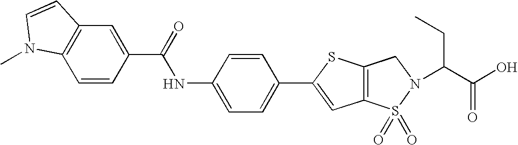 |
| AS2-120 | 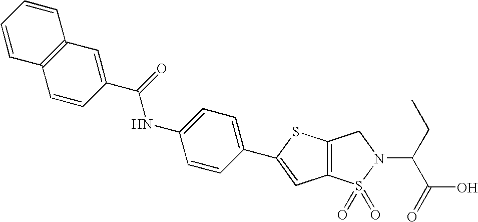 |
| AS2-121 | 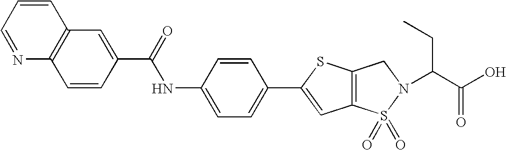 |
| AS2-122 | 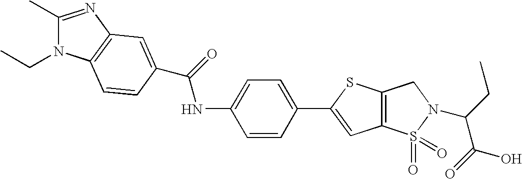 |
| AS2-123 | 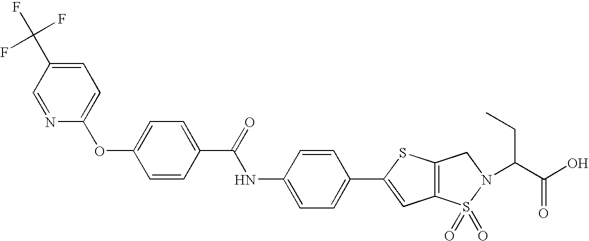 |
| AS2-124 | 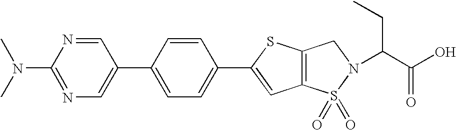 |
| AS2-125 | 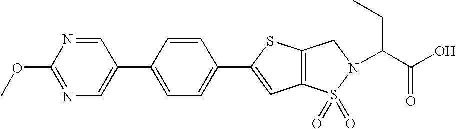 |
| AS2-126 | 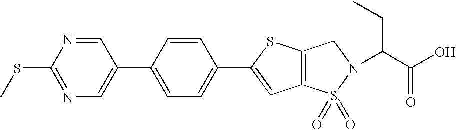 |
| AS2-127 | 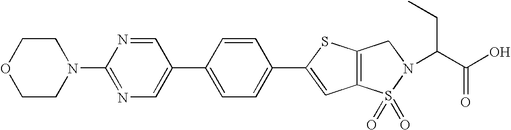 |
| AS2-128 | 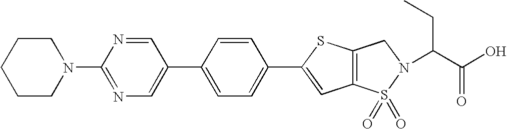 |
| AS1-129 | 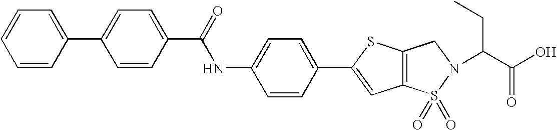 |
| AS2-130 | 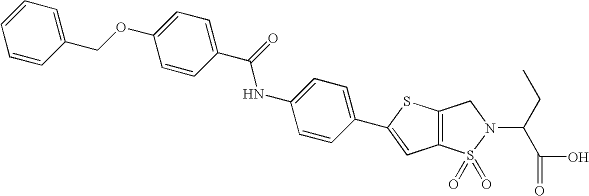 |
| AS2-131 | 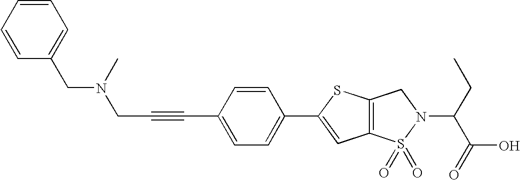 |

However, the scope of the present invention is not limited to these preferable examples. In addition, a possible stereoisomer or a racemic form, or a pharmacologically acceptable salt, a hydrate, a solvate, or a prodrug of the compound is also within the scope of the present invention.

The compounds of the present invention are novel compounds that have not been disclosed in literatures. The compounds that are represented by formula (1) can be prepared according to the following method, for example. However, the method for the production of the compounds of the present invention is not specifically limited.

For each reaction, reaction time is not specifically limited. Since a progress state of the reaction can be easily followed by a known analytical method, the reaction can be terminated at the time point at which yield of a target compound reaches the maximum.

Among the compounds of the present invention that are represented by formula (1), a compound in which Y¹¹ represents —NHOH, an optionally substituted alkylamino group, an optionally substituted dialkylamino group, an optionally substituted arylamino group, an optionally substituted alkylarylamino group, or an optionally substituted diarylamino group has the same meaning as the compound that is represented by the following formula (S1) [in formula (S1), Ar¹, R¹¹, R¹², R¹³, R²¹, R²², R³¹ n, and m have the same meanings as those described in the aforementioned and Y¹² represents —NHOH, an optionally substituted alkylamino group, an optionally substituted dialkylamino group, an optionally substituted arylamino group, an optionally substituted alkylarylamino group, or an optionally substituted diarylamino group], and such compounds can be prepared according to a retro-synthetic route described below, for example.

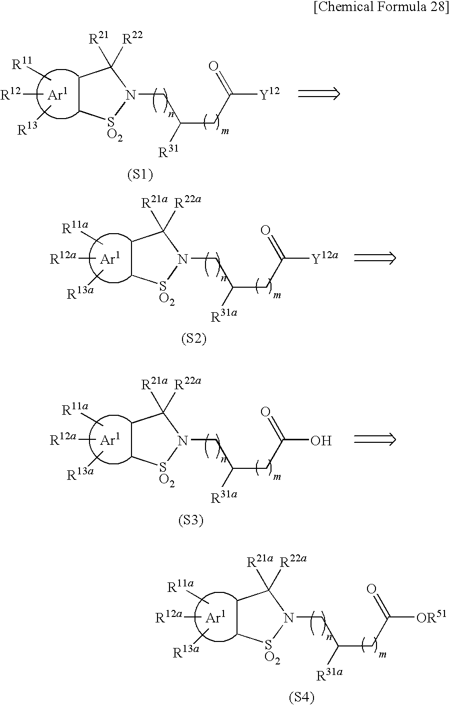

For example, the compound represented by formula (S1) can be produced by simultaneous or sequential deprotection of all protecting groups comprised in the compound represented by formula (S2) [in formula (S2), Ar¹, n and m have the same meanings as those described in the above and R^{11a}, R^{12a}, R^{13a}, R^{21a}, R^{22a}, R^{31a}, and Y² may have the same meanings as the above-described R¹¹, R¹², R¹³, R²¹, R²², R³¹, and Y¹², or at least one of these groups is protected] The deprotection reaction may be performed according to a known method, for example, the method as described in Protective Groups in Organic Synthesis published by John Wiley and Sons (2007) and the like.

In particular, by treatment of a compound in which Y^{12a} represents —NHOC(CH₃)₂OCH₃ with an acid, a compound in which Y² represents —NHOH can be prepared. In this case, the acid may be a mineral acid such as hydrochloric acid, nitric acid or sulfuric acid, or an organic acid such as acetic acid, trifluoroacetic acid, methanesulfonic acid, or p-toluenesulfonic acid. The acid is preferably hydrochloric acid. As for the amount of an acid, the acid can be used in an equivalent or excess amount relative to a compound in which Y^{12a} represents —NHOC(CH₃)₂OCH₃. Use of an excess amount of acid is preferable. Examples of a solvent that can be used for the reaction include water, methanol, ethanol, propanol, tetrahydrofuran, and 1,4-dioxane. In addition, two or more of these solvents can be used as a mixture. The solvent is preferably water, methanol, ethanol, or a mixture of these solvents. More preferably, it is a mixture comprising water and methanol. With respect to a combination of the acid and the solvent, it is preferable to mix hydrochloric acid and methanol for use. A mixture solvent comprising 1N hydrochloric acid and methanol in 1:1 (v/v) ratio is more preferable. The reaction temperature is typically in the range of 0° C. to 100° C. Preferably, it is 10° C. to 50° C. Although not being specifically limited, the reaction time is generally 1 to 96 hours, and preferably 3 to 60 hours.

The compound that is represented by formula (S2) can be prepared by reacting the compound that is represented by formula (S3) [in formula (S3), Ar¹, n, m, R^{11a}, R^{12a}, R^{13a}, R^{21a}, R^{22a} and R^{31a} have the meanings the same as those described in the above] with ammonia, primary amine, secondary amine, or hydroxylamine in the presence of a condensing agent, and if necessary, in the presence of a base. The use amount of ammonia, primary amine, secondary amine, or hydroxylamine is 1/10 to 100 equivalent, and preferably 1 to 10 equivalents relative to the compound represented by formula (S3). The condensing agent may be dicyclohexylcarbodiimide (DCC), O-(7-azabenzotriazol-1-yl) N,N,N′,N′-tetramethyluroniumhexafluorophosphate (HATU), or hydrochloric acid salt of 1-ethyl-3-[3-dimethylaminopropyl]carbodiimide (EDC). The use amount of the condensing agent can be an equivalent to excess amount relative to the compound that is represented by formula (S3). For example, 1 to 10 equivalents can be used. Preferably, it is 1 to 5 equivalents. It is preferable to use an auxiliary agent for the condensing reaction and the auxiliary agent may be hydroxybenzotriazole (HOBT). Examples of the base include trimethylamine, triethylamine, diisopropylethylamine, N-methylmorpholine, and pyridine. Preferably, the base is triethylamine, diisopropylethylamine, N-methylmorpholine, or pyridine. Examples of a solvent which can be used for the reaction include dichloromethane, chloroform, carbon tetrachloride, benzene, toluene, xylene, diethyl ether, tetrahydrofuran, dioxane, dimethoxy ethane, pyridine, and dimethyl form amide. In addition, two or more of these solvents can be used as a mixture. Preferably, the solvent is dimethyl form amide, or tetrahydrofuran. The reaction temperature is typically in the range of −80° C. to 100° C. Preferably, it is 0° C. to 50° C. Although not being specifically limited, the reaction time is generally 1 to 96 hours, and preferably 2 to 48 hours. Ammonia, primary amine, secondary amine, or hydroxyamine in the above-described reaction can be protected with a protecting group. For example, as a protected hydroxyamine, O-(2-methoxypropan-2-yl)hydroxyamine can be mentioned, and the protecting group can be deprotected by a known method (Mori, K., Tetrahedron, 44, 6013 (1988)).

The compound that is represented by formula (S3) can be prepared by subjecting the protecting group for the carboxylic acid of the compound represented by formula (S4) [in formula (S4), Ar¹, n, m, R^{11a}, R^{12a}, R^{13a}, R^{21a}, R^{22a}, and R^{31a} have the meanings the same as those described in the above, and R⁵¹ represents a protecting group for carboxylic acid] to deprotection reaction. In formula (S4), R⁵¹ represents a protecting group for carboxylic acid and may be an optionally substituted alkyl group. For example, a methyl group, an ethyl group, an isopropyl group, a t-butyl group, a benzyl group, or 2-(trimethylsilyl)ethyl group is preferred. A methyl group or a t-butyl group is more preferred. The deprotection reaction of the protecting group for carboxylic acid group may be performed according to a known method, for example, the method as described in Protective Groups in Organic Synthesis published by John Wiley and Sons (2007). For example, when R⁵¹ represents a methyl group, or an ethyl group, hydrolysis under a basic condition can be mentioned. In this case, as the base, sodium hydroxide, potassium hydroxide, lithium hydroxide or ammonia can be mentioned. Preferably, sodium hydroxide or lithium hydroxide can be used. The use amount of the base is an equivalent amount or an excess amount relative to the compound represented by formula (S4). Preferably, 1 to 5 equivalents is used. Examples of a solvent that can be used for the reaction include water, methanol, ethanol, propanol, tetrahydrofuran, and 1,4-dioxane. In addition, two or more of these solvents can be used as a mixture. The solvent is preferably water, methanol, ethanol, 1,4-dioxane or a mixture of these solvents. More preferably, it is a mixture comprising water and methanol. The reaction temperature is typically in the range of 0° C. to 200° C. Preferably, it is 10° C. to 100° C. Although not being specifically limited, the reaction time is generally 1 to 96 hours, and preferably 3 to 60 hours. In addition, when R⁵¹ represents a t-butyl group, a method including causing an acid to act on the compound represented by formula (S4) can be used. In this case, the acid may be a mineral acid such as hydrochloric acid, nitric acid or sulfuric acid, or an organic acid such as acetic acid, trifluoroacetic acid, methanesulfonic acid, or p-toluenesulfonic acid. The acid is preferably hydrochloric acid or trifluoroacetic acid. The amount of the acid can be an equivalent or excess amount relative to the compound represented by formula (S4). For example, it can be 1 to 10 equivalents, and is more preferably 1 to 20 equivalents. In another example, the amount of the acid is preferably an excess amount, e.g., a solvent amount. Examples of a solvent that can be used for the reaction include dichloromethane, chloroform, carbon tetrachloride, benzene, toluene, xylene, diethyl ether, tetrahydrofuran, dioxane, dimethoxyethane, ethyl acetate, butyl acetate and acetonitrile, and the solvent is preferably chloroform, toluene, or tetrahydrofuran. In addition, two or more of these solvents can be used as a mixture. In addition, the acid that is employed for the above reaction can also be preferably used as the solvent. As for preferable reaction conditions, use of 4N hydrochloric acid-1,4-dioxane solution in a solvent amount is preferable. The reaction temperature is typically in the range of 0° C. to 200° C. Preferably, it is 10° C. to 100° C. Although not being specifically limited, the reaction time is generally 1 to 200 hours, and preferably 1 to 96 hours.

Among the compounds that are represented by formula (1) of the present invention, the compound in which Y¹¹ represents —OH group can be prepared by simultaneous or sequential deprotection of all protecting groups comprised in the compound represented by formula (S3). The deprotection reaction may be performed according to a known method, for example, the method as described in Protective Groups in Organic Synthesis published by John Wiley and Sons (2007).

Among the compounds of the present invention that are represented by formula (S4), the compound in which R^{11a} represents —NHCO—R^{112a}—R^{113a}—R^{114a} [in the formula, R^{112a}, R^{113a}, and R^{114a} have the same meanings as the aforementioned R¹¹², R¹¹³, and R¹¹⁴, or one of these groups is protected] has the same meaning as the compound that is represented by formula (S5) described below [in the formula, Ar¹, n, m, R^{112a}, R^{113a}, R^{114a}, R^{12a}, R^{13a}, R^{21a}, R^{22a}, R^{31a}, and R⁵¹ have the meanings the same as those described in the aforementioned], and such compounds can be prepared according to a retro-synthetic route described below, for example.

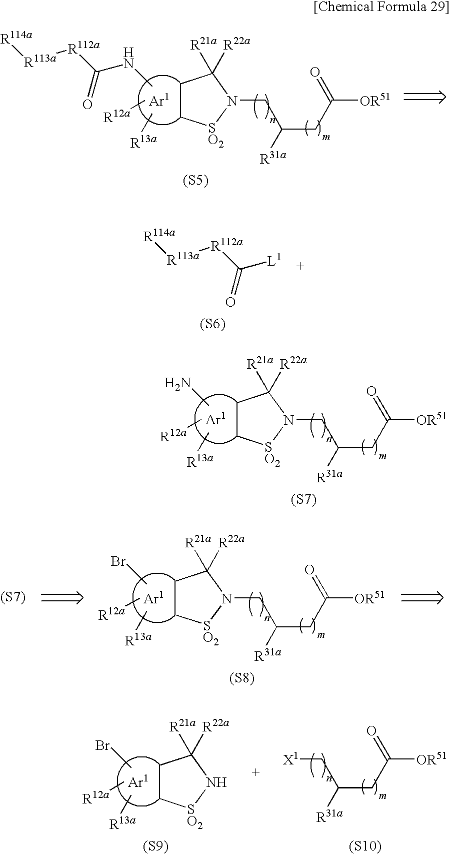

The compound that is represented by formula (S5) can be prepared by reacting the compound that is represented by formula (S6) [in the formula, R^{112a}, R^{113a}, R^{114a} have the meanings the same as those described in the above, L¹ represents a hydroxyl group, a chlorine atom or a bromine atom] with the compound that is represented by formula (S7) [in the formula, Ar¹, n, m, R^{12a}, R^{13a}, R^{21a}, R^{22a}, R^{31a}, and R⁵¹ have the meanings the same as those described in the aforementioned] if necessary, in the presence of a condensing agent and/or a base. The use amount of the compound that is represented by formula (S6) is 1/10 to 100 equivalents, and preferably 1 to 10 equivalents relative to the compound represented by formula (S7). Among the compounds that are represented by formula (S6), for a compound in which L¹ represents a hydroxyl group, it is preferable to use a condensing agent in the reaction. As the condensing agent, dicyclohexylcarbodiimide (DCC), O-(7-azabenzotriazol-1-yl) N,N,N′,N′-tetramethyluroniumhexafluorophosphate (HATU), or hydrochloric acid salt of 1-ethyl-3-[3-dimethylaminopropyl]carbodiimide (EDC) can be mentioned. The use amount of the condensing agent can be an equivalent to excess amount relative to the compound that is represented by formula (S6). For example, 1 to 10 equivalents can be used. Preferably, it is 1 to 5 equivalents. It is also preferable to use an auxiliary agent for the condensing reaction and the auxiliary agent may be hydroxybenzotriazole (HOBT). Examples of the base which can be used in the reaction include trimethylamine, triethylamine, diisopropylethylamine, N-methylmorpholine, and pyridine. Preferably, the base is triethylamine, diisopropylethylamine, N-methylmorpholine, or pyridine. The use amount of the base can be 0.1 to 10 equivalents relative to the compound that is represented by formula (S6). Preferably, it is 0.5 to 5 equivalents. Examples of a solvent which can be used in the reaction include dichloromethane, chloroform, carbon tetrachloride, benzene, toluene, xylene, diethyl ether, tetrahydrofuran, dioxane, dimethoxyethane, pyridine, and dimethyl form amide. In addition, two or more of these solvents can be used as a mixture. Preferably, the solvent is dimethyl form amide, dichloromethane, or tetrahydrofuran. The reaction temperature is typically in the range of −80° C. to 100° C. Preferably, it is 0° C. to 50° C. Although not being specifically limited, the reaction time is generally 1 to 96 hours, and preferably 2 to 48 hours.

For the compound that is represented by formula (S6), for example, commercially available benzoyl chloride (manufactured by Aldrich Company), 4-(dimethylamino)benzoyl chloride (manufactured by Aldrich Company), 4-phenylbenzoyl Chloride (manufactured by Lancaster Company), 4-benzoylbenzoic acid (manufactured by Aldrich Company) and the like can be used.

The compound that is represented by formula (S7) can be prepared by subjecting the compound represented by formula (S8) [in the formula, Ar¹, n, m, R^{12a}, R^{13a}, R^{21a}, R^{22a}, R^{31a}, and R⁵¹ have the meanings the same as those described in the aforementioned] to known Buchwald-Hartwig reaction. Reaction conditions are the same as those described in published literatures (A. R Muci, S. L. Buchwald, Top. Curr. Chem., 219, 131 (2002)., J. F. Hartwig, Angew. Chem., Int. Ed., 37, 2046 (1998)., D. Baranano, G. Mann, Hartwig, J. F. Curr. Org. Chem. 1, 287 (1997)., and C. G. Frost, P. Mendonca, J. Chem. Soc. Perkin Trans. 1, 1998, 2615.). Depending on the conditions of the reaction, a product with a protected amino group can be obtained. For such case, by carrying out appropriate deprotection reaction, derivatives having an amino group can be prepared. For example, a method in which Buchwald-Hartwig reaction is performed using benzophenonimine, and in which the resulting imine can be deprotected using hydroxylamine hydrochloric acid salt and sodium acetate in methanol to obtain an amine can be used.

The compound that is represented by formula (S8) can be prepared by reacting the compound that is represented by formula (S9) [in the formula, Ar¹, R^{12a}, R^{13a}, R^{21a}, and R^{22a} have the meanings the same as those described in the aforementioned] with the compound that is represented by formula (S10) [in the formula, n, m, R^{31a}, and R⁵¹ have the meanings the same as those described in the aforementioned, X¹ represents a bromine atom, a chlorine atom, an iodine atom, p-toluenesulfonyloxy group, or methanesulfonyloxy group] in the presence of a base. The use amount of the compound that is represented by formula (S10) is 1/10 to 10 equivalents, preferably ⅕ to 5 equivalents, and more preferably 1 to 2 equivalents relative to the compound represented by formula (S9). For the base used in the reaction, a metal alkoxide such as sodium methoxide or sodium ethoxide, carbonate salts such as sodium carbonate, potassium carbonate, or cesium carbonate, a metal hydride compound such as sodium hydride, sodium hydroxide, or potassium hydroxide can be exemplified. Preferably, the base is sodium hydride. The use amount of the base is ⅕ to 10 equivalents, and preferably ½ to 3 equivalents relative to the compound represented by formula (S9). The solvent that can be used in the reaction may be ethanol, methanol, propanol, isopropanol, ethylene glycol, propylene glycol, diethylene glycol monomethyl ether, dichloromethane, chloroform, carbon tetrachloride, benzene, toluene, xylene, diethyl ether, tetrahydrofuran, dioxane, dimethoxyethane, diethylene glycol dimethyl ether, ethyl acetate, butyl acetate, dimethylform amide or acetonitrile. Preferably, it is dimethylform amide. In addition, two or more of these solvents can be used as a mixture. The reaction temperature is typically in the range of 0° C. to 200° C. Preferably, it is 10° C. to 150° C. Although not being specifically limited, the reaction time is generally 1 to 96 hours, and preferably 3 to 36 hours.

Among the compounds represented by Formula (S9), a compound wherein both of R^{21a} and R^{22a} are hydrogen atoms can be produced, for example, by conducting reduction reaction of 5-bromo-2,3-dihydrothieno[2,3-d]isothiazol-3-one-1,1-dioxide as a raw material. The reduction reaction is, for example, a reaction using a complex metal hydride. Preferable examples of the complex metal hydride include aluminum lithium hydride. The dose of the complex metal hydride used is preferably 1 equivalent to 10 equivalents, and preferably 1 equivalent to 5 equivalents relative to the raw material. Examples of the solvent used in the reaction include diethyl ether, tetrahydrofuran, dioxane, and dimethoxyethane, and the solvent is preferably tetrahydrofuran. In addition, two or more of these solvents can be used as a mixture. The reaction temperature is generally 0° C. from 100° C., and preferably 10° C. to 70° C. The reaction time is not particularly limited, but generally 1 hour to 96 hours, and preferably 3 hours to 24 hours. 5-Bromo-2,3-dihydrothieno[2,3-d]isothiazol-3-one-1,1-dioxide as the raw material can be produced according to a known method of synthesizing 5-bromo-2,3-dihydrobenzo[d]isothiazol-3-one-1,1-dioxide (Tetrahedron, 62, 33, 7902-7910, 2006., Bioorg. Med. Chem., 13, 949-962 (2005).) and the like.

In addition, the compound that is represented by formula (S8) can be prepared by subjecting the compound that is represented by formula (S7) to Sandmeyer reaction. Sandmeyer reaction can be carried out by simultaneously or sequentially causing a diazotization agent such as sodium nitrite, t-butyl nitrite, or amyl nitrite and a halogenating agent such as copper bromide to act. The use amount of each of the reactants is 1 to 10 equivalents, and preferably 1 to 5 equivalents relative to the compound that is represented by formula (S7). The solvent that can be used in the reaction may be water, ethanol, methanol, propanol, isopropanol, or acetonitrile. Preferably, it is water or acetonitrile. In addition, two or more of these solvents can be used as a mixture. The reaction temperature is typically in the range of −30° C. to 150° C. Preferably, it is 0° C. to 100° C. Although not being specifically limited, the reaction time is generally 1 to 96 hours, and preferably 3 to 36 hours.

For the compound that is represented by formula (S10), for example, commercially available methyl bromoacetate

(manufactured by Aldrich Company), ethyl 2-bromo-3-methylbutyrate (manufactured by AVOCADO company), ethyl α-bromophenylacetate (manufactured by Aldrich Company) or the like can be used.

As an alternative method, the compound that is represented by formula (S7) can be prepared according to a retro-synthetic route described below, for example.

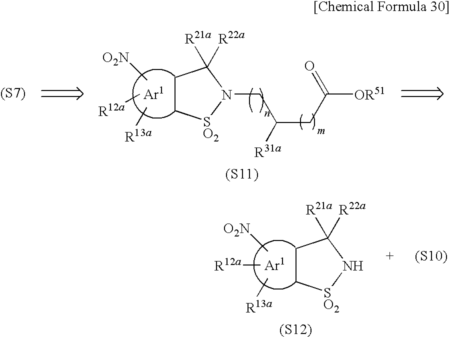

The compound that is represented by formula (S7) can be prepared by subjecting the compound represented by formula (S11) [in the formula, Ar¹, n, m, R^{12a}, R^{13a}, R^{21a}, R^{22a}, R^{31a}, and R⁵¹ have the meanings the same as those described in the aforementioned] to reductive reaction. The reductive reaction may be catalytic hydrogenation. The catalytic hydrogenation can be conducted using a catalyst in the presence of a solvent under hydrogen atmosphere. Hydrogen gas can be used under an atmospheric pressure or increased pressure. Preferably, an atmospheric pressure is employed. Examples of the catalyst include palladium-carbon, platinum oxide, platinum-carbon and palladium hydroxide. The solvent that can be used in the reaction include dichloromethane, chloroform, carbon tetrachloride, benzene, toluene, xylene, diethyl ether, tetrahydrofuran, dioxane, dimethoxyethane, methanol, and ethanol. Preferably, the solvent is tetrahydrofuran or methanol. In addition, two or more of these solvents can be used as a mixture. The reaction temperature is typically in the range of −80° C. to 100° C. Preferably, it is 0° C. to 50° C. Although not being specifically limited, the reaction time is generally 1 to 96 hours, and preferably 1 to 24 hours.

The compound that is represented by formula (S11) can be prepared by reacting the compound that is represented by formula (S12) [in the formula, Ar¹, R^{12a}, R^{13a}, R^{21a}, and R^{22a} have the meanings the same as those described in the aforementioned] with the compound that is represented by formula (S10) in the presence of a base. The use amount of the compound that is represented by formula (S10) is 1/10 to 10 equivalents, preferably ⅕ to 5 equivalents, and more preferably 1 to 2 equivalents relative to the compound represented by formula (S12). The base used in the reaction may be a metal alkoxide such as sodium methoxide or sodium ethoxide, carbonate such as sodium carbonate, potassium carbonate, or cesium carbonate, a metal hydride compound such as sodium hydride, sodium hydroxide, or potassium hydroxide.

Preferably, it is sodium hydride. The use amount of the base is ⅕ to 10 equivalents, and preferably ½ to 3 equivalents relative to the compound represented by formula (S12). The solvent that can be used in the reaction may be ethanol, methanol, propanol, isopropanol, ethylene glycol, propylene glycol, diethylene glycol monomethyl ether, dichloromethane, chloroform, carbon tetrachloride, benzene, toluene, xylene, diethyl ether, tetrahydrofuran, dioxane, dimethoxyethane, diethylene glycol dimethyl ether, ethyl acetate, butyl acetate, dimethylform amide or acetonitrile. Preferably, it is dimethylform amide. In addition, two or more of these solvents can be used as a mixture. The reaction temperature is typically in the range of 0° C. to 200° C. Preferably, it is 10° C. to 150° C. Although not being specifically limited, the reaction time is generally 1 to 96 hours, and preferably 3 to 36 hours.

The compound that is represented by formula (S12) can be prepared with reference to the method for producing the compound that is represented by formula (S9), for example.

Among the compounds represented by Formula (S4), a compound wherein R^{11a} is represented by —R^{111a}—R^{112a}—NHCO—R^{114a} [Wherein R^{111a} has the same meaning as R¹¹¹ or may be a protectable group, and R^{112a}, R^{113a} and R^{114a} have the same meanings as the aforementioned] is the same as a compound represented by Formula (S5-2) described below [Wherein Ar¹, n, m, R^{111a}, R^{112a}, R^{114a}, R^{12a}, R^{13a}, R^{21a}, R^{22a}, R^{31a} and R⁵¹ have the same meanings as the aforementioned], and such a compound may be produced according to, for example, an retro-synthetic route described below.

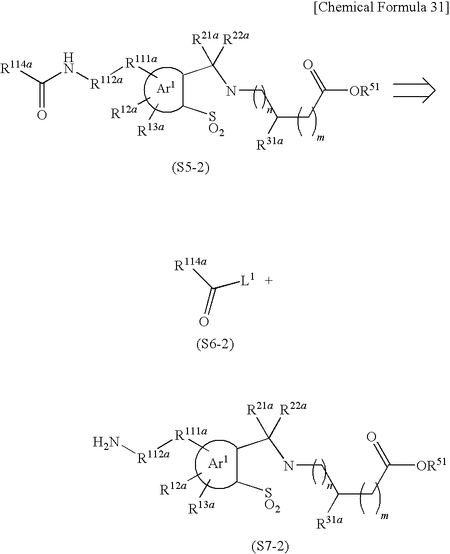

The compound represented by Formula (S5-2) can be produced by subjecting a compound represented by Formula (S6-2) [Wherein R^{114a} and L¹ have the same meanings as the aforementioned] and a compound represented by Formula (S7-2) [Wherein Ar¹, n, m, R^{111a}, R^{112a}, R^{12a}, R^{13a}, R^{21a}, R^{22a}, R^{31a} and R⁵¹ have the same meanings as the aforementioned] to a condensation reaction. The conditions of the condensation reaction are, for example, the same as the conditions of the method of producing the compound represented by Formula (S5) using the compound represented by Formula (S6) and the compound represented by Formula (S7).

As the compound represented by Formula (S6-2), commercially available compounds may be used, and for example, it is the same as the examples of the compound represented by the (S6).

The compound represented by Formula (S7-2) can be produced, for example, using commercially available ethyl 3-amino-5-(3-nitrophenyl)thiophen-2-carboxylate (ENAMINE Ltd.) as a starting material on the basis of the aforementioned method of producing the compound represented by Formula (ω-7)

Among the compounds represented by Formula (S4), a compound wherein R^{11a} is represented by —NH_CH₂—R^{1121a}—R^{113a}—R^{114a} [Wherein R^{113a} and R^{114a} are respectively the same as the aforementioned and R^{1121a}, together with —CH₂—, is the same as a part of R^{112a}], may be produced, for example, according to the retro-synthetic route described below.

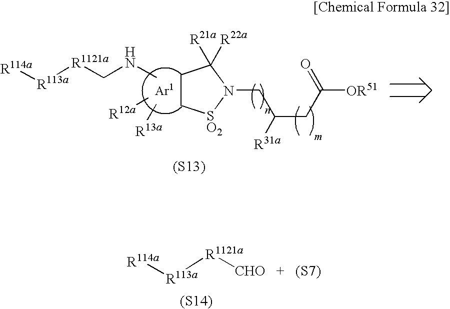

Among the compounds represented by Formula (S4), a compound wherein R^{11a} is represented by —NH_CH₂—R^{1121a}—R^{113a}—R^{114a} [Wherein R^{113a} and R^{114a} are respectively the same as the aforementioned and R^{1121a}, together with —CH₂—, is the same as a part of R^{112a}] is the same as a compound represented by Formula (S13) [Wherein Ar¹, n, m, R^{1121a}, R^{113a}, R^{114a}, R^{12a}, R^{13a}, R^{21a}, R^{22a}, R^{31a} and R⁵¹ are the same as the aforementioned], and can be produced by subjecting a compound represented by Formula (S14) [Wherein R^{1121a}, R^{113a} and R^{114a} are the same as the above-mentioned] and the compound represented by Formula (S7) to a reductive amination reaction. A solvent used in the reductive amination reaction may be diethyl ether, tetrahydrofuran, dioxane, dimethoxyethane, methanol, ethanol, or acetonitrile. The solvent is preferably methanol or acetonitrile. In addition, two or more of these solvents may be used as a mixture. The compound represented by Formula (S14) is preferably used in equivalent or excessive amount relative to the compound represented by Formula (S7), and more preferably in 1 equivalent to 10 equivalents. If necessary, an acid is preferably used in the reaction, and the acid is preferably acetic acid or an acidic buffer solution. Alternatively, if necessary, a base is preferably used, and the base is for example, tertiary amine, preferably, triethylamine. A reducing agent to be used is preferably sodium cyanoborohydride or sodium triacetoxyborohydride, and more preferably sodium cyanoborohydride. The reducing agent is preferably used in equivalent or excessive amount relative to the compound represented by Formula (S7), and more preferably in 1 equivalent to 10 equivalents. The reaction temperature is generally −20° C. to 100° C., and preferably −10° C. to 50° C. The reaction time is not particularly limited, but generally 0.2 hour to 36 hours, and preferably 1 hour to 24 hours.

As the compound represented by Formula (S14), for example, commercially available biphenyl-4-carboxaldehyde (manufactured by Aldrich), 4-phenoxybenzaldehyde (manufactured by Aldrich) and the like can be used.

Among the compounds represented by Formula (S4), a compound wherein R^{11a} is represented by —R^{1122a}—R^{113a}—R^{114a} [Wherein R^{113a} and R^{114a} are respectively the same as the aforementioned and R^{1122a} represents an optionally substituted arylene group and may be protected], may be produced, for example, according to the retro-synthetic route described below.

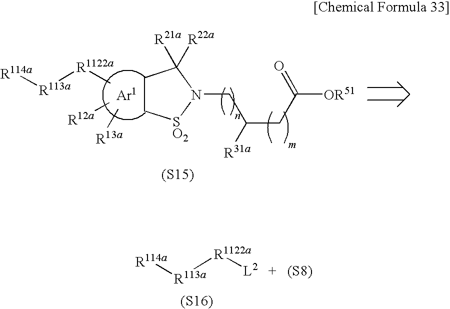

Among the compounds represented by Formula (S4), a compound wherein R^{11a} is represented by —R^{1122a}—R^{113a}—R^{114a} [Wherein R^{113a} and R^{114a} are respectively the same as the aforementioned and R^{1122a} represents an optionally substituted arylene group] is the same as the compound represented by Formula (S15) [Wherein Ar¹, n, m, R^{1122a}, R^{113a}, R^{114a}, R^{12a}, R^{13a}, R^{21a}, R^{22a}, R^{31a} and R⁵¹ are the same as the aforementioned], and can be produced by conducting Suzuki reaction of a compound represented by Formula (S16) [Wherein R^{1122a}, R^{113a} and R^{114a} are respectively the same as the aforementioned, and L² represents boronic acid —B(OH)₂ or boronic acid ester] and the compound represented by Formula (S8). The conditions of Suzuki reaction are as described in published documents (N. Miyaura et, al., J. Am. Chem. Soc., 107, 972 (1985)., N. Miyaura, A. Suzuki, Chem. Rev. 95, 2457 (1995)) and the like. Suzuki reaction may be performed in the presence of a catalyst, if necessary using a base and an additive. Specifically, a solvent used in Suzuki reaction may be tetrahydrofuran, dioxane, toluene, dimethoxyethane, methanol, ethanol, or acetonitrile. In addition, two or more of these solvents may be used as a mixture, or a mixture of two or more of the solvents and water may also be used. The solvent is preferably a mixed solvent of tetrahydrofuran and water, a mixed solvent of toluene, methanol and water, or dioxane. The compound represented by Formula (S16) is preferably used in equivalent or excessive amount relative to the compound represented by Formula (S8), and more preferably in 1 equivalent to 10 equivalents. If necessary, a base is preferably used in the reaction. The base is, for example, sodium carbonate, cesium carbonate, or potassium carbonate, and preferably sodium carbonate or cesium carbonate. The dose of the base to be used is 1 equivalent to 10 equivalents, and preferably 1 equivalent to 5 equivalents relative to the compound represented by Formula (S8). The catalyst used is preferably PdCl₂(dppf), Pd₂(dba)₃, or Pd(PPh₃)₄, and may be used in 0.01 equivalent to 1 equivalent, and preferably 0.1 to 0.5 equivalent relative to the compound represented by Formula (S8). The additive, which is used if necessary, may be rac-BINAP, and may be used in 0.01 equivalent to 1 equivalent, and preferably in 0.1 to 0.5 equivalent relative to the compound represented by Formula (S8). The reaction temperature is generally 0° C. to 200° C., and preferably 10° C. to 100° C. The reaction time is not particularly limited, but generally 0.2 hour to 48 hours, and preferably 1 hour to 36 hours. The reaction may be performed under microwave irradiation conditions.

As the compound represented by Formula (S16), for example, commercially available 4-biphenylboronic acid (manufactured by Aldrich), 4-phenoxyphenylboronic acid (manufactured by Aldrich), 4-benzoylphenylboronic acid (manufactured by Aldrich) and the like can be used.

In addition, a deprotection reaction of the protective group R⁵¹ for the carboxylic acid may further proceed in the Suzuki reaction. In this case, a compound wherein R^{11a} is represented by —R^{1122a}—R^{113a}—R^{114a} [Wherein R^{1122a}, R^{113a} and R^{114a} are respectively the same as the aforementioned] may be obtained as a product among the compounds represented by formula (S3)

Among the compounds represented by Formula (S4), a compound wherein R^{11a} is represented by —CH═CH—R^{112a}—R^{113a}—R^{114a} [Wherein R^{112a}, R^{113a} and R^{114a} are respectively the same as the aforementioned] may be produced, for example, according to the retro-synthetic route described below.

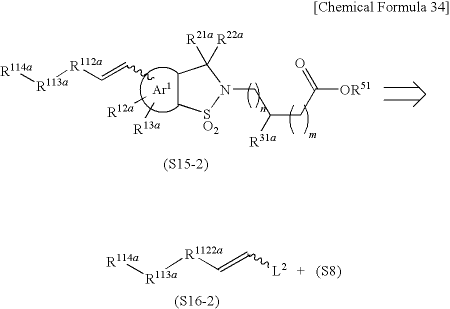

Among the compounds represented by Formula (S4), a compound wherein R^{11a} is represented by —CH═CH—R^{112a}—R^{113a}—R^{114a} [Wherein R^{112a} R^{113a} and R^{114a} are respectively the same as the aforementioned] is the same as the compound represented by Formula (S15-2) [Wherein Ar¹, n, m, R^{112a}, R^{113a}, R^{114a}, R^{12a}, R^{13a}, R^{21a}, R^{22a}, R^{31a} and R⁵¹ are the same as the aforementioned], and can be produced by conducting Suzuki reaction of a compound represented by Formula (S16-2) [Wherein R^{112a}, R^{113a}, R^{114a}, and L² are respectively the same as the aforementioned] and the compound represented by Formula (S8). The conditions of the Suzuki reaction are the same as the conditions in the method of producing the compound represented by Formula (S15).

Incidentally, deprotection reaction of the protective group R⁵¹ for the carboxylic acid may further proceed in the Suzuki reaction. In this case, a compound wherein R^{11a} is represented by CH═CH—R^{112a}—R^{113a}—R^{114a} [Wherein R^{112a}, R^{113a} and R^{114a} are respectively the same as the aforementioned] may be obtained as a product among the compounds represented by formula (S3).

Among the compounds represented by Formula (S4), a compound wherein R^{11a} is represented by —C≡C—R^{112a}—R^{113a}—R^{114a} [Wherein R^{112a}, R^{113a} and R^{114a} are respectively the same as the aforementioned] may be produced, for example, according to the retro-synthetic route described below.

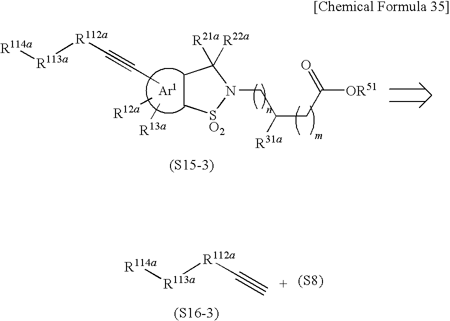

Among the compounds represented by Formula (S4), a compound wherein R^{11a} is represented by —C≡C—R^{112a}—R^{113a}—R^{114a} [Wherein R^{12a} R^{113a} and R^{114a} are respectively the same as the aforementioned] is the same as the compound represented by Formula (S15-3) [Wherein Ar¹, n, m, R^{112a}, R^{113a}, R^{114a}, R^{12a}, R^{13a}, R^{21a}, R^{22a}, R^{31a} and R⁵¹ are the same as the aforementioned], and can be produced by conducting Sonogashira reaction of a compound represented by Formula (S16-3) [Wherein R^{112a}, R^{113a}, R^{114a}, and L² are respectively the same as the aforementioned] and the compound represented by Formula (S8). The reaction conditions of the Sonogashira reaction are as described in a published document (R. Chinchilla et. al., Chem. Rev., 107 (3), 874-922 (2007).) and the like.

Among the compounds represented by Formula (S4), a compound wherein R^{11a} is represented by —R^{111a}—R^{1122a}—R^{1142a} [Wherein R^{1142a} represents an optionally substituted arylene group, and R^{1142a} may be protected, and R^{111a} and R^{1122a} are the same as the aforementioned] may be produced, for example, according to the retro-synthetic route described below.

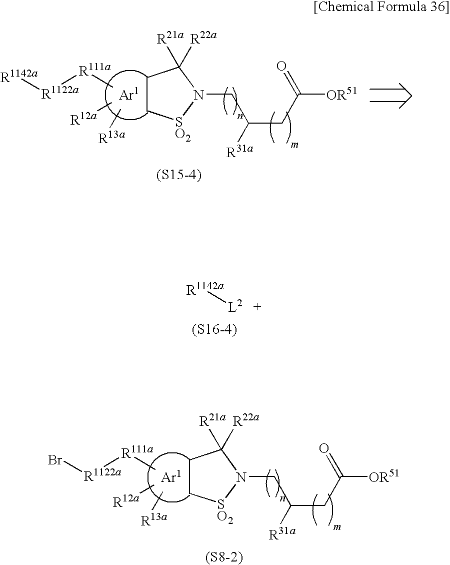

Among the compounds represented by Formula (S4), a compound wherein R^{11a} is represented by —R^{111a}—R^{1122a}—R^{1142a} [Wherein R^{111a}, R^{1122a} and R^{1142a} are the same as the aforementioned] is the same as the compound represented by Formula (S15-4) [Wherein Ar¹, n, m, R^{111a}, R^{1122a}, R^{1142a}, R^{12a}, R^{13a}, R^{21a}, R^{22a}, R^{31a} and R⁵¹ are the same as the aforementioned], and can be produced by conducting Suzuki reaction of a compound represented by Formula (S16-4) [Wherein R^{1142a} and L² are the same as the aforementioned] and a compound represented by Formula (S8-2) [Wherein Ar¹, n, m, R^{111a}, R^{1122a}, R^{12a}, R^{13a}, R^{21a}, R^{22a}, R^{31a} and R⁵¹ are the same as the aforementioned]. The conditions of the Suzuki reaction are the same as the conditions in the method of producing the compound represented by Formula (S15).

Among the compounds represented by Formula (S4), a compound wherein R^{11a} is represented by —R^{111a}—R^{112a}—CH═CH—R^{114a} [Wherein R^{111a}, R^{122a} and R^{114a} are the same as the aforementioned] may be produced, for example, according to the retro-synthetic route described below.

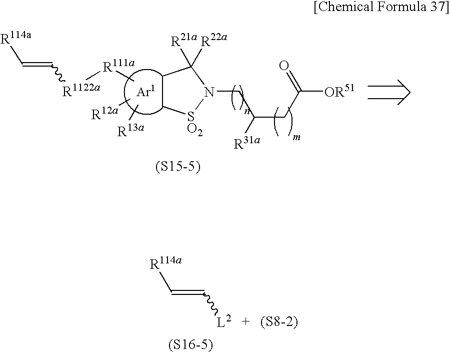

Among the compounds represented by Formula (S4), a compound wherein R^{11a} is represented by —R^{111a}—R^{112a}—CH═CH—R^{114a} [Wherein R^{111a}, R^{1122a} and R^{114a} are the same as the aforementioned] is the same as the compound represented by Formula (S15-5) [Wherein Ar¹, n, m, R^{111a}, R^{122a}, R^{114a}, R^{12a}, R^{13a}, R^{21a}, R^{22a}, R^{31a} and R⁵¹ are the same as the aforementioned], and can be produced by conducting Suzuki reaction of a compound represented by Formula (S16-5) [Wherein R^{114a} and L² are the same as the aforementioned] and the compound represented by Formula (S8-2). The conditions of the Suzuki reaction are the same as the conditions in the method of producing the compound represented by Formula (S15).

Among the compounds represented by Formula (S4), a compound wherein R^{11a} is represented by —R^{111a}—R^{1122a}—C≡C—R^{114a} [Wherein R^{111a}, R^{1122a} and R^{114a} are respectively the same as the aforementioned] may be produced, for example, according to the retro-synthetic route described below.

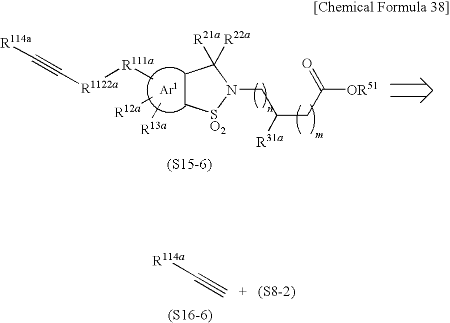

Among the compounds represented by Formula (S4), a compound wherein R^{11a} is represented by —R^{111a}—R^{1122a}—C≡C—R^{114a} [Wherein R^{111a}, R^{1122a} and R^{114a} are respectively the same as the aforementioned] is the same as the compound represented by Formula (S15-6) [Wherein Ar¹, n, m, R^{111a}, R^{1122a}, R^{114a}, R^{12a}, R^{13a}, R^{21a}, R^{22a}, R^{31a} and R⁵¹ are the same as the aforementioned], and can be produced by conducting Sonogashira reaction of a compound represented by Formula (S16-6) [Wherein R^{114a} is the same as the aforementioned] and the compound represented by Formula (S8-2) The reaction conditions of the Sonogashira reaction are as described in a published document (R. Chinchilla et. al., Chem. Rev., 107 (3), 874-922 (2007).) and the like. Among the compounds represented by Formula (S8), a compound represented by Formula (S17) wherein both of R^{21a} and R^{22a} are hydrogen atoms and R⁵¹ is a t-butyl group, may be produced, for example, according to the retro-synthetic route described below.

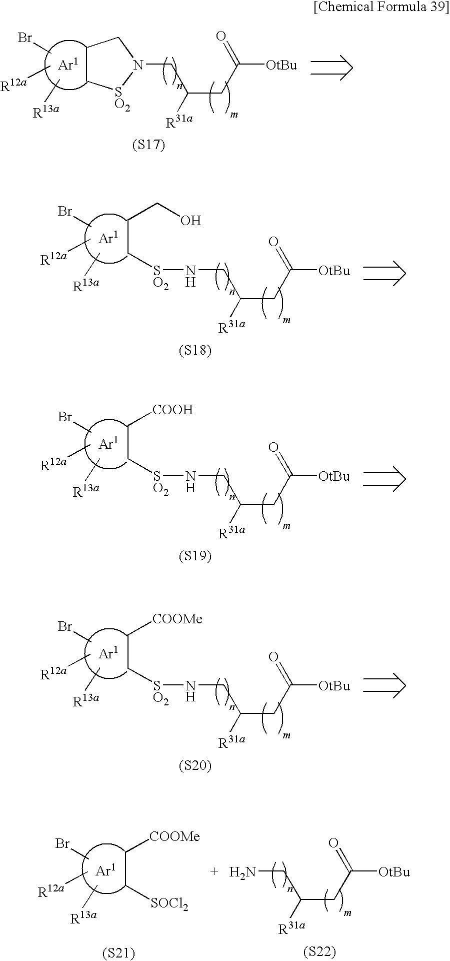

The compound that is represented by formula (S17) can be prepared by conducting Mitsunobu reaction of the compound that is represented by formula (S18) [in the formula, Ar¹, n, m, R^{12a}, R^{13a}, and R^{31a} have the meanings the same as those described in the aforementioned]. The reaction conditions of Mitsunobu reaction are as described in the literatures (Mitsunobu, O., Synthesis, 1981, 1., Hughes, D. L., Org. React., 42, 335 (1992)). Specifically, the solvent that can be used in Mitsunobu reaction may be tetrahydrofuran, dioxane, toluene, dimethoxyethane, or acetonitrile. In addition, two or more of these solvents can be used as a mixture. Preferably, the solvent is toluene. The reaction can be carried out by using a phosphorus reagent such as triphenylphosphine or tributylphosphine and an azo reagent such as diethylazodicarboxylate, diisopropylazodicarboxylate or TMAD. Each of the reagents is preferably used in an equivalent amount or an excess amount relative to the compound represented by formula (S18). More preferably, the amount is 1 to 10 equivalents. The reaction temperature is typically in the range of −50° C. to 200° C. Preferably, it is 0° C. to 100° C. Although not being specifically limited, the reaction time is generally 0.2 to 48 hours, and preferably 1 to 36 hours.

The compound that is represented by formula (S18) can be prepared by subjecting the compound represented by formula (S19) [in the formula, Ar¹, n, m, R^{12a}, R^{13a}, and R^{31a} have the meanings the same as those described in the aforementioned] to reductive reaction. A reducing agent used in the reaction may be a borane complex such as borane tetrahydrofuran, borane dimethylsulfide, borane pyridine, borane trimethylamine, or borane triphenylphosphine, or diborane. The reducing agent is generally used in an equivalent amount or an excess amount relative to the compound that is represented by formula (19). Preferably, the amount is 1 to 100 equivalents, and more preferably 1 to 10 equivalents. Depending on necessity, a Lewis acid such as SnCl₄ can be added. As a solvent, an inactive solvent can be used. Examples of the inactive solvent include dichloromethane, chloroform, carbon tetrachloride, benzene, toluene, xylene, diethyl ether, tetrahydrofuran, dioxane, and dimethoxyethane. Preferably, the solvent is dichloromethane, diethyl ether, or tetrahydrofuran. In addition, two or more of these solvents can be used as a mixture. The reaction temperature is typically in the range of −20° C. to 100° C. Preferably, it is −10° C. to 50° C. The reaction time is generally 0.2 to 96 hours, and preferably 1 to 48 hours.

The compounds that are represented by formula (S19) can be prepared by hydrolysis of methyl ester of the compounds that are represented by formula (S20) [in the formula, Ar¹, n, m, R^{12a}, R^{13a}, and R^{31a} have the meanings the same as those described in the aforementioned]. The hydrolysis reaction can be performed by using a base. In this case, the base may be alkali metal carbonates such as potassium carbonate or sodium carbonate, or alkaline metal hydroxide such as potassium hydroxide, sodium hydroxide, or lithium hydroxide. The use amount of the base is an equivalent amount or an excess amount relative to the compound represented by formula (S20). Preferably, the amount is 1 to 10 equivalents. As a solvent, a polar solvent, or a mixture solvent comprising a polar solvent and water can be used. As the polar solvent, alcohols such as ethanol, or methanol, ethers such as dioxane, tetrahydrofuran, or diethyl ether, dimethylsulfoxide, or acetone can be mentioned. Preferably, the solvent is a mixture of solvent comprising water and a polar solvent such as ethanol, methanol, dioxane, or acetone. The reaction temperature is typically in the range of −20° C. to 150° C. Preferably, it is −10° C. to 100° C. Although not being specifically limited, the reaction time is generally 0.2 to 48 hours, and preferably 1 to 24 hours.

The compound that is represented by formula (S20) can be prepared by reacting the compound that is represented by formula (S21) [in the formula, Ar¹, R^{12a}, and R^{13a} have the meanings the same as those described in the aforementioned] with the compound that is represented by formula (S22) [in the formula, n, m and R^{31a} have the meanings the same as those described in the aforementioned] in the presence of a base. The use amount of the compound that is represented by formula (S22) is an equivalent amount to an excess amount, and preferably 1 to 5 equivalents relative to the compound represented by formula (S21). The base used in the reaction may be trimethylamine, triethylamine, diisopropylethylamine, N-methylmorpholine, or pyridine. Preferably, it is triethylamine, diisopropylethylamine, N-methylmorpholine, or pyridine. The use amount of the base is an equivalent amount to an excess amount, and preferably 1 to 10 equivalents relative to the compound represented by formula (S21). The solvent that can be used in the reaction may be dichloromethane, chloroform, carbon tetrachloride, benzene, toluene, xylene, diethyl ether, tetrahydrofuran, dioxane, dimethoxyethane, pyridine, or dimethylformamide. In addition, two or more of these solvents can be used as a mixture. Preferably, the solvent is dichloromethane, or tetrahydrofuran. The reaction temperature is typically in the range of −80° C. to 100° C. Preferably, it is 0° C. to 50° C. Although not being specifically limited, the reaction time is generally 1 to 96 hours, and preferably 2 to 48 hours.

For the compound that is represented by formula (S22), for example, commercially available valine t-butyl ester (manufactured by Wako Pure Chemical Industries, Ltd) and the like can be used.

Among the compounds that are represented by formula (S4), a compound in which R^{11a} represents —O—R^{112a}—R^{113a}—R^{114a} [in the formula, each of R^{112a}, R^{113a}, and R^{114a} has the meaning the same as those described in the aforementioned] has the same meaning as the compound that is represented by formula (S23) [in the formula, Ar¹, n, m, R^{112a}, R^{113a}, R^{114a}, R^{12a}, R^{13a}, R^{21a}, R^{22a}, R^{31a}, and R⁵¹ have the meanings the same as those described in the aforementioned], and such compounds can be prepared according to a retro-synthetic route described below, for example.

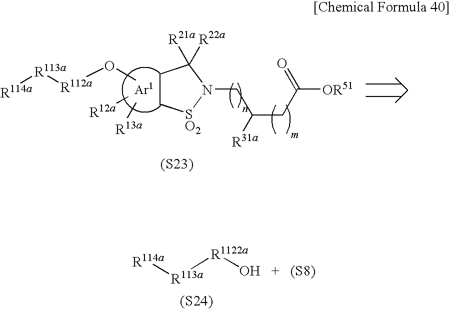

The compound that is represented by formula (S23) can be prepared by conducting known Buchwald-Hartwig reaction of the compound represented by formula (S24) [in the formula, R^{112a}, R^{113a}, and R^{114a} have the meanings the same as those described in the aforementioned] and the compound represented by formula (S8). The reaction conditions are the same as those described in published literature (A. R Muci, S. L. Buchwald, Top. Curr. Chem., 219, 131 (2002)., J. F. Hartwig, Angew. Chem., Int. Ed., 37, 2046 (1998)., D. Baranano, G. Mann, Hartwig, J. F. Curr. Org. Chem. 1, 287 (1997)., C. G. Frost, P. Mendonca, J. Chem. Soc. Perkin Trans. 1, 1998, 2615.) and the like. Specifically, the reaction can be carried out by using a metal catalyst in the presence of a base as shown below. The use amount of the compound that is represented by formula (S24) is an equivalent amount to an excess amount, and preferably 1 to 5 equivalents relative to the compound represented by formula (S8). The base used in the reaction may be potassium carbonate, sodium carbonate, cesium carbonate, potassium phosphate, trimethylamine, triethylamine, diisopropylethylamine, N-methylmorpholine, or pyridine. Preferably, it is potassium phosphate. The use amount of the base is an equivalent amount to an excess amount, and preferably 1 to 10 equivalents relative to the compound represented by formula (S24). As the metal catalyst, palladium acetate and the like can be used, and a ligand such as 2-(di-t-butylphosphino)biphenyl is preferably added. Examples of the solvent that can be used in the reaction include dichloromethane, chloroform, carbon tetrachloride, benzene, toluene, xylene, diethyl ether, tetrahydrofuran, dioxane, dimethoxyethane, pyridine, and dimethylformamide. In addition, two or more of these solvents can be used as a mixture. Preferably, the solvent is toluene. The reaction temperature is typically in the range of 0° C. to 200° C. Preferably, it is 30° C. to 150° C. Although not being specifically limited, the reaction time is generally 1 to 96 hours, and preferably 2 to 48 hours.

Among the compounds represented by Formula (S4), a compound wherein R^{11a} is represented by —NR¹¹¹¹—R^{112a}—R^{113a}—R^{114a} [Wherein R¹¹¹¹, R^{112a}, R^{113a} and R^{114a} are respectively the same as the aforementioned] may be produced, for example, according to the retro-synthetic route described below.

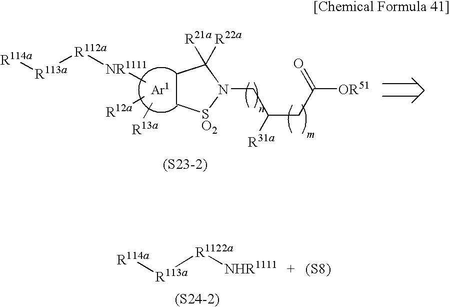

Among the compounds represented by Formula (S4), a compound wherein R^{11a} is represented by —NR¹¹¹¹—R^{112a}—R^{113a}—R^{114a} [Wherein R^{113a}, R^{112a} and R^{114a} are respectively the same as the aforementioned] is the same as the compound represented by Formula (S23-2) [Wherein R¹¹¹¹, Ar¹, n, m, R^{112a}, R^{113a}, R^{114a}, R^{12a}, R^{13a}, R^{21a}, R^{22a}, R^{31a} and R⁵¹ are the same as the aforementioned], and can be produced by known Buchwald-Hartwig reaction of a compound represented by Formula (S24-2) [Wherein R¹¹¹¹, R^{112a}, R^{113a} and R^{114a} are respectively the same as the aforementioned] and the compound represented by Formula (S8). The conditions of the Buchwald-Hartwig reaction are the same as the conditions in the method of producing the compound represented by Formula (S23).

Among the compounds represented by Formula (S4), a compound wherein R^{11a} is represented by —R^{1123a}—R^{113a}—R^{114a} [Wherein R^{1123a} represents an optionally substituted arylene group that is bonded to Ar¹ on the nitrogen atom, R^{1123a} may be protected, and R^{113a} and R^{114a} are respectively the same as the aforementioned] may be produced, for example, according to the retro-synthetic route described below.

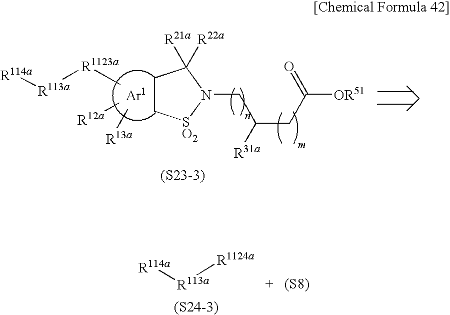

Examples of R^{1123a} include the examples of the aforementioned arylene group wherein V¹ is bonded to Ar¹ and V² is bonded to R^{113a} in EL-19, EL-58, EL-59, EL-60, EL-119 and EL-220.

Among the compounds represented by Formula (S4), a compound wherein R^{11a} is represented by —R^{1123a}—R^{113a}—R^{114a} [Wherein R^{113a}, R^{1123a} and R^{114a} are respectively the same as the aforementioned] is the same as the compound represented by Formula (S23-3) [Wherein Ar¹, n, m, R^{1123a}, R^{113a}, R^{114a}, R^{12a}, R^{13a}, R^{21a}, R^{22a}, R³¹ and R⁵¹ are the same as the aforementioned], and can be produced by conducting known Ullman reaction of a compound represented by Formula (S24-3) [Wherein R^{1124a} represents an arylene group containing —NH— in the structure, R^{1124a} may be protected, and R^{113a} and R^{114a} are respectively the same as the aforementioned] and the compound represented by Formula (S8). The conditions of the Ullman reaction are as described in a published document (A. W. Thomas et. al., Angew. Chem. Int. Ed., 42, 5400 (2003).) and the like. The reaction may also be favor ably performed under microwave irradiation conditions.

Among the compounds that are represented by formula (S4), the compound of formula (S25) in which both R^{21a} and R^{22a} represent hydrogen atoms [in the formula, Ar¹, n, m, R^{11a}, R^{12a}, R^{13a}, R^{31a}, and R⁵¹ have meanings the same as those described in the aforementioned] can be prepared according to a retro-synthetic route described below, for example.

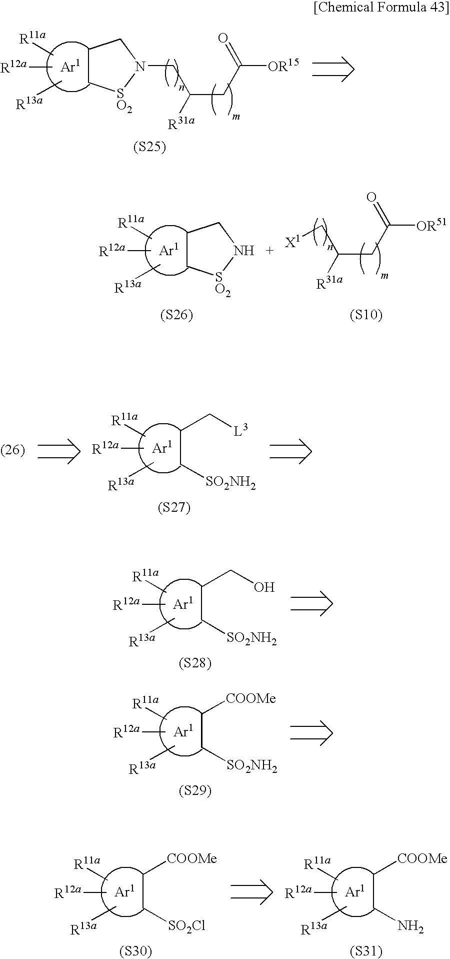

The compound that is represented by formula (S25) can be prepared by reacting the compound that is represented by formula (S26) [in the formula, Ar¹, R^{11a}, R^{12a}, and R^{13a} have the meanings the same as those described in the aforementioned] with the compound that is represented by formula (S10) [in the formula, symbols have the meanings the same as those described in the aforementioned] in the presence of a base. The use amount of the compound that is represented by formula (S10) is 1/10 to 10 equivalents, and preferably ⅕ to 5 equivalents, and more preferably 1 to 2 equivalents relative to the compound represented by formula (S26). For the base used in the reaction, a metal alkoxide such as sodium methoxide or sodium ethoxide, carbonate salts such as sodium carbonate, potassium carbonate, or cesium carbonate, a metal hydride compound such as sodium hydride, sodium hydroxide, or potassium hydroxide can be exemplified. Preferably, the base is sodium hydride. The use amount of the base is ⅕ to 10 equivalents, and preferably ½ to 3 equivalents relative to the compound represented by formula (S26). Examples of the solvent that can be used in the reaction include ethanol, methanol, propanol, isopropanol, ethylene glycol, propylene glycol, diethylene glycol monomethyl ether, dichloromethane, chloroform, carbon tetrachloride, benzene, toluene, xylene, diethyl ether, tetrahydrofuran, dioxane, dimethoxyethane, diethylene glycol dimethyl ether, ethyl acetate, butyl acetate, dimethylformamide and acetonitrile. Preferably, the solvent is dimethylformamide. In addition, two or more of these solvents can be used as a mixture. The reaction temperature is typically in the range of 0° C. to 200° C. Preferably, it is 10° C. to 150° C. Although not being specifically limited, the reaction time is generally 1 to 96 hours, and preferably 3 to 36 hours.

The compound that is represented by formula (S26) can be prepared by reacting the compound that is represented by formula (S27) [in the formula, Ar¹, R^{11a}, R^{12a}, and R^{13a} have meanings the same as those described in the aforementioned and L³ represents a bromine atom] in the presence of a base. For the base used in the reaction, a metal alkoxide such as sodium methoxide or sodium ethoxide, carbonate salts such as sodium carbonate, potassium carbonate, or cesium carbonate, a metal hydride compound such as sodium hydride, sodium hydroxide, or potassium hydroxide can be exemplified. Preferably, the base is sodium hydride. The use amount of the base is ⅕ to 10 equivalents, and preferably ½ to 3 equivalents relative to the compound represented by formula (S27). Examples of the solvent that can be used in the reaction include ethanol, methanol, propanol, isopropanol, ethylene glycol, propylene glycol, diethylene glycol monomethyl ether, dichloromethane, chloroform, carbon tetrachloride, benzene, toluene, xylene, diethyl ether, tetrahydrofuran, dioxane, dimethoxyethane, diethylene glycol dimethyl ether, ethyl acetate, butyl acetate, dimethylformamide and acetonitrile. Preferably, the solvent is dimethylformamide. In addition, two or more of these solvents can be used as a mixture. The reaction temperature is typically in the range of 0° C. to 200° C. Preferably, it is 10° C. to 150° C. Although not being specifically limited, the reaction time is generally 1 to 96 hours, and preferably 3 to 36 hours.

The compound that is represented by formula (S27) can be prepared by reacting the compound that is represented by formula (S28) [in the formula, Ar¹, R^{11a}, R^{12a}, and R^{13a} have meanings the same as those described in the aforementioned] under bromination conditions. A reagent used in the bromination may be a phosphorus reagent such as triphenylphosphine, or tributylphosphine, or a bromination compound such as carbon tetrabromide. The use amount of the phosphorous reagent is an equivalent amount to an excess amount, and preferably 1 to 4 equivalents relative to the compound represented by formula (S28). The use amount of the bromination compound is an equivalent amount to an excess amount, and preferably 1 to 4 equivalents relative to the compound represented by formula (S28). Examples of the solvent that can be used in the reaction include dichloromethane, chloroform, carbon tetrachloride, benzene, toluene, xylene, diethyl ether, tetrahydrofuran, dioxane, dimethoxyethane, diethylene glycol dimethyl ether, ethyl acetate, butyl acetate, dimethylformamide and acetonitrile. Preferably, the solvent is tetrahydrofuran. In addition, two or more of these solvents can be used as a mixture. The reaction temperature is typically in the range of 0° C. to 200° C. Preferably, it is 10° C. to 150° C. Although not being specifically limited, the reaction time is generally 1 to 96 hours, and preferably 3 to 36 hours.

The compound represented by Formula (S28) can be produced by conducting reduction reaction of a compound represented by Formula (S29) [Wherein Ar¹, R^{11a}, R^{12a} and R^{13a} are the same as the aforementioned]. The reduction reaction may be a reaction using a complex metal hydride. Preferable examples of the complex metal hydride include aluminum lithium hydride and diisobutyl aluminum hydride. The dose of the complex metal hydride used is, for example, 1 equivalent to 10 equivalents, and preferably 1 equivalent to 5 equivalents relative to the raw material. Examples of the solvent used in the reaction include diethyl ether, tetrahydrofuran, dioxane, and dimethoxyethane, and the solvent is preferably tetrahydrofuran. In addition, two or more of these solvents can be used as a mixture. The reaction temperature is generally −50° C. to 100° C., and preferably −20° C. to 70° C. The reaction time is not particularly limited, but generally 0.1 hour to 96 hours, and preferably 0.5 hours to 24 hours.

The compound represented by Formula (S29) can be produced by conducting sulfonamidation reaction of a compound represented by Formula (S30) [Wherein Ar¹, R^{11a}, R^{12a} and R^{13a} are the same as the aforementioned]. Examples of the sulfonamidation reaction include a method of reacting ammonia with the compound of Formula (S30) in a solvent. Examples of the solvent used in the reaction include water, ethanol, methanol, propanol, isopropanol, ethylene glycol, propylene glycol, diethylene glycol monomethyl ether, dichloromethane, chloroform, carbon tetrachloride, benzene, toluene, xylene, diethyl ether, tetrahydrofuran, dioxane, dimethoxyethane, diethylene glycol dimethyl ether, ethyl acetate, butyl acetate, dimethyl formamide and acetonitrile, and the solvent is preferably dioxane, ethanol, methanol and/or water. The amount of ammonia used is, for example, equivalent to excessive amount, and preferably 1 equivalent to 10 equivalents. The reaction temperature is generally 0° C. to 100° C. and preferably 10° C. to 70° C. The reaction time is not particularly limited, but generally 0.1 hour to 96 hours, and preferably 0.5 hour to 24 hours.

The compound represented by Formula (S30) can be produced by conducting chlorosulfonylation reaction of a compound represented by Formula (S31) [Wherein Ar¹, R^{11a}, R^{12a} and R^{13a} are the same as the aforementioned]. The conditions of the chlorosulfonylation reaction may be the based on, for example, those of the method described in a published document (Heterocyclic Chem., 41, 435-438 (2004).) and the like.

As the compound represented by Formula (S31), for example, commercially available methyl 3-amino-5-phenyl-2-thiophenecarboxylate (manufactured by AK Scientific, Inc.) and the like can be used.

Among the compounds represented by Formula (S9), a compound represented by Formula (S32) described below wherein both of R^{21a} and R^{22a} are hydrogen atoms [Wherein Ar¹, R^{12a} and R^{13a} are the same as the aforementioned] may be produced, for example, according to the retro-synthetic route described below.

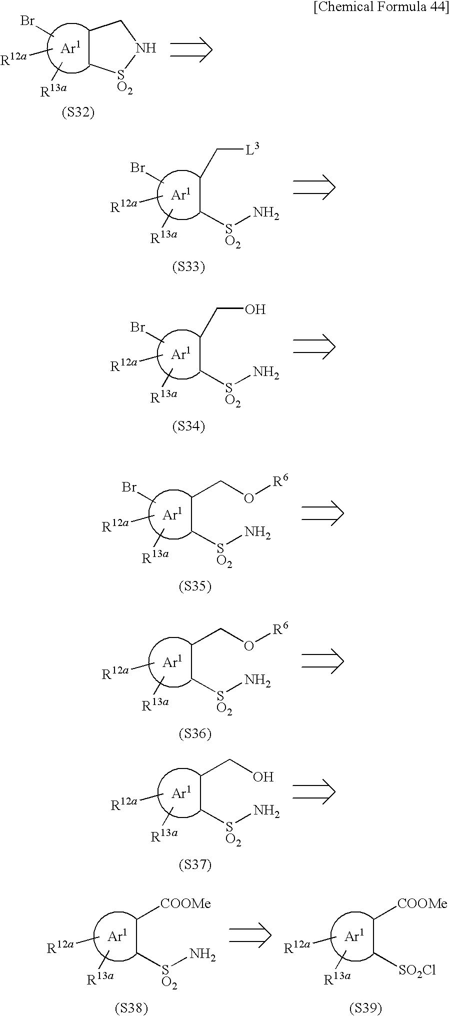

The compound represented by Formula (S32) can be produced from a compound represented by Formula (S33) [Wherein Ar¹, R^{12a}, R^{13a}, and L³ are the same as the aforementioned] according to the method of producing the compound represented by Formula (S26) from the compound represented by Formula (S27).

The compound represented by Formula (S33) can be produced from a compound represented by Formula (S34) [Wherein Ar¹, R^{12a} and R^{13a} are the same as the aforementioned] according to the method of producing the compound represented by Formula (S27) from the compound represented by Formula (S28).

The compound represented by Formula (S34) can be produced by conducting deprotection reaction of a compound represented by Formula (S35) [Wherein R⁶ represents a silyl protective group, and Ar¹, R^{12a} and R^{13a} are the same as the aforementioned]. R⁶ is a silyl protective group, and the silyl protective group may be a t-butyldimethylsilyl group, or a t-butyldiphenylsilyl group and is preferably a t-butyldiphenylsilyl group. The deprotection reaction may be performed on the basis of a known method, for example, the method as described in Protective Groups in Organic Synthesis published by John Wiley and Sons (2007) and the like.

The compound represented by Formula (S35) can be produced by reacting a lithiation agent with a compound represented by Formula (S36) [Wherein Ar¹, R^{12a}, R^{13a} and R⁶ are the same as the aforementioned], and then reacting a bromination compound with the resultant product. As a solvent that can be used in the successive reactions, an inactive solvent can be used. The inactive solvent is, for example, benzene, toluene, xylene, diethyl ether, tetrahydrofuran, dioxane, or dimethoxyethane. The inactive solvent is preferably tetrahydrofuran. In addition, two or more these solvents may be used as a mixture. The reaction temperature is generally −100° C. to 0° C., and preferably −90° C. to 0° C. The reaction time of reacting the lithiation agent with the compound of Formula (S36) is, for example, 1 minute to 24 hours, and preferably 5 minutes to 30 minutes. The reaction time of reacting the bromination compound with the product is, for example, 0.1 to 24 hours and preferably 0.5 to 10 hours. The lithiation agent may be t-butyl lithium, n-butyl lithium, and/or lithium diisopropyl amide, and is preferably lithium diisopropylamide. The bromination compound may be carbon tetrabromide.

The compound represented by Formula (S36) can be produced by conducting silylation reaction of a compound represented by Formula (S37) [Wherein Ar¹, R^{12a} and R^{13a} are the same as the aforementioned]. Examples of the silylation reaction include a method of causing a silylation agent and a base to act on the compound of Formula (S37) in a solvent. The solvent used in the reaction is, for example, dichloromethane, chloroform, carbon tetrachloride, benzene, toluene, xylene, diethyl ether, tetrahydrofuran, dioxane, dimethoxyethane, diethylene glycol dimethyl ether, ethyl acetate, butyl acetate, dimethyl formamide, dimethyl acetamide or acetonitrile and preferably dimethyl formamide. The silylation agent used is, for example, t-butyldimethylsilyl chloride, or t-butyldiphenylsilyl chloride, and preferably t-butyldiphenylsilyl chloride. The amount of the silylation agent used is equivalent to excessive amount, and preferably 1 equivalent to 10 equivalents. The base used is, for example, trimethylamine, triethylamine, diisopropylethylamine, N-methylmorpholine, imidazole or pyridine, and preferably imidazole. The amount of the base used is equivalent to excessive amount, and preferably 1 equivalent to 10 equivalents. The reaction temperature is generally 0° C. to 100° C., and preferably 10° C. to 70° C. The reaction time is not particularly limited, but generally 0.1 hour to 96 hours, and preferably 0.5 hours to 24 hours.

The compound represented by Formula (S37) can be produced by conducting reduction reaction of a compound represented by Formula (S38) [Wherein Ar¹, R^{12a} and R^{13a} are the same as the aforementioned]. The conditions of the reduction reaction are similar to those of the method of producing the compound represented by Formula (S28) from the compound represented by Formula (S29)

The compound represented by Formula (S38) can be produced by conducting sulfonamidation reaction of a compound represented by Formula (S39) [Wherein Ar¹, R^{12a} and R^{13a} are the same as the aforementioned]. The conditions of the sulfonamidation reaction are similar to those of the method of producing the compound represented by Formula (S29) from the compound represented by Formula (S30).

As the compound represented by Formula (S39), for example, commercially available methyl 3-(chlorosulfonyl)-2-thiophenecarboxylate (manufactured by Wako Pure Chemical Industries, Ltd.) and the like can be used.

Moreover, in an alternative method, the compound represented by Formula (S37) may be produced, for example, according to the retro-synthetic route described below.

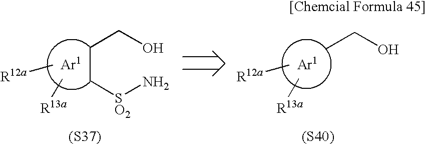

The compound represented by Formula (S37) can be produced by conducting aminosulfonylation reaction of a compound represented by Formula (S40) [Wherein Ar¹, R^{12a} and R^{13a} are the same as the aforementioned]. The aminosulfonylation reaction includes a reaction of first causing a lithiation agent to act on the compound of Formula (S40), and then causing sulfur dioxide to act on the resultant compound, and further causing hydroxylamine-O-sulfonic acid to act on the resulting product. A solvent that can be used in the steps of causing the lithiation agent and the sulfur dioxide to act on the above compound may be an inactive solvent. The inactive solvent is, for example, benzene, toluene, xylene, diethyl ether, tetrahydrofuran, dioxane, or dimethoxyethane. The inactive solvent is preferably tetrahydrofuran. In addition, two or more of these solvents may be used as a mixture. The reaction temperature in the step of causing the lithiation agent to act on the compound of Formula (S40) is generally −100° C. to 0° C., and preferably −90° C. to 0° C. The reaction time in the step of causing the lithiation agent to act on the compound of Formula (S40) is, for example, 10 minutes to 24 hours, and preferably 20 minutes to 2 hours. The lithiation agent may be t-butyl lithium, n-butyl lithium, or lithium diisopropyl amide, and is preferably n-butyl lithium. The amount of sulfur dioxide used is equivalent or excessive amount, and preferably 1 to 5 equivalents. The reaction temperature in the step of causing sulfur dioxide to act on the product obtained by reacting the lithiation agent with the compound of Formula (S40) is generally −100° C. to 50° C., and preferably −90° C. to 40° C. The reaction time in the step of causing sulfur dioxide to act on the product obtained by reacting the lithiation agent with the compound of Formula (S40) is, for example, for 1 hour to 24 hours, and preferably 2 hours to 12 hours. The amount of hydroxylamine-O-sulfonic acid used is equivalent or excessive amount, and preferably 1 to 5 equivalents. The reaction temperature in the step of causing the hydroxylamine-O-sulfonic acid to act on the product obtained by reacting sulfur dioxide with the aforementioned product is generally −50° C. to 50° C., and preferably 0° C. to 40° C. A solvent that can be used in the step of causing the hydroxylamine-O-sulfonic acid to act on the product obtained by reacting sulfur dioxide with the aforementioned product may be water or alcohol, or a mixed solvent thereof. The reaction time in the step of causing sulfur dioxide to act on the product obtained by reacting the lithiation agent with the compound of Formula (S40) is 10 minutes to 24 hours, and preferably 30 minutes to 12 hours. As the compound represented by Formula (S40), for example, commercially available 3-thiophene methanol (manufactured by Wako Pure Chemical Industries, Ltd.) and the like can be used.

R^{11a}, R^{12a}, R^{13a}, R^{21a}, R^{22a}, R^{31a}, Y^{12a}, R^{112a}, R^{113a}, R^{114a}, R^{111a}, R^{1121a}, R^{1122a}, R^{1123a}, R^{1124a} and R^{1142a} represent optionally protected groups respectively, and regarding the protective groups in the groups, they may be subjected to protection reaction or deprotection reaction if necessary in the course of the production processes. R⁵¹ is the protective group of a carboxyl group in the production processes of the compound represented by Formula (1) wherein Y¹¹ represents a carboxyl group (—COOH), and may be subjected to protection reaction, or deprotection reaction if necessary in the course of the production processes. The protection reaction and the deprotection reaction may be performed on the basis of a known method, for example, a method as described in Protective Groups in Organic Synthesis published by John Wiley and Sons (2007) and the like.

The compounds of the present invention that are thus obtained, and the raw materials and the intermediates thereof can be isolated and purified by a general method such as extraction, distillation, or chromatography.

From the compounds that are represented by the above-described formula (1), salts thereof can be prepared. Although the method for producing the salt is not specifically limited, an exemplary method for producing an acid addition salt may be a method in which the compound that is represented by formula (1) is dissolved in alcohol such as methanol, or ethanol followed by addition of an acid component in an equivalent amount to several equivalents to obtain an acid addition salt. The acid component that can be used may be an acid component which corresponds to the later-described acid addition salt. The acid component is preferably pharmaceutically acceptable mineral acid and/or organic acid such as hydrochloric acid, hydrobromic acid, sulfuric acid, hydrogen sulfate, dihydrogen phosphate, citric acid, maleic acid, tartaric acid, fumaric acid, gluconic acid or methanesulfonic acid. Further, with respect to a method for preparing a basic addition salt, a method which is the same as the method for producing an acid addition salt except that a base component is used instead of the acid component can be carried out. As for the base component which can be used, any of base components which correspond to the later-described base addition salts can be used. Preferred examples thereof include pharmaceutically acceptable bases such as sodium hydroxide, potassium hydroxide, N-methyl-D-glucamine, N,N′-dibenzylethylene diamine, 2-aminoethanol, tris(hydroxymethyl)aminomethane, arginine and lysine.

The type of the salts of the compounds of the present invention is not specifically limited, and the salt can be an acid addition salt or a base addition salt. In addition, it can be in the form of a zwitterion in a molecule. The acid addition salts include salts with hydrochloric acid, hydrobromic acid, sulfuric acid, hydrogen sulfate, dihydrogen phosphate, citric acid, maleic acid, tartaric acid, fumaric acid, gluconic acid or methanesulfonic acid and an addition salt with an optically active acid such as camphor sulfonic acid, mandelic acid, or substituted mandelic acid. The base addition salts include metal salts such as sodium salt, and potassium salt, and an addition salt with an organic base such as N-methyl-D-glucamine, N,N′-dibenzylethylene diamine, 2-aminoethanol, tris(hydroxymethyl)aminomethane, arginine or lysine. However, the type of the salt is not limited to the above-described salts, and it can be appropriately selected by a skilled person in the art. Among these, pharmaceutically acceptable salts are preferred. In addition, the compounds of the present invention may be present as a hydrate or a solvate, and these are also within the scope of the present invention.

When a prodrug is formed from the compound of formula (1), for example, a group which can yield a prodrug is introduced into at least one of groups selected from a hydroxy group and an amino group of the compound by a conventional means and by using a corresponding halide as a reagent for obtaining a prodrug and, if desired, isolation and purification are carried out according to conventional methods. Thus, the prodrug can be prepared. In addition, a group which can yield a prodrug can be introduced into the carboxyl group of the compound of formula (1) according to conventional methods by using a reagent for obtaining a prodrug such as corresponding alcohol or amine. Alternatively, in order to obtain a prodrug, the production can be carried out while using the protective group present on the compound of formula (S2).

Although not specifically limited, the prodrug of the compound of Formula (1) can be a compound wherein a group for obtaining a prodrug is introduced into at least one group selected from a hydroxyl group, an amino group, and a carboxy group. The group for obtaining a prodrug with respect to a hydroxy group and an amino group may be an acyl group or an alkoxycarbonyl group. It is preferably an acetyl group, a propionyl group, a methoxycarbonyl group, or an ethoxycarbonyl group. It is more preferably an ethoxycarbonyl group. In another embodiment, an acetyl group is preferred. In still another embodiment, a propionyl group is preferred. In still another embodiment, a methoxycarbonyl group is preferred. The group for obtaining a prodrug with respect to a carboxy group may be a methyl group, an ethyl group, a n-propyl group, an isopropyl group, a n-butyl group, an isobutyl group, a s-butyl group, a t-butyl group, an amino group, a methylamino group, an ethylamino group, a dimethylamino group, or a diethylamino group. It is preferably an ethyl group, a n-propyl group, or an isopropyl group. It is more preferably an ethyl group. In another embodiment, a n-propyl group is preferred. In still another embodiment, an isopropyl group is preferred.

The compound represented by Formula (1) may have an asymmetric carbon. The configuration of such an asymmetric carbon atom is not specifically limited. It can be S configuration or R configuration, or a mixture of both. Optically active substances based on the asymmetric carbon, stereoisomers such as diastereomers, mixtures of stereoisomers and racemates are all included in the scope of the present invention.

Since the compound of the present invention represented by Formula (1), a salt thereof, or a prodrug thereof has a strong inhibitory effect on aggrecanase activity as described later, they are useful as a pharmaceutically active ingredient. Inhibition of aggrecanase activity blocks degradation of cartilage aggrecan, and therefore suppresses destruction of a joint cartilage. Thus, it is thought that the compounds of the present invention are useful for treating disorders that are characterized by destruction of a joint cartilage. Examples of the disorders that are characterized by destruction of a joint cartilage include osteoarthritis, joint injury, reactive arthritis, acute pyrophosphate arthritis, psoriatic arthritis, and rheumatoid arthritis.

The fact that the compound of the present invention represented by Formula (1), a salt thereof, or a prodrug thereof can suppress degradation of cartilage aggrecan can be verified, for example, using articular cartilage ecplant and the like (Yamada, et al.: Journal of Rheumatology, 26, p 654-662, 1999, Pratta M. A., et al.: J. Biol. Chem. 278, p 45539-45545, 2003). The action of suppressing aggrecan degradation can be verified by incubating the explant cartilage of a rabbit or bovine with addition of the compound of the present invention and interleukin 1, and after two days, measuring the amount of aggrecan fragment, or the amount of glycosaminoglycan that is released into the incubated supernatant.

The fact that the compound of the present invention represented by Formula (1), a salt thereof, or a prodrug thereof is useful as an active ingredient for preventing and/or treating osteoarthritis can be verified using an osteoarthritis model such as rat anterior cruciate ligament resection (Hayami T: Arthritis Rheum, 50, p 1193-1206, 2004) or meniscal cutting (Janusz M. J., et al.: Osteoarthritis Cartilage, 10, p 785-791, 2002), or mouse meniscal destabilization (Glasson S S: Nature, 434, p 644-648, 2005). The compound of the present invention, a salt thereof, or a prodrug thereof can be verified for its usefulness as a pharmaceutical agent of treating osteoarthritis by administering a dose of 0.1 to 1000 mg/kg to a model animal or ally, intravenously, intraperitoneally or intra-articulately, and analyzing knee cartilage destruction proceeding histopathologically or measuring the amount of the cartilage matris ingredients such as aggrecan and collagen. The fact that the compound of the present invention represented by Formula (1), a salt thereof, or a prodrug thereof is useful as an active ingredient for preventing and/or treating rheumatoid arthritis can be verified using, for example, a collagen-induced arthritis model of rat or mouse (Griffith M. M., et al., Arthritis Rheumatism, 24, p 781, 1981; Wooley P. H. et al., J. Exp. Med., 154, p 688, 1981.) and the like. The compound of the present invention, a salt thereof, or a prodrug thereof can be verified for its usefulness as a pharmaceutical agent of treating chronic rheumatic arthritis by administering a dose of 0.1 to 1000 mg/kg to a model animal or ally, intravenously, intraperitoneally or intra-articularly, and measuring the heel volume or measuring bone destruction proceeding.

The fact that the compound of the present invention represented by Formula (1), a salt thereof, or a prodrug thereof does not cause an undesirable side effect, i.e., musculoskeletal syndrome can be verified using, for example, Lewis rat and the like (Richard R. et al, Arthritis Rheumatism, 48, 6, p 1742-1749, 2003). The presence or absence of any undesirable side effect of the compound of the present invention can be confirmed by administering a dose of 0.1 to 1000 mg/kg to a model animal or ally, or intra-articularly, or continuously administering by using an osmotic pump, and, two weeks after the administration, observing the occurrence of swelling or pathological change (e.g., over production of synovium) of a hind limb knees joint.

With respect to the compound of the present invention represented by Formula (1), a salt thereof, or a prodrug thereof, by investigating selectivity between aggrecanase and various matrix metalloproteinase (MMP-2, MMP-3, MMP-14 and the like), no correlation between efficacy and undesirable side effect such as musculoskeletal syndrome or increased level of arthritis can be shown. Selectivity for aggrecanase and various MMPs can be presented as an inhibitory activity according to conventional enzyme assays.

The pharmaceutical agent of the present invention can be prepared so as to include the compound of Formula (1) or the salt thereof as an active ingredient. When a compound that is administered as a prodrug or a salt thereof is metabolized in a living body to produce the compound of Formula (1) or a pharmaceutically acceptable salt thereof, such a compound or salt is also included in the scope of the pharmaceutical agent of the present invention.

Regarding the pharmaceutical agent of the present invention, the compound of Formula (1) or pharmaceutically acceptable salt thereof or a mixture including at least two types of the compounds or pharmaceutically acceptable salts thereof can be used as it is. However, it is preferable that by adding one, or at least two types of pharmaceutically acceptable carriers to the compound of Formula (1) or pharmaceutically acceptable salt thereof or the mixture including at least two types of the compounds or pharmaceutically acceptable salts thereof, a pharmaceutical composition is prepared and used for administration. The type of the pharmaceutically acceptable carrier is not specifically limited. For example, a vehicle, a binding agent, a disintegrating agent, a lubricant or an additive, etc. can be used as such a carrier. The compound(s) or pharmaceutically acceptable salt(s) thereof can be suspended or dissolved in purified water which includes 0.5% of carboxymethylcellulose.

To use the compound(s) or pharmaceutically acceptable salt(s) thereof as the pharmaceutical agent described above, an effective amount of the compound(s) or pharmaceutically acceptable salt(s) thereof can be used as it is, or can be used in a pharmaceutical composition obtained by mixing it or them with a pharmaceutically acceptable carrier. Such a carrier may be a suspending agent such as carboxymethylcellulose, or, in some cases, purified water, or physiological saline. Further, other known carriers can also be used. For example, the compound(s) of Formula (1) or pharmaceutically acceptable salt (s) thereof can be suspended or dissolved in purified water which includes 0.5% of carboxymethylcellulose.

The formulation type for formulating the above-described pharmaceutical composition may be a tablet, powdered medicine, granules, syrup, suspension agent, a capsule, or an injection solution. For preparation of the formulation, various carriers are used depending on the type of the formulation. For example, a carrier for an or ally administered formulation may be a vehicle, a binding agent, a lubricant, an agent for promoting flowability, or a color ant.

When the compound(s) of the present invention are used in a parenteral agent such as an injection solution, distilled water for an injection solution, physiological saline, glucose solution, vegetable oil for an injection solution, propylene glycol, polyethylene glycol and the like can be used generally as a diluent. In addition, if required, a bactericidal agent, a preservative, a stabilizing agent, a tonicity agent, a soothing agent and the like can be added.

When the compound(s) of the present invention are administered to a mammal, for example, a human, they can be or ally administered in the form of a tablet, powdered medicine, granules, suspension agent, or a capsule. In addition, it can be parenterally administered in the form of an injection solution including instillation, a suppository, a gel, a lotion, an ointment, cream or spray. The administration dose varies depending on symptom to be treated, administration type, age, weight and severeness of symptom of a patient. Generally, it can be administered in a dose of 0.01 to 1000 mg per day. The dose can be administered one time or may be divided into 2 or 3 portions. Administration period is generally from several days to two months and administering everyday is general. However, depending on symptom of a patient, daily dose and administration period can be either increased or decreased.

Further, to enhance the therapeutic effect, the pharmaceutical agent of the present invention can be used in combination with other drugs and supplements that do not impair the activity of the pharmaceutical agent of the present invention. Examples of such drugs for combination use include, but are not limited to, a standard non-steroidal anti-inflammatory compound such as Piroxicam, Diclofenac, propionates such as Naproxen, Flurbiprofen, Fenoprofen, Ketoprofen and Ibuprofen; phenamates such as Mefenamic Acid, Indomethacin, Sulindac, Apazone, pyrazolones such as Phenylbutazone, salicylates such as Aspirin; COX-2 inhibitor such as Celecoxib, Valdecoxib, Paracoxib, Rofecoxib; a painkiller such as LTD-4, LTB-4 and 5-LO inhibiting agent, p 38 kinase inhibiting agent and an intra-articular therapy such as using corticosteroid and hyaluronic acid including Hyalgan and Synvisc. Examples of the supplement for combination use include, but are not limited to, glucosamine and chondroitin sulfate.

Administration time period for the above-described agent for combination use is not specifically limited. The pharmaceutical agent of the present invention and the agent(s) for combination use can be administered to a subject either simultaneously or with a time interval. The dose of the agent for combination use can be similar to the dose that is clinically used. It can be appropriately selected according to a subject to whom the pharmaceutical agent and the agent for combination use are administered, an administration route, a disorder to be treated, and the type of a combination of the pharmaceutical agent of the present invention and the agent for combination use.

The administration form of the agent for combination use is not specifically limited as long as the pharmaceutical agent of the present invention and the agent(s) for combination use are combined at the time of administration. Examples of such administration form include 1) administering a single formulation that is obtained by formulating the compound of the present invention, a salt thereof, or a prodrug thereof as an active ingredient of the pharmaceutical agent of the present invention together with the agent(s) for combination use, 2) administering simultaneously two kinds of formulations that are obtained by separate formulation of the pharmaceutical agent of the present invention and the agent(s) for combination use via the same administration route, 3) administering with time interval two kinds of formulations that are obtained by separate formulation of the pharmaceutical agent of the present invention and the agent(s) for combination use via the same administration route, 4) administering simultaneously two kinds of formulations that are obtained by separate formulation of the pharmaceutical agent of the present invention and the agent (s) for combination use via different administration routes, and 5) administering with time interval two kinds of formulations that are obtained by separate formulation of the pharmaceutical agent of the present invention and the agent(s) for combination use via different administration routes (for example, the pharmaceutical agent of the present invention is administered first followed by the administration of the agent(s) for combination use, or vice versa).

The mixing ratio between the pharmaceutical agent of the present invention and the agent(s) for combination use can be appropriately selected according to a subject to whom the pharmaceutical agent of the present invention and the agent(s) for combination use are administered, an administration route, a disorder to be treated, and the like.

The usefulness of the pharmaceutical agent of the present invention can be verified by using the effects and/or blood drug concentration transition (for example, maximum blood drug concentration, effective blood concentration duration, half-life in blood, or AUC) of the compound of the present invention, a salt thereof, or a prodrug thereof and using a mammalian animal (for example, mouse, rat, guinea pig, rabbit, dog, or monkey) including human or cells thereof (cartilage cells, primary cells, live cells and the like). In addition, the compound of the present invention, a salt thereof, or a prodrug thereof can be verified for its further high usefulness as a pharmaceutical agent by checking low toxicity of the compound of the present invention, a salt thereof, or a prodrug thereof. Furthermore, the usefulness can be verified by blood concentration transition, enzyme induction, enzyme inhibition, and stability to microsome in human or other animals. In particular, verification of the usefulness of the compound of the present invention or a salt thereof can be performed with stability to microsome in an exemplary preferable embodiment.

### EXAMPLES

The invention will be more specifically explained below by Examples and Test Examples. However, the scope of the present invention is not limited to those examples described below.

Various kinds of analysis were performed as follows in Examples described below unless otherwise stated. Thin-layer chromatography (TLC) was performed using Precoated silicagel 60 F254 (manufactured by Merck & Co., Inc.), and the spot was checked by UV (254 nm) irradiation. The silica gel column chromatography was performed using Wakogel C-300 (manufactured by Wako Pure Chemical Industries, Ltd.) or silica gel 60N (spherical, neutral, 40 to 100 μm, manufactured by KANTO CHEMICAL CO., INC.) as a filler, or performed using “Quad preparative isolation system” (manufactured by Biotage AB) and one or several pieces of any one of the cartridge columns, i.e., KP-Sil-12S, 12M, 40S and 40M manufactured by the same company as a column. In the same manner, the silica gel column chromatography may be performed using “Flash column system” (manufactured by Biotage AB). Among the procedures in Examples, “condensation” means distilling away of solvent or excess reagent using an evaporator (manufactured by TOKYO RIKAKIKAI CO., LTD.) under reduced pressure.

For HPLC of LC-MS, specific elution conditions are as follows:

LC Condition 1; The target product was eluted using a column manufactured by Waters Corporation (ACQUITY HPLC BEH C18 1.7 μM 2.1×50 mm) by a gradient elution of water-acetonitrile (containing 0.1% (v/v) acetic acid) shown below.
Flow rate: 0.6 ml/min
Solvent: Solution A containing water and 0.1% (v/v) acetic acid, Solution B containing acetonitrile and 0.1% (v/v) acetic acid From 0 minute to 2 minutes: Linear gradient of from [Solution A 95%+Solution B 5% (v/v)] to [Solution A 10%+Solution B 90% (v/v)]
From 2 minutes to 2.5 minutes: Linear gradient of from [Solution A 10%+Solution B 90% (v/v)] to [Solution A 2%+Solution B 98% (v/v)]
From 2.6 minutes to 3.2 minutes: Linear gradient to [Solution A 2%+Solution B 98% (v/v)]
LC Condition 2; The target product was eluted using a column manufactured by NOMURA CHEMICAL CO., LTD. (Develosil C30-UG-5 4.6×50 mm) by a gradient elution of water-acetonitrile (containing 0.1% (v/v) acetic acid) shown below.
Flow rate: 2 ml/min
Solvent: Solution A containing water and 0.1% (v/v) acetic acid; Solution B containing acetonitrile and 0.1% (v/v) acetic acid From 0 minutes to 5 minutes: Linear gradient of from Solution A 95%+Solution B 5% (v/v) to Solution A 2%+Solution B 98% (v/v)
From 5 minutes to 6 minutes: Maintaining at Solution A 2%+Solution B 98% (v/v)
From 6 minutes to 7.5 minutes: Maintaining at Solution A 95%+Solution B 5% (v/v)

### Reference Example 1

### Methyl 3-chlorosulfonyl-5-phenyl-2-thiophenecarboxylate

Methyl 3-amino-5-phenyl-2-thiophenecarboxylate (5 g, manufactured by AK Scientific, Inc.) was dissolved in 20% hydrochloric acid (12 ml), and sodium nitrite (1.5 g, manufactured by Wako Pure Chemical Industries, Ltd.) dissolved in water (3.2 ml) under ice cooling was dripped to the resultant solution, and the resultant reaction solution was stirred for 1 hour. An acetic acid solution in which sulfur dioxide (2.5 g) and copper chloride (530 mg, manufactured by KANTO CHEMICAL CO., INC.) were dissolved was slowly added to the solution, and the resultant reaction solution was stirred for 5 hours. The reaction mixture was poured into ice water (100 ml), and a product was extracted with dichloromethane (100 ml), dried with magnesium sulfate and then concentrated to give 6.5 g of the titled compound.

### Reference Example 2

### Methyl 5-phenyl-3-sulfamoylthiophen-2-carboxylate

The compound of Reference Example 1 (2 g) was dissolved in THF (10 ml) and 0.5M ammonia-dioxane solution (40 ml, manufactured by Aldrich) and the resultant solution was stirred at room temperature for 3 hours. The reaction mixture was concentrated and the obtained residue was washed with ethyl acetate (20 ml) to give 1.7 g of the titled compound. LC-MS: HPLC retention time 2.80 minutes (LC Condition 2), m/z 298 (MH+)

### Reference Example 3

### 2-(hydroxymethyl)-5-phenylthiophen-3-sulfonamide

The compound of Reference Example 2 (634 mg) was dissolved in tetrahydrofuran (8 ml), and lithium aluminum hydride (122 mg, manufactured by Wako Pure Chemical Industries, Ltd.) was added thereto under ice cooling, and the resultant solution was stirred for 30 minutes. 5N hydrochloric acid (2 ml) was added to the reaction mixture, and the resultant blend was filtered with celite, and washed with ethyl acetate. The obtained solution was washed with saturated saline, dried with magnesium sulfate and then concentrated to give 151 mg of the titled compound. LC-MS: HPLC retention time 1.14 minutes (LC Condition 1), m/z 270 (MH+).

### Reference Example 4

### 2-bromomethyl-5-phenylthiophen-3-sulfonamide

The compound of Reference Example 3 (151 mg) was dissolved in dichloromethane (1.5 ml) and tetrahydrofuran (0.5 ml), and triphenylphosphine (176 mg, manufactured by Tokyo Chemical Industry Co., Ltd.) and carbon tetrabromide (223 mg, manufactured by Wako Pure Chemical Industries, Ltd.) were added thereto under ice cooling, and the resultant mixture was stirred for 30 minutes. Water (2 ml) and ethyl acetate (4 ml) were added to the reaction mixture, and a product was extracted with ethyl acetate, washed with saturated saline, dried with magnesium sulfate and then concentrated. The obtained residue was put into a silica gel column (hexane:ethyl acetate=5:1) to give 51 mg of the titled compound. LC-MS: HPLC retention time 3.95 minutes (LC Condition 2)

### Reference Example 5

### 5-phenyl-1,1-dioxo-2,3-dihydrothieno[2,3-d]isothiazole

The compound of Reference Example 4 (520 mg) was dissolved in N,N-dimethylformamide (8 ml), and sodium hydride (63 mg, manufactured by KANTO CHEMICAL CO., INC.) was added thereto and the resultant mixture was stirred at 55° C. for 16 hours. Water (8 ml) and ethylacetate (10 ml) were added to the reaction mixture, and a product was extracted with ethyl acetate, washed with saturated saline, dried with magnesium sulfate and then concentrated. The obtained residue was put into a silica gel column (hexane:ethyl acetate=2:1) to give 138 mg of the titled compound. LC-MS: HPLC retention time 3.56 minutes (LC Condition 2), m/z 252 (MH+)

### Reference Example 6

### Ethyl 3-methyl-2-(5-phenyl-1,1-dioxothieno[2,3-d]isothiazol-2(3H)-yl)butyrate

The compound of Reference Example 5 (67 mg) and sodium hydride (13 mg, Wako Pure Chemical Industries, Ltd., containing 40% mineral oil) were dissolved in N,N-dimethylformamide (1 ml), and the resultant solution was stirred at 60° C. for 1 hour. Then, ethyl 2-bromoisovalerate (68 μl, manufactured by Wako Pure Chemical Industries, Ltd.) was added thereto and the reaction solution was further stirred for 24 hours. The reaction mixture was stood to cool to room temperature and then concentrated, and ethyl acetate (2 ml) and water (2 ml) were added thereto. A product was extracted with ethyl acetate, washed with saturated saline, dried with magnesium sulfate and then concentrated. The obtained residue was put into a silica gel column (hexane:ethyl acetate=5:1) to give 16 mg of the titled compound. LC-MS: HPLC retention time 4.94 minutes (LC Condition 2), m/z 380 (MH+)

### Reference Example 7

### N-(2-methoxypropan-2-yloxy)-3-methyl-2-(5-phenyl-1,1-dioxothieno[2,3-d]isothiazol-2(3H)-yl)butanoic Acid Amide

The compound of Example 1 (8 mg) was dissolved in N,N-dimethylformamide (0.5 ml), and O-(2-methoxypropan-2-yl)hydroxylamine (7.9 mg), N-hydroxybenzotriazole (5.1 mg, manufactured by KOKUSAN CHEMICAL CO., LTD), 1-ethyl-3-(3-dimethylaminopropyl)-carbodiimide hydrochloride (7.2 mg, manufactured by KOKUSAN CHEMICAL CO., LTD) and triethylamine (5.3 μl, manufactured by Wako Pure Chemical Industries, Ltd.) were added thereto and the resultant mixture was stirred at room temperature for 12 hours. To the reaction mixture, ethyl acetate (2 ml) and water (2 ml) were added. A product was extracted with ethylacetate, sequentially washed with saturated sodium bicarbonate aqueous solution and saturated saline, dried with magnesium sulfate and then concentrated to give 9.2 mg of the titled compound.

### Example 1

### 3-methyl-2-(5-phenyl-1,1-dioxothieno[2,3-d]isothiazol-2(3H)-yl)butanoic Acid

The compound of Reference Example 6 (16 mg) was dissolved in 1N sodium hydroxide aqueous solution (0.1 ml) and methanol (0.4 ml), and the resultant solution was stirred for 2.5 hours. 1N Hydrochloric acid (0.1 ml) was added to the reaction mixture, and the resulting mixture was concentrated. The obtained residue was put into a silica gel column (dichloromethane:methanol=10:1) to give 11 mg of the titled compound. LC-MS: HPLC retention time 4.10 minutes (LC Condition 2), m/z 352 (MH+).

### Example 2

### N-hydroxy-3-methyl-2-(5-phenyl-1,1-dioxothieno[2,3-d]isothiazol-2(3H)-yl)butanoic Acid Amide

The compound of Reference Example 7 (9.2 mg) was dissolved in methanol (0.3 ml), and 2N hydrochloric acid (0.3 ml) was added thereto and the resultant mixture was stirred at room temperature for 1.5 hours. 2N sodium hydroxide aqueous solution (0.3 ml) was added to the reaction mixture and the resultant mixture was concentrated. The obtained residue was put into a silica gel column (dichloromethane:methanol=10:1) to give 4.6 mg of the titled compound. LC-MS: HPLC retention time 3.51 minutes (LC Condition 2), m/z 367 (MH+).

### Examples 3 to 14

Synthesis of each of the compounds of Examples 3 to 14 is shown below. Details of Examples 3 to 14 are shown in Table 1-1. Meanings of the symbols in Table 1-1 are as follows: “Exp.”; Example number, “Structure”; Example compound, “LCRT”; retention time (minutes) of liquid chromatography in LCMS, “LCMT”; LC Condition, “MS”; mass spectral data in LCMS, “Syn.”; method of synthesizing corresponding intermediate and Example compound.

The compound having the mark “*” in an asymmetric carbon atom among the structural formulae in the “Structure” section represents an optically active substance having α configuration or β configuration in the asymmetric carbon.

The symbols in the section of the symbol “Syn.” represent a production method described below in the present invention unless otherwise stated, as apparent to the skilled in the art. “A”; the production method shown in Reference Example 1, “B”; the production method shown in Reference Example 2, “C”; the production method shown in Reference Example 3, “D”; the production method shown in Reference Example 4, “E”; the production method shown in Reference Example 5, “F”; the production method shown in Reference Example 6, “G”; the production method shown in Reference Example 7, “a”; the production method shown in Example 1, and “b”; the production method shown in Example 2.

TABLE 1-1

| Exp. | Structure | LCRT | LCMT | MS | Ref. |
|---|---|---|---|---|---|
| 3 | 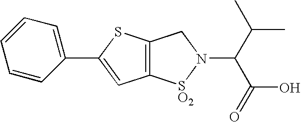 | 4.1 | 2 | 352 (MH+) | A, B, C, D, E, F, a |
| 4 | 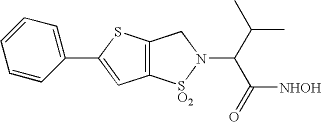 | 3.51 | 2 | 367 (MH+) | A, B, C, D, E, F, a, G, b |
| 5 | 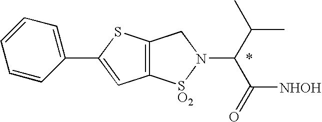 | 3.51 | 2 | 367 (MH+) | A, B, C, D, E, F, a, G, b |
| 6 | 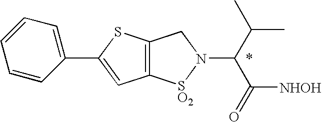 | 3.51 | 2 | 367 (MH+) | A, B, C, D, E, F, a, G, b |
| 7 | 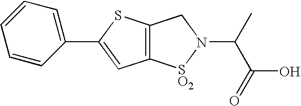 | 1.38 | 1 | 324 (MH+) | A, B, C, D, E, F, a |
| 8 | 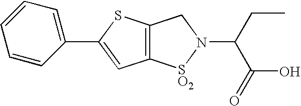 | 1.48 | 1 | 338 (MH+) | A, B, C, D, E, F, a |
| 9 | 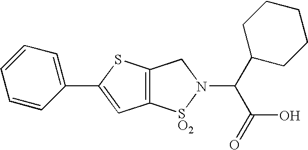 | 1.79 | 1 | 392 (MH+) | A, B, C, D, E, F, a |
| 10 | 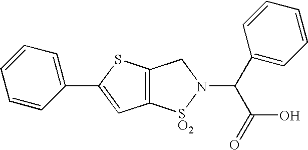 | 1.63 | 1 | 386 (MH+) | A, B, C, D, E, F, a |
| 11 | 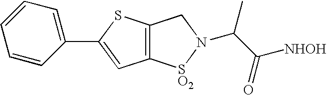 | 1.24 | 1 | 339 (MH+) | A, B, C, D, E, F, a, G, b |
| 12 | 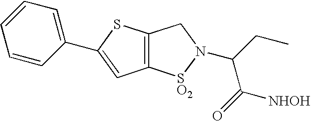 | 1.32 | 1 | 353 (MH+) | A, B, C, D, E, F, a, G, b |
| 13 | 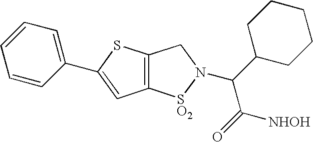 | 1.6 | 1 | 407 (MH+) | A, B, C, D, E, F, a, G, b |
| 14 | 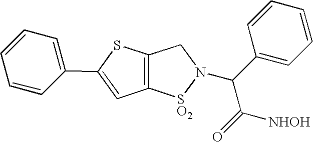 | 1.5 | 1 | 401 (MH+) | A, B, C, D, E, F, a, G, b |

### Reference Example 8-1

### Methyl 2-(5-(4-(1-methyl-1H-indol-5-yl)phenyl)-1,1-dioxothieno[2,3-d]isothiazol-2(3H)-yl)butyrate

Methyl 2-(5-(4-bromophenyl)-1,1-dioxothieno[2,3-d]isothiazol-2(3H)-yl)butyrate (64.5 mg) was dissolved in N,N-dimethylformamide ml), and bis(dibenzylideneacetone) palladium (27.5 mg, manufactured by Aldrich), tripotassium phosphate (63.7 mg, manufactured by Wako Pure Chemical Industries, Ltd.), tri-o-tolylphosphine (18.3 mg, manufactured by KANTO CHEMICAL CO., INC.) and 1-methyl-5-indole boronic acid (31.5 mg, manufactured by Frontier Corporation) were added thereto. The resultant mixture was stirred at 140° C. for 30 minutes while it was irradiated with microwave. Water (1 ml), 1N hydrochloric acid (1 ml) and ethyl acetate (1 ml) were added to the reaction mixture, and a product was extracted with ethyl acetate, washed with saturated saline, dried with magnesium sulfate and then concentrated. The obtained residue was put into a silica gel column (hexane:ethyl acetate=3:1) to give 24.9 mg of the titled compound. LC-MS: HPLC retention time 2.04 minutes (LC Condition 1), m/z 481 (MH+).

### Reference Example 8-2

### Methyl(E)-2-(5-(4-(4-methoxystyryl) phenyl)-1,1-dioxothieno[2,3-d]isothiazol-2(3H)-yl)butyrate

Methyl 2-(5-(4-bromophenyl)-1,1-dioxothieno[2,3-d]isothiazol-2(3H)-yl)butyrate (64.5 mg) was dissolved in N,N-dimethylformamide (0.75 ml), and bis(dibenzylideneacetone) palladium (27.5 mg, manufactured by Aldrich), tripotassium phosphate (63.7 mg, manufactured by Wako Pure Chemical Industries, Ltd.), tri-o-tolylphosphine (18.3 mg, manufactured by KANTO CHEMICAL CO., INC.) and 4-methoxyphenylvinyl boronic acid (32.0 mg, manufactured by Aldrich) were added thereto. The resultant mixture was stirred at 140° C. for 30 minutes while it was irradiated with microwave. Water (1 ml), 1N hydrochloric acid (1 ml) and ethyl acetate (1 ml) were added to the reaction mixture, and a product was extracted with ethyl acetate, washed with saturated saline, dried with magnesium sulfate and then concentrated. The obtained residue was put into a silica gel column (hexane:ethyl acetate=3:1) to give 30.5 mg of the titled compound. LC-MS: HPLC retention time 2.09 minutes (LC Condition 1), m/z 484 (MH+)

### Reference Example 8-3

### Methyl (E)-2-(5-(2-((biphenyl-4-yl)vinyl)-1,1-dioxothieno[3,2-d]isothiazol-2(3H)-yl)butyrate

Methyl 2-(5-bromo-1,1-dioxothieno[3,2-d]isothiazol-2(3H)-yl) butyrate (22.5 mg) was dissolved in N,N-dimethylformamide (0.32 ml), and bis(dibenzylideneacetone) palladium (11.7 mg, manufactured by Aldrich), tripotassium phosphate (27.2 mg, manufactured by Wako Pure Chemical Industries, Ltd.), tri-o-tolylphosphine (7.8 mg, manufactured by KANTO CHEMICAL CO., INC.) and 4-methoxyphenylvinyl boronic acid (32.0 mg, manufactured by Aldrich) were added thereto. The resultant mixture was stirred at 140° C. for 15 minutes while it was irradiated with microwave. Water (1 ml), 1N hydrochloric acid (1 ml) and ethyl acetate (1 ml) were added to the reaction mixture, and a product was extracted with ethyl acetate, washed with saturated saline, dried with magnesium sulfate and then concentrated. The obtained residue was put into a silica gel column (hexane:ethyl acetate=4:1) to give 9.4 mg of the titled compound. LC-MS: HPLC retention time 5.96 minutes (LC Condition 2), m/z 454 (MH+)

### Reference Example 9

### Methyl 2-(5-(6-morpholinopyridin-3-yl)-1,1-dioxothieno[2,3-d]isothiazol-2(3H)-yl)butyrate

Tetrakistriphenylphosphine palladium (879 mg, manufactured by Tokyo Chemical Industry Co., Ltd.), 6-(morpholin-4-yl)pyridin-3-boronic acid pinacol ester (65 mg, manufactured by Aldrich) and 2M sodium carbonate aqueous solution (200 μl) were added to a solution of the compound of Reference Example 18 (40 mg) in dimethylformamide (560 μl, manufactured by KANTO CHEMICAL CO., INC.), and the resultant mixture was stirred at 100° C. over night. 1M Hydrochloric acid (400 μl, manufactured by KANTO CHEMICAL CO., INC.) was added to the reaction solution for neutralization, and a product was extracted with ethyl acetate (3×5 ml), washed with saturated saline (10 ml), dried (MgSO₄), and then the solvent was distilled away. The obtained residue was purified with preparative thin-layer chromatography (CHCl₃:MeOH=9:1) to give 51 mg of the titled compound.

### Reference Example 10

### Methyl 2-(5-(phenylethynyl)-1,1-dioxothieno[2,3-d]isothiazol-2(3H)-yl)butyrate

To a solution of the compound of Reference Example 18 (40 mg) in tetrahydrofuran (500 μl, manufactured by KANTO CHEMICAL CO., INC.), phenylacethylene (17 mg, manufactured by Tokyo Chemical Industry Co., Ltd.), dichlorobis(triphenylphosphine) palladium (4 mg, manufactured by KANTO CHEMICAL CO., INC.), triphenylphosphine (1 mg, manufactured by Wako Pure Chemical Industries, Ltd.) and triethylamine (24 μl, manufactured by Wako Pure Chemical Industries, Ltd.) were added at room temperature. The resultant mixture was stirred at the same temperature for 20 minutes. Copper iodide (1 mg, manufactured by KANTO CHEMICAL CO., INC.) was added to the reaction solution, and the resultant mixture was irradiated with microwave at 80° C. for 2 hours. The reaction solution was purified with silica gel column chromatography (hexane:ethyl acetate=2:1) to give 9.4 mg of the titled compound.

### Reference Example 11

### Methyl 3-sulfamoylthiophen-2-carboxylate

To a solution of methyl 3-chlorosulfonyl-2-thiophene carboxylate (20 g, manufactured by Wakou kagaku CO., INC.) in 1,4-dioxane (40 ml, manufactured by Wako kagaku CO., INC.), 25% ammonia water (0.5 ml, manufactured by KANTO CHEMICAL CO., INC.) was added at room temperature. The resultant mixture was stirred for 5 hours, and then the reaction solution was distilled away. Water (100 ml) was added thereto, and a product was extracted with chloroform (3×100 ml), washed with saturated saline (100 ml), dried (MgSO₄), and then the solvent was distilled away to give 12.3 g of the titled compound.

### Reference Example 12

### 2-(hydroxymethyl)thiophen-3-sulfonamide

To a solution of the compound of Reference Example 11 (12.3 g) in dichloromethane (556 ml, manufactured by Wako Pure Chemical Industries, Ltd.), diisobutyl aluminum hydride (1.02M hexane solution, 82 ml, manufactured by KANTO CHEMICAL CO., INC.) was added under ice cooling. The resultant mixture was stirred at room temperature for 5 hours. Saturated saline (50 ml) was dripped into the reaction solution, and then magnesium sulfate was added thereto. The reaction solution was stirred at room temperature for 1 hour, and then filtered with celite. The solvent was distilled away, and the obtained residue was purified with silica gel column chromatography (hexane:ethyl acetate 2:1) to give 9.2 g of the titled compound.

### Reference Example 13

### 2-((tert-butyldiphenylsiloxy)methyl)thiophen-3-sulfonamide

To a solution of the compound of Reference Example 12 (9.2 g) in dimethylformamide (450 ml, manufactured by KANTO CHEMICAL CO., INC.), imidazole (6.38 g, manufactured by KANTO CHEMICAL CO., INC.) and tert-butyldiphenylchlorosilane (24.5 ml, manufactured by Tokyo Chemical Industry Co., Ltd.) were added at room temperature. The resultant mixture was stirred over night. The reaction solution was distilled away, and the obtained residue was purified with silica gel column chromatography (hexane:ethyl acetate=2:1) to give 6.5 g of the titled compound.

### Reference Example 14

### 5-bromo-2-((tert-butyldiphenylsiloxy)methyl)thiophen-3-sulfonamide

To a solution of the compound of Reference Example 13 (3.0 g) in tetrahydrofuran (70 ml, manufactured by KANTO CHEMICAL CO., INC.), lithium diisopropyl amide (23% tetrahydrofuran/ethylbenzene/heptane solution, 15.5 ml, manufactured by Aldrich) was added at −78° C. The resultant mixture was stirred for 10 minutes. Then, carbon tetrabromide (9.22 g, manufactured by Wako Pure Chemical Industries, Ltd.) was added thereto at the same temperature, and the resultant mixture was stirred for 2 hours. The mixture was warmed to room temperature, and water (50 ml, manufactured by KANTO CHEMICAL CO., INC.) was added to the reaction solution. A product was extracted with ethyl acetate (3×50 ml), washed with saturated saline (50 ml), and dried (MgSO₄), and then the solvent was distilled away. The obtained residue was purified with silica gel column chromatography (hexane:ethyl acetate=2:1) to give 3.1 g of the titled compound.

### Reference Example 15

### 5-bromo-2-(hydroxymethyl)thiophen-3-sulfonamide

To a solution of the compound of Reference Example 14 (4.98 g) in tetrahydrofuran (100 ml, manufactured by KANTO CHEMICAL CO., INC.), tetrabutylammoniumfluoride (1.0M toluene solution, 19.5 ml, manufactured by Tokyo Chemical Industry Co., Ltd.) was added at room temperature. The resultant mixture was stirred for 1 hour. The reaction solution was distilled away, and the obtained residue was purified with silica gel column chromatography (hexane:ethyl acetate=2:1) to give 2.6 g of the titled compound.

### Reference Example 16

### 5-bromo-2-(bromomethyl)thiophen-3-sulfonamide

To a solution of the compound of Reference Example 15 (1.0 g) in dichloromethane (37 ml, manufactured by KANTO CHEMICAL CO., INC.), phosphorus tribromide (416 μl, manufactured by Aldrich) and pyridine (582 mg, manufactured by Wako Pure Chemical Industries, Ltd.) were added at −20° C. The resultant mixture was stirred at the same temperature for 2 hours. The reaction solution was warmed to room temperature, and then water (20 ml) was added thereto. A product was extracted with chloroform (3×20 ml), washed with saturated saline (20 ml), and dried (MgSO₄), and then the solvent was distilled away. The obtained residue was purified with silica gel column chromatography (hexane:ethyl acetate=2:1) to give 1.1 g of the titled compound.

### Reference Example 17

### 5-bromo-2,3-dihydro-1,1-dioxothieno[2,3-d]isothiazole

To a solution of the compound of Reference Example 16 (50 mg) in dimethylformamide (1.5 ml, manufactured by KANTO CHEMICAL CO., INC.), sodium hydride (containing 40% mineral oil, 12 mg, manufactured by KANTO CHEMICAL CO., INC.) was added at 50° C. The resultant mixture was stirred at the same temperature for 30 minutes. The reaction solution was cooled to room temperature, and 1M hydrochloric acid (3 ml) was added to the reaction solution. A product was extracted with ethyl acetate (3×10 ml), washed with saturated saline (20 ml), and dried (MgSO₄), and then the solvent was distilled away. The obtained residue was purified with silica gel column chromatography (hexane:ethyl acetate=2:1) to give 22.2 mg of the titled compound.

### Reference Example 18

### Methyl 2-(5-bromo-1,1-dioxothieno[2,3-d]isothiazol-2(3H)-yl) Butyrate

To a solution of the compound of Reference Example 17 (734 mg) in dimethylformamide (29 ml, manufactured by KANTO CHEMICAL CO., INC.), sodium hydride (40% mineral oil added, 231 mg, manufactured by KANTO CHEMICAL CO., INC.) was added under ice cooling. Methyl 2-bromo-n-butyrate (1.05 g, manufactured by Tokyo Chemical Industry Co., Ltd.) was added thereto at 50° C., and the resultant mixture was stirred for 20 minutes. Water (30 ml) was added to the reaction solution, and a product was extracted with ethyl acetate (3×30 ml), washed with saturated saline (30 ml), and dried (MgSO₄), and then the solvent was distilled away. The obtained residue was purified with silica gel column chromatography (hexane:ethyl acetate=2:1) to give 449 mg of the titled compound.

### Reference Example 19

### Methyl 2-(5-(4-phenylpiperidin-1-yl)-1,1-dioxothieno[2,3-d]isothiazol-2(3H)-yl)butyrate

To a solution of the compound of Reference Example 18 (22 mg) in 1,4-dioxane (300 μl, manufactured by KANTO CHEMICAL CO., INC.), cesium carbonate (40 mg, manufactured by Wako Pure Chemical Industries, Ltd.), XANTPHOS (14 mg, manufactured by Strem Chemicals, Inc.), palladium acetate (3 mg, manufactured by KANTO CHEMICAL CO., INC.), and 4-phenylpiperidine (12 mg, manufactured by Wako Pure Chemical Industries, Ltd.) were added. The resultant mixture was irradiated with microwave at 80° C. for 2 hours. The reaction solution was cooled to room temperature, and then water (1 ml) was added thereto. A product was extracted with ethyl acetate (3×2 ml), washed with saturated saline (10 ml), and dried (MgSO₄), and then the solvent was distilled away. The obtained residue was purified with silica gel column chromatography (hexane:ethyl acetate=4:1) to give 5.0 mg of the titled compound.

### Reference Example 20

### Methyl 2-(5-(1H-indazol-1-yl)-1,1-dioxothieno[2,3-d]isothiazol-2(3H)-yl)butyrate

To a solution of the compound of Reference Example 18 (25 mg) in N,N-dimethylacetamide (177 μl, manufactured by KANTO CHEMICAL CO., INC.), potassium phosphate (30 mg, manufactured by Wako Pure Chemical Industries, Ltd.), (1S,2S)-(+)-N,N-dimethylcyclohexan-1,2-diamine (4.0 mg, manufactured by Tokyo Chemical Industry Co., Ltd.), copper iodide (3.0 mg, manufactured by KANTO CHEMICAL CO., INC.) and indazole (10 mg, manufactured by Tokyo Chemical Industry Co., Ltd.) were added. The resultant mixture was irradiated with microwave for 15 minutes at 180° C. The reaction solution was cooled to room temperature, and then purified with silica gel column chromatography (hexane:ethyl acetate=2:1) to give 8.5 mg of the titled compound.

### Reference Example 21

### (2-hydrosulfonylthiophen-3-yl) Methanol

Into a solution of thiophen-3-methanol (14.5 ml, manufactured by AlfaAesar) in tetrahydrofuran (230 ml, manufactured by KANTO CHEMICAL CO., INC.), n-butyllithium (1.59M n-hexane solution, 212 ml, manufactured by KANTO CHEMICAL CO., INC.) was dripped over 45 minutes at −78° C. The resultant mixture was stirred for 30 minutes at the same temperature. Then sulfur dioxide (16 ml, manufactured by SUMITOMO SEIKA CHEMICALS CO., LTD.) was added thereto. While the temperature of the resultant mixture was gradually elevated to room temperature, the mixture was stirred for 2 hours. The reaction solution was distilled away to give the titled compound.

### Reference Example 22

### 3-(hydroxymethyl)thiophen-2-sulfonylamide

To a solution of the compound of Reference Example 21 (25 g) in water (400 ml), sodium acetate (37.8 g, manufactured by Wako Pure Chemical Industries, Ltd.) and hydroxylamine-O-sulfonic acid (32 g, manufactured by Wako Pure Chemical Industries, Ltd.) were added at room temperature. The resultant mixture was stirred over night. To the reaction solution, ethyl acetate (200 ml) was added. A product was extracted with ethyl acetate (3×200 ml), washed with saturated saline (200 ml), and dried (MgSO₄), and then the solvent was distilled away. The obtained residue was purified with silica gel column chromatography (hexane:ethyl acetate=2:1) to give 25.0 g of the titled compound.

### Reference Example 23

### Methyl 2-(5-(3-aminophenyl)-1,1-dioxothieno[2,3-d]isothiazol-2(3H)-yl)butyrate

Methyl 2-(5-(3-nitrophenyl)-1,1-dioxothieno[2,3-d]isothiazol-2(3H)-yl)butyrate (81 mg) was dissolved in tetrahydrofuran (1.0 ml) and methanol (0.5 ml), and 10 wt % palladium-carbon (20 mg, manufactured by Aldrich) was added to the resultant solution and the obtained mixture was stirred under hydrogen atmosphere at room temperature for 2 hours. The reaction mixture was filtered with celite, and washed with ethyl acetate. The mother solution was concentrated to give 74 mg of the titled compound. LC-MS: HPLC retention time 1.47 minutes (LC Condition 1), m/z 367 (MH+)

### Reference Example 24-1

### Methyl 2-(5-(3-(cyclohexane carboxyamide)phenyl)-1,1-dioxothieno[2,3-d]isothiazol-5(3H)-yl)butyrate

Methyl 2-(5-(3-aminophenyl)-1,1-dioxothieno[2,3-d]isothiazol-2(3H)-yl)butyrate (25 mg) was dissolved in dichloromethane (0.5 ml), and triethylamine (14.2 μl, manufactured by Tokyo Chemical Industry Co., Ltd.) and cyclohexane carbonyl chloride (10.9 μl, manufactured by Tokyo Chemical Industry Co., Ltd.) were added to the resultant solution under ice cooling and the obtained mixture was stirred for 10 minutes. To the reaction mixture, water (1 ml) and ethyl acetate (2 ml) were added. A product was extracted with ethyl acetate, washed with saturated saline, dried with magnesium sulfate and then concentrated. The obtained residue was put into a silica gel column (hexane:ethyl acetate=2:1) to give 20.4 mg of the titled compound. LC-MS: HPLC retention time 1.85 minutes (LC Condition 1), m/z 477 (MH+)

### Reference Example 24-2

### Methyl 2-(5-(4-(benzyloxy)benzamide) phenyl-1,1-dioxothieno[2,3-d]isothiazol-2(3H)-yl)butyrate

To a solution of 4-benzyloxybenzoic acid (33.7 mg, manufactured by Tokyo Chemical Industry Co., Ltd.) in N,N-dimethylformamide (500 μl, manufactured by KANTO CHEMICAL CO., INC.), 1-hydroxybenzotriazole hydrate (27.2 mg, manufactured by KOKUSAN CHEMICAL CO., LTD) and 1-ethyl-3-(3-dimethylaminopropyl)-carbodiimide hydrochloride (38.3 mg, manufactured by KOKUSAN CHEMICAL CO., LTD) were added at room temperature. The resultant mixture was stirred for 5 minutes. Then, the solution of the compound of Reference Example 25 (49 mg) in dimethylformamide (800 μl, manufactured by KANTO CHEMICAL CO., INC.) and triethylamine (28.1 μl) were added thereto, and the resulting mixture was stirred over night. To the reaction solution, saturated sodium bicarbonate (500 μl) was added. A product was extracted with chloroform (3×5 ml), washed with saturated saline (10 ml), and dried (MgSO₄), and then the solvent was distilled away. The obtained residue was purified with silica gel column chromatography (hexane:ethyl acetate=2:1 (CHCl₃:MeOH=9:1) to give 21.5 mg of the titled compound.

### Reference Example 25

### Methyl 2-(5-(4-aminophenyl)-1,1-dioxothieno[2,3-d]isothiazol-2(3H)-yl)butyrate

To a solution of methyl 2-(5-(4-(tert-butoxycarbonylamino)phenyl)-1,1-dioxothieno[2,3-d]isothiazol-2(3H)-yl)butyrate (360 mg), which was synthesized from the compound of Reference Example 18 by the method of Reference Example 9, in dichloromethane (7.7 ml, manufactured by KANTO CHEMICAL CO., INC.), trifluoroacetic acid (879 mg, manufactured by Wako Pure Chemical Industries, Ltd.) was added under ice cooling. The resultant mixture was stirred at room temperature for 3 hours. To the reaction solution, 25% ammonia water (10 ml, manufactured by KANTO CHEMICAL CO., INC.) was added for neutralization. A product was extracted with chloroform (3×20 ml), washed with saturated saline (10 ml), and dried (MgSO₄), and then the solvent was distilled away to give 154 mg of the titled compound.

### Reference Example 26

### 2-(5-bromo-1,1-dioxothieno[2,3-d]isothiazol-2(3H)-yl) Butanoic Acid

Methyl 2-(5-bromo-1,1-dioxothieno[2,3-d]isothiazol-2(3H)-yl) butyrate (44.3 mg) was dissolved in methanol (1.0 ml), and 5N sodium hydroxide aqueous solution (0.1 ml, manufactured by Wako Pure Chemical Industries, Ltd.) was added thereto and the resultant mixture was stirred at room temperature for 3 hours. To the reaction mixture, 5N hydrochloric acid (0.1 ml, manufactured by Wako Pure Chemical Industries, Ltd.) was added. Then the resultant mixture was concentrated. The obtained residue was purified with thin-layer chromatography (dichloromethane:methanol=10:1) to give 32.9 mg of the titled compound. LC-MS: HPLC retention time 3.47 (LC Condition 2), m/z 339 (M-1).

### Example 15

### 2-(5-((biphenyl-4-yl)-1,1-dioxothieno[2,3-d]isothiazol-2(3H)-yl)butanoic Acid

Methyl 2-(5-(4-(-bromophenyl)-1,1-oxothieno[2,3-d]isothiazol-2(3H)-yl)butyrate (45 mg) was dissolved in dimethoxyethane (0.5 ml) and water (0.1 ml), and tetrakistriphenylphosphine palladium (12.1 mg, manufactured by Aldrich), sodium carbonate (13.2 mg, manufactured by Wako Pure Chemical Industries, Ltd.) and phenylboronic acid (15.2 mg, manufactured by Aldrich) were added thereto and the resultant mixture was stirred at 100° C. for 2 days. To the reaction mixture, water (1 ml), 1N hydrochloric acid (1 ml) and ethyl acetate (1 ml) were added. A product was extracted with ethyl acetate, washed with saturated saline, dried with magnesium sulfate and then concentrated. The obtained residue was put into a silica gel column (dichloromethane:methanol=10:1) to give 16.1 mg of the titled compound. LC-MS: HPLC retention time 1.85 minutes (LC Condition 1), m/z 411 (MH+).

### Example 16

### 2-(5-(4-bromophenyl)thieno[2,3-d]isothiazol-2-[3H]-yl)-N-hydroxybutan Amide

Methyl 2-(5-(4-bromophenyl)thieno[2,3-d]isothiazol-2-[3H]-yl) butyrate (21.6 mg) was dissolved in a mixed solvent of methanol (0.5 ml) and chloroform (0.5 ml), and a hydroxylamine-methanol solution (1.76 M, 284 μl) was added to the resultant solution, and the obtained mixture was stirred at room temperature for 1 hour. 1N hydrochloric acid (0.5 ml) was added thereto, and the reaction solution was diluted with ethyl acetate, washed with water and saturated saline, dried with magnesium sulfate and then concentrated. The obtained residue was purified with thin-layer chromatography (hexane:ethyl acetate=1:2) to give 6.6 mg of the titled compound. LC-MS: HPLC retention time 1.59 minutes (LC Condition 1), m/z 431 (MH+)

### Example 17

### 2-(5-(2-phenoxypyrimidin-5-yl)-1,1-dioxothieno[2,3-d]isothiazol-2(3H)-yl)butanoic Acid

2-(5-bromo-1,1-dioxothieno[2,3-d]isothiazol-2(3H)-yl) butanoic acid (32.9 mg) was dissolved in N,N-dimethylformamide (0.4 ml) and water (0.1 ml), and tetrakistriphenylphosphine palladium (22.4 mg, manufactured by Aldrich), sodium carbonate (20.6 mg, manufactured by Wako Pure Chemical Industries, Ltd.) and 5-phenoxypyrimidin-2-boronic acid pinacol ester (28.9 mg) were stirred at 100° C. for 24 hours. To the reaction mixture, water (1 ml), 1N hydrochloric acid (5 ml) and dichloromethane (1 ml) were added. A product was extracted with dichloromethane, washed with saturated saline, dried with magnesium sulfate and then concentrated. The obtained residue was purified with thin-layer chromatography (dichloromethane:methanol=10:1) to give 6.5 mg of the titled compound. LC-MS: HPLC retention time 1.49 minutes (LC Condition 1), m/z 432 (MH+).

### Examples 18 to 148

Synthesis of the compounds of Examples 18 to 148 will be shown below. Details of Examples 18 to 148 are shown in Table 1-2. Meanings of the symbols in Table 1-2 are as follows: “Exp.”; Example number, “Structure”; Example compound, “LCRT”; retention time (minutes) of liquid chromatography in LCMS, “LCMT”; LC Condition, “MS”; mass spectral data in LCMS, “Syn.”; method of synthesizing corresponding intermediate and Example compound.

The compound having the mark “*” in an asymmetric carbon atom among the structural formulae in the “Structure” section represents an optically active substance having α configuration or β configuration in the asymmetric carbon.

The symbols in the section of the symbol “Syn.” represent a production method described below in the present invention unless otherwise stated, as apparent to the skilled in the art. “A”; the production method shown in Reference Example 1, “B”; the production method shown in Reference Example 2, “C”; the production method shown in Reference Example 3, “D”; the production method shown in Reference Example 4, “E”; the production method shown in Reference Example 5, “F”; the production method shown in Reference Example 6, “G”; the production method shown in Reference Example 7, “H”; the production method shown in Reference Examples 8-1, 8-2, or 8-3, “J”; the production method shown in Reference Example 9, “K”; the production method shown in Reference Example 10, “L”; the production method shown in Reference Example 11, “M”; the production method shown in Reference Example 12, “N”; the production method shown in Reference Example 13, “P”; the production method shown in Reference Example 14, “Q”; the production method shown in Reference Example 15, “R”; the production method shown in Reference Example 16, “S”; the production method shown in Reference Example 17, “T”; the production method shown in Reference Example 18, “U”; the production method shown in Reference Example 19, “V”; the production method shown in Reference Example 20, “W”; the production method shown in Reference Example 21, “X”; the production method shown in Reference Example 22, “Y”; the production method shown in Reference Example 23, “Z”; the production method shown in Reference Examples 24-1 or 24-2, “AA”; the production method shown in Reference Example 25, “AB”; the production method shown in Reference Example 26, “a”; the production method shown in Example 1, “b”; the production method shown in Example 2, “c”; the production method shown in Example 15, “d”; the production method shown in Example 16 and “e”; the production method shown in Example 17.

TABLE 1-2

| Exp. | Structure | LCRT | LCMT | MS | Ref. |
|---|---|---|---|---|---|
| 18 | 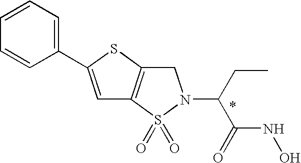 | 1.32 | 1 | 353 (MH+) | A, B, C, D, E, F, a, G, b |
| 19 | 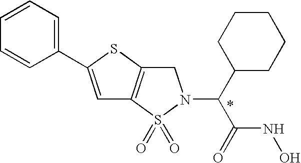 | 1.60 | 1 | 407 (MH+) | A, B, C, D, E, F, a, G, b |
| 20 | 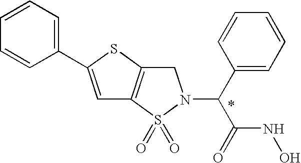 | 1.50 | 1 | 401 (MH+) | A, B, C, D, E, F, a, G, b |
| 21 | 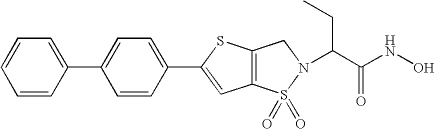 | 1.70 | 1 | 429 (MH+) | A, B, M, R, S, T, c, G, b |
| 22 | 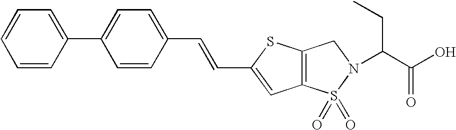 | 1.95 | 1 | 440 (MH+) | L, M, N, P, Q, R, S, T, H, a |
| 23 | 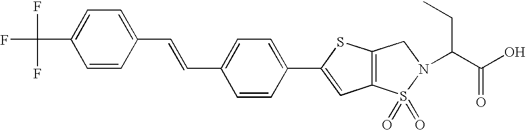 | 1.68 | 1 | 508 (MH+) | A, B, M, R, S, T, H, a |
| 24 | 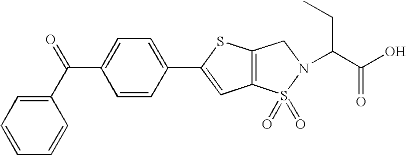 | 1.82 | 1 | 442 (MH+) | L, M, N, P, Q, R, S, T, H, a |
| 25 | 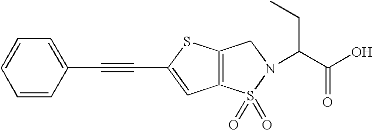 | 1.69 | 1 | 362 (MH+) | L, M, N, P, Q, R, S, T, K, a |
| 26 | 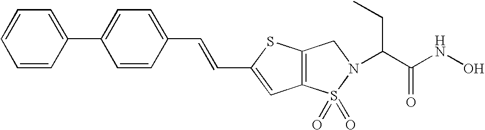 | 1.83 | 1 | 455 (MH+) | L, M, N, P, Q, R, S, T, H, a, G, b |
| 27 | 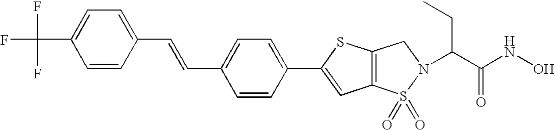 | 1.69 | 1 | 447 (MH+) | A, B, M, R, S, T, H, a, G, b |
| 28 | 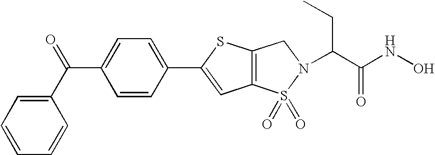 | 1.54 | 1 | 457 (MH+) | L, M, N, P, Q, R, S, T, H, a, G, b |
| 29 | 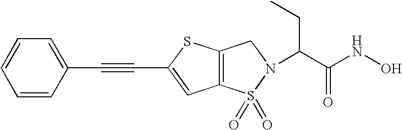 | 1.53 | 1 | 377 (MH+) | L, M, N, P, Q, R, S, T, K, a, G, b |
| 30 | 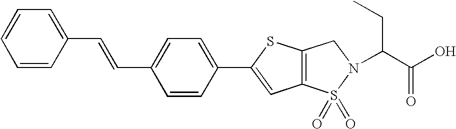 | 1.95 | 1 | 440 (MH+) | A, B, M, R, S, T, H, a |
| 31 | 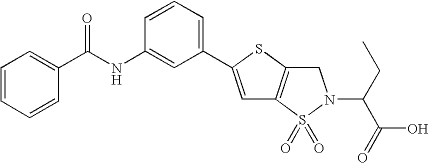 | 1.54 | 1 | 457 (MH+) | A, B, M, R, S, T, Y, Z, a |
| 32 | 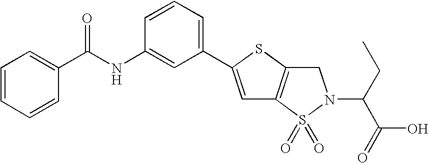 | 1.40 | 1 | 472 (MH+) | A, B, M, R, S, T, Y, Z, a |
| 33 | 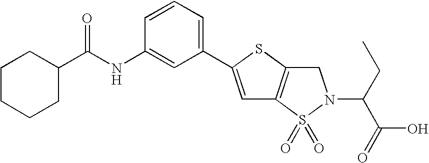 | 1.65 | 1 | 463 (MH+) | A, B, M, R, S, T, Y, Z, a |
| 34 | 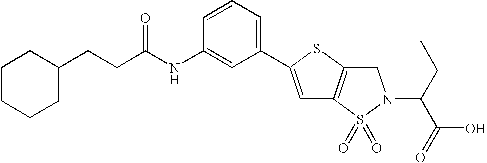 | 1.61 | 1 | 485 (MH+) | A, B, M, R, S, T, Y, Z, a |
| 35 | 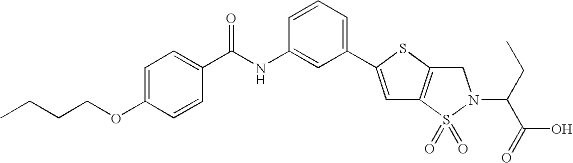 | 1.86 | 1 | 529 (MH+) | A, B, M, R, S, T, Y, Z, a |
| 36 | 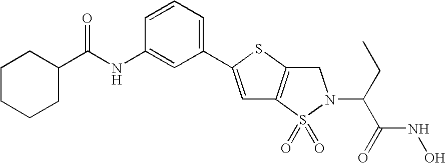 | 1.51 | 1 | 478 (MH+) | A, B, M, R, S, T, Y, Z, a, G, b |
| 37 | 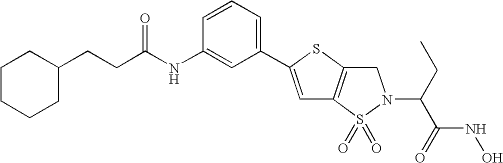 | 1.48 | 1 | 500 (MH+) | A, B, M, R, S, T, Y, Z, a, G, b |
| 38 | 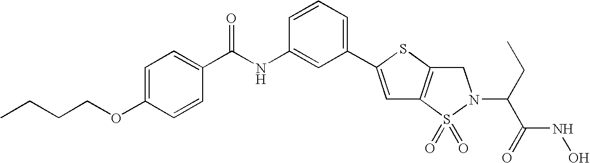 | 1.75 | 1 | 544 (MH+) | A, B, M, R, S, T, Y, Z, a, G, b |
| 39 | 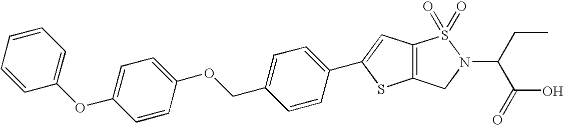 | 2.05 | 1 | 536 (MH+) | L, M, N, P, Q, R, S, T, H, a |
| 40 | 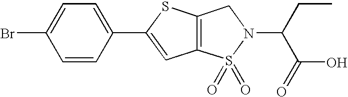 | 1.73 | 1 | 417 (MH+) | A, B, M, R, S, T, a |
| 41 | 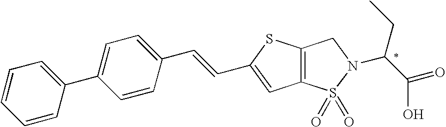 | 1.95 | 1 | 439 (MH+) | L, M, N, P, Q, R, S, T, H, a |
| 42 | 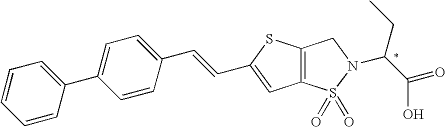 | 1.95 | 1 | 439 (MH+) | L, M, N, P, Q, R, S, T, H, a |
| 43 | 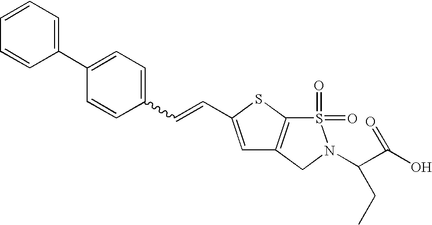 | 1.93 | 1 | 440 (MH+) | W, X, N, P, Q, R, S, T, H, a |
| 44 | 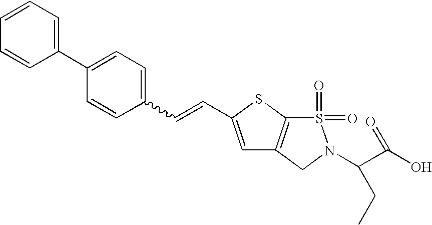 | 1.93 | 1 | 440 (MH+) | W, X, N, P, Q, R, S, T, H, a |
| 45 | 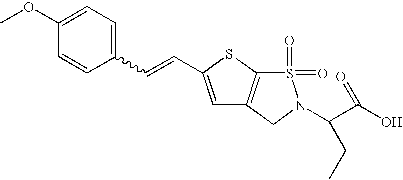 | 1.66 | 1 | 392 (MH−) | W, X, N, P, Q, R, S, T, H, a |
| 46 | 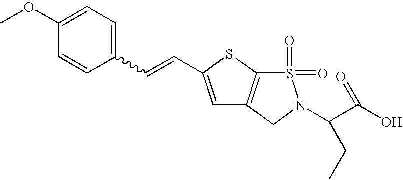 | 1.66 | 1 | 392 (MH−) | W, X, N, P, Q, R, S, T, H, a |
| 47 | 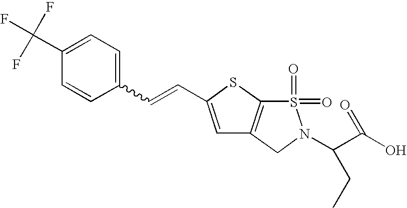 | 1.85 | 1 | 430 (MH−) | W, X, N, P, Q, R, S, T, H, a |
| 48 | 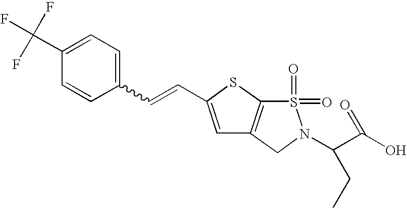 | 1.85 | 1 | 430 (MH−) | W, X, N, P, Q, R, S, T, H, a |
| 49 | 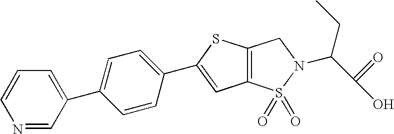 | 1.31 | 1 | 415 (MH+) | A, B, M, R, S, T, H, a |
| 50 | 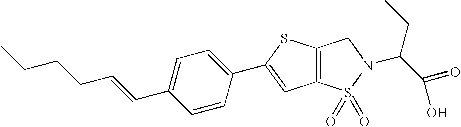 | 2.14 | 1 | 420 (MH+) | A, B, M, R, S, T, H, a |
| 51 | 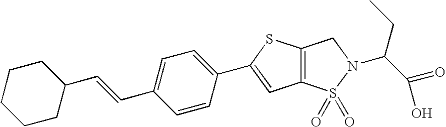 | 2.28 | 1 | 446 (MH+) | A, B, M, R, S, T, H, a |
| 52 | 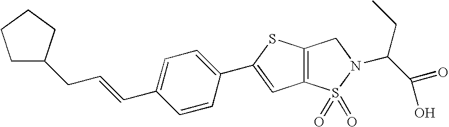 | 2.28 | 1 | 446 (MH+) | A, B, M, R, S, T, H, a |
| 53 | 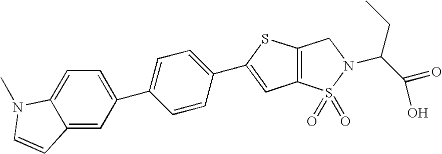 | 1.87 | 1 | 467 (MH+) | A, B, M, R, S, T, H, a |
| 54 | 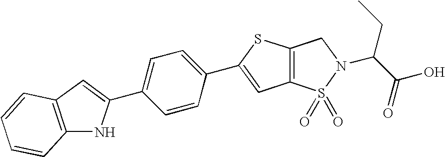 | 1.82 | 1 | 453 (MH+) | A, B, M, R, S, T, H, a |
| 55 | 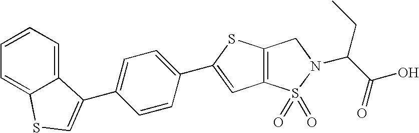 | 2.02 | 1 | 470 (MH+) | A, B, M, R, S, T, H, a |
| 56 | 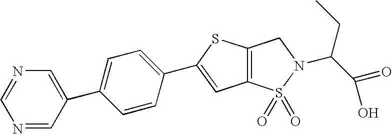 | 1.31 | 1 | 416 (MH+) | A, B, M, R, S, T, H, a |
| 57 | 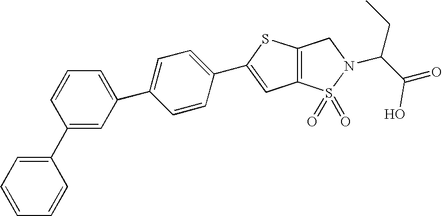 | 2.11 | 1 | 488 (MH−) | A, B, M, R, S, T, H, a |
| 58 | 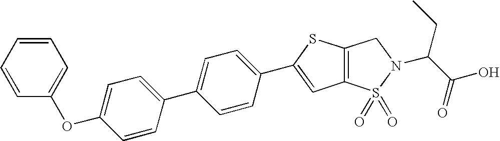 | 2.09 | 1 | 504 (MH−) | A, B, M, R, S, T, H, a |
| 59 | 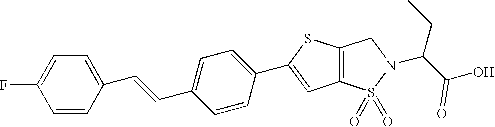 | 1.96 | 1 | 458 (MH+) | A, B, M, R, S, T, H, a |
| 60 | 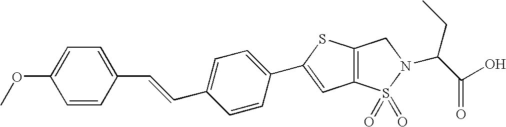 | 1.91 | 1 | 470 (MH+) | A, B, M, R, S, T, H, a |
| 61 | 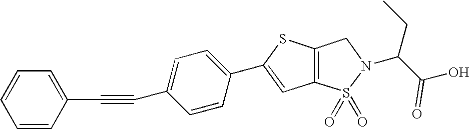 | 1.91 | 1 | 438 (MH+) | A, B, M, R, S, T, K, a |
| 62 | 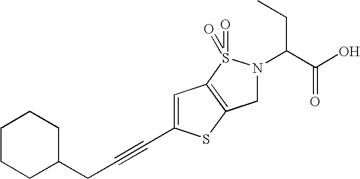 | 1.72 | 1 | 380 (MH−) | L, M, N, P, Q, R, S, T, K, a |
| 63 | 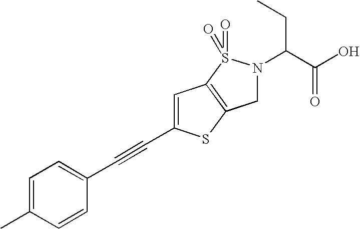 | 1.47 | 1 | 374 (MH−) | L, M, N, P, Q, R, S, T, K, a |
| 64 | 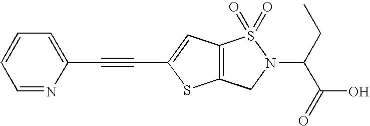 | 0.79 | 1 | 363 (MH−) | L, M, N, P, Q, R, S, T, K, a |
| 65 | 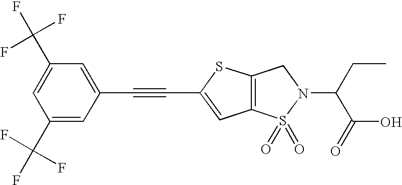 | 1.72 | 1 | 498 (MH+) | L, M, N, P, Q, R, S, T, K, a |
| 66 | 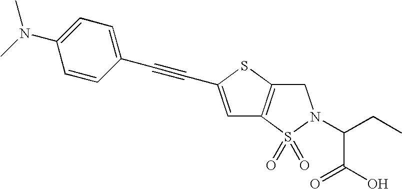 | 1.41 | 1 | 405 (MH+) | L, M, N, P, Q, R, S, T, K, a |
| 67 | 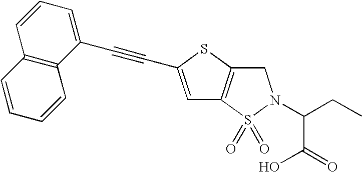 | 1.55 | 1 | 412 (MH+) | L, M, N, P, Q, R, S, T, K, a |
| 68 | 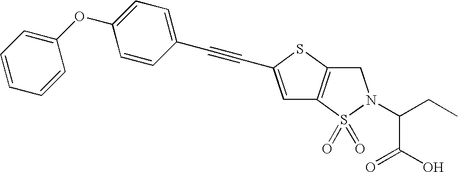 | 1.68 | 1 | 454 (MH+) | L, M, N, P, Q, R, S, T, K, a |
| 69 | 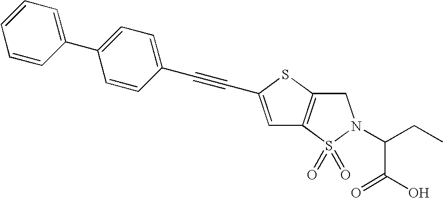 | 1.70 | 1 | 438 (MH+) | L, M, N, P, Q, R, S, T, K, a |
| 70 | 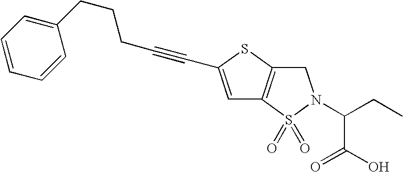 | 1.53 | 1 | 404 (MH+) | L, M, N, P, Q, R, S, T, K, a |
| 71 | 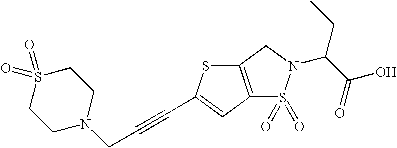 | 1.07 | 1 | 433 (MH+) | L, M, N, P, Q, R, S, T, K, a |
| 72 | 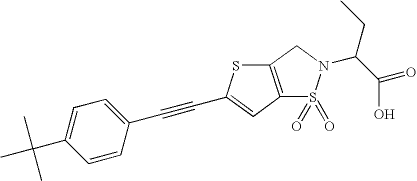 | 2.01 | 1 | 418 (MH+) | L, M, N, P, Q, R, S, T, K, a |
| 73 | 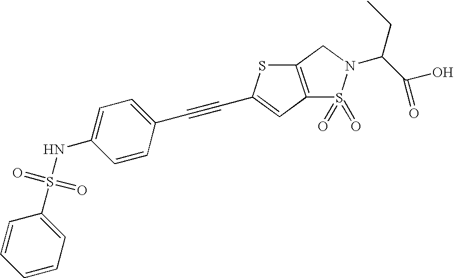 | 1.57 | 1 | 517 (MH+) | L, M, N, P, Q, R, S, T, K, a |
| 74 | 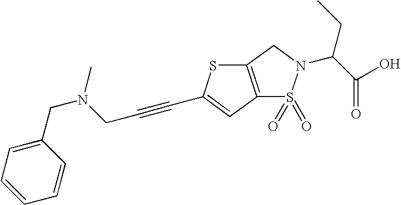 | 1.01 | 1 | 419 (MH+) | L, M, N, P, Q, R, S, T, K, a |
| 75 | 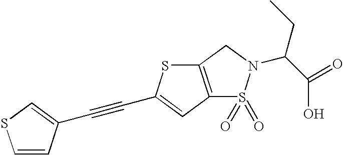 | 1.59 | 1 | 368 (MH+) | L, M, N, P, Q, R, S, T, K, a |
| 76 | 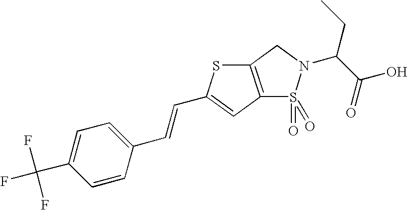 | 1.78 | 1 | 432 (MH+) | L, M, N, P, Q, R, S, T, H, a |
| 77 | 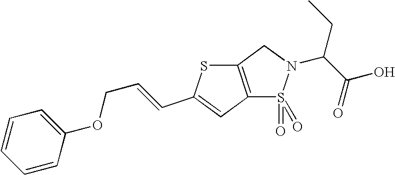 | 1.63 | 1 | 394 (MH+) | L, M, N, P, Q, R, S, T, H, a |
| 78 | 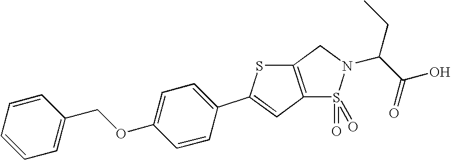 | 1.80 | 1 | 444 (MH+) | L, M, N, P, Q, R, S, T, H, a |
| 79 | 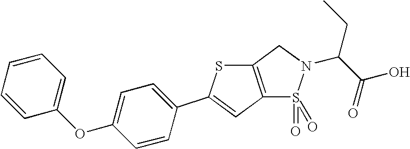 | 1.81 | 1 | 430 (MH+) | L, M, N, P, Q, R, S, T, H, a |
| 80 | 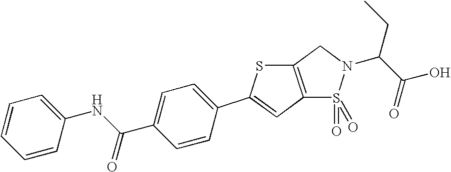 | 1.52 | 1 | 457 (MH+) | L, M, N, P, Q, R, S, T, H, a |
| 81 | 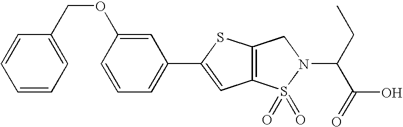 | 1.80 | 1 | 444 (MH+) | L, M, N, P, Q, R, S, T, H, a |
| 82 | 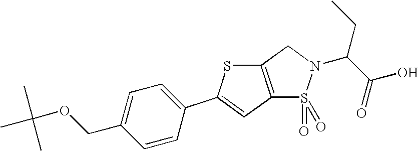 | 1.78 | 1 | 424 (MH+) | L, M, N, P, Q, R, S ,T, H, a |
| 83 | 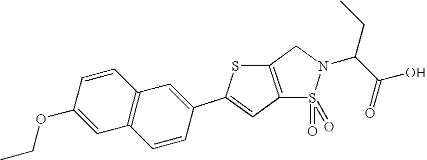 | 1.81 | 1 | 432 (MH+) | L, M, N, P, Q, R, S, T, H, a |
| 84 | 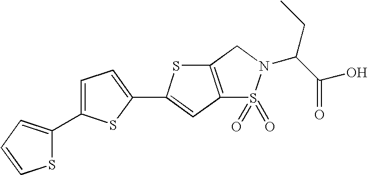 | 1.80 | 1 | 424 (MH−) | L, M, N, P, Q, R, S, T, J, a |
| 85 | 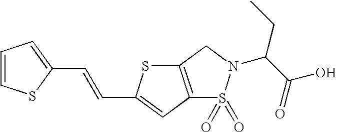 | 1.58 | 1 | 370 (MH+) | L, M, N, P, Q, R, S, T, J, a |
| 86 | 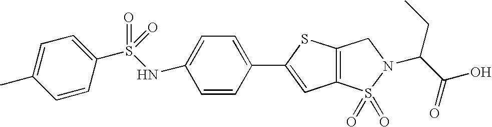 | 1.56 | 1 | 507 (MH+) | L, M, N, P, Q, R, S, T, J, a |
| 87 | 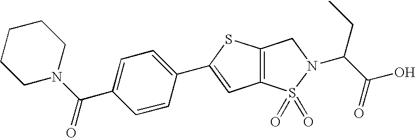 | 1.44 | 1 | 449 (MH+) | L, M, N, P, Q, R, S, T, J, a |
| 88 | 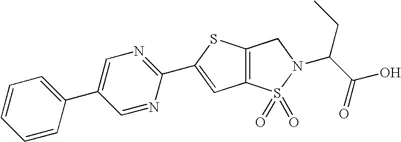 | 1.63 | 1 | 416 (MH+) | L, M, N, P, Q, R, S, T, J, a |
| 89 | 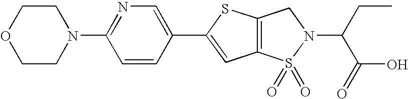 | 0.74 | 2 | 424 (MH+) | L, M, N, P, Q, R, S, T, H, a |
| 90 | 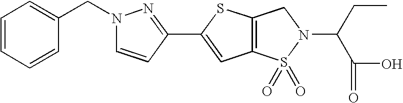 | 0.99 | 2 | 418 (MH+) | L, M, N, P, Q, R, S, T, H, a |
| 91 | 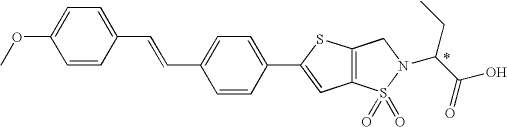 | 1.91 | 1 | 470 (MH+) | A, B, M, R, S, T, H, a |
| 92 | 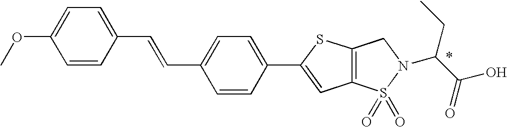 | 1.91 | 1 | 470 (MH+) | A, B, M, R, S, T, H, a |
| 93 | 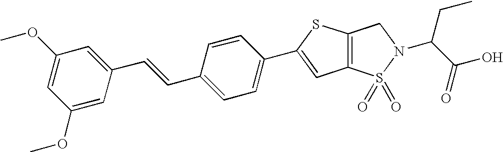 | 1.88 | 1 | 500 (MH+) | A, B, M, R, S, T, H, a |
| 94 | 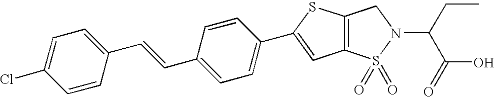 | 2.07 | 1 | 474 (MH+) | A, B, M, R, S, T, H, a |
| 95 | 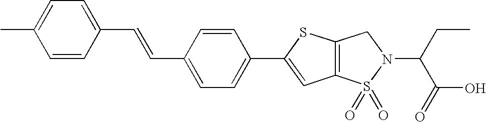 | 2.05 | 1 | 454 (MH+) | A, B, M, R, S, T, H, a |
| 96 | 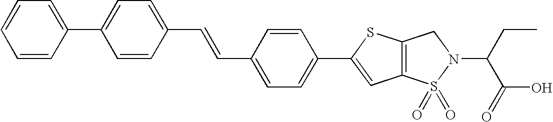 | 2.20 | 1 | 516 (MH+) | A, B, M, R, S, T, H, a |
| 97 | 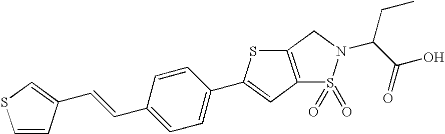 | 1.89 | 1 | 446 (MH+) | A, B, M, R, S, T, H, a |
| 98 | 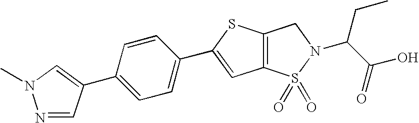 | 1.38 | 1 | 418 (MH+) | A, B, M, R, S, T, H, a |
| 99 | 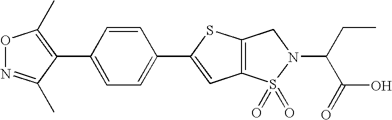 | 1.57 | 1 | 433 (MH+) | A, B, M, R, S, T, H, a |
| 100 | 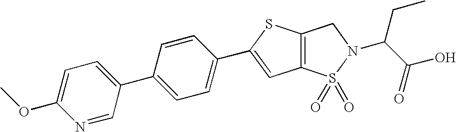 | 1.69 | 1 | 445 (MH+) | A, B, M, R, S, T, H, a |
| 101 | 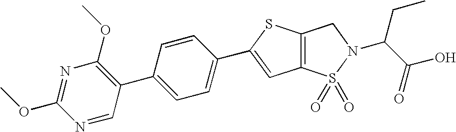 | 1.59 | 1 | 476 (MH+) | A, B, M, R, S, T, H, a |
| 102 | 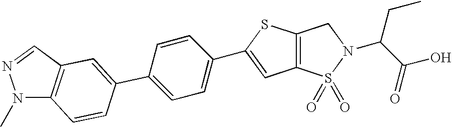 | 1.64 | 1 | 468 (MH+) | A, B, M, R, S, T, H, a |
| 103 | 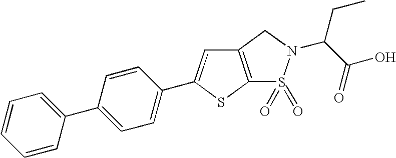 | 1.89 | 1 | 412 (MH−) | W, X, N, P, Q, R, S, T, H, a |
| 104 | 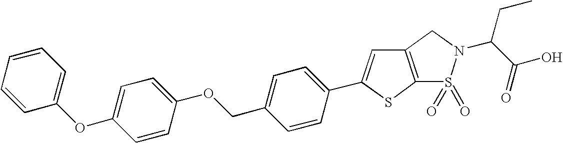 | 2.04 | 1 | 536 (MH+) | W, X, N, P, Q, R, S, T, H, a |
| 105 | 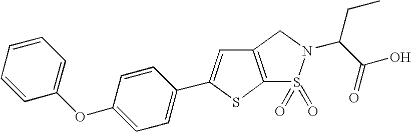 | 1.85 | 1 | 430 (MH+) | W, X, N, P, Q, R, S, T, H, a |
| 106 | 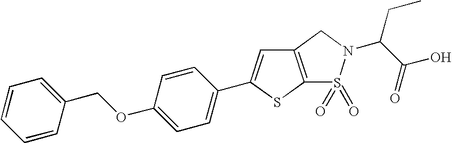 | 1.84 | 1 | 444 (MH+) | W, X, N, P, Q, R, S, T, H, a |
| 107 | 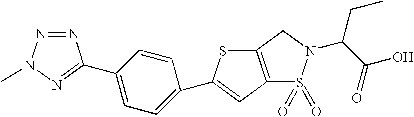 | 1.43 | 1 | 418 (MH−) | W, X, N, P, Q, R, S, T, H, a |
| 108 | 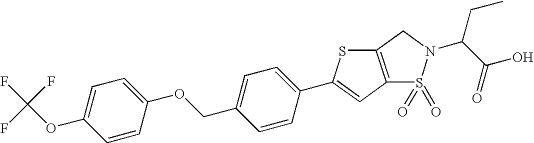 | 1.96 | 1 | 526 (MH−) | W, X, N, P, Q, R, S, T, H, a |
| 109 | 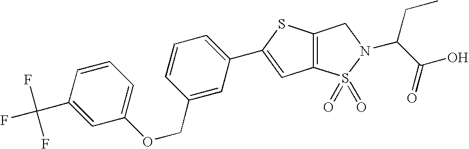 | 1.95 | 1 | 512 (MH+) | W, X, N, P, Q, R, S, T, H, a |
| 110 | 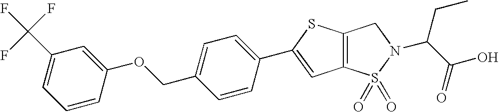 | 1.93 | 1 | 512 (MH+) | W, X, N, P, Q, R, S, T, H, a |
| 111 | 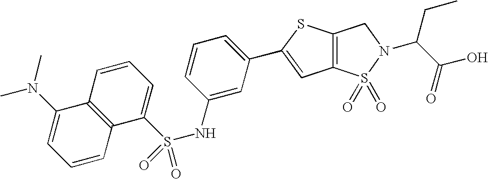 | 1.73 | 1 | 586 (MH+) | W, X, N, P, Q, R, S, T, H, a |
| 112 | 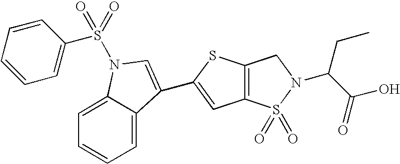 | 1.77 | 1 | 515 (MH+) | W, X, N, P, Q, R, S, T, H, a |
| 113 | 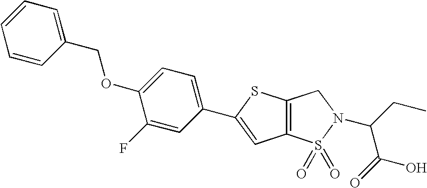 | 1.85 | 1 | 460 (MH−) | W, X, N, P, Q, R, S, T, H, a |
| 114 | 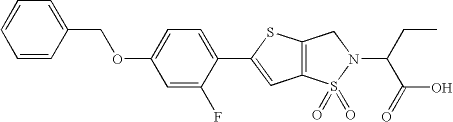 | 1.85 | 1 | 460 (MH−) | W, X, N, P, Q, R, S, T, H, a |
| 115 | 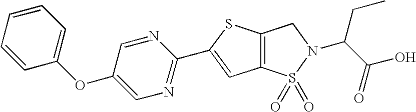 | 1.49 | 1 | 432 (MH+) | W, X, N, P, Q, R, S, T, H, a |
| 116 | 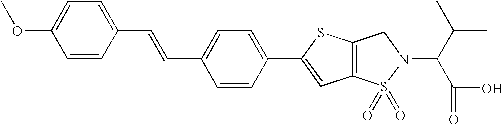 | 1.96 | 1 | 484 (MH+) | A, B, M, R, S, T, J, a |
| 117 | 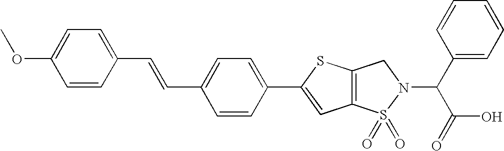 | 1.99 | 1 | 518 (MH+) | A, B, M, R, S, T, J, a |
| 118 | 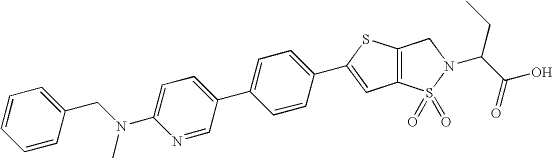 | 1.84 | 1 | 534 (MH+) | A, B, M, R, S, T, H, a |
| 119 | 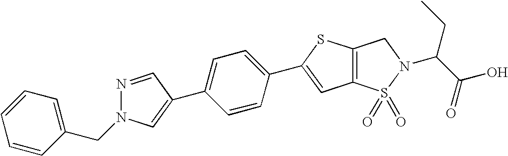 | 1.68 | 1 | 494 (MH+) | A, B, M, R, S, T, H, a |
| 120 | 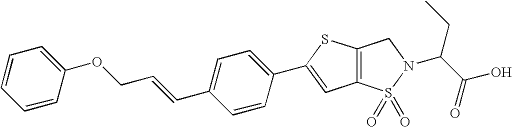 | 1.92 | 1 | 470 (MH+) | A, B, M, R, S, T, H, a |
| 121 | 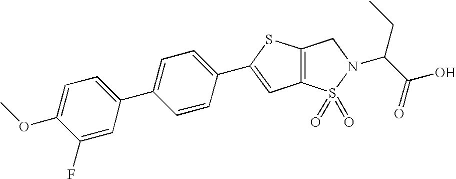 | 1.79 | 1 | 462 (MH+) | A, B, M, R, S, T, H, a |
| 122 | 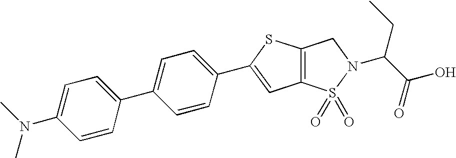 | 1.91 | 1 | 457 (MH+) | A, B, M, R, S, T, H, a |
| 123 | 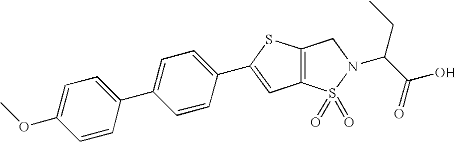 | 1.81 | 1 | 444 (MH+) | A, B, M, R, S, T, H, a |
| 124 | 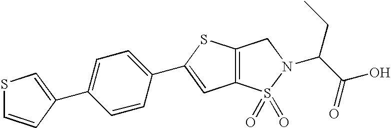 | 1.79 | 1 | 420 (MH+) | A, B, M, R, S, T, H, a |
| 125 | 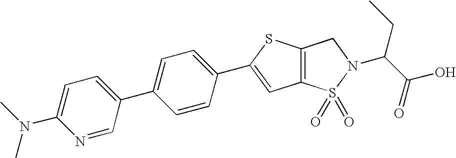 | 1.27 | 1 | 458 (MH+) | A, B, M, R, S, T, H, a |
| 126 | 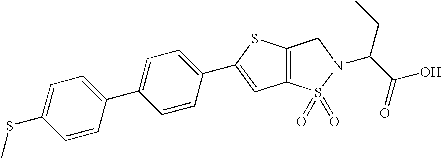 | 1.93 | 1 | 460 (MH+) | A, B, M, R, S, T, H, a |
| 127 | 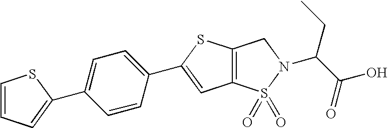 | 1.82 | 1 | 420 (MH+) | A, B, M, R, S, T, H, a |
| 128 | 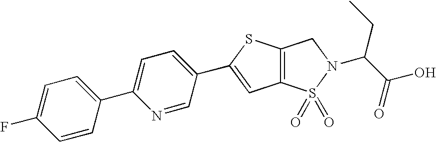 | 1.67 | 1 | 433 (MH+) | L, M, N, P, Q, R, S, T, H, a |
| 129 | 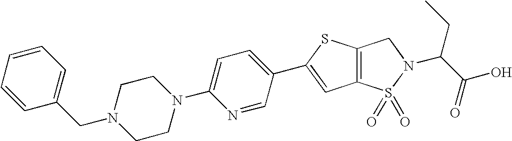 | 1.23 | 1 | 513 (MH+) | L, M, N, P, Q, R, S, T, H, a |
| 130 | 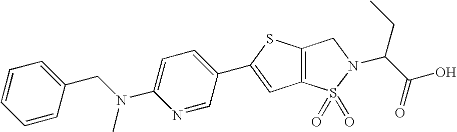 | 1.73 | 1 | 458 (MH+) | L, M, N, P, Q, R, S, T, H, a |
| 131 | 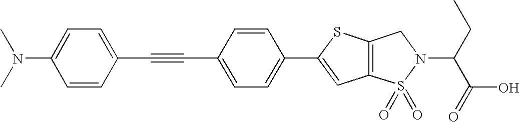 | 2.01 | 1 | 481 (MH+) | L, M, N, P, Q, R, S, T, K, a |
| 132 | 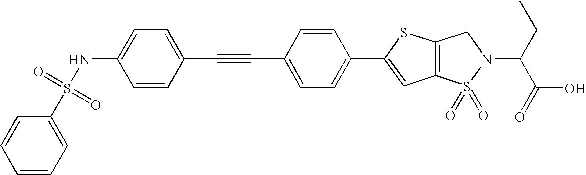 | 1.77 | 1 | 591 (MH−) | L, M, N, P, Q, R, S, T, K, a |
| 133 | 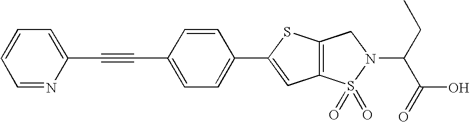 | 1.58 | 1 | 439 (MH+) | L, M, N, P, Q, R, S, T, K, a |
| 134 | 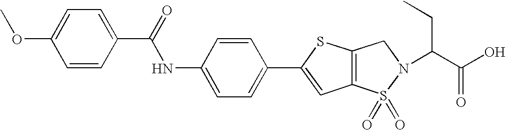 | 1.07 | 2 | 487 (MH+) | L, M, N, P, Q, R, S, T, H, AA, Z, a |
| 135 | 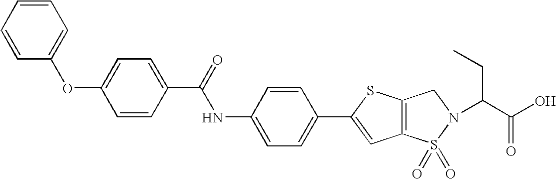 | 1.48 | 2 | 549 (MH+) | L, M, N, P, Q, R, S, T, H, AA, Z, a |
| 136 | 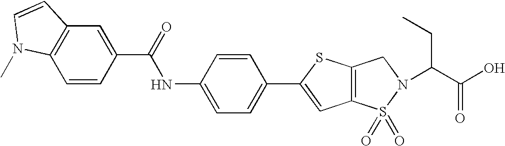 | 1.14 | 2 | 510 (MH+) | L, M, N, P, Q, R, S, T, H, AA, Z, a |
| 137 | 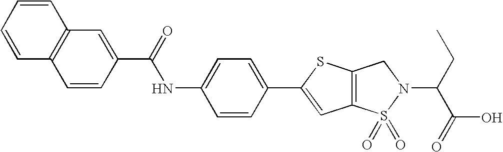 | 1.36 | 2 | 507 (MH+) | L, M, N, P, Q, R, S, T, H, AA, Z, a |
| 138 | 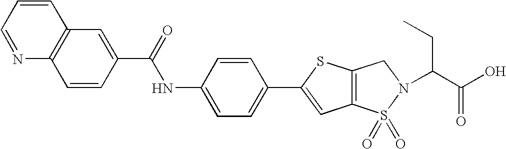 | 0.92 | 2 | 508 (MH+) | L, M, N, P, Q, R, S, T, H, AA, Z, a |
| 139 | 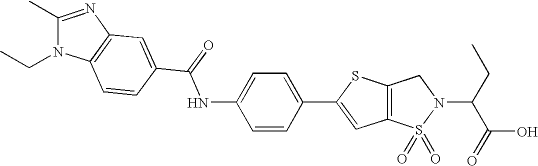 | 0.62 | 2 | 539 (MH+) | L, M, N, P, Q, R, S, T, H, AA, Z, a |
| 140 | 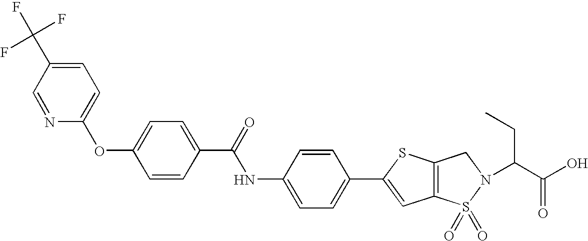 | 1.46 | 2 | 618 (MH+) | L, M, N, P, Q, R, S, T, H, AA, Z, a |
| 141 | 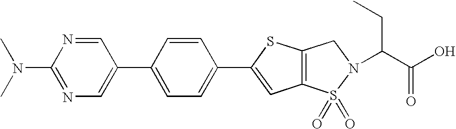 | 1.63 | 1 | 459 (MH+) | A, B, M, R, S, T, H, e |
| 142 | 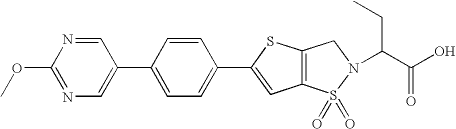 | 1.46 | 1 | 446 (MH+) | A, B, M, R, S, T, H, e |
| 143 | 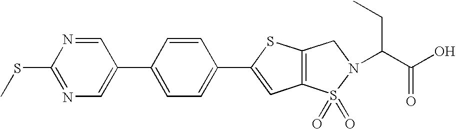 | 1.65 | 1 | 462 (MH+) | A, B, M, R, S, T, H, e |
| 144 | 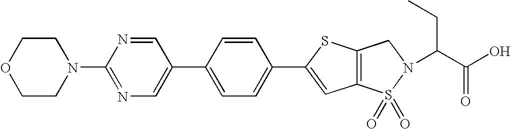 | 1.58 | 1 | 501 (MH+) | A, B, M, R, S, T, H, e |
| 145 | 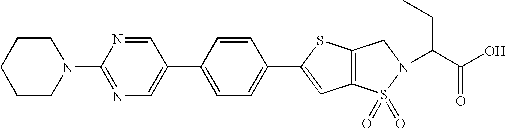 | 1.95 | 1 | 499 (MH+) | A, B, M, R, S, T, H, e |
| 146 | 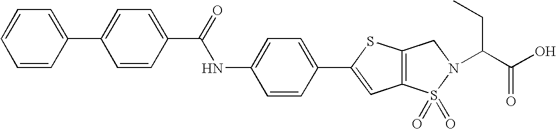 | 1.54 | 2 | 533 (MH+) | L, M, N, P, Q, R, S, T, H, AA, Z, a |
| 147 | 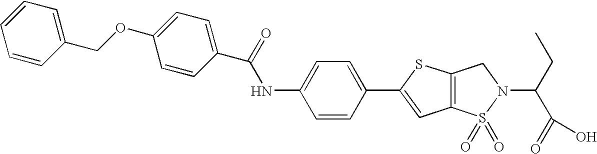 | 1.48 | 2 | 563 (MH+) | L, M, N, P, Q, R, S, T, H, AA, Z, a |
| 148 | 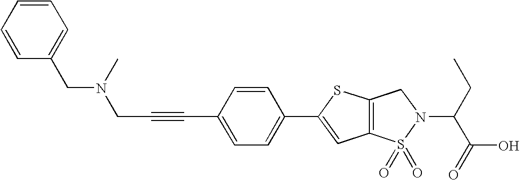 | 1.45 | 1 | 495 (MH+) | L, M, N, P, Q, R, S, T, K, a |

### Test Example 1

### Inhibitory Action for Aggrecanase Enzyme Activity

### (1) Preparation of Human Recombinant ADAMTS-5

The enzyme protein used for measurement of ADAMTS-5 activity was human recombinant ADAMTS-5. An insect cell line was prepared that stably expressed human recombinant ADAMTS-5 in which 6×His tag was added to the C-end side of Asn624. The culture supernatant of this cell line was collected, and the obtained culture supernatant was purified using Ni-NTA agarose (Qiagen)

### (2) Preparation of Interglobular Domain of Human Recombinant Aggrecan

A matrix peptide used for measurement of aggrecanase activity was Interglobular domain of human recombinant aggrecan. With reference to the method of Hughes, et al. (J. Biol. Chem. 272: pp. 20269-20274, 1997), the peptide of Thr331 to Gly457 of human aggrecan was contained, and the FLAG epitope peptide was added at the N-terminal, and the C-terminal was modified with biotin. This peptide was expressed using Escherichia coli, and the obtained Escherichia coli was subjected to lysis, and then the peptide was purified with an affinity column (Promega)

### (3) Measurement of Inhibitory Action for ADAMTS-5 Activity

Desired concentration of the test compound was reacted in 40 μl of a mixed reaction solution containing 50 mmol/L Tris-HCl (pH 7.5), 100 mmol/L NaCl, 10 mmol/L CaCl₂, 0.05% Brij35 and human recombinant ADAMTS-5 and Interglobular domain peptide of human recombinant aggrecan at 37° C. for 60 minutes. To the mixed reaction solution was added 10 μl of 0.2 mol/L EDTA to stop the enzyme reaction. Five μl of this mixed reaction solution was harvested and transferred to a 384 well plate. 0.2% Bovine albumin, cryptate-labeled Streptavidin (CIS bio) and XL 665-labeled anti-FLAG (M2) antibody (CIS bio) suitably diluted with PBS (−)-containing 800 mmol/L potassium fluoride were added thereto so that the amount of each of the added matters was 2.5 μl. The resultant solution was incubated at room temperature. After 60 minutes, the fluorescence value was measured at 340 nm excitation wavelength and 615 nm and 665 nm measurement wavelengths with a fluorescence plate reader. When the matrix peptide was not cleaved, the fluorescence resonance energy transfer from cryptate to XL665 occurs and long life fluorescence of 665 nm wavelength is generated. Meanwhile, fluorescence resonance energy transfer does not occur and long life fluorescence is not generated when the matrix peptide was cleaved. Therefore, the aggrecanase activity was calculated using the change of the ratio of the fluorescence value at 665 nm and the fluorescence value at 615 nm. As a result, the present invention compound or a salt thereof exhibited excellent inhibitory action for aggrecanase activity as shown representatively in Table 2. The inhibitory action for aggrecanase activity was represented by IC₅₀ value (μmol/L). Exp. No. in Table 2 represents Example number.

TABLE 2

| | ADAMTS-5 |
|---|---|
| Exp. No. | IC50 (μM) |
| 5 | 2.1 |
| 11 | 4 |
| 12 | 2 |
| 13 | 2 |
| 14 | 7 |

In addition, all of the compounds of Example Nos. 18, 22, 26, 30, 41, 42, 53, 54, 56, 60, 61, 66, 69, 92, 94, 95, 96, 97, 102, 104, and 147 exhibited an inhibitory activity of 2 μmol/L or smaller IC₅₀ value.

### (4) Measurement of Inhibitory Action for ADAMTS-4 Activity

Desired concentration of the test compound was reactecd in 40 μl of a mixed reaction solution containing 50 mmol/L Tris-HCl (pH7.5), 100 mmol/L NaCl, 10 mmol/LCaCl₂, 0.05% Brij35 and human recombinant ADAMTS-4 (R&D) and Interglobular domain peptide of human recombinant aggrecan at 37° C. for 60 minutes. To the mixed reaction solution, 10 μl of 0.2 mol/L EDTA was added to stop the enzyme reaction. Five 111 of this mixed reaction solution was harvested and transferred to a 384 well plate. 0.2% Bovine albumin, cryptate-labeled Streptavidin (CIS bio) and XL 665-labeled anti-FLAG (M2) antibody (CIS bio) suitably diluted with PBS (−)-containing 800 mmol/L potassium fluoride were added thereto so that the amount of each of the added matters was 2.5 μl. The reaction solution was incubated at room temperature. After 60 minutes, the fluorescence value was measured at 340 nm excitation wavelength and 615 nm and 665 nm measurement wavelengths with a fluorescence plate reader. The aggrecanase activity was calculated using the change of the ratio of these fluorescence values.

### Test Example 2

### Inhibitory Action for MMP Enzyme Activity

### (1) Measurement of Inhibitory Action for MMP-2 Activity

Desired concentration of the test compound was reacted in 100 μl of a mixed reaction solution containing 50 mmol/L Tris-HCl (pH7.5), 150 mmol/L NaCl, 10 mmol/LCaCl₂, 0.05% Brij35 and activated human recombinant MMP-2 (R&D) and fluorescence peptide matrix Mca-ProLeuGlyLeuDpaAlaArg-NH2 (R&D) at 30° C. for 45 minutes. To the mixed reaction solution, 10 μl of 0.2 mol/L EDTA was added to stop the enzyme reaction. Then, the fluorescence value was measured at 320 nm excitation wavelength and 405 nm measurement wavelength with a fluorescence plate reader.

The compounds of Example Nos. 96, 104, 113 and 147 had 30 μmol/L or more IC50 value.

### (2) Measurement of Inhibitory Action for MMP-3 Activity

Desired concentration of the test compound was reacted in 100 μl of a mixed reaction solution containing 50 mmol/L Tris-HCl (pH7.5), 150 mmol/LNaCl, 10 mmol/LCaCl₂, 0.05% Brij35 and activated human recombinant MMP-3 (R&D) and fluorescence peptide matrix Mca-ArgProLysProValGlu-Nval-TrpArgLys (Dnp) —NH2 (R&D) at 30° C. for 45 minutes. To the mixed reaction solution was added 10 μl of 0.2 mol/L EDTA to stop the enzyme reaction. Then, the fluorescence value was measured at 320 nm excitation wavelength and 405 nm measurement wavelength with a fluorescence plate reader.

The compounds of Example Nos. 96, 102, 104, 113 and 147 had 30 μmol/L or more IC50 value.

### (3) Measurement of Inhibitory Action for MMP-14 Activity

Desired concentration of the test compound was reacted in 100 μl of a mixed reaction solution containing 50 mmol/L Tris-HCl (pH7.5), 150 mmol/LNaCl, 10 mmol/LCaCl₂, 0.05% Brij35 and activated human recombinant MMP-14 catalytic domain (Calbiochem) and fluorescence peptide matrix (R&D) at 30° C. for 45 minutes. To the mixed reaction solution was added 10 μl of 0.2 mol/L EDTA to stop the enzyme reaction. Then, the fluorescence value was measured at 320 nm excitation wavelength and 405 nm measurement wavelength with a fluorescence plate reader.

The compounds of Example Nos. 96, 102, 104, 113 and 147 had 30 μmol/L or more IC50 value.

1. A compound of the following formula (1), or a stereoisomer, racemate, pharmaceutically acceptable salt, hydrate, or a prodrug thereof, 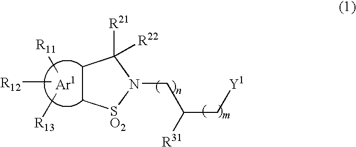
wherein,
Ar¹ represents a heteroaryl ring,
R11 represents —R¹¹¹, —R¹¹², —R¹¹³, —R¹¹⁴
(R¹¹¹ represents a single bond, —O—, —NR¹¹¹¹—, —NR¹¹¹¹CO—, —CONR¹¹¹¹—, —NR¹¹¹¹SO₂—, —SO₂NR¹¹¹¹— (R¹¹¹¹ represents a hydrogen atom or an optionally substituted alkyl group), —(C═O)—, —C≡C— or —CH═CH—,
R¹¹² represents an optionally substituted alkylene group or an optionally substituted arylene group, or may bond to R¹¹¹¹ to form a ring structure,
R¹¹³ represents a single bond, —O—, —NR¹¹³¹—, —NR¹¹³¹CO—, CONR¹¹³¹—, —NR¹¹³¹SO₂—, —SO₂NR¹¹³¹— (R¹¹³¹ represents a hydrogen atom or an optionally substituted alkyl group), —(C═O)—, —C≡C— or —CH═CH—,
R¹¹⁴ represents an optionally substituted aryl group, an optionally substituted alkyl group, or a hydrogen atom, or may bond to R¹¹³¹ to form a ring structure),
provided that in the case that R¹¹² is a nitrogen atom-containing group, and the bonding position of R¹¹¹ is on the nitrogen atom of R¹¹², R¹¹¹ represents a single bond, —NR¹¹¹¹CO—, NR¹¹¹¹SO₂—, —(C═O)—, —C≡C— or —CH═CH—,
in the case that R¹¹² is a nitrogen atom-containing group, and the bonding position of R¹¹³ is on the nitrogen atom of R¹¹², R¹¹³ represents a single bond, —CONR¹¹³¹—, —SO₂NR¹¹³¹—, —-(C═O)—,
—C≡C— or —CH═CH—,
in the case that R¹¹⁴ is a nitrogen atom-containing group, and the bonding position of R¹¹³ is on the nitrogen atom of R¹¹⁴, R113 represents a single bond, —NR¹¹³¹CO—, —NR¹¹³¹SO₂—, —(C═O)—, C≡C— or —CH═CH—;
provided that in the case that Ar¹ is a nitrogen atom-containing group, and the substituted position of R¹¹ is on the nitrogen atom on Ar¹, R¹¹¹ represents a single bond, —CONR¹¹¹¹—, —SO₂NR¹¹¹¹—, —(C═O)—, —C≡C— or —CH═CH—;
R¹² and R¹³ may be the same or different and independently represent,
a hydrogen atom, a halogen atom, a hydroxyl group, an optionally substituted alkyl group, an optionally substituted alkenyl group, an optionally substituted alkynyl group, an optionally substituted alkoxy group or an optionally substituted amino group;
or may be absent;
R²¹ and R²² may be the same or different and independently represent,
a hydrogen atom, an optionally substituted alkyl group, an optionally substituted aryl group, an optionally substituted alkoxy group, an optionally substituted aryloxy group or an optionally substituted amino group;
R³¹ represents
a hydrogen atom, an optionally substituted alkyl group, an optionally substituted aryl group, an optionally substituted alkoxy group or an optionally substituted aryloxy group;
R²¹ and R³¹, or R²² and R³¹ each may form a ring together;
n and m may be the same or different and independently represent 0 or 1;
Y¹ represents —COY₁₁ (Y¹¹ represents —NHOH, —OH, —NH₂, an amino group substituted with one or two optionally substituted alkyl groups, an amino group substituted with one or two optionally substituted aryl groups or an amino group substituted with an optionally substituted aryl group and an optionally substituted alkyl group).

2. The compound, or a stereoisomer, racemate, pharmaceutically acceptable salt, hydrate, or a prodrug thereof according to claim 1, wherein Ar¹ represents thiophene ring.

3. The compound, or a stereoisomer, racemate, pharmaceutically acceptable salt, hydrate, or a prodrug thereof according to claim 1 or 2, wherein R¹¹¹ represents a single bond.

4. The compound, or a stereoisomer, racemate, pharmaceutically acceptable salt, hydrate, or a prodrug thereof according to claim 1 or 2, wherein R¹¹¹ represents —CH═CH.

5. The compound, or a stereoisomer, racemate, pharmaceutically acceptable salt, hydrate, or a prodrug thereof according to claim 1 wherein R¹¹² represents an optionally substituted arylene group.

6. The compound, or a stereoisomer, racemate, pharmaceutically acceptable salt, hydrate, or a prodrug thereof according to any one of claim 1 wherein R¹¹² represents an optionally substituted phenylene group.

7. The compound, or a stereoisomer, racemate, pharmaceutically acceptable salt, hydrate, or a prodrug thereof according claim 1 wherein R¹¹³ represents a single bond, —CH═CH— or —C≡C—.

8. The compound, or a stereoisomer, racemate, pharmaceutically acceptable salt, hydrate, or a prodrug thereof according to claim 1, wherein R¹¹⁴ represents an optionally substituted aryl group.

9. The compound, or a stereoisomer, racemate, pharmaceutically acceptable salt, hydrate, or a prodrug thereof according to claim 1, wherein R¹², if present, is a hydrogen atom and R¹³, if present, is a hydrogen atom.

10. The compound, or a stereoisomer, racemate, pharmaceutically acceptable salt, hydrate, or a prodrug thereof according to claim 1, wherein both R²¹ and R²² represent a hydrogen atom.

11. The compound, or a stereoisomer, racemate, pharmaceutically acceptable salt, hydrate, or a prodrug thereof according to claim 1 wherein R³¹ represents an optionally substituted alkyl group.

12. The compound, or a stereoisomer, racemate, pharmaceutically acceptable salt, hydrate, or a prodrug thereof according to claim 1, wherein R³¹ is an isopropyl group.

13. The compound, or a stereoisomer, racemate, pharmaceutically acceptable salt, hydrate, or a prodrug thereof according to claim 1, wherein the asymmetric carbon to which R³¹ is bonded has steric configuration of the following formula (5):

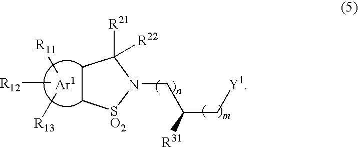

14. The compound, or a stereoisomer, racemate, pharmaceutically acceptable salt, hydrate, or a prodrug thereof according to claim 1, wherein R²¹ and R³¹, and R²² and R³¹ do not form a ring.

15. The compound, or a stereoisomer, racemate, pharmaceutically acceptable salt, hydrate, or a prodrug thereof according to claim 1 wherein both n and m represent 0.

16. The compound, or a stereoisomer, racemate, pharmaceutically acceptable salt, hydrate, or a prodrug thereof according to claim 1 wherein Y¹¹ represents —NHOH.

17. The compound, or a stereoisomer, racemate, pharmaceutically acceptable salt, hydrate, or a prodrug thereof according to claim 1, wherein Y¹¹ represents —OH.

18. A pharmaceutical agent which comprises the compound, or a stereoisomer, racemate, pharmaceutically acceptable salt, hydrate, or a prodrug thereof described in claim 1 as an active ingredient.

19. The pharmaceutical agent according to claim 18, which is used for prevention and/or treatment of a disease characterized by destruction of a joint cartilage.

20. The pharmaceutical agent according to claim 18, which is used for prevention and/or treatment of osteoarthritis.

21. An inhibitor for aggrecanase activity which comprises the compound, or a stereoisomer, racemate, pharmaceutically acceptable salt, hydrate, or a prodrug thereof described in claim 1 as an active ingredient.

22. A method for prevention and/or treatment of a disease characterized by destruction of a joint cartilage of a mammal, comprising administering an effective amount of the compound, or a stereoisomer, racemate, pharmaceutically acceptable salt, hydrate, or a prodrug thereof described in claim 1 to the mammal.

23. Use of the compound, or a stereoisomer, racemate, pharmaceutically acceptable salt, hydrate, or a prodrug thereof described in claim 1 for preparation of a pharmaceutical agent which is used for prevention and/or treatment of a disease characterized by destruction of a joint cartilage.
